(19) United States
(12) Patent Application Publication
(10) Pub. No.: US 2009/0069340 A1
(43) Pub. Date: Mar. 12, 2009

(54) Pyrazolone Compounds As Metabotropic Glutamate Receptor Agonists For The Treatment Of Neurological And Psychiatric Disorders

(75) Inventors: Michael Balestra, Wilmington, DE (US); Heather Bunting, Wilmington, DE (US); Deborah Chen, Biopolis Way (SG); Ian Egle, North York (CA); Janet Forst, Wilmington, DE (US); Jennifer Frey, Brampton (CA); Methvin Isaac, Brampton (CA); Fupeng Ma, Brampton (CA); David Nugiel, Wilmington, DE (US); Abdelmalik Slassi, Mississauga (CA); Gary Steelman, Wilmington, DE (US); Guang-Ri Sun, Markham (CA); Babu Sundar, Wilmington, DE (US); Radhakrishnan Ukkiramapandian, Salt Lake City, UT (US); Rebecca Urbanek, Wilmington, DE (US); Sally Walsh, Wilmington, DE (US)

Correspondence Address: BIRCH, STEWART, KOLASCH & BIRCH, LLP, P.O. BOX 747, FALLS CHURCH, VA, 22040-0747, US

(73) Assignee: AstraZeneca AB, Sodertalje (SE)

(21) Appl. No.: 11/793,050

(22) PCT Filed: Dec. 22, 2005
(86) PCT No.: PCT/US05/46606

§ 371 Date: Aug. 15, 2007

Related U.S. Application Data
(60) Provisional application No. 60/638,369, filed on Dec. 27, 2004.

(51) Int. Cl. A61K 31/496; C07D 471/10; A61K 31/438; C07D 401/06; A61P 25/16; A61P 25/28; A61K 31/454; C07D 403/06

(52) U.S. Cl. 514/254.05; 546/20; 514/278; 546/211; 514/326; 544/371

(57) ABSTRACT

Compounds of Formula (I), wherein R¹, R², R³, R⁴, R⁵, R⁶, X, and n are as defined for Formula (I) in the description, processes for the preparation of the compounds and new intermediates employed in the preparation, pharmaceutical compositions containing the compounds, and the use of the compounds in the treatment or prevention of neurological and psychiatric disorders associated with glutamate dysfunction.

### BACKGROUND OF THE INVENTION

The present invention relates to novel compounds that function as potentiators of glutamate receptors, methods for their preparation, pharmaceutical compositions containing them and their use in therapy.

The metabotropic glutamate receptors (mGluR) constitute a family of GTP-binding-protein (G-protein) coupled receptors that are activated by glutarnate, and have important roles in synaptic activity in the central nervous system, including neural plasticity, neural development and neurodegeneration.

Activation of mGluRs in intact mammalian neurons elicits one or more of the following responses: activation of phospholipase C; increases in phosphoinositide (PI) hydrolysis; intracellular calcium release; activation of phospholipase D; activation or inhibition of adenyl cyclase; increases or decreases in the formation of cyclic adenosine monophosphate (cAMP); activation of guanylyl cyclase; increases in the formation of cyclic guanosine monophosphate (cGMP); activation of phospholipase A₂; increases in arachidonic acid release; and increases or decreases in the activity of voltage- and ligand-gated ion channels (Schoepp et al., 1993, Trends Pharmacol. Sci., 14:13; Schoepp, 1994, Neurochem. Int., 24:439; Pin et al., 1995, Neuropharmacology 34:1; Bordi & Ugolini, 1999, Prog. Neurobiol. 59:55).

Eight mGluR subtypes have been identified, which are divided into three groups based upon primary sequence similarity, signal transduction linkages, and pharmacological profile. Group-I includes mGluR1 and mGluR5, which activate phospholipase C and the generation of an intracellular calcium signal. The Group-II (mGluR2 and mGluR3) and Group-III (mGluR4, mGluR6, mGluR7, and mGluR8) mGluRs mediate an inhibition of adenylyl cyclase activity and cyclic AMP levels. For a review, see Pin er al., 1999, Eur. J. Pharmacol., 375:277-294.

Members of the mGluR family of receptors are implicated in a number of normal processes in the mammalian CNS, and are important targets for compounds for the treatment of a variety of neurological and psychiatric disorders. Activation of mGluRs is required for induction of hippocampal long-term potentiation and cerebellar long-term depression (Bashir et al., 1993, Nature, 363:347; Bortolotto et al., 1994, Nature, 368:740; Aiba et al., 1994, Cell, 79:365; Aiba et al., 1994, Cell, 79:377). A role for mGluR activation in nociception and analgesia also has been demonstrated (Meller et al., 1993, Neuroreport, 4: 879; Bordi & Ugolini, 1999, Brain Res., 871:223). In addition, mGluR activation has been suggested to play a modulatory role in a variety of other normal processes including synaptic transmission, neuronal development, apoptotic neuronal death, synaptic plasticity, spatial learning, olfactory memory, central control of cardiac activity, waking, motor control and control of the vestibulo-ocular reflex (Nakanishi, 1994, Neuron, 13:1031; Pin et al., 1995, Neuropharmacology, supra; Knopfel et al., 1995, J. Med. Chem., 38:1417).

Recent advances in the elucidation of the neurophysiological roles of mGluRs have established these receptors as promising drug targets in the therapy of acute and chronic neurological and psychiatric disorders and chronic and acute pain disorders. Because of the physiological and pathophysiological significance of the mGluRs, there is a need for new drugs and compounds that can modulate mGluR function.

### SUMMARY OF THE INVENTION

The present invention satisfies this need and others by providing a compound of Formula I, or a pharmaceutically acceptable salt, hydrate, solvate, optical isomer, or combination thereof:

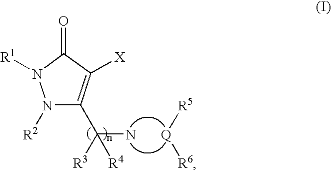

wherein
X is selected from the group consisting of F, Cl, Br, I, cyano, OC₁₋₆-alkyl, C₁₋₆-alkylhalo, OC₁₋₆-alkylhalo;
Q is selected from the group consisting of C, O, S, and N, such that when
   Q is C, then at least one of R⁵ and R⁶ is present,
   Q is N, then one of R⁵ and R⁶ is present, and
   Q is O or S, then R⁵ and R⁶ are both absent;

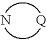

represents a 5- to 7-membered ring, wherein said ring is optionally fused with one or more 5- to 7-membered rings each containing atoms independently selected from the group consisting of C, N, O and S, wherein each of said rings may be substituted by one or more A;
R¹ is selected from the group consisting of C₁₋₆-alkyl, C₂₋₆-alkenyl, C₂₋₆-alkynyl, aryl, heteroaryl, heterocycloalkyl, C₃₋₈-cycloalkyl, C₁₋₆-alkyl-aryl, C₁₋₆-alkyl-heteroaryl, C₁₋₆-alkyl-heterocycloalkyl, C₁₋₆-alkyl-C₃₋₈-cycloalkyl, wherein R¹ may be substituted by one or more A;
R² is selected from the group consisting of H, C₁₋₆-alkyl, C₂₋₆-alkenyl, and C₂₋₆-alkynyl, wherein R² may be substituted by one or more A;
R³ and R⁴ each are independently selected from the group consisting of H, C₁₋₆-alkyl, C₂₋₆-alkenyl, C₂₋₆-alkynyl, aryl, heteroaryl, heterocycloalkyl, C₃₋₈-cycloalkyl, C₁₋₆-alkyl-aryl, C₁₋₆-alkyl-heteroaryl, C₁₋₆-alkyl-heterocycloalkyl, C₁₋₆-alkyl-C₃₋₈-cycloalkyl, wherein R³ and R⁴ may be substituted by one or more A;
R⁵ and R⁶, when present, are independently selected from the group consisting of H, hydroxy, F, Cl, Br, I, nitro, cyano, C₁₋₆-alkyl, C₁₋₆-alkylhalo, OC₁₋₆alkyl, OC₁₋₆-alkylhalo, C₂₋₆-alkenyl, OC₂₋₆-alkenyl, C₂₋₆-alkynyl, OC₂₋₆-alkynyl, C₃₋₈-cycloalkyl, C₁₋₆-alkyl-C₃₋₈-cycloalkyl, OC₀₋₆-alkyl-C₃₋₈-cycloalkyl, aryl, C₁₋₆-alkylaryl, OC₀₋₆-alkylaryl, heteroaryl, C₁₋₆-alkylheteroaryl, OC₀₋₆-alkylheteroaryl, C(O)H, (CO)R⁷, O(CO)R⁷, O(CO)OR⁷, C(O)OR⁷, OC(NH)OR⁷, C₁₋₆-alkylOR⁷, OC₂₋₆-alkylOR⁷, C₁₋₆-alkyl(CO)R⁷, OC₁₋₆-alkyl(CO)R⁷, C₁₋₆-alkylCO₂R⁷, OC₁₋₆-alkylCO₂R⁷, C₁₋₆ alkylcyano, OC₂₋₆-alkylcyano, CO₀₋₆-alkylNR⁷R⁸, OC₂₋₆-alkylNR⁷R⁸, C₀₋₆-alkyl(CO)NR⁷R⁸, OC₀₋₆-alkyl(CO)NR⁷R⁸, C₀₋₆-alkylNR⁷(CO)R⁸, OC₂₋₆-alkylNR⁷(CO)R⁸, C₀₋₆-alkylNR⁷(CO)NR⁷R⁸, CO₀₋₆-alkylSR⁷, OC₂₋₆-alkylSR⁷, C₀₋₆-alkyl(SO)R⁷, OC₂₋₆-alkyl(SO)R⁷, C₀₋₆-alkylSO₂R⁷, OC₂₋₆-alkylSO₂R⁷, C₀₋₆-alkyl(SO₂)NR⁷R⁸, OC₂₋₆-alkyl(SO₂)NR⁷R⁸, C₀₋₆-alkylNR⁷(SO₂)R⁸, OC₂₋₆-alkylNR⁷(SO₂)R⁸, C₀₋₆-alkylNR⁷(SO₂)NR⁷R⁸, OC₂₋₆-alkylNR⁷(SO₂)NR⁷R⁸, (CO)NR⁷R⁸, O(CO)NR⁷R⁸, NR⁷OR⁸, C₀₋₆-alkylNR⁷(CO)OR⁸, OC₂₋₆-alkylNR⁷(CO)OR⁸, SO₃R⁷ and a 5- to 7-membered ring containing atoms independently selected from the group consisting of C, N, O and S, wherein R⁵ and R⁶ may be substituted by one or more A, and wherein any cycloalkyl or aryl is optionally fused to a 5- to 7-membered ring containing atoms independently selected from the group consisting of C, N, O and S;
   or, optionally, when Q is C, then R⁵ and R⁶, together with Q, may form a 5- to 7-membered ring, which may be unsaturated, containing atoms independently selected from the group consisting of C, N, O and S, wherein
      i) said ring is optionally fused with one or more 5- to 7-membered rings each containing atoms independently selected from the group consisting of C, N, O and S, and wherein
      ii) said rings each may be substituted by one or more A;
R⁷ and R⁸ are independently selected from the group consisting of hydrogen, C₁₋₆-alkyl, C₃₋₇-cycloalkyl, C(O)C₁₋₆-alkyl, aryl, C₁₋₆-alkylaryl, heterocycloalkyl, and heteroaryl, wherein R⁷ and R⁸ may be substituted by one or more A;
A is selected from the group consisting of hydroxy, F, Cl, Br, I, nitro, cyano, oxo, C₁₋₆-alkyl, C₁₋₆-alkylhalo, OC₁₋₆alkyl, OC₁₋₆-alkylhalo, C₂₋₆-alkenyl, OC₂₋₆-alkenyl, C₂₋₆-alkynyl, OC₂₋₆-alkynyl, C₃₋₈-cycloalkyl, C₁₋₆-alkyl-C₃₋₈-cycloalkyl, OC₀₋₆-alkyl-C₃₋₈-cycloalkyl, aryl, C₁₋₆-alkylaryl, OC₀₋₆-alkylarylheteroaryl, C₁₋₆-alkylheteroaryl, OC₀₋₆-alkylheteroaryl, (CO)R⁹, O(CO)R⁹, O(CO)OR⁹, OC(NH)OR⁹, C₁₋₆-alkylOR⁹, OC₂₋₆-alkylOR⁹, C₁₋₆-alkyl(CO)R⁹, OC₁₋₆-alkyl(CO)R⁹, C₀₋₆-alkylCO₂R⁹, OC₁₋₆-alkylCO₂R⁹, C₁₋₆-alkylcyano, OC₂₋₆-alkylcyano, C₀₋₆-alkylNR⁹R¹⁰, OC₂₋₆-alkylNR⁹R¹⁰, C₁₋₆-alkyl(CO)NR⁹R¹⁰, OC₁₋₆-alkyl(CO)NR⁹R¹⁰, C₀₋₆-alkylNR⁹(CO)R¹⁰, OC₂₋₆-alkylNR⁹(CO)R¹⁰, C₀₋₆-alkylNR⁹(CO)NR⁹R¹⁰, C₀₋₆-alkylSR⁹, OC₂₋₆-alkylSR⁹, C₀₋₆-alkyl(SO)R⁹, OC₂₋₆-alkyl(SO)R⁹, C₀₋₆-alkylSO₂R⁹, OC₂₋₆-alkylSO₂R⁹, C₀₋₆-alkyl(SO₂)NR⁹R¹⁰, OC₂₋₆-alkyl(SO₂)NR⁹R¹⁰, C₀₋₆-alkylNR⁹(SO₂)R¹⁰, OC₂₋₆-alkylNR⁹(SO₂)R¹⁰, C₀₋₆-alkylNR⁹(SO₂)NR⁹R¹⁰, OC₂₋₆-alkylNR⁹(SO₂)NR⁹R¹⁰, (CO)NR⁹R¹⁰, O(CO)NR⁹R¹⁰, NR⁹OR¹⁰, C₀₋₆-alkylNR⁹(CO)OR¹⁰, OC₂₋₆-alkylNR⁹(CO)OR¹⁰, OC(NH)OR⁹, SO₃R⁹, wherein any ring is optionally substituted with one or more B, and a 5- to 7-membered ring containing atoms independently selected from the group consisting of C, N, O and S, wherein said ring is optionally substituted by one or more of R⁹ and R¹⁰;
R⁹ and R¹⁰ are independently selected from the group consisting of H, hydroxy, F, Cl, Br, I, nitro, cyano, oxo, C₁₋₆-alkyl, C₁₋₆-alkylhalo, OC₁₋₆alkyl, OC₁₋₆-alkylhalo, C₂₋₆-alkenyl, OC₂₋₆-alkenyl, C₂₋₆-alkynyl, OC₂₋₆-alkynyl, C₃₋₈-cycloalkyl, C₁₋₆-alkyl-C₃₋₈-cycloalkyl, OC₀₋₆-alkyl-C₃₋₈-cycloalkyl, aryl, C₁₋₆-alkylaryl, OC₀₋₆-alkylaryl, heterocycloalkyl, and heteroaryl, and any ring is optionally substituted with one or more B;
B is selected from the group consisting of F, Cl, Br, I, C₁₋6-alkyl and OC₁₋₆alkyl; and
   n is selected from the group consisting of 1, 2, 3, 4, 5, and 6.

A further aspect of the invention provides a compound of Formula II, or a pharmaceutically acceptable salt, hydrate, solvate, optical isomer, or combination thereof:

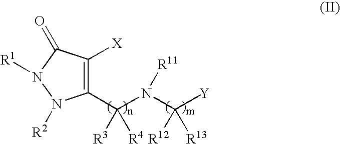

wherein
X is selected from the group consisting of F, Cl, Br, I, cyano, OC₁₋₆-alkyl, C₁₋₆-alkylhalo, OC₁₋₆-alkylhalo;
R¹ is selected from the group consisting of C₁₋₆-alkyl, C₂₋₆-alkenyl, C₂₋₆-alkynyl, aryl, heteroaryl, heterocycloalkyl, C₃₋₈-cycloalkyl, C₁₋₆-alkyl-aryl, C₁₋₆-alkyl-heteroaryl, C₁₋₆-alkyl-heterocycloalkyl, C₁₋₆-alkyl-C₃₋₈-cycloalkyl, wherein R¹ may be substituted by one or more A;
R² is selected from the group consisting of H, C₁₋₆-alkyl, C₂₋₆-alkenyl, and C₂₋₆-alkynyl, wherein R¹ may be substituted by one or more A;
R³, R⁴, R¹² and R¹³ are each independently selected from the group consisting of H, C₁₋₆-alkyl, C₂₋₆-alkenyl, C₂₋₆-alkynyl, aryl, heteroaryl, heterocycloalkyl, C₃₋₈-cycloalkyl, C₁₋₆-alkyl-aryl, C₁₋₆-alkyl-heteroaryl, C₁₋₆-alkyl-heterocycloalkyl, C₁₋₆-alkyl-C₃₋₈-cycloalkyl, wherein R³ and R⁴ may be substituted by one or more A;
R¹¹ is selected from the group consisting of H, C₁₋₆-alkyl, C₁₋₆-alkylhalo, C₂₋₆-alkenyl, C₂₋₆-alkynyl, C₃₋₈-cycloalkyl, C₁₋₆-alkyl-C₃₋₈-cycloalkyl, C₃₋₈-heterocycloalkyl, C₁₋₆-alkyl-C₃₋₈-heterocycloalkyl aryl, C₁₋₆-alkylaryl, heteroaryl, C₁₋₆-alkylheteroaryl, C(O)H, (CO)R⁷, C(O)OR⁷, C₁₋₆-alkylOR⁷, C₁₋₆-alkyl(CO)R⁷, C₁₋₆-alkylCO₂R⁷, C₁₋₆-alkylcyano, C₁₋₆-alkylNR⁷R⁸, C₁₋₆-alkyl(CO)NR⁷R⁸, C₁₋₆-alkylNR⁷(CO)R⁸, C₁₋₆-alkylNR⁷(CO)NR⁷R⁸, C₁₋₆-alkylSR⁷, C₀₋₆-alkyl(SO)R⁷, C₀₋₆-alkylSO₂R⁷, C₀₋₆-alkyl(SO₂)NR⁷R⁸, C₀₋₆-alkylNR⁷(SO₂)R⁸, C₀₋₆-alkylNR⁷(SO₂)NR⁷R⁸, (CO)NR⁷R⁸, C₀₋₆-alkylNR⁷(CO)OR⁸, C₀₋₆-alkyl SO₃R⁷ and a 5- to 7-membered ring containing atoms independently selected from the group consisting of C, N, O and S, wherein R¹¹ may be substituted by one or more A, and wherein any cycloalkyl or aryl is optionally fused to a 5- to 7-membered ring containing atoms independently selected from the group consisting of C, N, O and S;
R⁷ and R⁸ are independently selected from the group consisting of hydrogen, C₁₋₆-alkyl, C₃₋₇-cycloalkyl, C(O)C₁₋₆-alkyl, aryl, C₁₋₆-alkylaryl, heterocycloalkyl, and heteroaryl, wherein R⁷ and R⁸ may be substituted by one or more A;
A is selected from the group consisting of hydroxy, F, Cl, Br, I, nitro, cyano, oxo, C₁₋₆-alkyl, C₁₋₆-alkylhalo, OC₁₋₆alkyl, OC₁₋₆-alkylhalo, C₂₋₆-alkenyl, OC₂₋₆-alkenyl, C₂₋₆-alkynyl, OC₂₋₆-alkynyl, C₃₋₈-cycloalkyl, C₁₋₆-alkyl-C₃₋₈-cycloalkyl, OC₀₋₆-alkyl-C₃₋₈-cycloalkyl, aryl, C₁₋₆-alkylaryl, OC₀₋₆-alkylarylheteroaryl, C₁₋₆-alkylheteroaryl, OC₀₋₆-alkylheteroaryl, (CO)R⁹, O(CO)R⁹, O(CO)OR⁹, OC(NH)OR⁹, C₁₋₆-alkylOR⁹, OC₂₋₆-alkylOR⁹, C₁₋₆-alkyl(CO)R⁹, OC₁₋₆-alkyl(CO)R⁹, C₀₋₆-alkylCO₂R⁹, OC₁₋₆-alkylCO₂R⁹, C₁₋₆-alkylcyano, OC₂₋₆-alkylcyano, C₀₋₆-alkylNR⁹R¹⁰, OC₂₋₆-alkylNR⁹R¹⁰, C₁₋₆-alkyl(CO)NR⁹R¹⁰, OC₁₋₆-alkyl(CO)NR⁹R¹⁰, C₀₋₆-alkylNR⁹(CO)R¹⁰, OC₂₋₆-alkylNR⁹(CO)R¹⁰, C₀₋₆-alkylNR⁹(CO)NR⁹R¹⁰, C₀₋₆-alkylSR⁹, OC₂₋₆-alkylSR⁹, C₀₋₆-alkyl(SO)R⁹, OC₂₋₆-alkyl(SO)R⁹, C₀₋₆-alkylSO₂R⁹, OC₂₋₆-alkylSO₂R⁹, C₀₋₆-alkyl(SO₂)NR⁹R¹⁰, OC₂₋₆-alkyl(SO₂)NR⁹R¹⁰, C₀₋₆-alkylNR⁹(SO₂)R¹⁰, OC₂₋₆-alkylNR⁹(SO₂)R¹⁰, C₀₋₆-alkylNR⁹(SO₂)NR⁹R¹⁰, OC₂₋₆-alkylNR⁹(SO₂)NR⁹R¹⁰, (CO)NR⁹R¹⁰, O(CO)NR⁹R¹⁰, NR⁹OR¹⁰, C₀₋₆-alkylNR⁹(CO)OR¹⁰, OC₂₋₆-alkylNR⁹(CO)OR¹⁰, OC(NH)OR⁹, SO₃R⁹, wherein any ring is optionally substituted with one or more B, and a 5- to 7-membered ring containing atoms independently selected from the group consisting of C, N, O and S, wherein said ring is optionally substituted by one or more of R⁹ and R¹⁰;
R⁹ and R¹⁰ are independently selected from the group consisting of H, hydroxy, F, Cl, Br, I, nitro, cyano, oxo, C₁₋₆-alkyl, C₁₋₆-alkylhalo, OC₁₋₆alkyl, OC₁₋₆-alkylhalo, C₂₋₆-alkenyl, OC₂₋₆-alkenyl, C₂₋₆-alkynyl, OC₂₋₆-alkynyl, C₃₋₈-cycloalkyl, C₁₋₆-alkyl-C₃₋₈-cycloalkyl, OC₀₋₆-alkyl-C₃₋₈-cycloalkyl, aryl, C₁₋₆-alkylaryl, OC₀₋₆-alkylaryl, heterocycloalkyl, and heteroaryl, and any ring is optionally substituted with one or more B;
B is selected from the group consisting of F, Cl, Br, I, C₁₋₆-alkyl and OC₁₋₆alkyl;
m is selected from the group consisting of 0, 1, 2, 3, 4, 5, and 6;
n is selected from the group consisting of 1, 2, 3, 4, 5, and 6; and
Y is selected from the group consisting of alkyl, alkenyl, alkynyl, aryl, heteroaryl, heterocycloalkyl and C₃₋₁₀-cycloalkyl, wherein Y may be substituted by one or more A;
   or a pharmaceutically acceptable salt, hydrate, solvate, optical isomer, or combination thereof.

The invention also provides a method for the treatment or prevention of neurological and psychiatric disorders associated with glutamate dysfunction in an animal in need of such treatment. The method comprises the step of administering to the animal a therapeutically effective amount of a compound of Formula I or Formula II or a pharmaceutical composition thereof according to this invention.

Additionally, the invention also contemplates the use of a compound according to Formula I or Formula II, or a pharmaceutically acceptable salt or solvate thereof, for the manufacture of a medicament for the treatment of any of the conditions discussed herein.

Also provided by the invention is a compound of Formula I or Formula II, or a pharmaceutically acceptable salt or solvate thereof, for use in therapy.

The invention additionally provides processes for the preparation of compounds of Formula I or Formula II. General and specific processes are discuss in more detail below.

### DETAILED DESCRIPTION OF THE PREFERRED EMBODIMENTS

The present invention is based upon the discovery of compounds that exhibit activity as pharmaceuticals, in particular as modulators of metabotropic glutamate receptors. More particularly, the compounds of the present invention exhibit activity as potentiators of the mGluR2 receptor, and are useful in therapy, in particular for the treatment of neurological and psychiatric disorders associated with glutamate dysfunction.

### DEFINITIONS

Unless specified otherwise within this specification, the nomenclature used in this specification generally follows the examples and rules stated in Nomenclature of Organic Chemistry, Sections A, B, C, D, E, F, and H, Pergamon Press, Oxford, 1979, which is incorporated by references herein for its exemplary chemical structure names and rules on naming chemical structures. Optionally, a name of a compound may be generated using a chemical naming program: ACD/ChemSketch, Version 5.09/September 2001, Advanced Chemistry Development, Inc., Toronto, Canada.

The term “Cₘ₋ₙ” or “Cₘ₋ₙ group” used alone or as a prefix, refers to any group having m to n carbon atoms, inclusive, and having 0 to n multivalent heteroatoms selected from O, S and N, wherein m and n are 0 or positive integers, and n>m. For example, “C₁₋₆” would refer to a chemical group having 1 to 6 carbon atoms, and having 0 to 6 multivalent heteroatoms selected from O, S and N.

The term “hydrocarbon” used alone or as a suffix or prefix, refers to any structure comprising only carbon and hydrogen atoms up to 14 carbon atoms.

The term “hydrocarbon radical” or “hydrocarbyl” used alone or as a suffix or prefix, refers to any structure as a result of removing one or more hydrogens from a hydrocarbon.

The term “alkyl” used alone or as a suffix or prefix, refers to monovalent straight or branched chain hydrocarbon radicals comprising 1 to about 12 carbon atoms.

The term “alkylene” used alone or as suffix or prefix, refers to divalent straight or branched chain hydrocarbon radicals comprising 1 to about 12 carbon atoms, which serves to links two structures together.

The term “alkenyl” used alone or as suffix or prefix, refers to a monovalent straight or branched chain hydrocarbon radical having at least one carbon-carbon double bond and comprising at least 2 up to about 12 carbon atoms.

The term “alkynyl” used alone or as suffix or prefix, refers to a monovalent straight or branched chain hydrocarbon radical having at least one carbon-carbon triple bond and comprising at least 2 up to about 12 carbon atoms.

The term “cycloalkyl,” used alone or as suffix or prefix, refers to a monovalent ring-containing hydrocarbon radical comprising at least 3 up to about 12 carbon atoms.

The term “cycloalkenyl” used alone or as suffix or prefix, refers to a monovalent ring-containing hydrocarbon radical having at least one carbon-carbon double bond and comprising at least 3 up to about 12 carbon atoms.

The term “cycloalkynyl” used alone or as suffix or prefix, refers to a monovalent ring-containing hydrocarbon radical having at least one carbon-carbon triple bond and comprising about 7 up to about 12 carbon atoms.

The term “aryl” used alone or as suffix or prefix, refers to a monovalent hydrocarbon radical having one or more polyunsaturated carbon rings having aromatic character, (e.g., 4n+2 delocalized electrons) and comprising 5 up to about 14 carbon atoms.

The term “arylene” used alone or as suffix or prefix, refers to a divalent hydrocarbon radical having one or more polyunsaturated carbon rings having aromatic character, (e.g., 4n+2 delocalized electrons) and comprising 5 up to about 14 carbon atoms, which serves to links two structures together.

The term “heterocycle” used alone or as a suffix or prefix, refers to a ring-containing structure or molecule having one or more multivalent heteroatoms, independently selected from N, O and S, as a part of the ring structure and including at least 3 and up to about 20 atoms in the ring(s). Heterocycle may be saturated or unsaturated, containing one or more double bonds, and heterocycle may contain more than one ring. When a heterocycle contains more than one ring, the rings may be fused or unfused. Fused rings generally refer to at least two rings share two atoms therebetween. Heterocycle may have aromatic character or may not have aromatic character.

The term “heteroalkyl” used alone or as a suffix or prefix, refers to a radical formed as a result of replacing one or more carbon atom of an alkyl with one or more heteroatoms selected from N, O and S.

The term “heteroaromatic” used alone or as a suffix or prefix, refers to a ring-containing structure or molecule having one or more multivalent heteroatoms, independently selected from N, O and S, as a part of the ring structure and including at least 3 and up to about 20 atoms in the ring(s), wherein the ring-containing structure or molecule has an aromatic character (e.g., 4n+2 delocalized electrons).

The term “heterocyclic group,” “heterocyclic moiety,” “heterocyclic,” or “heterocyclo” used alone or as a suffix or prefix, refers to a radical derived from a heterocycle by removing one or more hydrogens therefrom.

The term “heterocyclyl” used alone or as a suffix or prefix, refers to a monovalent radical derived from a heterocycle by removing one hydrogen therefrom.

The term “heterocyclylene” used alone or as a suffix or prefix, refers to a divalent radical derived from a heterocycle by removing two hydrogens therefrom, which serves to links two structures together.

The term “heteroaryl” used alone or as a suffix or prefix, refers to a heterocyclyl having aromatic character.

The term “heterocylcoalkyl” used alone or as a suffix or prefix, refers to a heterocyclyl that does not have aromatic character.

The term “heteroarylene” used alone or as a suffix or prefix, refers to a heterocyclylene having aromatic character.

The term “heterocycloalkylene” used alone or as a suffix or prefix, refers to a heterocyclylene that does not have aromatic character.

The term “six-membered” used as prefix refers to a group having a ring that contains six ring atoms.

The term “five-membered” used as prefix refers to a group having a ring that contains five ring atoms.

A five-membered ring heteroaryl is a heteroaryl with a ring having five ring atoms wherein 1, 2 or 3 ring atoms are independently selected from N, O and S.

Exemplary five-membered ring heteroaryls are thienyl, furyl, pyrrolyl, imidazolyl, thiazolyl, oxazolyl, pyrazolyl, isothiazolyl, isoxazolyl, 1,2,3-triazolyl, tetrazolyl, 1,2,3-thiadiazolyl, 1,2,3-oxadiazolyl, 1,2,4-triazolyl, 1,2,4-thiadiazolyl, 1,2,4-oxadiazolyl, 1,3,4-triazolyl, 1,3,4-thiadiazolyl, and 1,3,4-oxadiazolyl.

A six-membered ring heteroaryl is a heteroaryl with a ring having six ring atoms wherein 1, 2 or 3 ring atoms are independently selected from N, O and S.

Exemplary six-membered ring heteroaryls are pyridyl, pyrazinyl, pyrimidinyl, triazinyl and pyridazinyl.

The term “substituted” used as a prefix refers to a structure, molecule or group, wherein one or more hydrogens are replaced with one or more C₁₋₁₂hydrocarbon groups, or one or more chemical groups containing one or more heteroatoms selected from N, O, S, F, Cl, Br, I, and P. Exemplary chemical groups containing one or more heteroatoms include heterocyclyl, —NO₂, —OR, —R′OR, —Cl, —Br, —I, —F, —CF₃, —C(═O)R, —C(—O)OH, —NH₂, —SH, —NHR, —NR₂, —SR, —SO₃H, —SO₂R, —S(═O)R, —CN, —OH, —C(═O)OR, —C(═O)NR₂—NRC(═O)R, —NRC(═O)OR, —R′NR₂, oxo (═O), imino (═NR), thio (═S), and oximino (═N—OR), wherein each “R” is hydrogen or a C₁₋₁₂hydrocarbyl and “R′” is a C₁₋₁₂hydrocarbyl. For example, substituted phenyl may refer to nitrophenyl, pyridylphenyl, methoxyphenyl, chlorophenyl, aminophenyl, etc., wherein the nitro, pyridyl, methoxy, chloro, and amino groups may replace any suitable hydrogen on the phenyl ring.

The term “substituted” used as a suffix of a first structure, molecule or group, followed by one or more names of chemical groups refers to a second structure, molecule or group, which is a result of replacing one or more hydrogens of the first structure, molecule or group with the one or more named chemical groups. For example, a “phenyl substituted by nitro” refers to nitrophenyl.

The term “optionally substituted” refers to groups, structures, or molecules that are substituted and to those that are not substituted.

Heterocycle includes, for example, monocyclic heterocycles such as: aziridine, oxirane, thiirane, azetidine, oxetane, thietane, pyrrolidine, pyrroline, imidazolidine, pyrazolidine, pyrazoline, dioxolane, sulfolane 2,3-dihydrofuran, 2,5-dihydrofuran tetrahydrofuran, thiophane, piperidine, 1,2,3,6-tetrahydro-pyridine, piperazine, morpholine, thiomorpholine, pyran, thiopyran, 2,3-dihydropyran, tetrahydropyran, 1,4-dihydropyridine, 1,4-dioxane, 1,3-dioxane, dioxane, homopiperidine, 2,3,4,7-tetrahydro-1H-azepine homopiperazine, 1,3-dioxepane, 4,7-dihydro-1,3-dioxepin, and hexamethylene oxide.

In addition, heterocycle includes aromatic heterocycles, for example, pyridine, pyrazine, pyrimidine, pyridazine, thiophene, furan, furazan, pyrrole, imidazole, thiazole, oxazole, pyrazole, isothiazole, isoxazole, 1,2,3-triazole, tetrazole, 1,2,3-thiadiazole, 1,2,3-oxadiazole, 1,2,4-triazole, 1,2,4-thiadiazole, 1,2,4-oxadiazole, 1,3,4-triazole, 1,3,4-thiadiazole, and 1,3,4-oxadiazole.

Additionally, heterocycle encompass polycyclic heterocycles, for example, indole, indoline, isoindoline, quinoline, tetrahydroquinoline, isoquinoline, tetrahydroisoquinoline, 1,4-benzodioxan, coumarin, dihydrocoumarin, benzofuran, 2,3-dihydrobenzofuran, isobenzofuran, chromene, chroman, isochroman, xanthene, phenoxathiin, thianthrene, indolizine, isoindole, indazole, purine, phthalazine, naphthyridine, quinoxaline, quinazoline, cinnoline, pteridine, phenanthridine, perimidine, phenanthroline, phenazine, phenothiazine, phenoxazine, 1,2-benzisoxazole, benzothiophene, benzoxazole, benzthiazole, benzimidazole, benztriazole, thioxanthine, carbazole, carboline, acridine, pyrolizidine, and quinolizidine.

In addition to the polycyclic heterocycles described above, heterocycle includes polycyclic heterocycles wherein the ring fusion between two or more rings includes more than one bond common to both rings and more than two atoms common to both rings. Examples of such bridged heterocycles include quinuclidine, diazabicyclo[2.2.1]heptane and 7-oxabicyclo[2.2.1]heptane.

Heterocyclyl includes, for example, monocyclic heterocyclyls, such as: aziridinyl, oxiranyl, thiiranyl, azetidinyl, oxetanyl, thietanyl, pyrrolidinyl, pyrrolinyl, imidazolidinyl, pyrazolidinyl, pyrazolinyl, dioxolanyl, sulfolanyl, 2,3-dihydrofuranyl, 2,5-dihydrofuranyl, tetrahydrofuranyl, thiophanyl, piperidinyl, 1,2,3,6-tetrahydro-pyridinyl, piperazinyl, morpholinyl, thiomorpholinyl, pyranyl, thiopyranyl, 2,3-dihydropyranyl, tetrahydropyranyl, 1,4-dihydropyridinyl, 1,4-dioxanyl, 1,3-dioxanyl, dioxanyl, homopiperidinyl, 2,3,4,7-tetrahydro-1H-azepinyl, homopiperazinyl, 1,3-dioxepanyl, 4,7-dihydro-1,3-dioxepinyl, and hexamethylene oxidyl.

In addition, heterocyclyl includes aromatic heterocyclyls or heteroaryl, for example, pyridinyl, pyrazinyl, pyrimidinyl, pyridazinyl, thienyl, furyl, furazanyl, pyrrolyl, imidazolyl, thiazolyl, oxazolyl, pyrazolyl, isothiazolyl, isoxazolyl, 1,2,3-triazolyl, tetrazolyl, 1,2,3-thiadiazolyl, 1,2,3-oxadiazolyl, 1,2,4-triazolyl, 1,2,4-thiadiazolyl, 1,2,4-oxadiazolyl, 1,3,4-triazolyl, 1,3,4-thiadiazolyl, and 1,3,4 oxadiazolyl.

Additionally, heterocyclyl encompasses polycyclic heterocyclyls (including both aromatic or non-aromatic), for example, indolyl, indolinyl, isoindolinyl, quinolinyl, tetrahydroquinolinyl, isoquinolinyl, tetrahydroisoquinolinyl, 1,4-benzodioxanyl, coumarinyl, dihydrocoumarinyl, benzofuranyl, 2,3-dihydrobenzofuranyl, isobenzofuranyl, chromenyl, chromanyl, isochromanyl, xanthenyl, phenoxathiinyl, thianthrenyl, indolizinyl, isoindolyl, indazolyl, purinyl, phthalazinyl, naphthyridinyl, quinoxalinyl, quinazolinyl, cinnolinyl, pteridinyl, phenanthridinyl, perimidinyl, phenanthrolinyl, phenazinyl, phenothiazinyl, phenoxazinyl, 1,2-benzisoxazolyl, benzothiophenyl, benzoxazolyl, benzthiazolyl, benzimidazolyl, benztriazolyl, thioxanthinyl, carbazolyl, carbolinyl, acridinyl, pyrolizidinyl, and quinolizidinyl.

In addition to the polycyclic heterocyclyls described above, heterocyclyl includes polycyclic heterocyclyls wherein the ring fusion between two or more rings includes more than one bond common to both rings and more than two atoms common to both rings. Examples of such bridged heterocycles include quinuclidinyl, diazabicyclo[2.2.1]heptyl; and 7-oxabicyclo[2.2.1]heptyl.

The term “alkoxy” used alone or as a suffix or prefix, refers to radicals of the general formula —O—R, wherein R is selected from a hydrocarbon radical. Exemplary alkoxy includes methoxy, ethoxy, propoxy, isopropoxy, butoxy, t-butoxy, isobutoxy, cyclopropylmethoxy, allyloxy, and propargyloxy.

The term “amine” or “amino” used alone or as a suffix or prefix, refers to radicals of the general formula —NRR′, wherein R and R′ are independently selected from hydrogen or a hydrocarbon radical.

“Acyl” used alone, as a prefix or suffix, means —C(═O)—R, wherein R is an optionally substituted hydrocarbyl, hydrogen, amino or alkoxy. Acyl groups include, for example, acetyl, propionyl, benzoyl, phenyl acetyl, carboethoxy, and dimethylcarbamoyl.

“Halogen” includes fluorine, chlorine, bromine and iodine.

“Halogenated,” used as a prefix of a group, means one or more hydrogens on the group is replaced with one or more halogens.

“RT” or “rt” means room temperature.

A first ring group being “fused” with a second ring group means the first ring and the second ring share at least two atoms therebetween.

“Link,” “linked,” or “linking,” unless otherwise specified, means covalently linked or bonded.

### Compounds

Compounds of the invention conform generally to Formula I:

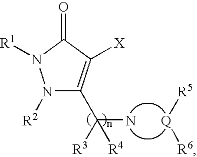

wherein R¹, R², R³, R⁴, R⁵, R⁶, X, Q, and n are defined hereinabove. The cyclic moiety

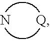

consistent with the definition set forth above, generally represents a heterocycle that contains at least one nitrogen atom. The moiety can be fully saturated, partially saturated, or aromatic when appropriate, and can be substituted by one or more substituents A. Thus in some embodiments of the invention,

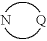

can represent any of the following core structures:

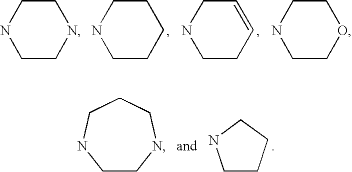

In other embodiments,

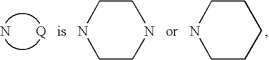

and still other embodiments

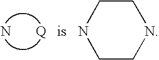

It will therefore be appreciated by those who are skilled in the art that R⁵ or R⁶, both, or neither will be present depending upon the identity and thus valency of atom Q. Thus, for example, in those embodiments where Q is a carbon atom, one of R⁵ and R⁶ may be present if Q is involved in an unsaturated bond. Alternatively, both of R⁵ and R⁶ are present where Q is carbon that shares only fully saturated, i.e., single, bonds with neighboring atoms. Other embodiments provide for Q being a nitrogen atom, in which case at most one of R⁵ and R⁶ can be present. In this context, the nitrogen atom may form part of an aromatic ring system or otherwise participate in an unsaturated bond. Consequently, in these compounds, neither of R⁵ and R⁶ would be present. In still other embodiments, Q represents an oxygen or sulfur atom, thereby precluding the presence of R⁵ and R⁶.

The ring

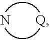

as contemplated herein, may contain heteroatoms, such as N, O, and S, other than those represented by Q to form a heterocycle as defined herein. It should be understood that, consistent with the definitions given above,

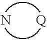

may be fused with one or more other appropriate cyclic moieties to form a fused ring system as defined herein.

Other embodiments of the invention contemplate compounds according to Formula I wherein X is Br, Cl, or OC₁₋₆-alkyl. Preferably, X is Br or Cl. When X is OC₁₋₆-alkyl, X can be, for example, methoxy or ethoxy.

Another subset of compounds are those in which R¹ is selected from the group consisting of aryl, C₃₋₈-cycloalkyl, C₁₋₆-alkyl-aryl, and C₁₋₆-alkyl-C₃₋₈-cycloalkyl. Each of these groups may be substituted by one or more A. In some embodiments, R¹ is selected from aryl and C₃₋₈-cycloalkyl groups. Preferably, R¹ is an aryl group, such as, for example, phenyl. Alternatively, R¹ can be a C₃₋₈-cycloalkyl group, including, for example, cyclohexyl.

The invention contemplates another embodiment where R² is H or a C₁₋₆-alkyl group. Preferably, R² is C₁₋₆-alkyl such as, for example, methyl or ethyl.

Other embodiments of the invention provide for compounds of Formula I in which R⁵ and R⁶, when at least one is present, are selected from the group consisting of H, aryl, and C₃₋₈-cycloalkyl.

A preferred subset of compounds are those wherein Q is C. Preferably, both R⁵ and R⁶ are present. Thus, some embodiments provide for R⁵ and R⁶, together with Q, to combine to form a 5- to 7-membered ring containing atoms independently selected from the group consisting of C, N, O and S. Suitable 5- to 7-membered rings in this regard include any appropriate cyclic moiety as defined hereinabove.

Preferred rings in this regard include but are not limited to the substructures

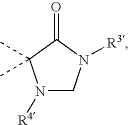

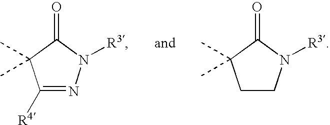

In this regard, the person who is skilled in the art will appreciate that the dashed lines represent bonds with the

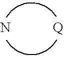

ring to indicate that atom Q is common to

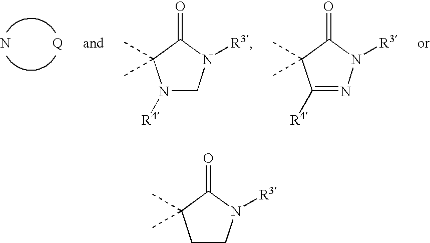

engender spiro fusion between the two rings. Substituents R^{3′} and R^{4′} have the same definitions as R³ and R⁴, respectively, as set forth above. In some embodiments, R^{3′} and R^{4′} are independently selected from the group consisting of H, C₁₋₆-alkyl, C₁₋₆-alkyl-aryl, aryl, and heteroaryl, wherein R^{3′} and R^{4′} may be substituted by one or more A. A preferred value for R^{4′}, when present, is aryl, such as phenyl.

Another preferred embodiment according to the invention provides for those compounds in which X is selected from the group consisting of Cl, Br, and OC₁₋₆-alkyl and ring

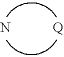

is

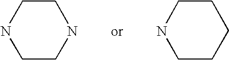

that may be substituted by one or more A. In this embodiment, R¹ is selected from aryl and C₃₋₈-cycloalkyl, wherein R¹ may be substituted by one or more A; R² is selected from H and C₁₋₆-alkyl; R⁵ and R⁶, when one or more is present, are independently selected from the group consisting of H, aryl, and C₃₋₈-cycloalkyl, wherein R⁵ and R⁶ maybe substituted by one or more A; and n is 1.

In another embodiment, R⁵ and R⁶, together with Q, combine to form

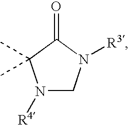

wherein R^{3′} is selected from the group consisting of H, C₁₋₆-alkyl, C₁₋₆-alkyl-aryl, aryl, and heteroaryl; R^{4′} is phenyl; and wherein R^{3′} and R^{4′} may be substituted by one or more A.

In another embodiment, the invention compounds of the invention conform generally to Formula II:

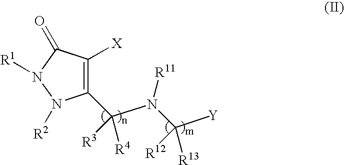

wherein
X is selected from the group consisting of F, Cl, Br, I, cyano, OC₁₋₆-alkyl, C₁₋₆-alkylhalo, OC₁₋₆-alkylhalo;
R¹ is selected from the group consisting of C₁₋₆-alkyl, C₂₋₆-alkenyl, C₂₋₆-alkynyl, aryl, heteroaryl, heterocycloalkyl, C₃₋₈-cycloalkyl, C₁₋₆-alkyl-aryl, C₁₋₆-alkyl-heteroaryl, C₁₋₆-alkyl-heterocycloalkyl, C₁₋₆-alkyl-C₃₋₈-cycloalkyl, wherein R¹ may be substituted by one or more A;
R² is selected from the group consisting of H, C₁₋₆-alkyl, C₂₋₆-alkenyl, and C₂₋₆-alkynyl, wherein R² may be substituted by one or more A;
R³, R⁴, R¹² and R¹³ are each independently selected from the group consisting of H, C₁₋₆-alkyl, C₂₋₆-alkenyl, C₂₋₆-alkynyl, aryl, heteroaryl, heterocycloalkyl, C₃₋₈-cycloalkyl, C₁₋₆-alkyl-aryl, C₁₋₆-alkyl-heteroaryl, C₁₋₆-alkyl-heterocycloalkyl, C₁₋₆-alkyl-C₃₋₈-cycloalkyl, wherein R³ and R⁴ may be substituted by one or more A;
R¹¹ is selected from the group consisting of H, C₁₋₆-alkyl, C₁₋₆-alkylhalo, C₂₋₆-alkenyl, C₂₋₆-alkynyl, C₃₋₈-cycloalkyl, C₁₋₆-alkyl-C₃₋₈-cycloalkyl, C₃₋₈-heterocycloalkyl, C₁₋₆-alkyl-C₃₋₈-heterocycloalkyl aryl, C₁₋₆-alkylaryl, heteroaryl, C₁₋₆-alkylheteroaryl, C(O)H, (CO)R⁷, C(O)OR⁷, C₁₋₆-alkylOR⁷, C₁₋₆-alkyl(CO)R⁷, C₁₋₆-alkylCO₂R⁷, C₁₋₆-alkylcyano, C₁₋₆-alkylNR⁷R⁸, C₁₋₆-alkyl(CO)NR⁷R⁸, C₁₋₆-alkylNR⁷(CO)R⁸, C₁₋₆-alkylNR⁷(CO)NR⁷R⁸, C₁₋₆-alkylSR⁷, C₀₋₆-alkyl(SO)R⁷, C₀₋₆-alkylSO₂R⁷, C₀₋₆-alkyl(SO₂)NR⁷R⁸, C₀₋₆-alkylNR⁷(SO₂)R⁸, C₀₋₆-alkylNR⁷(SO₂)NR⁷R⁸, (CO)NR⁷R⁸, C₀₋₆-alkylNR⁷(CO)OR⁸, C₀₋₆-alkyl SO₃R⁷ and a 5- to 7-membered ring containing atoms independently selected from the group consisting of C, N, O and S, wherein R¹ may be substituted by one or more A, and wherein any cycloalkyl or aryl is optionally fused to a 5- to 7-membered ring containing atoms independently selected from the group consisting of C, N, O and S;
R⁷ and R⁸ are independently selected from the group consisting of hydrogen, C₁₋₆-alkyl, C₃₋₇-cycloalkyl, C(O)C₁₋₆-alkyl, aryl, C₁₋₆-alkylaryl, heterocycloalkyl, and heteroaryl, wherein R⁷ and R⁸ may be substituted by one or more A;
A is selected from the group consisting of hydroxy, F, Cl, Br, I, nitro, cyano, oxo, C₁₋₆-alkyl, C₁₋₆-alkylhalo, OC₁₋₆alkyl, OC₁₋₆-alkylhalo, C₂₋₆-alkenyl, OC₂₋₆-alkenyl, C₂₋₆-alkynyl, OC₂₋₆-alkynyl, C₃₋₈-cycloalkyl, C₁₋₆-alkyl-C₃₋₈-cycloalkyl, OC₀₋₆-alkyl-C₃₋₈-cycloalkyl, aryl, C₁₋₆-alkylaryl, OC₀₋₆-alkylarylheteroaryl, C₁₋₆-alkylheteroaryl, OC₀₋₆-alkylheteroaryl, (CO)R⁹, O(CO)R⁹, O(CO)OR⁹, OC(NH)OR⁹, C₁₋₆-alkylOR⁹, OC₂₋₆-alkylOR⁹, C₁₋₆-alkyl(CO)R⁹, OC₁₋₆-alkyl(CO)R⁹, C₀₋₆-alkylCO₂R⁹, OC₁₋₆-alkylCO₂R⁹, C₁₋₆-alkylcyano, OC₂₋₆-alkylcyano, C₀₋₆-alkylNR⁹R¹⁰, OC₂₋₆-alkylNR⁹R¹⁰, C₁₋₆-alkyl(CO)NR⁹R¹⁰, OC₁₋₆-alkyl(CO)NR⁹R¹⁰, C₀₋₆-alkylNR⁹(CO)R¹⁰, OC₂₋₆-alkylNR⁹(CO)R¹⁰, C₀₋₆-alkylNR⁹(CO)NR⁹R¹⁰, C₀₋₆-alkylSR⁹, OC₂₋₆-alkylSR⁹, C₀₋₆-alkyl(SO)R⁹, OC₂₋₆-alkyl(SO)R⁹, C₀₋₆-alkylSO₂R⁹, OC₂₋₆-alkylSO₂R⁹, C₀₋₆-alkyl(SO₂)NR⁹R¹⁰, OC₂₋₆-alkyl(SO₂)NR⁹R¹⁰, C₀₋₆-alkylNR⁹(SO₂)R¹⁰, OC₂₋₆-alkylNR⁹(SO₂)R¹⁰, C₀₋₆-alkylNR⁹(SO₂)NR⁹R¹⁰, OC₂₋₆-alkylNR⁹(SO₂)NR⁹R¹⁰, (CO)NR⁹R¹⁰, O(CO)NR⁹R¹⁰, NR⁹OR¹⁰, C₀₋₆-alkylNR⁹(CO)OR¹⁰, OC₂₋₆-alkylNR⁹(CO)OR¹⁰, OC(NH)OR⁹, SO₃R⁹, wherein any ring is optionally substituted with one or more B, and a 5- to 7-membered ring containing atoms independently selected from the group consisting of C, N, O and S, wherein said ring is optionally substituted by one or more of R⁹ and R¹⁰;
R⁹ and R¹⁰ are independently selected from the group consisting of H, hydroxy, F, Cl, Br, I, nitro, cyano, oxo, C₁₋₆-alkyl, C₁₋₆-alkylhalo, OC₁₋₆alkyl, OC₁₋₆-alkylhalo, C₂₋₆-alkenyl, OC₂₋₆-alkenyl, C₂₋₆-alkynyl, OC₂₋₆-alkynyl, C₃₋₈-cycloalkyl, C₁₋₆-alkyl-C₃₋₈-cycloalkyl, OC₀₋₆-alkyl-C₃₋₈-cycloalkyl, aryl, C₁₋₆-alkylaryl, OC₀₋₆-alkylaryl, heterocycloalkyl, and heteroaryl, and any ring is optionally substituted with one or more B;
B is selected from the group consisting of F, Cl, Br, I, C₁₋₆-alkyl and OC₁₋₆alkyl;
m is selected from the group consisting of 0, 1, 2, 3, 4, 5, and 6;
n is selected from the group consisting of 1, 2, 3, 4, 5, and 6; and
Y is selected from the group consisting of alkyl, alkenyl, alkynyl, aryl, heteroaryl, heterocycloalkyl and C₃₋₁₀-cycloalkyl, wherein Y may be substituted by one or more A;
   or a pharmaceutically acceptable salt, hydrate, solvate, optical isomer, or combination thereof.

It will be understood by those of skill in the art that when compounds of the present invention contain one or more chiral centers, the compounds of the invention may exist in, and be isolated as, enantiomeric or diastereomeric forms, or as a racemic mixture. The present invention includes any possible enantiomers, diastereomers, racemates or mixtures thereof, of a compound of Formula I or Formula II. The optically active forms of the compound of the invention may be prepared, for example, by chiral chromatographic separation of a racemate, by synthesis from optically active starting materials or by asymmetric synthesis based on the procedures described thereafter.

It will also be appreciated by those of skill in the art that certain compounds of the present invention may exist as geometrical isomers, for example E and Z isomers of alkenes. The present invention includes any geometrical isomer of a compound of Formula I or Formula II. It will further be understood that the present invention encompasses tautomers of the compounds of Formula I or Formula II.

It will also be understood by those of skill in the art that certain compounds of the present invention may exist in solvated, for example hydrated, as well as unsolvated forms. It will further be understood that the present invention encompasses all such solvated forms of the compounds of Formula I or Formula II.

Within the scope of the invention are also salts of the compounds of Formula I or Formula II. Generally, pharmaceutically acceptable salts of compounds of the present invention are obtained using standard procedures well known in the art, for example, by reacting a sufficiently basic compound, for example an alkyl amine with a suitable acid, for example, HCl or acetic acid, to afford a physiologically acceptable anion. It is also possible to make a corresponding alkali metal (such as sodium, potassium, or lithium) or an alkaline earth metal (such as a calcium) salt by treating a compound of the present invention having a suitably acidic proton, such as a carboxylic acid or a phenol with one equivalent of an alkali metal or alkaline earth metal hydroxide or alkoxide (such as the ethoxide or methoxide), or a suitably basic organic amine (such as choline or meglumine) in an aqueous medium, followed by conventional purification techniques.

In one embodiment of the present invention, the compound of Formula I or Formula II may be converted to a pharmaceutically acceptable salt or solvate thereof, particularly, an acid addition salt such as a hydrochloride, hydrobromide, phosphate, acetate, fumarate, maleate, tartrate, citrate, methanesulphonate or p-toluenesulphonate.

Specific examples of the present invention include the following compounds, their pharmaceutically acceptable salts, hydrates, solvates, optical isomers, and combinations thereof:

| Example No. | Structure |
|---|---|
| 373 | 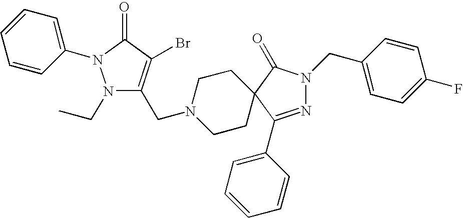 |
| 376 | 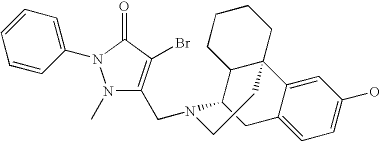 |
| 384 | 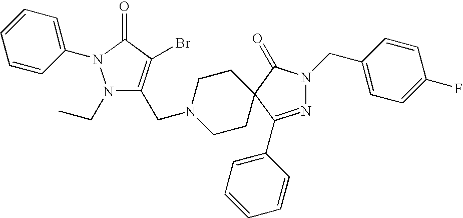 |
| 389 | 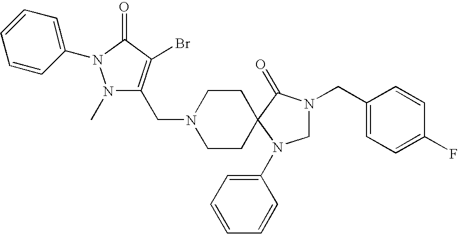 |
| 390 | 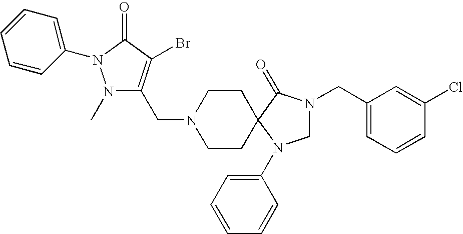 |
| 391 | 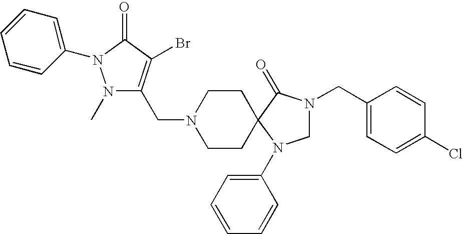 |
| 392 | 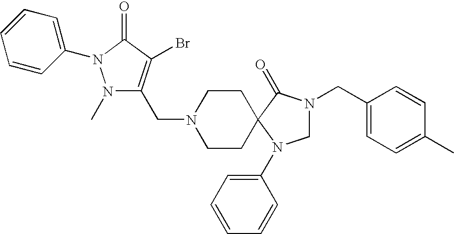 |
| 397 | 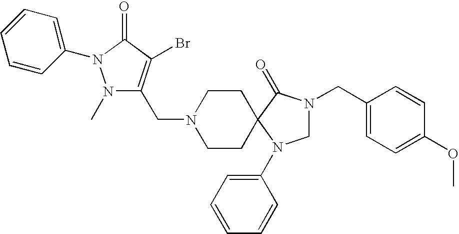 |
| 399 | 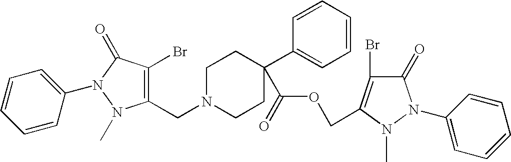 |
| 408 | 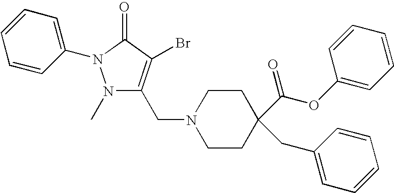 |
| 416a | 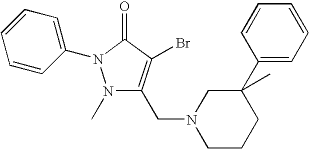 |
| 418 | 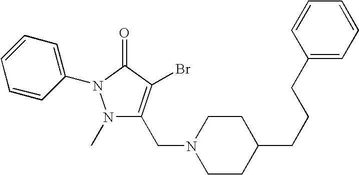 |
| 419 | 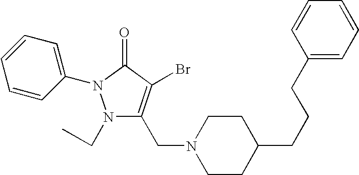 |
| 434 | 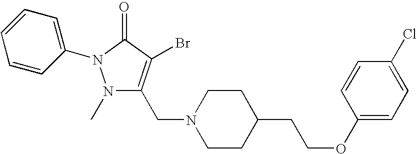 |
| 437 | 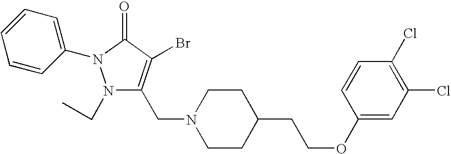 |
| 440 | 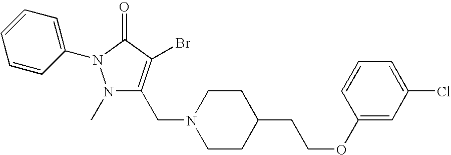 |
| 441 | 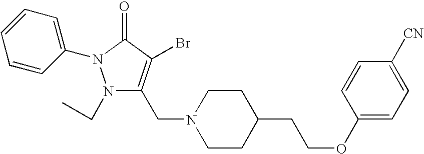 |
| 444 | 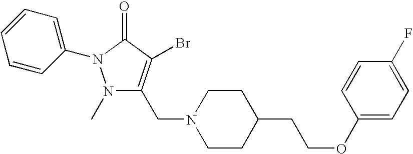 |
| 454 | 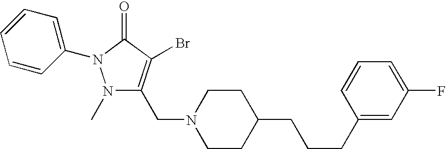 |
| 457 | 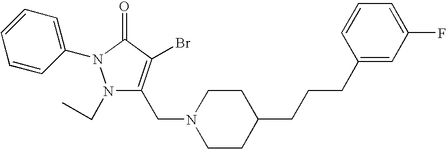 |
| 458 | 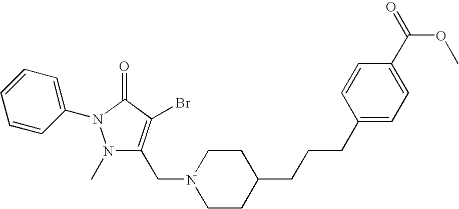 |
| 459 | 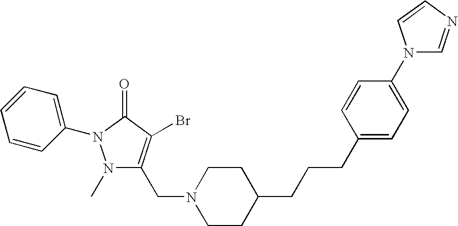 |
| 463 | 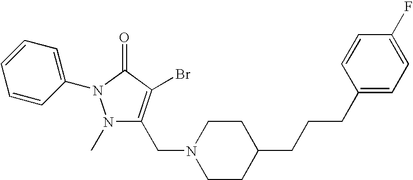 |
| 464 | 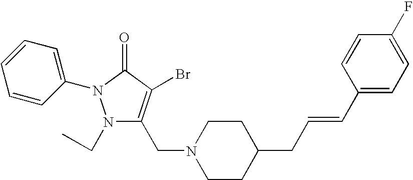 |
| 465 | 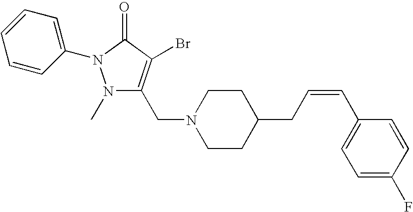 |
| 526 | 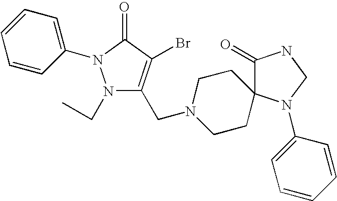 |
| 528 | 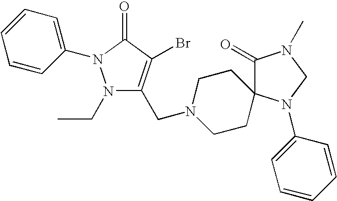 |
| 559 | 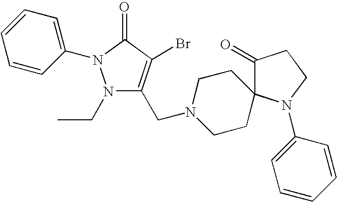 |
| 561 | 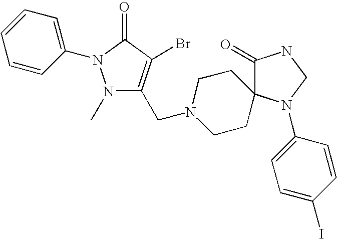 |
| 459 | 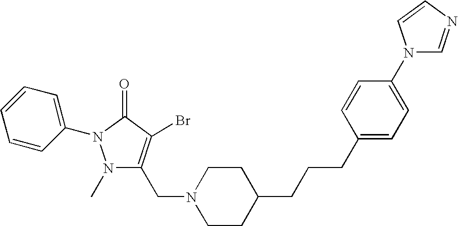 |
| 459a | 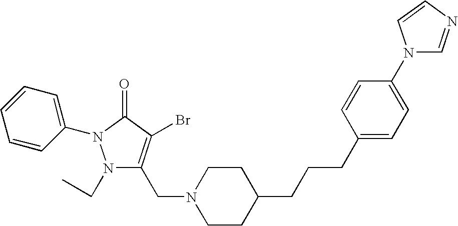 |
| 134 | 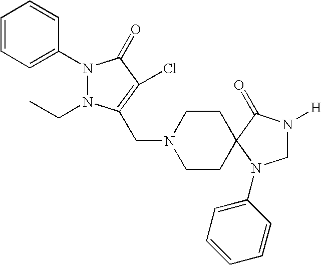 |
| 147 | 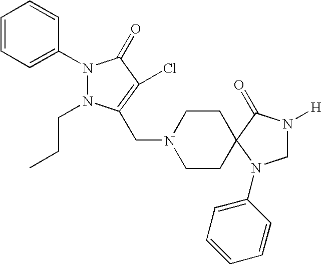 |
| 182 | 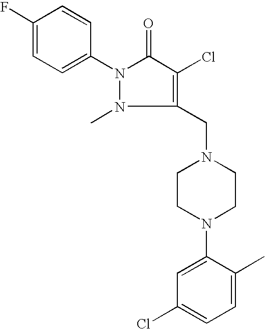 |
| 221 | 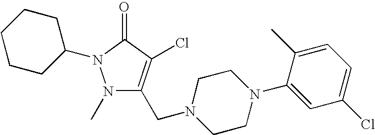 |
| 196 | 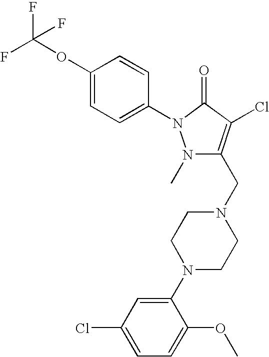 |
| 198 | 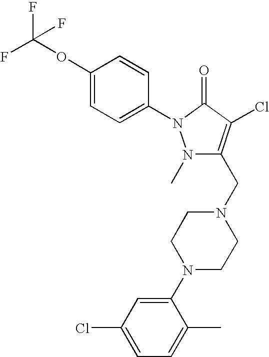 |
| 200 | 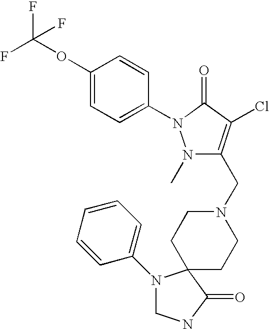 |
| 210 | 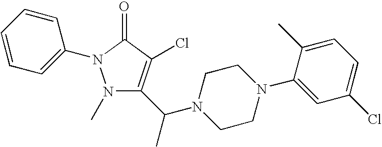 |
| 268 | 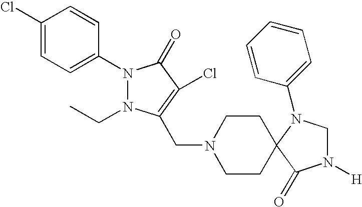 |
| 149 | 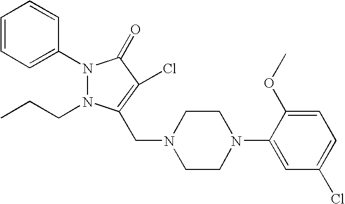 |
| 348 | 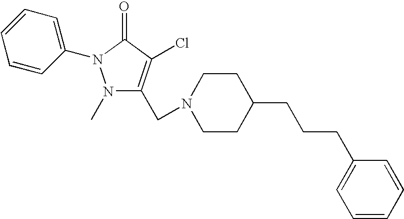 |
| 349 | 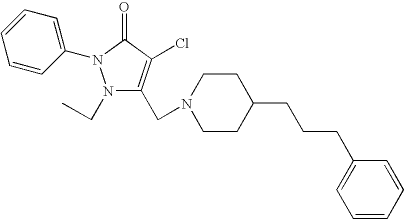 |
| 232 | 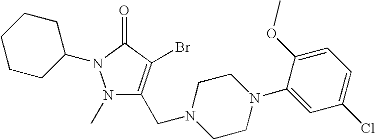 |
| 235 | 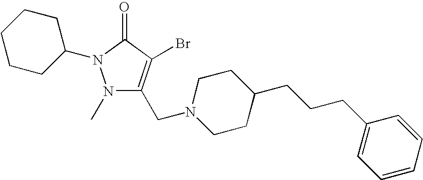 |
| 169 | 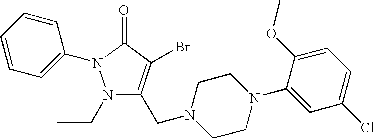 |
| 242 | 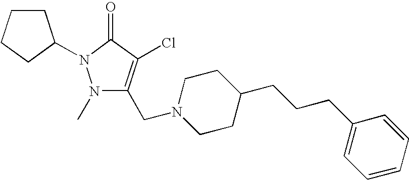 |
| 284 | 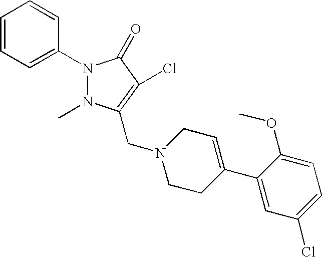 |
| 189 | 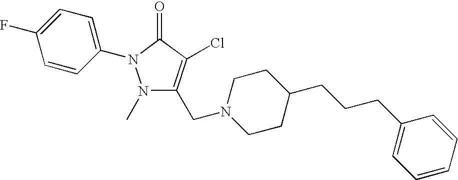 |
| 201 | 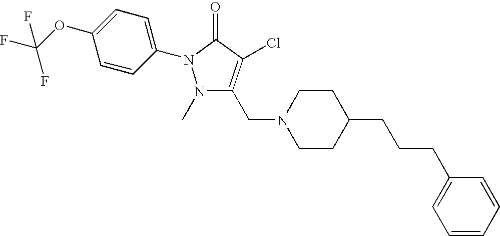 |
| 287 | 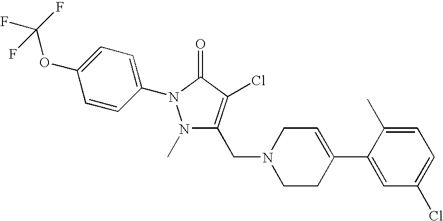 |
| 314 | 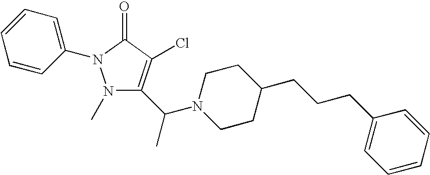 |
| 216 | 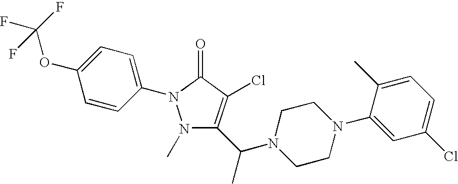 |
| 217 | 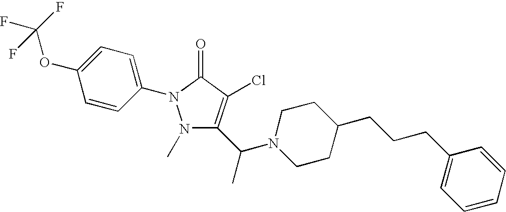 |
| 316 | 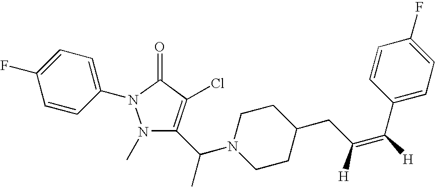 |
| 317 | 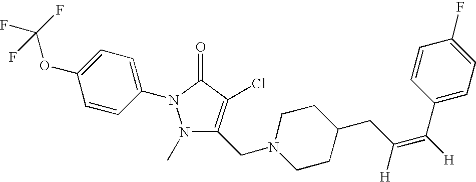 |
| 318 | 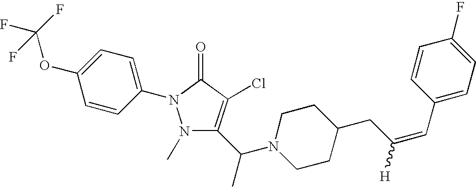 |
| 320 | 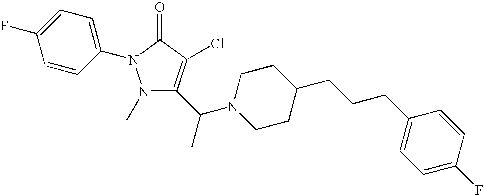 |
| 321 | 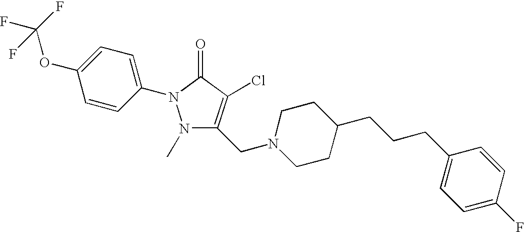 |

An embodiment includes the following exemplary compounds:

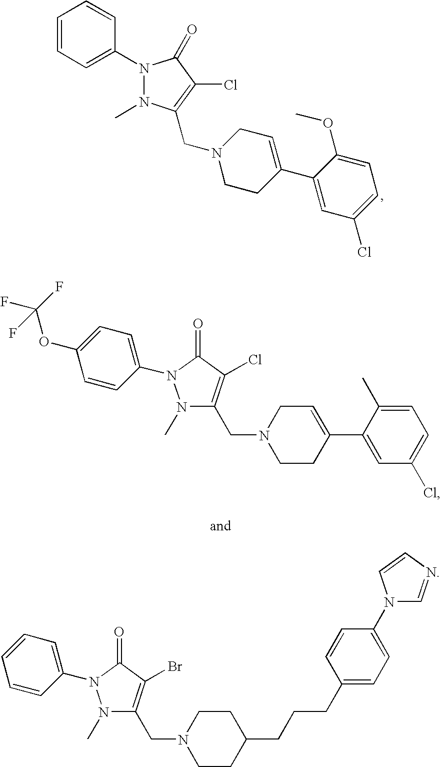

### Pharmaceutical Composition

The compounds of the present invention may be formulated into conventional pharmaceutical composition comprising a compound of Formula I or Formula II, or a pharmaceutically acceptable salt or solvate thereof, in association with a pharmaceutically acceptable carrier or excipient. The pharmaceutically acceptable carriers can be either solid or liquid. Solid form preparations include, but are not limited to, powders, tablets, dispersible granules, capsules, cachets, and suppositories.

A solid carrier can be one or more substances, which may also act as diluents, flavoring agents, solubilizers, lubricants, suspending agents, binders, or table disintegrating agents. A solid carrier can also be an encapsulating material.

In powders, the carrier is a finely divided solid, which is in a mixture with the finely divided compound of the invention, or the active component. In tablets, the active component is mixed with the carrier having the necessary binding properties in suitable proportions and compacted in the shape and size desired.

For preparing suppository compositions, a low-melting wax such as a mixture of fatty acid glycerides and cocoa butter is first melted and the active ingredient is dispersed therein by, for example, stirring. The molten homogeneous mixture is then poured into convenient sized moulds and allowed to cool and solidify.

Suitable carriers include, but are not limited to, magnesium carbonate, magnesium stearate, talc, lactose, sugar, pectin, dextrin, starch, tragacanth, methyl cellulose, sodium carboxymethyl cellulose, low-melting wax, cocoa butter, and the like.

The term composition is also intended to include the formulation of the active component with encapsulating material as a carrier providing a capsule in which the active component (with or without other carriers) is surrounded by a carrier which is thus in association with it. Similarly, cachets are included.

Tablets, powders, cachets, and capsules can be used as solid dosage forms suitable for oral administration.

Liquid form compositions include solutions, suspensions, and emulsions. For example, sterile water or water propylene glycol solutions of the active compounds may be liquid preparations suitable for parenteral administration. Liquid compositions can also be formulated in solution in aqueous polyethylene glycol solution.

Aqueous solutions for oral administration can be prepared by dissolving the active component in water and adding suitable colorants, flavoring agents, stabilizers, and thickening agents as desired. Aqueous suspensions for oral use can be made by dispersing the finely divided active component in water together with a viscous material such as natural synthetic gums, resins, methyl cellulose, sodium carboxymethyl cellulose, and other suspending agents known to the pharmaceutical formulation art. Exemplary compositions intended for oral use may contain one or more coloring, sweetening, flavoring and/or preservative agents.

Depending on the mode of administration, the pharmaceutical composition will include from about 0.05% w (percent by weight) to about 99% w, more particularly, from about 0.10% w to 50% w, of the compound of the invention, all percentages by weight being based on the total weight of the composition.

A therapeutically effective amount for the practice of the present invention can be determined by one of ordinary skill in the art using known criteria including the age, weight and response of the individual patient, and interpreted within the context of the disease which is being treated or which is being prevented.

### Medical Use

We have discovered that the compounds of the present invention exhibit activity as pharmaceuticals, in particular as modulators of metabotropic glutamate receptors. More particularly, the compounds of the present invention exhibit activity as potentiators of the mGluR2 receptor, and are useful in therapy, in particular for the treatment of neurological and psychiatric disorders associated with glutamate dysfunction in an animal. Compounds of the present invention are active in assays of mGluR function with EC₅₀ values of less than about 10 □m.

More specifically, the neurological and psychiatric disorders include, but are not limited to, disorders such as cerebral deficit subsequent to cardiac bypass surgery and grafting, stroke, cerebral ischemia, spinal cord trauma, head trauma, perinatal hypoxia, cardiac arrest, hypoglycemic neuronal damage, dementia (including AIDS-induced dementia), Alzheimer's disease, Huntington's Chorea, amyotrophic lateral sclerosis, ocular damage, retinopathy, cognitive disorders, idiopathic and drug-induced Parkinson's disease, muscular spasms and disorders associated with muscular spasticity including tremors, epilepsy, convulsions, cerebral deficits secondary to prolonged status epilepticus, migraine (including migraine headache), urinary incontinence, substance tolerance, substance withdrawal (including, substances such as opiates, nicotine, tobacco products, alcohol, benzodiazepines, cocaine, sedatives, hypnotics, etc.), psychosis, schizophrenia, anxiety (including generalized anxiety disorder, panic disorder, social phobia, obsessive compulsive disorder, and post-traumatic stress disorder (PTSD)), mood disorders (including depression, mania, bipolar disorders), circadian rhythm disorders (including jet lag and shift work), trigeminal neuralgia, hearing loss, tinnitus, macular degeneration of the eye, emesis, brain edema, pain (including acute and chronic pain states, severe pain, intractable pain, neuropathic pain, inflammatory pain, and post-traumatic pain), tardive dyskinesia, sleep disorders (including narcolepsy), attention deficit/hyperactivity disorder, and conduct disorder.

The invention thus provides a use of any of the compounds according to Formula I or Formula II, or a pharmaceutically acceptable salt or solvate thereof, for the manufacture of a medicament for the treatment of any of the conditions discussed above.

Additionally, the invention provides a method for the treatment of a subject suffering from any of the conditions discussed above, whereby an effective amount of a compound according to Formula I or Formula II or a pharmaceutically acceptable salt or solvate thereof, is administered to a patient in need of such treatment. The invention also provides a compound of Formula I or Formula II or pharmaceutically acceptable salt or solvate thereof, as hereinbefore defined for use in therapy.

In the context of the present specification, the term “therapy” also includes “prophylaxis” unless there are specific indications to the contrary. The term “therapeutic” and “therapeutically” should be construed accordingly. The term “therapy” within the context of the present invention further encompasses the administration of an effective amount of a compound of the present invention, to mitigate either a pre-existing disease state, acute or chronic, or to mitigate a recurring condition. This definition also encompasses prophylactic therapies for prevention of recurring conditions and continued therapy for chronic disorders.

In use for therapy in a warm-blooded animal such as a human, the compounds of the present invention may be administered in the form of a conventional pharmaceutical composition by any route including orally, intramuscularly, subcutaneously, topically, intranasally, intraperitoneally, intrathoracially, intravenously, epidurally, intrathecally, intracerebroventricularly and by injection into the joints. In preferred embodiments of the invention, the route of administration is oral, intravenous, or intramuscular.

The dosage will depend on the route of administration, the severity of the disease, age and weight of the patient and other factors normally considered by the attending physician, who determines the individual regimen and dosage level for a particular patient.

As mentioned above, the compounds described herein may be provided or delivered in a form suitable for oral use, for example, in a tablet, lozenge, hard and soft capsule, aqueous solution, oily solution, emulsion, and suspension. Alternatively, the compounds may be formulated into a topical administration, for example, as a cream, ointment, gel, spray, or aqueous solution, oily solution, emulsion or suspension. The compounds described herein also may be provided in a form that is suitable for nasal administration, for example, as a nasal spray, nasal drops, or dry powder. The compounds can be administered to the vagina or rectum in the form of a suppository. The compounds described herein also may be administered parentally, for example, by intravenous, intravesicular, subcutaneous, or intramuscular injection or infusion. The compounds can be administered by insufflation (for example as a finely divided powder). The compounds may also be administered transdermally or sublingually.

In addition to their use in therapeutic medicine, the compounds of Formula I or Formula II, or salts thereof, are useful as pharmacological tools in the development and standardisation of in vitro and in vivo test systems for the evaluation of the effects of inhibitors of mGluR-related activity in laboratory animals as part of the search for new therapeutics agents. Such animals include, for example, cats, dogs, rabbits, monkeys, rats and mice.

### Process for Preparing

Compounds of the present invention can be prepared by various synthetic processes. The selection of a particular process to prepare a given compound is within the purview of the person of skill in the art. The choice of particular structural features and/or substituents may therefore influence the selection of one process over another.

Within these general guidelines, the following processes can be used to prepare the compounds described herein. Unless indicated otherwise, the variables described in the following schemes and processes have the same definitions as those given for Formula I or Formula II above.

### Synthesis of Final Compounds

The general synthesis of 4-halopyrazolones is depicted in Scheme 1. A monosubstituted hydrazine i is cyclized with an appropriate □-ketoseter ii by heating under acidic conditions to yield pyrazolone iii. This intermediate can be N-alkylated to iv with the desired alkyl iodide in acetonitrile with heat in an autoclave to prevent evapourative loss of the alkylating agent. Halogenation with either N-chlorosuccinimide and/or N-bromosuccinimide in a chlorinated solvent with mild heating provides the electrophile vi. This can then be alkylated with the desired amines

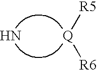

using potassium carbonate as the base to yield the final compounds vii.

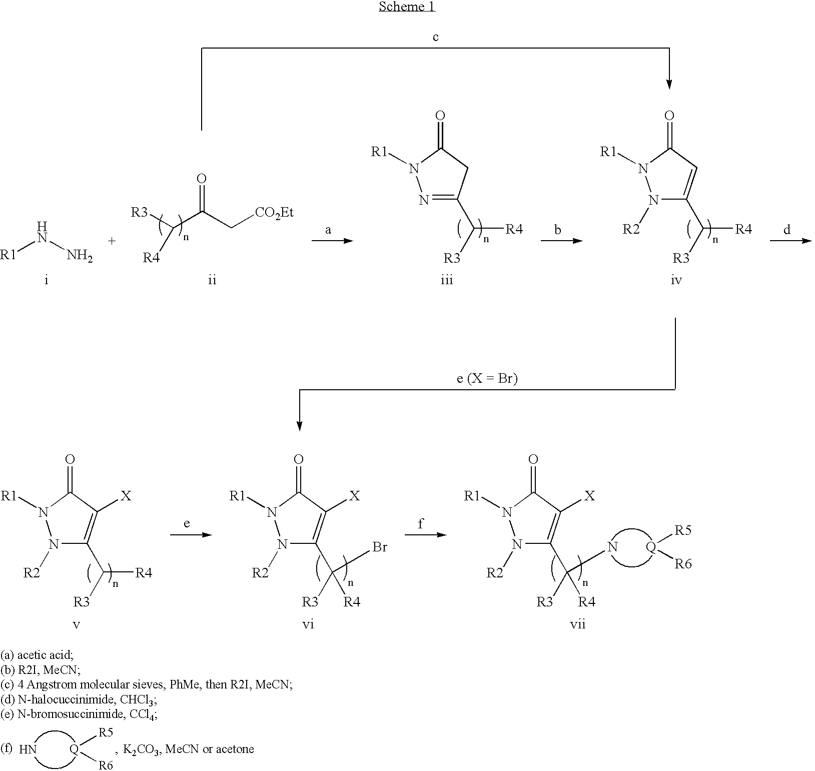

The synthesis of 4-alkoxypyrazolones is depicted in Scheme 2. 4-Bromopyrazolone is hydrolyzed with KOH and Triton B to the 4-hydroxypyrazolone viii. This could be alkylated with simple electrophiles under basic conditions to yield intermediates ix. To synthesize the difluoromethoxy derivative viii was first alkylated with ethyl bromodifluoroacetate. In the same pot, the ester was hydrolyzed under basic conditions, and the resultant acid decarboxylated by vigorous heating. Intermediates ix and x could then be advanced as depicted in Scheme 1.

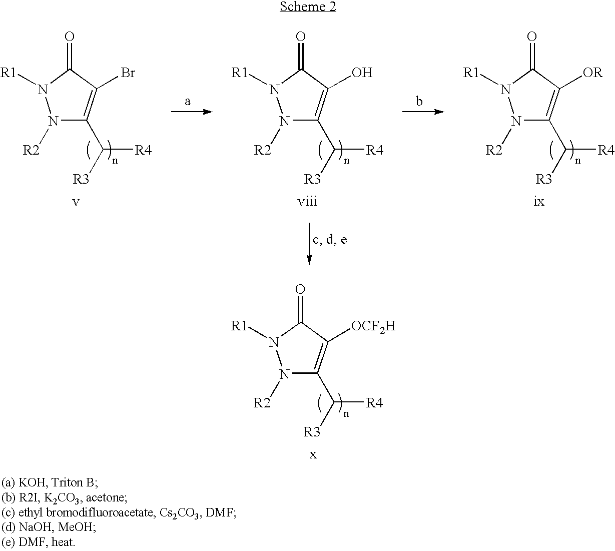

### Synthesis of Intermediate Amines

Many of the amines used in the synthesis of these compounds were not available from commercial sources. Some arylpiperazines were prepared as depicted in Scheme 3. The nitroarene xi was reduced with ferrum, and the aniline xii thus produced was cyclized with bis(2-chloroethyl)amine under basic conditions to yield the desired arylpiperazines xiii.

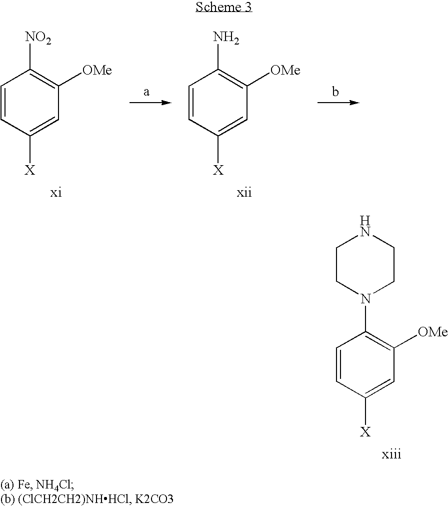

The synthesis of substituted arylpiperidines is depicted in Scheme 4. N-Boc-piperidone is converted to the vinyl boronate xvi vis the vinyl triflate xv. The boronate is reacted with appropriate aryl halides to generate xvii. These were either deprotected to yield tetrahydropyridines xx, or first hydrogenated, then deprotected to yield the fully saturated arylpiperidines xix.

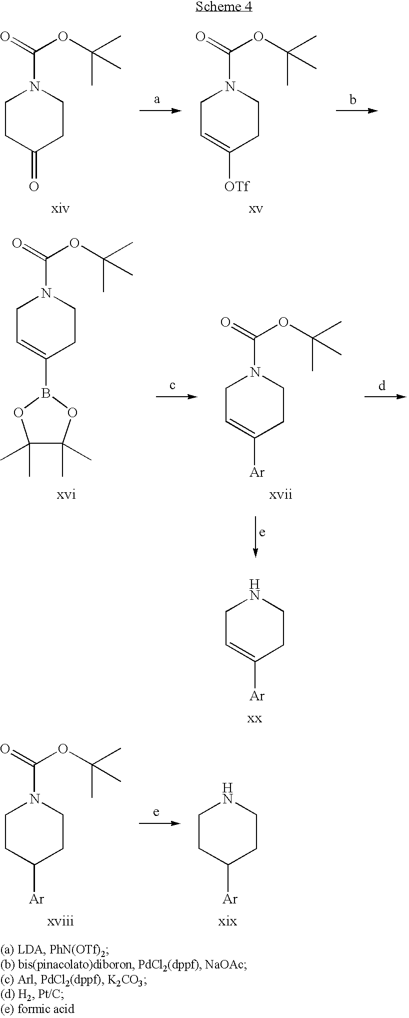

(Phenoxyethyl)piperidines were prepared as depicted in Scheme 5. Alcohol xxi was brominated with N-bromosuccinimide, then the bromide was displaced with appropriate phenols under basic conditions. Removal of the Boc protecting group yielded the desired intermediates xxiv.

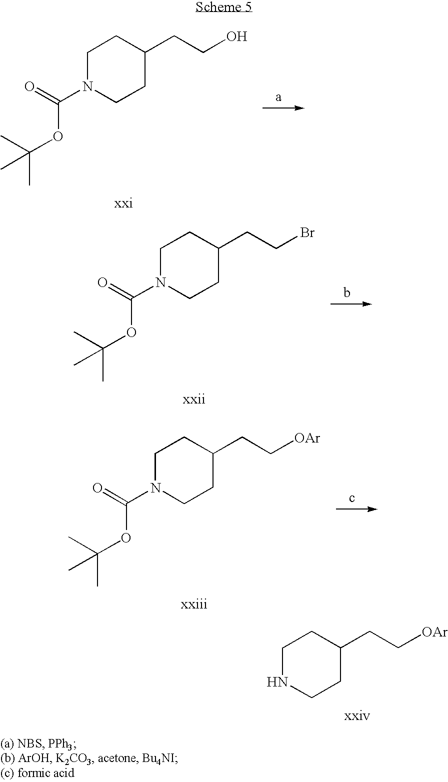

The (arylpropyl)piperidines were prepared as depicted in Scheme 6. Wittig reaction with aldehyde xxv and the appropriate (arylmethyl)triphenylphosphonium bromide yielded alkene xxvi as a mixture of geometric isomers. This compound was either deprotected directly to provide xxvii, or first hydrogenated to the saturated alkane xxviii, then deprotected to provide xix.

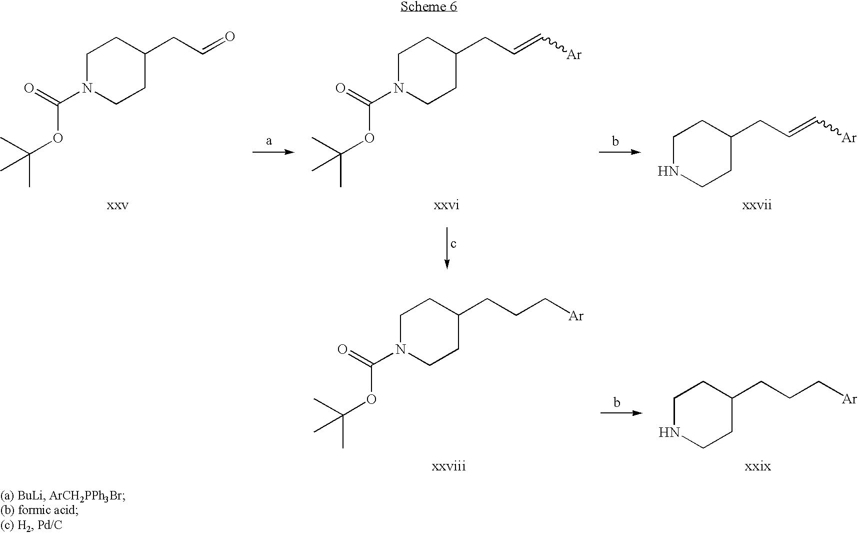

piperazine amide compounds were prepared as depicted in Scheme 7. Bromide vi was condensed with N-Boc-piperazine followed by deprotection to provide amine xxxi. This was acylated with the appropriate carboxylic acid under typical conditions to yield amides xxxii.

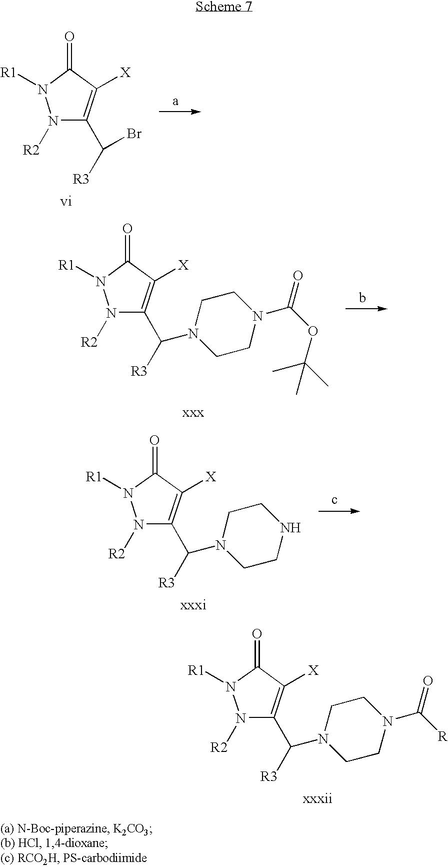

Spirocyclic piperidines xxxviii were synthesized as depicted in Scheme 8. xxxiii was first aroylated. The □-ketoester xxxiv was cyclized with hydrazine to provide pyrazolone xxxv. This intermediate could either be directly deprotected to yield piperidine xxxvi, or first alkylated with an appropriate benzyl halide under basic conditions, then deprotected to provide xxxviii.

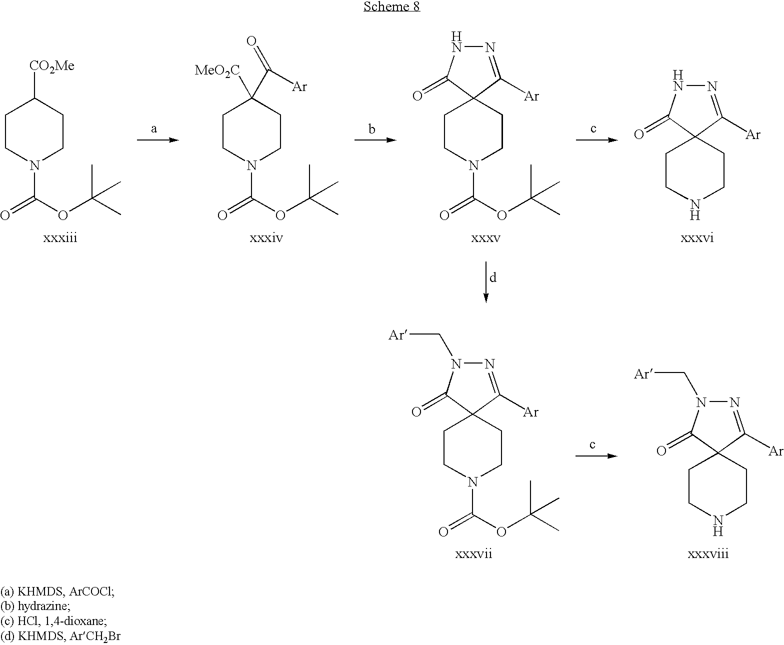

Spirocyclic piperidines xlii were prepared as depicted in Scheme 9. Amine xxxix was first protected with a Boc group, then alkylated with the appropriate benzyl amine under basic conditions. Deprotection provided the desired compounds xlii.

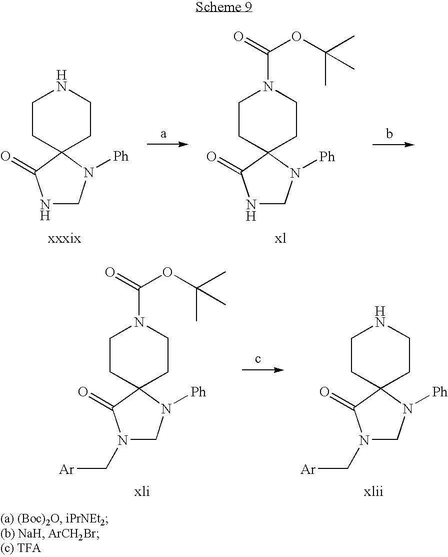

The invention is further illustrated by way of the following examples, which are intended to elaborate several embodiments of the invention. These examples are not intended to, nor are they to be construed to, limit the scope of the invention. It will be clear that the invention may be practiced otherwise than as particularly described herein. Numerous modifications and variations of the present invention are possible in view of the teachings herein and, therefore, are within the scope of the invention.

### General Methods

All starting materials are commercially available or earlier described in the literature. The ¹H and ¹³C NMR spectra were recorded either on Bruker 300, Bruker DPX400 or Varian +400 spectrometers operating at 300, 400 and 400 MHz for ¹H NMR respectively, using TMS or the residual solvent signal as reference, in deuterated chloroform as solvent unless otherwise indicated. All reported chemical shifts are in ppm on the delta-scale, and the fine splitting of the signals as appearing in the recordings (s: singlet, br s: broad singlet, d: doublet, t: triplet, q: quartet, m: multiplet).

Analytical in line liquid chromatography separations followed by mass spectra detections, were recorded on a Waters LCMS consisting of an Alliance 2795 (LC) and a ZQ single quadropole mass spectrometer. The mass spectrometer was equipped with an electrospray ion source operated in a positive and/or negative ion mode. The ion spray voltage was +3 kV and the mass spectrometer was scanned from m/z 100-700 at a scan time of 0.8 s. To the column, X-Terra MS, Waters, C8, 2.1×50 mm, 3.5 mm, was applied a linear gradient from 5% to 100% acetonitrile in 10 mM ammonium acetate (aq.), or in 0.1% TFA (aq.).

Preparative reversed phase chromatography was run on a Gilson autopreparative HPLC with a diode array detector using an XTerra MS C8, 19×300 mm, 7 mm as column.

Purification by a chromatotron was performed on rotating silica gel/gypsum (Merck, 60 PF-254 with calcium sulphate) coated glass sheets, with coating layer of 1, 2, or 4 mm using a TC Research 7924T chromatotron.

Purification of products were also done using Chem Elut Extraction Columns (Varian, cat #1219-8002), Mega BE-SI (Bond Elut Silica) SPE Columns (Varian, cat #12256018; 12256026; 12256034), or by flash chromatography in silica-filled glass columns.

Microwave heating was performed in a Smith Synthesizer Single-mode microwave cavity producing continuous irradiation at 2450 MHz (Personal Chemistry AB, Uppsala, Sweden).

The pharmacological properties of the compounds of the invention can be analyzed using standard assays for functional activity. Examples of glutamate receptor assays are well known in the art as described in, for example, Aramori et al., 1992, Neuron, 8:757; Tanabe et al., 1992, Neuron, 8:169; Miller et al., 1995, J. Neuroscience, 15:6103; Balazs, et al., 1997, J. Neurochemistry, 1997, 69:151. The methodology described in these publications is incorporated herein by reference. Conveniently, the compounds of the invention can be studied by means of an assay that measures the mobilization of intracellular calcium, [Ca²⁺]ᵢ in cells expressing mGluR2.

Fluorometric Imaging Plate Reader (FLIPR) analysis was used to detect allosteric activators of mGluR2 via calcium mobilization. A clonal HEK 293 cell line expressing a chimeric mGluR2/CaR construct comprising the extracellular and transmembrane domains of human mGluR2 and the intracellular domain of the human calcium receptor, fused to the promiscuous chimeric protein G_{□qi5} was used. Activation of this construct by agonists or allosteric activators resulted in stimulation of the PLC pathway and the subsequent mobilization of intracellular Ca²⁺ which was measured via FLIPR analysis. At 24-hours prior to analysis, the cells were trypsinized and plated in DMEM at 100,000 cells/well in black sided, clear-bottom, collagen I coated, 96-well plates. The plates were incubated under 5% CO₂ at 37° C. overnight. Cells were loaded with 6 μM fluo-3 acetoxymethylester (Molecular Probes, Eugene Oreg.) for 60 minutes at room temperature. All assays were performed in a buffer containing 126 mM NaCl, 5 mM KCl, 1 mM MgCl₂, 1 mM CaCl₂, 20 mM Hepes, 0.06 μM DCG-IV (a Group II mGluR selective agonist), supplemented with 1.0 mg/ml D-glucose and 1.0 mg/ml BSA fraction IV (pH 7.4).

FLIPR experiments were done using a laser setting of 0.8 W and a 0.4 second CCD camera shutter speed. Extracellular fluo-3 was washed off and cells were maintained in 160 μL of buffer and placed in the FLIPR. An addition of test compound (0.01 μM to 30 μM in duplicate) was made after 10 seconds of baseline fluorescent readings were recorded on FLIPR. Fluorescent signals were then recorded for an additional 75 seconds at which point a second addition of DCG-IV (0.2 μM) was made and fluorescent signals were recorded for an additional 65 seconds. Fluorescent signals were measured as the peak height of the response within the sample period. Data was analyzed using Assay Explorer, and EC₅₀ and Eₘₐₓ values (relative to maximum DCG-IV effect) were calculated using a four parameter logistic equation.

A [³⁵S]-GTPγS binding assay was used to functionally assay mGluR2 receptor activation. The allosteric activator activity of compounds at the human mGluR2 receptor were measured using a [³⁵S]-GTPγS binding assay with membranes prepared from CHO cells which stably express the human mGluR2. The assay is based upon the principle that agonists bind to G-protein coupled receptors to stimulate GDP-GTP exchange at the G-protein. Since [³⁵S]-GTPγS is a non-hydrolyzable GTP analog, it can be used to provide an index of GDP-GTP exchange and, thus, receptor activation. The GTPγS binding assay therefore provides a quantitative measure of receptor activation.

Membranes were prepared from CHO cells stably transfected with human mGluR2. Membranes (30 μg protein) were incubated with test compound (3 nM to 300 μM) for 15 minutes at room temperature prior to the addition of 1 μM glutamate, and incubated for 30 min at 30° C. in 500 μl assay buffer (20 mM HEPES, 100 mM NaCl, 10 mM MgCl₂), containing 30 μM GDP and 0.1 nM [³⁵S]-GTPγS (1250 Ci/mmol). Reactions were carried out in triplicate in 2 ml polypropylene 96-well plates. Reactions were terminated by vacuum filtration using a Packard 96-well harvester and Unifilter-96, GF/B filter microplates. The filter plates were washed 4×1.5 ml with ice-cold wash buffer (10 mM sodium phosphate buffer, pH 7.4). The filter plates were dried and 35 μl of scintillation fluid (Microscint 20) was added to each well. The amount of radioactivity bound was determined by counting plates on the Packard TopCount. Data was analyzed using GraphPad Prism, and EC₅₀ and Eₘₐₓ values (relative to the maximum glutamate effect) were calculated using non-linear regression

### Preparation of Intermediates I

### Pyrazalone Ring Formation

### General Procedure A

A hydrazine (1.0 equiv.) dissolved in acetic acid was treated with ethyl acetoacetate (1.0 equiv.). This mixture was left stirring at room temperature for a half hour and then for two hours at 50° C. and finally left overnight at 80° C. The acetic acid was concentrated and the residue was partitioned in ethyl acetate and saturated sodium bicarbonate solution. The organic was dried over anhydrous sodium sulphate, filtered and concentrated. Sometimes the crude mixture was purified using column chromatography in a solvent mixture of methanol and dichloromethane. NMR was used to determine the purity of the isolated compounds.

Intermediate compounds of Examples 1 through 72 inclusive were synthesized using a method analogous to general procedure A for pyrazolone ring formation.

### Example 1

### 5-Methyl-2-phenyl-1,2-dihydropyrazol-3-one

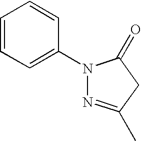

5-Methyl-2-phenyl-1,2-dihydropyrazol-3-one was obtained with phenyl hydrazine (21.6 g, 0.2 mol) in acetic acid (200 mL) and ethyl acetoacetate (29 mL, 0.23 mol) as brown crude solid. The crude product was triturated with hexane/ether (20:1) to afford yellow solid product (30.5 g, 86%). ¹H NMR (300 MHz, CDCl₃): [symbol] (ppm) 7.87 (d, 2H), 7.41 (dd, 2H), 7.20 (t, 1H), 3.45 (s, 2H), 2.22 (s, 3H).

### Example 2

### 2-(4-Fluorophenyl)-5-methyl-2,4-dihydropyrazol-3-one

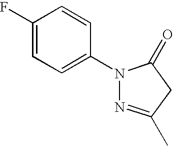

2-(4-Fluorophenyl)-5-methyl-2,4-dihydropyrazol-3-one was obtained with (4-fluorophenyl)hydrazine hydrochloride (1 g, 6.15 mmol) in acetic acid (1.5 mL) and ethyl acetoacetate (0.784 mL, 6.15 mmol) as brown solid. The crude product was chromatographed in 1% methanol and dichloromethane to yield a brown solid 500 mg (45%). ¹H NMR (300 MHz, CDCl₃): δ(ppm) 7.84 (t, 2H), 7.10 (t, 2H), 3.45 (s, 2H), 2.21 (s, 3H).

### Example 3

### 2-(4-Chloro-phenyl)-5-methyl-2,4-dihydro-pyrazol-3-one

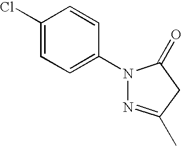

2-(4-Chloro-phenyl)-5-methyl-2,4-dihydro-pyrazol-3-one was synthesized from (4-chloro-phenyl)-hydrazine (2 g, 14.0 mmol), ethylacetoacetate (1.826 g, 14.0 mmol), and acetic acid (50 ml) to give yield 55% of product. (The product may be in two different forms due to tautomerization.) ¹H NMR (300 MHz, CDCl₃) δ (ppm): 7.86 (m, 2H), 7.36 (m, 2H), 3.44 (s, 1H), 2.21 (s, 1H).

### Example 4

### 2-(3-Chloro-4-fluoro-phenyl)-5-methyl-2,4-dihydro-pyrazol-3-one

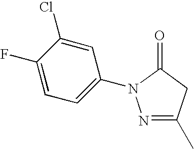

2-(3-Chloro-4-fluoro-phenyl)-5-methyl-2,4-dihydro-pyrazol-3-one was synthesized with general procedure from (3-chloro-4-fluoro-phenyl)-hydrazine (5 g, 31.1 mmol), ethyl acetoacetate (4.05 g, 31.1 mmol), and ethanol (8.0 ml) was used to give yield 10% of crude product as a yellow solid. ¹H NMR (300 MHz, CDCl₃) δ (ppm): 7.98-8.01 (m, 1H), 7.78-7.83 (m, 1H), 7.12 (t, 1H), 3.45 (s, 2H), 2.24 (s, 3H).

### Example 5

### 5-Methyl-2-(4-trifluoromethylphenyl)-2,4-dihydropyrazol-3-one

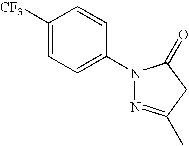

5-Methyl-2-(4-trifluoromethylphenyl)-2,4-dihydropyrazol-3-one was obtained with (4-trifluoromethylphenyl)hydrazine hydrochloride (5 g, 28.4 mmol) in acetic acid (90 mL) and ethyl acetoacetate (3.62 mL, 28.4 mmol) as a brown solid. The crude product was chromatographed in dichloromethane to yield a brown solid 5.76 g (84%). ¹H NMR (300 MHz, CDCl₃): δ(ppm) 7.91 (d, 2H), 7.55 (d, 2H), 3.41 (s, 2H), 2.13 (s, 3H).

### Example 6

### 5-Methyl-2-(4-trifluoromethoxyphenyl)-2,4-dihydropyrazol-3-one

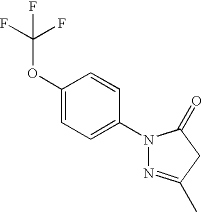

5-Methyl-2-(4-trifluoromethoxyphenyl)-2,4-dihydropyrazol-3-one was obtained with (4-trifluoromethoxyphenyl)hydrazine hydrochloride (2.255 g, 9.86 mmol) in acetic acid (40 mL) and ethyl acetoacetate (1.294 g, 9.86 mmol) as an off-white solid. The crude product was chromatographed in dichloromethane to yield an off-white solid (1.06 g, 42%). ¹H NMR (300 MHz, CDCl₃): δ(ppm) 7.94 (d, 2H), 7.26 (d, 2H), 3.47 (s, 2H), 2.23 (s, 3H).

### Example 7

### 5-Ethyl-2-phenyl-2,4-dihydro-pyrazol-3-one

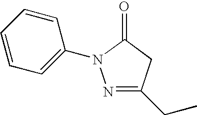

5-Ethyl-2-phenyl-2,4-dihydro-pyrazol-3-one was synthesized using the general procedure from phenyl hydrazine (5.0 g, 34.7 mmol), ethylpropionylacetate (3.75 g, 34.7 mmol) and acetic acid (50 ml) was used to give 6.5 g of a crude brown solid. ¹H NMR (300 MHz, CDCl₃) δ (ppm): 7.88 (d, 2H), 7.40 (t, 2H), 7.18 (t, 1H), 3.42 (s, 2H), 2.52 (q, 2H), 1.27 (t, 3H).

### Example 8

### 2-Cyclohexyl-1-methyl-2,4-dihydro-pyrazol-3-one

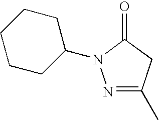

2-Cyclohexyl-1-methyl-2,4-dihydro-pyrazol-3-one was synthesized with general procedure from cyclohexyl hydrazine HCl (5.0 g, 33.2 mmol), ethyl acetoacetate (4.32 g, 33.2 mmol) and acetic acid (50 ml) was used to give 5.79 g (97%) as a pale yellow solid. ¹H NMR (300 MHz, CDCl₃) δ (ppm): 3.96-4.07 (m, 1H), 3.22 (s, 2H), 2.09 (s, 3H), 1.65-1.87 (m, 6H), 1.21-1.43 (m, 4H).

### Example 9

### 2-Cyclopentyl-1-methyl-2,4-dihydro-pyrazol-3-one

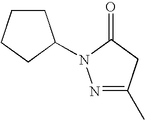

2-Cyclopentyl-1-methyl-2,4-dihydro-pyrazol-3-one was synthesized with general procedure from cyclopentyl hydrazine HCl (5.0 g, 36.6 mmol), ethyl acetoacetate (4.76 g, 36.6 mmol) and acetic acid (50 ml) was used to give 5.50 g (90%) as a brown solid. ¹H NMR (300 MHz, CDCl₃) δ (ppm): 4.51-4.62 (m, 1H), 3.22 (s, 2H), 2.11 (s, 3H), 1.81-2.01 (m, 5H), 1.62-1.79 (m, 3H).

### Example 10

### 2-Isopropyl-1-methyl-2,4-dihydro-pyrazol-3-one

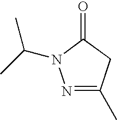

2-Isopropyl-1-methyl-2,4-dihydro-pyrazol-3-one was synthesized with general procedure from isopropyl-hydrazine (5.0273 g, 45.46 mmol), ethyl acetoacetate (5.92 g, 45.46 mmol) and acetic acid (60 ml). ¹H NMR (300 MHz, CDCl₃) δ (ppm): 3.89 (q, 1H), 2.03 (d, 2H), 1.86 (s, 3H), 1.20 (m, 6H).

### General Procedure B

A reaction mixture containing hydrazine (2 mmol), methyl acetoacetate (2 mmol) molecular sieves (4A) and toluene (4 mL) was stirred at 110 C. After 17 h, the reaction mixture was cooled to RT. To this mixture acetonitrile (1 mL) and iodomethane (6 mmol) were added successively and stirred at 110 C for additional 17 h. TLC (Silicagel, 20:1 CHCl₃:MeOH) indicated the formation of product. The solution was taken up in dichloromethane and washed with satd. aq NaCl soln. The combined organic layers were dried over MgSO₄ and concentrated. The residue was subjected to column chromatography (Silicagel, 20:1 CHCl₃:MeOH) to give the product.

Intermediate compounds of Examples 11 and 12 were synthesized using a method analogous to general procedure B for pyrazolone ring formation.

### Example 11

### 2-(2-Chloro-phenyl)-1,5-dimethyl-1,2-dihydro-pyrazol-3-one

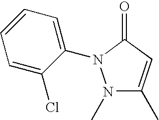

2-(2-Chloro-phenyl)-1,5-dimethyl-1,2-dihydro-pyrazol-3-one was obtained with 2-chlorophenyl hydrazine (2 mmol, 0.366 g), methyl acetoacetate (2 mmol, 0.215 mL), toluene (4 mL), iodomethane (5 mmol, 0.307 mL) and acetonitrile (2 mL). The product was purified using column chromatography to afford solid product (0.317 g, 76%). ¹H NMR (300 MHz, CDCl₃): [symbol] (ppm) 7.39-7.60 (m, 4H), 5.38 (s, 1H), 3.05 (s, 3H), 2.24 (s, 3H).

### Example 12

### 2-(4-Chloro-phenyl)-1,5-dimethyl-1,2-dihydro-pyrazol-3-one

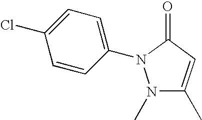

2-(4-Chloro-phenyl)-1,5-dimethyl-1,2-dihydro-pyrazol-3-one was obtained with 4-chlorophenyl hydrazine hydrochloride (2 mmol, 0.358 g), methyl acetoacetate (2 mmol, 0.215 mL), toluene (4 mL), iodomethane (5 mmol, 0.307 mL) and acetonitrile (2 mL). The product was purified using column chromatography to afford solid product (0.250 g, 57%). ¹H NMR (300 MHz, CDCl₃): [symbol] (ppm) 7.44 (dd, 2H), 7.29 (dd, 2H), 5.41 (s, 1H), 3.04 (s, 3H), 2.22 (s, 3H).

### Example 13

### 2-(3-Chloro-phenyl)-1,5-dimethyl-1,2-dihydro-pyrazol-3-one

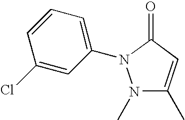

2-(3-Chloro-phenyl)-1,5-dimethyl-1,2-dihydro-pyrazol-3-one was obtained with 3-chlorophenyl hydrazine hydrochloride (2 mmol, 0.358 g), methyl acetoacetate (2 mmol, 0.215 mL), toluene (4 mL), iodomethane (5 mmol, 0.307 mL) and acetonitrile (2 mL). The product was purified using column chromatography to afford solid product (0.203 g, 46%). ¹H NMR (300 MHz, CDCl₃): [symbol] (ppm) 7.22-7.44 (m, 4H), 5.41 (s, 1H), 3.81 (s, 3H), 3.04 (s, 3H), 2.24 (s, 3H).

### Example 14

### 2-(3-Methoxy-phenyl)-1,5-dimethyl-1,2-dihydro-pyrazol-3-one

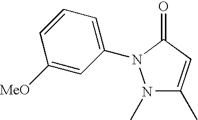

2-(3-Methoxy-phenyl)-1,5-dimethyl-1,2-dihydro-pyrazol-3-one was obtained with 3-methoxyphenyl hydrazine hydrochloride (2 mmol, 0.349 g), methyl acetoacetate (2 mmol, 0.215 mL), toluene (4 mL), iodomethane (5 mmol, 0.307 mL) and acetonitrile (2 mL). The product was purified using column chromatography to afford solid product (0.120 g, 28%). ¹H NMR was not recorded for this product.

### Example 15

### 2-(4-Methoxy-phenyl)-1,5-dimethyl-1,2-dihydro-pyrazol-3-one

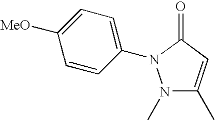

2-(4-Methoxy-phenyl)-1,5-dimethyl-1,2-dihydro-pyrazol-3-one was obtained with 4-methoxyphenyl hydrazine hydrochloride (2 mmol, 0.349 g), methyl acetoacetate (2 mmol, 0.215 mL), toluene (4 mL), iodomethane (5 mmol, 0.307 mL) and acetonitrile (2 mL). The product was purified using column chromatography to afford solid product (0.18 g, 41%). ¹H NMR (300 MHz, CDCl₃): [symbol] (ppm) 7.32 (d, 2H), 6.98 (d, 2H), 5.41 (s, 1H), 3.82 (s, 3H), 3.04 (s, 3H), 2.24 (s, 3H).

### Alkylation

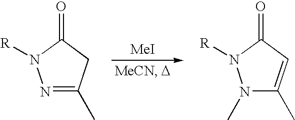

### General Procedure

In a stainless steel pressure bomb the pyrazalone (1.0 equiv.) in acetonitrile was set stirring with iodomethane (5.0 equiv.) in a 120° C. oil bath overnight. The crude product was added to saturated sodium bicarbonate and then extracted four times into ethyl acetate. After concentration, the crude product was chromatographed in a mixture of dichloromethane and methanol. NMR was used to determine the purity of the isolated compounds.

Intermediate compounds of Examples 16 through 28 were synthesized using a method analogous to the above general procedure for methylation.

### Example 16

### 1,5-Dimethyl-2-phenyl-1,2-dihydropyrazol-3-one

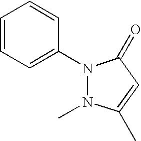

1,5-Dimethyl-2-phenyl-1,2-dihydropyrazol-3-one was obtained from 5-methyl-2-phenyl-1,2-dihydropyrazol-3-one (3.52 g, 20 mmol) and iodomethane (3.38 mL, 60 mmol) in acetonitrile (20 mL) as an off-white solid (2.2 g, 58%). ¹H NMR (300 MHz, CDCl₃): δ(ppm) 7.26-7.49 (m 5H), 5.41 (s, 1H), 3.07 (s, 3H), 2.25 (s, 3H).

### Example 17

### 1-Ethyl-5-methyl-2-phenyl-1,2-dihydropyrazol-3-one

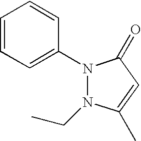

1-Ethyl-5-methyl-2-phenyl-1,2-dihydropyrazol-3-one was obtained from 5-methyl-2-phenyl-1,2-dihydropyrazol-3-one (3.52 g, 20 mmol) and iodoethane (4.8 mL, 60 mmol) in acetonitrile (20 mL) as an off-white solid (1.6 g, 35%). ¹H NMR (300 MHz, CDCl₃): δ(ppm) 7.26-7.49 (m, 5H), 5.44 (s, 1H), 3.58 (q, 2H), 2.25 (s, 3H), 0.89 (t, 3H).

### Example 18

### 2-(4-Fluorophenyl)-1,5-dimethyl-1,2-dihydropyrazol-3-one

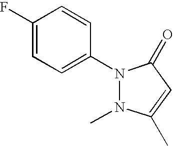

2-(4-Fluorophenyl)-1,5-dimethyl-1,2-dihydropyrazol-3-one was obtained from 2-(4-fluorophenyl)-5-methyl-2,4-dihydropyrazol-3-one (2.77 g, 14.41 mmol) in acetonitrile (50 mL) and iodomethane (4.49 mL, 72.06 mmol) as an off-white solid. The crude product was chromatographed in 5% methanol and dichloromethane to yield an off-white solid 2.23 g (75%). ¹H NMR (300 MHz, CDCl₃): δ(ppm) 7.39-7.33 (m, 2H), 7.20-7.14 (m, 2H), 5.32 (s, 1H), 3.07 (s, 3H), 2.25 (s, 3H).

### Example 19

### 2-(4-Chloro-phenyl)-1,5-dimethyl-1,2-dihydropyrazol-3-one

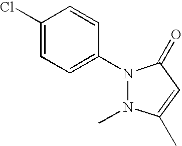

2-(4-Chloro-phenyl)-1,5-dimethyl-1,2-dihydropyrazol-3-one was obtained from (4-chloro-phenyl)-5-methyl-2,4-dihydro-pyrazol-3-one (1.5 g, 7.189 mmol), iodomethane (10.2 g, 71.89 mmol), and acetonitrile (30 ml) in 84.9% yield. ¹H NMR (300 MHz, CDCl₃) δ (ppm): 7.44 (d, 2H), 7.35 (d, 2H), 5.44 (s, 1H), 3.09 (s, 3H), 2.27 (s, 3H).

### Example 20

### 2-(3-Chloro-4-fluoro-phenyl)-1,5-dimethyl-1,2-dihydropyrazol-3-one

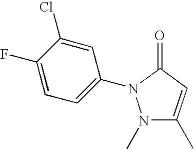

2-(3-Chloro-4-fluoro-phenyl)-1,5-dimethyl-1,2-dihydropyrazol-3-one was obtained from (3-chloro-4-fluoro-phenyl)-5-methyl-2,4-dihydro-pyrazol-3-one (0.735 g, 3.2 mmol), iodomethane (2.3 g, 16.2 mmol), and acetonitrile (7 ml) in 46% yield. ¹H NMR (300 MHz, CDCl₃) δ (ppm): 7.37 (d, 1H), 7.20 (d, 2H), 5.32 (s, 1H), 3.05 (s, 3H), 2.21 (s, 3H).

### Example 21

### 2-(4-Chloro-phenyl)-1-ethyl-5-methyl-1,2-dihydro-pyrazol-3-one

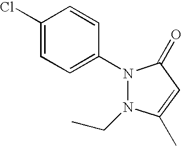

2-(4-Chloro-phenyl)-1-ethyl-5-methyl-1,2-dihydro-pyrazol-3-one was synthesized from -(4-Chloro-phenyl)-5-methyl-2,4-dihydro-pyrazol-3-one (1.5 g, 4.792 mmol), iodoethane (3.737 g, 23.965 mmol), and acetonitrile (20 ml) to give 43.2% yield product. ¹H NMR (300 MHz, CDCl₃) δ (ppm): 7.33 (m, 4H), 5.34 (s, 1H), 3.49 (q, 2H), 2.17 (s, 3H), 0.79 (t, 3H).

### Example 22

### 1,5-Dimethyl-2-(4-trifluoromethylphenyl)-1,2-dihydropyrazol-3-one

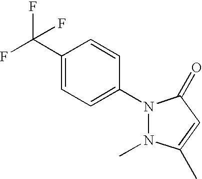

1,5-Dimethyl-2-(4-trifluoromethylphenyl)-1,2-dihydropyrazol-3-one was obtained from 5-methyl-2-(4-trifluoromethylphenyl)-2,4-dihydropyrazol-3-one (3.47 g, 14.3 mmol) in acetonitrile (50 mL) and iodomethane (4.46 mL, 71.6 mmol) as a brown solid. The crude product was chromatographed in 5% methanol and dichloromethane to yield a brown solid 2.28 g (62%). ¹H NMR (300 MHz, CDCl₃): δ(ppm) 7.74 (d, 2H), 7.55 (d, 2H), 5.50 (s, 1H), 3.11 (s, 3H), 2.29 (s, 3H).

### Example 23

### 1,5-Dimethyl-2-(4-trifluoromethoxyphenyl)-1,2-dihydropyrazol-3-one

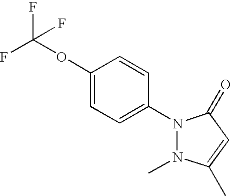

1,5-Dimethyl-2-(4-trifluoromethoxyphenyl)-1,2-dihydropyrazol-3-one was obtained from 5-methyl-2-(4-trifluoromethoxyphenyl)-2,4-dihydropyrazol-3-one (1.0 g, 3.87 mmol) in acetonitrile (40 mL) and iodomethane (1.207 mL, 19.4 mmol) as an off-white solid. The crude product was chromatographed in 1% methanol and dichloromethane to yield an off-white solid 302.5 mg (29%). ¹H NMR (300 MHz, CDCl₃): δ(ppm) 7.32 (d, 2H), 7.20 (d, 2H), 5.27 (s, 1H), 2.96 (s, 3H), 2.13 (s, 3H).

### Example 24

### 5-Ethyl-1-methyl-2-phenyl-1,2-dihydropyrazol-3-one

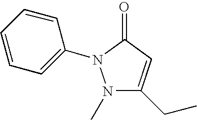

5-Ethyl-1-methyl-2-phenyl-1,2-dihydropyrazol-3-one was obtained from 5-ethyl-2-phenyl-2,4-dihydro-pyrazol-3-one (6.5 g, 34.5 mmol) in acetonitrile (50 mL) and iodomethane (16 mL, 259 mmol). The crude product was chromatographed in 5% methanol and dichloromethane to yield a brown oil 5.95 g (73%). ¹H NMR (300 MHz, CDCl₃): δ(ppm) 7.36-7.48 (m, 4H), 7.27 (t, 1H), 5.42 (s, 1H), 3.05 (s, 3H), 2.54 (q, 2H), 1.30 (t, 3H).

### Example 25

### 2-Cyclohexyl-1,5-dimethyl-1,2-dihydro-pyrazol-3-one

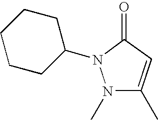

2-Cyclohexyl-1,5-dimethyl-1,2-dihydro-pyrazol-3-one was synthesized from cyclohexyl-1-methyl-2,4-dihydro-pyrazol-3-one (2.75 g, 15.26 mmol), iodomethane (16.25 g, 114.5 mmol), and acetonitrile (30 ml) and chromatographed with 50% ethyl acetate and hexanes to yield 570 mg (20%) of a reddish-brown oil. ¹H NMR (300 MHz, CDCl₃) δ (ppm): 5.19 (s, 1H), 4.00-4.10 (m, 1H), 3.16 (s, 3H), 2.07 (s, 3H), 1.78-1.89 (m, 6H), 1.63 (d, 1H), 1.13-1.35 (m, 3H).

### Example 26

### 2-Cyclohexyl-1-ethyl-5-methyl-1,2-dihydro-pyrazol-3-one

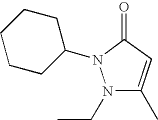

2-Cyclohexyl-1-ethyl-5-methyl-1,2-dihydro-pyrazol-3-one was synthesized from cyclohexyl-1-methyl-2,4-dihydro-pyrazol-3-one (1.0 g, 5.55 mmol), iodoethane (8.66 g, 55.5 mmol), and tetrahydrofuran (14 ml) and chromatographed with 3% methanol and ethyl acetate to yield 70 mg (6%) of a brown solid. ¹H NMR (300 MHz, CDCl₃) δ (ppm): 5.28 (s, 1H), 3.97-4.06 (m, 1H), 3.68 (q, 2H), 2.11 (s, 3H), 1.66-1.94 (m, 6H), 1.56 (d, 1H), 1.17-1.42 (m, 3H), 0.99 (t, 3H).

### Example 27

### 2-Cyclopentyl-1,5-dimethyl-1,2-dihydro-pyrazol-3-one

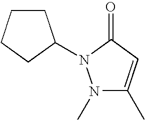

2-Cyclopentyl-1,5-dimethyl-1,2-dihydro-pyrazol-3-one was synthesized from cyclopentyl-1-methyl-2,4-dihydro-pyrazol-3-one (3.4 g, 20.45 mmol), iodomethane (29.03 g, 204.5 mmol), and acetonitrile (30 ml) and chromatographed with 50% ethyl acetate and hexanes to yield 1.42 g (38%) of an oil. ¹H NMR (300 MHz, CDCl₃) δ (ppm): 5.23 (s, 1H), 4.64 (q, 1H), 3.20 (s, 3H), 2.11 (s, 3H), 1.92-1.97 (m, 3H), 1.83-1.87 (m, 2H), 1.60-1.63 (m, 2H).

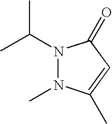

### Example 28

### 2-Isopropyl-1,5-dimethyl-1,2-dihydro-pyrazol-3-one

2-Isopropyl-1,5-dimethyl-1,2-dihydro-pyrazol-3-one was synthesized from isopropyl-1-methyl-2,4-dihydro-pyrazol-3-one (2.5 g, 17.833 mmol), iodomethane (12.656 g, 89.16 mmol), and acetonitrile (35 ml) in 48% yield. ¹H NMR (300 MHz, CDCl₃) δ (ppm): 5.24 (s, 1H), 4.56 (m, 1H), 3.20 (s, 3H), 3.19 (s, 3H), 1.39 (t, 6H).

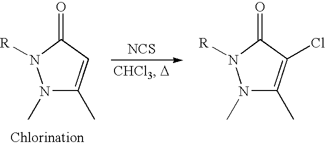

### General Procedure

The pyrazolone (1.0 equiv.) in chloroform and N-chlorosuccinimide (1.1 equiv.) were refluxed at 50° C. for 30 minutes. The solution was concentrated in vacuo. The crude mixture was dissolved in dichloromethane and washed three times with water. The desired compound was purified using column chromatography in a mixture of methanol and dichloromethane. NMR was used to confirm the purity of the isolated samples.

Intermediate compounds of Examples 29 through 39 were synthesized using a method analogous to the above general procedure for chlorination.

### Example 29

### 4-Chloro-2-(4-fluorophenyl)-1,5-dimethyl-1,2-dihydropyrazol-3-one

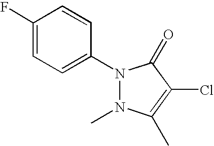

4-Chloro-2-(4-fluorophenyl)-1,5-dimethyl-1,2-dihydropyrazol-3-one was obtained from 2-(4-fluorophenyl)-1,5-dimethyl-1,2-dihydropyrazol-3-one (2.23 g, 10.81 mmol) in chloroform (42 mL) and N-chlorosuccinamide (1.59 g, 11.89 mmol) as an off-white solid. The crude product was chromatographed in 2% methanol and dichloromethane to yield an off-white solid 2.33 g (89%). ¹H NMR (300 MHz, CDCl₃): δ(ppm) 7.41-7.35 (m, 2H), 7.20-7.15 (m, 2H), 3.06 (s, 3H), 2.30 (s, 3H).

### Example 30

### 4-Chloro-2-(4-chloro-phenyl)-1,5-dimethyl-1,2-dihydro-pyrazol-3-one

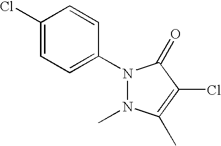

4-Chloro-2-(4-chloro-phenyl)-1,5-dimethyl-1,2-dihydro-pyrazol-3-one was synthesized from (4-Chloro-phenyl)-1,5-dimethyl-1,2-dihydropyrazol-3-one (1.36 g, 6.107 mmol), N-chlorosuccinimide (0.897 g, 6.778 mmol), and chloroform (35 ml) in 67% yield. The ¹H NMR (300 MHz, CDCl₃) δ (ppm): 7.44 (m, 2H), 7.35 (m, 2H), 3.01 (s, 3H), 2.31 (s, 3H).

### Example 31

### 4-Chloro-2-(3-chloro-4-fluoro-phenyl)-1,5-dimethyl-1,2-dihydro-pyrazol-3-one

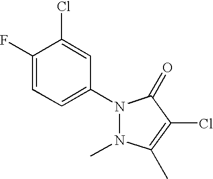

4-Chloro-2-(3-chloro-4-fluoro-phenyl)-1,5-dimethyl-1,2-dihydro-pyrazol-3-one was synthesized from (3-chloro-4-fluoro-phenyl)-1,5-dimethyl-1,2-dihydropyrazol-3-one (0.36 g, 1.5 mmol), N-chlorosuccinimide (0.220 g, 1.65 mmol), and chloroform (10 ml) to yield 176 mg (43%) of an off-white solid. ¹H NMR (300 MHz, CDCl₃) δ (ppm): 7.44-7.47 (m, 1H), 7.22-7.32 (m, 2H), 3.06 (s, 3H), 2.29 (s, 3H).

### Example 32

### 4-Chloro-2-(4-chloro-phenyl)-1-ethyl-5-methyl-1,2-dihydro-pyrazol-3-one

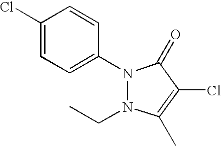

4-Chloro-2-(4-chloro-phenyl)-1-ethyl-5-methyl-1,2-dihydro-pyrazol-3-one was synthesized from -(4-Chloro-phenyl)-1-ethyl-5-methyl-1,2-dihydro-pyrazol-3-one (0.49 g, 2.07 mmol), N-chlorosuccinimide (0.304 g, 2.27 mmol) in 64.6% yield. The ¹H NMR (300 MHz, CDCl₃) δ (ppm): 7.46 (d, 2H), 7.39 (d, 2H), 3.59 (q, 2H), 2.30 (s, 3H), 0.87 (t, 3H).

### Example 33

### 4-Chloro-1,5-dimethyl-2-(4-trifluoromethylphenyl)-1,2-dihydropyrazol-3-one

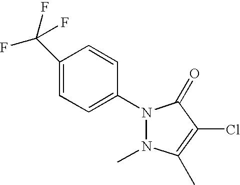

4-Chloro-1,5-dimethyl-2-(4-trifluoromethylphenyl)-1,2-dihydropyrazol-3-one was obtained from 1,5-dimethyl-2-(4-trifluoromethylphenyl)-1,2-dihydropyrazol-3-one (2.28 g, 8.91 mmol) in chloroform (40 mL) and N-chlorosuccinimide (1.30 mg, 9.8 mmol) as an off-white solid. The crude product was chromatographed in 2% methanol and dichloromethane to yield an off-white solid 1.36 g (52%). ¹H NMR (300 MHz, CDCl₃): δ (ppm) 7.75 (d, 2H), 7.58 (d, 2H), 3.10 (s, 3H), 2.34 (s, 3H).

### Example 34

### 4-Chloro-1,5-dimethyl-2-(4-trifluoromethoxyphenyl)-1,2-dihydropyrazol-3-one

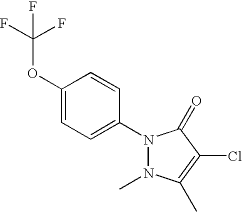

4-Chloro-1,5-dimethyl-2-(4-trifluoromethoxyphenyl)-1,2-dihydropyrazol-3-one was obtained from 1,5-dimethyl-2-(4-trifluoromethoxyphenyl)-1,2-dihydropyrazol-3-one (302 mg, 1.109 mmol) in chloroform (20 mL) and N-chlorosuccinamide (163 mg, 1.22 mmol) as a yellow sticky solid. The crude product was chromatographed in 2% methanol and dichloromethane to yield an off-white solid 250 mg (74%). ¹H NMR (300 MHz, CDCl₃): δ (ppm) 7.42 (d, 2H), 7.29 (d, 2H), 3.04 (s, 3H), 2.26 (s, 3H).

### Example 35

### 4-Chloro-5-ethyl-1-methyl-2-phenyl-1,2-dihydro-pyrazol-3-one

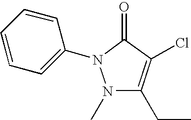

4-Chloro-5-ethyl-1-methyl-2-phenyl-1,2-dihydro-pyrazol-3-one was synthesized from 5-ethyl-1-methyl-2-phenyl-1,2-dihydropyrazol-3-one (5.95 g, 25.13 mmol), N-chlorosuccinimide (3.69 g, 27.64 mmol), and chloroform (60 ml). It was chromatographed using a mixture of ethyl acetate in hexanes to yield 4.75 g (85%) as a yellow solid. ¹H NMR (300 MHz, CDCl₃) δ (ppm): 7.32-7.50 (m, 5H), 3.08 (t, 3H), 2.71 (q, 2H), 1.31 (t, 3H).

### Example 36

### 4-Chloro-2-isopropyl-1,5-dimethyl-1,2-dihydro-pyrazol-3-one

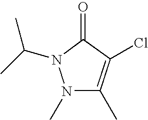

4-Chloro-2-isopropyl-1,5-dimethyl-1,2-dihydro-pyrazol-3-one was synthesized from 2-isopropyl-1,5-dimethyl-1,2-dihydro-pyrazol-3-one (1.19 g, 7.72 mmol) and N-chlorosuccinimide (1.13 g, 8.49 mmol) in chloroform (35 mL). The crude product was purified by column chromatography in 1% methanol and dichloromethane to yield 359.9 mg (26%) of the product as a dark red oil. ¹H NMR (300 MHz, CDCl₃): δ (ppm) 4.51 (sept, 1H), 3.19 (s, 3H), 2.178 (s, 3H), 1.43 (d, 6H).

### Example 37

### 4-Chloro-2-cyclohexyl-1,5-dimethyl-1,2-dihydro-pyrazol-3-one

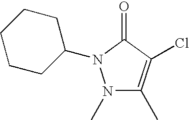

4-Chloro-2-cyclohexyl-1,5-dimethyl-1,2-dihydro-pyrazol-3-one was synthesized from 2-cyclohexyl-1,5-dimethyl-1,2-dihydropyrazol-3-one (0.57 g, 2.93 mmol), N-chlorosuccinimide (0.43 g, 3.22 mmol), and chloroform (10 ml) in to yield 0.650 g (97%) as a white solid. ¹H NMR (300 MHz, CDCl₃) δ (ppm): 3.91-4.01 (m, 1H), 3.15 (s, 3H), 2.12 (s, 3H), 1.74-1.88 (m, 6H), 1.63 (d, 1H), 1.16-1.32 (m, 3H).

### Example 38

### 4-Chloro-2-cyclohexyl-1-ethyl-5-methyl-1,2-dihydro-pyrazol-3-one

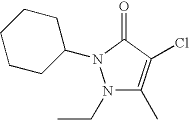

4-Chloro-2-cyclohexyl-1-ethyl-5-methyl-1,2-dihydro-pyrazol-3-one was synthesized from 2-cyclohexyl-1-ethyl-5-methyl-1,2-dihydropyrazol-3-one (70 mg, 0.222 mmol), N-chlorosuccinimide (33 mg, 0.244 mmol), and chloroform (3 ml) in to yield 59 mg (73%) as an oil. ¹H NMR (300 MHz, CDCl₃) δ (ppm): 3.92-4.00 (m, 1H), 3.69 (q, 2H), 2.20 (s, 3H), 1.82-1.98 (m, 6H), 1.67 (d, 1H), 1.20-1.35 (m, 3H), 0.95 (t, 3H).

### Example 39

### 4-Chloro-2-cyclopentyl-1,5-dimethyl-1,2-dihydro-pyrazol-3-one

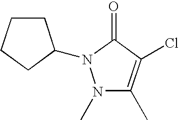

4-Chloro-2-cyclopentyl-1,5-dimethyl-1,2-dihydro-pyrazol-3-one was synthesized from 2-cyclopentyl-1,5-dimethyl-1,2-dihydro-pyrazol-3-one (0.7 g. 3.8 mmol) and N-chlorosuccinimide (0.56 g. 4.18 mmol) in chloroform (12 ml). The crude product was purified by column chromatography in 10% acetone, CH₂Cl₂ to yield 256 mg (31.38%) of the product as a yellow oil. ¹H NMR (300 MHz, CDCl₃) δ ppm: 1.62 (m, 2H), 1.87-2.00 (m, 6H), 2.02 (s, 3H), 3.22 (s, 3H), 4.57 (quintet, 1H).

### Bromination

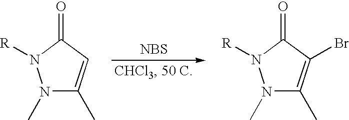

### General Procedure

The pyrazolone (1.0 equiv.) in chloroform and N-bromosuccinimide (1.1 equiv.) were refluxed at 50° C. for 30 minutes. The solution was concentrated in vacuo. The crude mixture was dissolved in dichloromethane and washed three times with water. The desired compound was purified using column chromatography in a mixture of methanol and dichloromethane. NMR was used to confirm the purity of the isolated samples.

Intermediate compounds of Examples 40 and 41 were synthesized using a method analogous to the above general procedure for chlorination.

### Example 40

### 4-Bromo-2-cyclohexyl-1,5-dimethyl-1,2-dihydro-pyrazol-3-one

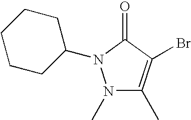

4-Bromo-2-cyclohexyl-1,5-dimethyl-1,2-dihydro-pyrazol-3-one was synthesized from 2-cyclohexyl-1,5-dimethyl-1,2-dihydro-pyrazol-3-one (1.49 g, 7.67 mmol), N-bromosuccinimide (1.50 g, 8.44 mmol) and chloroform (30 mL) to yield 1.97 g (94%) of a beige solid. ¹H NMR (300 MHz, CDCl₃) δ (ppm): 3.99-4.10 (m, 1H), 3.22 (s, 3H), 2.19 (s, 3H), 1.96 (qd, 2H), 1.72 (t, 4H), 1.69 (d, 1H), 1.22-1.39 (m, 3H).

### Example 41

### 4-Bromo-2-cyclopentyl-1,5-dimethyl-1,2-dihydro-pyrazol-3-one

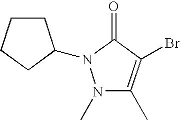

4-Bromo-2-cyclopentyl-1,5-dimethyl-1,2-dihydro-pyrazol-3-one was synthesized from 2-cyclopentyl-1,5-dimethyl-1,2-dihydro-pyrazol-3-one (0.7276 g, 4.04 mmol) and N-bromosuccinimide (0.719 g, 4.04 mmol) in chloroform (14 mL). The crude product was purified by column chromatography in a solution of 30% acetone and hexanes to yield 1.047 g (90%) of the product as a yellow oil. ¹H NMR (300 MHz, CDCl₃) δ ppm: 1.53-1.50 (m, 2H), 1.91-1.77 (m, 6H), 2.09 (s, 3H), 3.15 (s, 3H), 4.47 (quintet, 1H).

### Hydroxylation of Alpha Bromo Pyrazolones

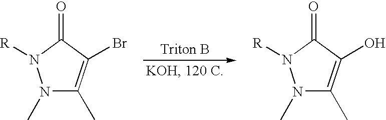

### General Procedure

The bromopyrazolone (1.0 equiv.), 3.0 M potassium hydroxide (aq., 20 equiv.) and benzyltrimethyl ammonium hydroxide (40% aq., 4.5 equiv.) in toluene was stirred at 120° C. for 48 hours. The pH of the reaction was adjusted to 6 with HCl and partitioned between dichloromethane and water. The organic was dried over anhydrous sodium sulphate and purified by column chromatography on silica gel. ¹H-NMR was used to confirm the purity of the isolated samples.

Intermediate compounds of Examples 42 and 43 were synthesized using a method analogous to the above general procedure for hydroxylation of alpha bromo pyrazolones.

### Example 42

### 2-Cyclohexyl-4-hydroxy-1,5-dimethyl-1,2-dihydro-pyrazol-3-one

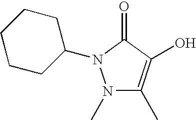

2-Cyclohexyl-4-hydroxy-1,5-dimethyl-1,2-dihydro-pyrazol-3-one was synthesized from 4-bromo-2-cyclohexyl-1,5-dimethyl-1,2-dihydro-pyrazol-3-one (500 mg, 1.83), benzyltrimethyl ammonium hydroxide (1.5 mL, 8.22 mmol) and potassium hydroxide (12.2 mL, 36.6 mmol) to yield 38 mg (10%) of a pale yellow semi-solid. ¹H NMR (300 MHz, CDCl₃) δ (Ppm): 9.21 (s, 1H), 3.89-3.99 (m, 1H), 2.95 (s, 3H), 1.81-1.98 (m, 7H), 1.67 (d, 1H), 1.22-1.36 (t, 3H).

### Example 43

### 2-Cyclopentyl-4-hydroxy-1,5-dimethyl-1,2-dihydro-pyrazol-3-one

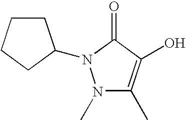

2-Cyclopentyl-4-hydroxy-1,5-dimethyl-1,2-dihydro-pyrazol-3-one was synthesized from 4-bromo-2-cyclopentyl-1,5-dimethyl-1,2-dihydro-pyrazol-3-one (0.943 g, 3.639 mmol), potassium hydroxide (72.78 mmol, 12.13 mL of 6.0M solution), and Triton B (7.278 mmol, 1.12 mL) were set stirring in 14 mL of methanol. The reaction yielded 586.4 mg (68.1%) of crude product.

### Methylation of Alpha Hydroxyl Pyrazolones

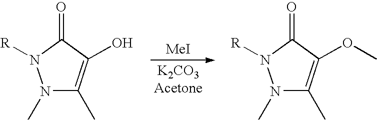

### General Procedure

The hydroxypyrazolone (1.0 equiv.), iodomethane (2.5 equiv.) and potassium carbonate (5.0 equiv.) in acetone was allowed to stir at reflux (65° C.) overnight. The solvent is removed in vacuo and the remaining mixture is dissolved in ethyl acetate, washed thrice with water and once with brine. The organic layer is dried over anhydrous sodium sulfate. The product is purified by column chromatography in 60% ethyl acetate and hexanes. ¹H-NMR was used to confirm the purity of the isolated samples.

Intermediate compounds of Examples 44 through 46 were synthesized using a method analogous to the above general procedure for alkylation of alpha hydroxyl pyrazolones.

### Example 44

### 4-Methoxy-1,5-dimethyl-2-phenyl-1,2-dihydro-pyrazol-3-one

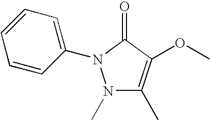

4-Methoxy-1,5-dimethyl-2-phenyl-1,2-dihydro-pyrazol-3-one was obtained from 4-hydroxyantipyrine (1.0 g, 4.896 mmol), iodomethane (1.74 g, 12.24 mmol), and potassium carbonate (3.38 g, 24.48 mmol) in acetone (30 mL) as a pale yellow solid (697.7 mg, 65%). 1H NMR (300 MHz, CDCl₃): δ(ppm) 7.45 (m, 4H), 7.28 (m, 1H), 3.94 (s, 3H), 2.93 (s, 3H), 2.20 (s, 3H).

### Example 45

### 2-Cyclohexyl-4-methoxy-1,5-dimethyl-1,2-dihydro-pyrazol-3-one

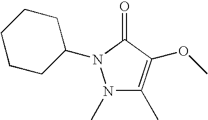

2-Cyclohexyl-4-methoxy-1,5-dimethyl-1,2-dihydro-pyrazol-3-one was obtained from 2-cyclohexyl-4-hydroxy-1,5-dimethyl-1,2-dihydro-pyrazol-3-one (38 mg, 0.181 mmol), iodomethane (64 mg, 0.453 mmol) and potassium carbonate (125 mg, 0.905 mmol) in acetone as a yellow oil (20.3 mg, 50%). 1H NMR (300 MHz, CDCl₃): δ (ppm) 3.89-3.98 (m, 1H), 3.87 (s, 3H), 2.99 (s, 3H), 2.05 (s, 3H), 1.80-1.96 (m, 7H), 1.66 (d, 1H), 1.21-1.37 (m, 3H).

### Example 46

### 2-Cyclopentyl-4-methoxy-1,5-dimethyl-1,2-dihydro-pyrazol-3-one

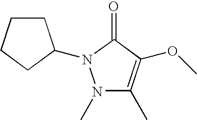

2-Cyclopentyl-4-methoxy-1,5-dimethyl-1,2-dihydro-pyrazol-3-one was synthesized from 2-Cyclopentyl-4-hydroxyl-1,5-dimethyl-1,2-dihydro-pyrazol-3-one (0.487 g, 2.48 mmol), iodomethane (0.88 g, 6.20 mmol) and potassium carbonate (1.713 g, 12.4 mmol) in acetone (12 mL). The crude material was purified by column chromatography in a solution of 15% acetone and hexanes to yield 204.4 mg (40%) of product. ¹H NMR (300 MHz, CDCl₃) δ ppm: 1.54-1.53 (m, 2H), 1.98-1.79 (m, 6H), 1.99 (s, 3H), 2.93 (s, 3H), 3.79 (s, 3H), 4.42 (quintet, 1H).

### Ethylation of Alpha Hydroxyl Pyrazolones

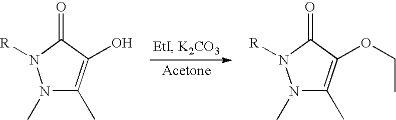

### General Procedure

The hydroxypyrazolone (1.0 equiv.), iodoethane (2.5 equiv.) and potassium carbonate (5.0 equiv.) in acetone was allowed to stir at reflux (65° C.) overnight. The solvent is removed in vacuo and the remaining mixture is dissolved in ethyl acetate, washed thrice with water and once with brine. The organic layer is dried over anhydrous sodium sulfate. The product is purified by column chromatography in 60% ethyl acetate and hexanes. ¹H-NMR was used to confirm the purity of the isolated samples.

Intermediate compound of Example 47 were synthesized using a method analogous to the above general procedure for alkylation of alpha hydroxyl pyrazolones.

### Example 47

### 4-Ethoxy-1,5-dimethyl-2-phenyl-1,2-dihydro-pyrazol-3-one

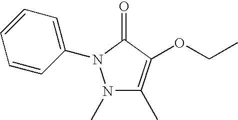

4-Ethoxy-1,5-dimethyl-2-phenyl-1,2-dihydro-pyrazol-3-one was obtained from 4-hydroxyantipyrine (1.0 g, 4.9 mmol), iodoethane (1.91 g, 12.25 mmol), and potassium carbonate (3.38 g, 24.5 mmol) in acetone (15 mL) as a yellow solid (1.09 g, 96%). 1H NMR (300 MHz, CDCl₃): δ (ppm) 7.44 (d, 4H), 7.25-7.29 (m, 1H), 4.21 (q, 2H), 2.92 (s, 3H), 2.20 (s, 3H) 1.32 (q, 3H).

### Synthesis of Alpha-Difluoromethoxy Pyrazalones

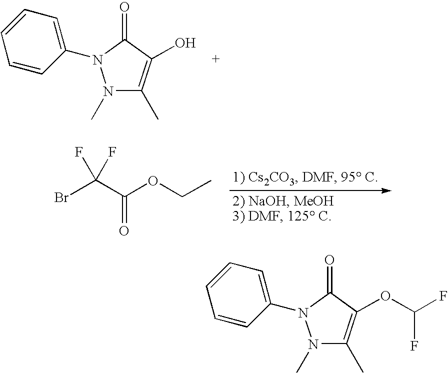

### Example 48

### 4-Difluoromethoxy-1,5-dimethyl-2-phenyl-1,2-dihydro-pyrazol-3-one

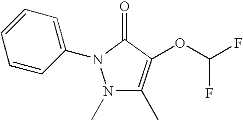

4-Difluoromethoxy-1,5-dimethyl-2-phenyl-1,2-dihydro-pyrazol-3-one was synthesized by the following procedure. The 4-hydroxyantipyrine (1.00 g, 4.90 mmol, 1.0 equiv.), and cesium carbonate (1.60 g, 4.90 mmol, 1.0 equiv.) in DMF (15 mL) were allowed to stir at room temperature for 15 minutes followed by 15 minutes at 95° C. The mixture was allowed to cool to room temperature at which time ethyl bromodifluoroacetate (789 μL, 6.12 mmol, 1.25 equiv.) was added slowly over 10 minutes. The resulting reaction mixture was allowed to stir at 95° C. Additional amounts of ethyl bromodifluoroacetate were added every 15 minutes until TLC showed the 4-hydroxyantipyrine was consumed. The mixture was partitioned between ethyl acetate and distilled water. The combined organic phases were dried over anhydrous sodium sulfate and the solvent removed in vacuo. Methanol (10 mL) was added to replace the DMF. To the solution was added 1M sodium hydroxide (1.83 mL, 1.83 mmol) and the resulting reaction mixture was allowed to stir at room temperature for one hour. The methanol was removed in vacuo and replaced by DMF (10 mL). Allowed the solution to stir for 1 hour at 100oC followed by 1 hour at 125oC. Diluted the solution with ethyl acetate and washed thrice with distilled water. Dried the organic layer over anhydrous sodium sulfate and removed the solvent in vacuo. The product was isolated by column chromatography in 50% ethyl acetate and hexanes as a yellow oil (135.1 mg, 29%). ¹H NMR (300 MHz, CDCl₃): δ(ppm) 7.32-7.51 (m, 5H), 6.89 (t, 1H), 3.05 (s, 3H), 2.27 (s, 3H).

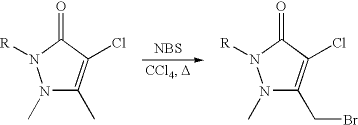

### Bromination

### General Procedure

The pyrazolone (1 equiv.) in carbon tetrachloride and N-bromosuccinimide (1.1 equiv.) was refluxed for 45 minutes. The crude reaction mixture was dissolved in dichloromethane and washed three times with water. The product was then isolated by column chromatography in a mixture of methanol and dichloromethane or ethyl acetate and hexane. NMR was used to confirm the purity of the isolated product.

Intermediate compounds of Examples 49 and 50 were synthesized using a method analogous to the above combined general procedure for chlorination and bromination.

### Example 49

### 5-Bromomethyl-4-chloro-1-methyl-2-phenyl-1,2-dihydropyrazol-3-one

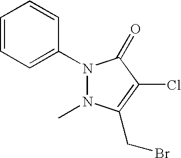

5-Bromomethyl-4-chloro-1-methyl-2-phenyl-1,2-dihydropyrazol-3-one was obtained in two steps from (1) 1,5-dimethyl-2-phenyl-1,2-dihydropyrazol-3-one (1.56 g, 8.2 mmol) and N-chlorosuccinimide (1.1 g, 8.2 mmol) in chloroform (25 ml), (2) chlorinated intermediate and N-bromosuccinimide (1.42 g, 8 mmol) in carbon tetrachloride (50 mL). The product was isolated by column chromatography in 50% ethyl acetate and hexane as an off-white solid (1.8 g, 74%). ¹H NMR (300 MHz, CDCl₃): δ (ppm) 7.37-7.54 (m, 5H), 3.21 (s, 3H), 4.41 (s, 2H).

### Example 50

### 5-Bromomethyl-4-chloro-1-ethyl-2-phenyl-1,2-dihydropyrazol-3-one

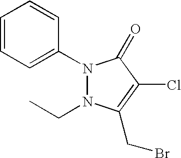

5-Bromomethyl-4-chloro-1-ethyl-2-phenyl-1,2-dihydropyrazol-3-one was obtained by two steps from (1) 1-ethyl-5-methyl-2-phenyl-1,2-dihydropyrazol-3-one (1.6 g, 7.8 mmol) and N-chlorosuccinimide (1.1 g, 8.2 mmol) in chloroform (25 ml), (2) chlorinated intermediate and N-bromosuccinimide (1.3 g, 7.3 mmol) in carbon tetrachloride (50 mL). The product was isolated by column chromatography in 50% ethyl acetate and hexane as an off-white solid (1.45 g, 60%). ¹H NMR (300 MHz, CDCl₃): δ (ppm) 7.37-7.51 (m, 5H), 4.39 (s, 2H), 3.74 (q, 2H), 0.93 (t, 3H).

Intermediate compounds of Examples 51 through 67 were synthesized using a method analogous to the above general procedure for bromination.

### Example 51

### 5-Bromomethyl-4-chloro-2-(4-fluorophenyl)-1-methyl-1,2-dihydropyrazol-3-one

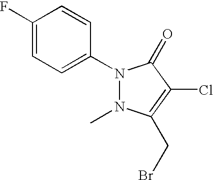

5-Bromomethyl-4-chloro-2-(4-fluorophenyl)-1-methyl-1,2-dihydropyrazol-3-one was obtained from 4-chloro-2-(4-fluorophenyl)-1,5-dimethyl-1,2-dihydropyrazol-3-one (2.33 g, 9.64 mmol) in carbon tetrachloride (82 mL) and N-bromosuccinamide (1.89 g, 10.60 mmol). The product was isolated by column chromatography in 50% ethyl acetate and hexane as an off-white solid 2.09 g (68%). ¹H NMR (300 MHz, CDCl₃): δ (ppm) 7.42-7.38 (m, 2H), 7.28-7.17 (m, 2H), 4.39 (s, 2H), 3.19 (s, 3H).

### Example 52

### 5-Bromomethyl-4-chloro-2-(4-chloro-phenyl)-1-methyl-1,2-dihydro-pyrazol-3-one

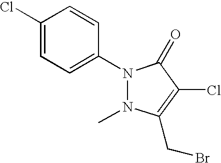

5-Bromomethyl-4-chloro-2-(4-chloro-phenyl)-1-methyl-1,2-dihydro-pyrazol-3-one was synthesized from 4-Chloro-2-(4-chloro-phenyl)-1,5-dimethyl-1,2-dihydro-pyrazol-3-one (0.5 g, 1.945 mmol), N-bromosuccinimide (0.380 g, 2.13 mmol), and carbontetrachloride (15 ml) to give 83.5% of the desired product. ¹H NMR (300 MHz, CDCl₃) δ (ppm): 7.47 (d, 2H), 7.38 (d, 2H), 4.39 (s, 2H), 3.18 (s, 3H).

### Example 53

### 5-Bromomethyl-4-chloro-2-(3-chloro-4-fluoro-phenyl)-1-methyl-1,2-dihydro-pyrazol-3-one

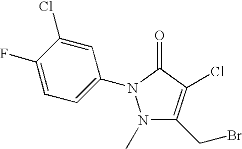

5-Bromomethyl-4-chloro-2-(3-chloro-4-fluoro-phenyl)-1-methyl-1,2-dihydro-pyrazol-3-one was synthesized from 4-chloro-2-(3-chloro-4-fluoro-phenyl)-1,5-dimethyl-1,2-dihydro-pyrazol-3-one (0.175 g, 0.64 mmol), N-bromosuccinimide (0.125 g, 0.7 mmol), and carbontetrachloride (5 ml) to give 165 mg (73%) of the desired product as a white solid. ¹H NMR (300 MHz, CDCl₃) δ (ppm): 7.40-7.43 (m, 1H), 7.20-7.27 (m, 2H), 4.34 (s, 2H), 3.15 (s, 3H).

### Example 54

### 5-Bromomethyl-4-chloro-2-(4-chloro-phenyl)-1-ethyl-1,2-dihydro-pyrazol-3-one

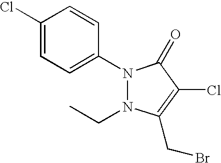

5-Bromomethyl-4-chloro-2-(4-chloro-phenyl)-1-ethyl-1,2-dihydro-pyrazol-3-one was synthesized from 4-Chloro-2-(4-chloro-phenyl)-1-ethyl-5-methyl-1,2-dihydro-pyrazol-3-one (0.363 g, 1.336 mmol), N-bromosuccinimide (0.262 g, 1.49 mmol), and carbontetrachloride (15 ml) in 68% yield. The ¹H NMR (300 MHz, CDCl₃) δ (ppm): 7.48 (d, 2H), 7.39 (d, 2H), 4.37 (s, 2H), 3.71 (q, 2H), 1.57 (s, 3H), 0.95 (t, 3H).

### Example 55

### 5-Bromomethyl-4-chloro-1-methyl-2-(4-trifluoromethylphenyl)-1,2-dihydropyrazol-3-one

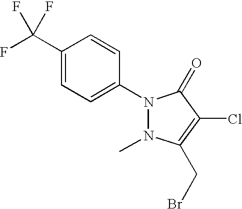

5-Bromomethyl-4-chloro-1-methyl-2-(4-trifluoromethylphenyl)-1,2-dihydropyrazol-3-one was obtained from 4-chloro-1,5-dimethyl-2-(4-trifluoromethylphenyl)-1,2-dihydropyrazol-3-one (1.36 g, 4.68 mmol) in carbon tetrachloride (45 mL) and N-bromosuccinimide (916 mg, 5.14 mmol). The product was isolated by column chromatography in 1% methanol and dichloromethane as yellow solid 437.4 mg (24%). ¹H NMR (300 MHz, CDCl₃): δ (ppm) 7.78 (d, 2H), 7.60 (d, 2H), 4.40 (s, 2H), 3.23 (s, 3H).

### Example 56

### 5-Bromomethyl-4-chloro-1-methyl-2-(4-trifluoromethoxyphenyl)-1,2-dihydropyrazol-3-one

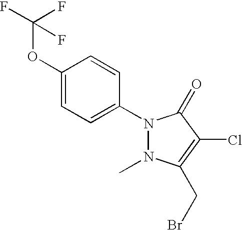

5-Bromomethyl-4-chloro-1-methyl-2-(4-trifluoromethoxyphenyl)-1,2-dihydropyrazol-3-one was obtained from 4-chloro-1,5-dimethyl-2-(4-trifluoromethoxyphenyl)-1,2-dihydropyrazol-3-one (250 mg, 0.82 mmol) in carbon tetrachloride (8 mL) and N-bromosuccinimide (160 mg, 0.897 mmol). The product was isolated by column chromatography in 2% methanol and dichloromethane as an off-white solid 179 mg (57%). ¹H NMR (300 MHz, CDCl₃): δ (ppm) 7.47-7.42 (m, 2H), 7.35-7.30 (m, 2H), 4.38 (s, 2H), 3.19 (s, 3H).

### Example 57

### 5-(1-Bromoethyl)-4-chloro-1-methyl-2-phenyl-1,2-dihydro-pyrazol-3-one

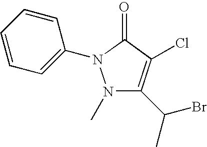

5-(1-Bromoethyl)-4-chloro-1-methyl-2-phenyl-1,2-dihydro-pyrazol-3-one was synthesized from 4-chloro-5-ethyl-1-methyl-2-phenyl-1,2-dihydro-pyrazol-3-one (1.5 g, 6.3 mmol), N-bromosuccinimide (1.23 g, 6.93 mmol), and carbon tetrachloride (30 ml). It was chromatographed using a mixture of ethyl acetate in hexanes to yield 1.6 g (80%) as a white solid. ¹H NMR (300 MHz, CDCl₃) δ (ppm): 7.34-7.53 (m, 5H), 5.24 (q, 1H), 3.23 (s, 3H), 2.14 (d, 3H).

### Example 58

### 5-Bromomethyl-4-chloro-2-cyclohexyl-1-methyl-1,2-dihydro-pyrazol-3-one

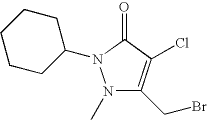

5-Bromomethyl-4-chloro-2-cyclohexyl-1-methyl-1,2-dihydro-pyrazol-3-one was synthesized from 4-chloro-2-cyclohexyl-1,5-dimethyl-1,2-dihydro-pyrazol-3-one (0.670 g, 2.9 mmol), N-bromosuccinimide (0.574 g, 3.2 mmol), and carbontetrachloride (10 ml) to give 75% of the desired product as a pale yellow solid. ¹H NMR (300 MHz, CDCl₃) δ (ppm): 4.26 (s, 2H), 3.99-4.13 (m, 1H), 3.29 (s, 3H), 1.82-2.02 (m, 6H), 1.79 (d, 1H), 1.20-1.35 (m, 3H).

### Example 59

### 5-Bromomethyl-4-chloro-2-cyclohexyl-1-ethyl-1,2-dihydro-pyrazol-3-one

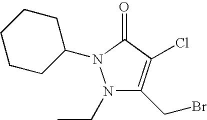

5-Bromomethyl-4-chloro-2-cyclohexyl-1-ethyl-1,2-dihydro-pyrazol-3-one was synthesized from 4-chloro-2-cyclohexyl-1-ethyl-5-methyl-1,2-dihydro-pyrazol-3-one (59 mg, 0.243 mmol), N-bromosuccinimide (48 mg, 0.267 mmol), and carbontetrachloride (2 ml) to give 72 mg (92%) of the desired product as a yellow foam. ¹H NMR (300 MHz, CDCl₃) δ (ppm): 4.26 (s, 2H), 3.81 (q, 2H), 2.01-2.09 (m, 3H), 1.86 (s, 4H), 1.69 (d, 1H), 1.22-1.36 (m, 3H), 1.08 (t, 3H).

### Example 60

### 4-Bromo-5-bromomethyl-2-cyclohexyl-1-methyl-1,2-dihydro-pyrazol-3-one

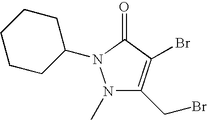

4-Bromo-5-bromomethyl-2-cyclohexyl-1-methyl-1,2-dihydro-pyrazol-3-one was synthesized from 4-bromo-2-cyclohexyl-1-ethyl-5-methyl-1,2-dihydro-pyrazol-3-one (300 mg, 1.09 mmol), N-bromosuccinimide (213 mg, 1.20 mmol), and carbontetrachloride (5 ml) to give 291 mg (76%) of the desired product as an off-white solid. ¹H NMR (300 MHz, CDCl3) δ (ppm): 4.28 (s, 2H), 4.00-4.13 (m, 1H), 3.33 (s, 3H), 2.01 (qd, 2H), 1.89 (t, 4H), 1.80 (d, 1H), 1.22-1.37 (m, 3H).

### Example 61

### 5-Bromomethyl-4-chloro-2-cyclopentyl-1-methyl-1,2-dihyrdo-pyrazol-3-one

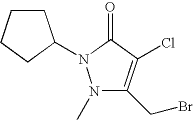

5-Bromomethyl-4-chloro-2-cyclopentyl-1-methyl-1,2-dihyrdo-pyrazol-3-one was synthesized from 4-Chloro-2-cyclopentyl-1,5-dimethyl-1,2-dihydro-pyrazol-3-one (256 mg, 1.19 mmol) and N-bromosuccinimide (0.233 mg, 1.31 mmol) in carbon tetrachloride (5.0 mL). The product was isolated by column chromatography in 10% acetone and dichloromethane to yield a yellow oil (281 mg, 80.5%). ¹H NMR (300 MHz, CDCl₃) δ ppm: 1.66-1.62 (m, 2H), 2.18-1.89 (m, 6H), 3.37 (s, 3H), 4.57 (quintet, 1H).

### Example 62

### 5-Bromomethyl-4-chloro-2-isopropyl-1-methyl-1,2-dihydro-pyrazol-3-one

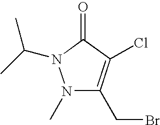

5-Bromomethyl-4-chloro-2-isopropyl-1-methyl-1,2-dihydro-pyrazol-3-one was synthesized from 4-chloro-2-isopropyl-1,5-dimethyl-1,2-dihydro-pyrazol-3-one (359.9 mg, 1.91 mmol), and N-bromosuccinimide (373.5 mg, 2.10 mmol) in carbon tetrachloride (10 mL) under argon. The product was isolated by column chromatography in 70% ethyl acetate and hexanes as a yellow oil (276.1 mg, 54%). ¹H NMR (300 MHz, CDCl₃): δ(ppm) 4.51 (m, 1H), 4.27 (s, 2H), 3.32 (s, 3H), 1.43 (s, 6H).

### Example 63

### 5-Bromomethyl-4-methoxy-1-methyl-2-phenyl-1,2-dihydro-pyrazol-3-one

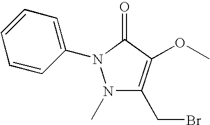

5-Bromomethyl-4-methoxy-1-methyl-2-phenyl-1,2-dihydro-pyrazol-3-one was synthesized from 4-methoxy-1,5-dimethyl-2-phenyl-1,2-dihydro-pyrazol-3-one (697.7 mg, 3.20 mmol), and N-bromosuccinimide (626 mg, 3.52 mmol) in carbon tetrachloride (20 mL). The product was isolated by column chromatography in 40% ethyl acetate and hexanes as a white solid (394.9 mg, 42%). ¹H NMR (300 MHz, CDCl₃): δ(ppm) 7.45 (m, 4H), 7.32 (m, 1H), 4.38 (s, 2H), 4.07 (s, 3H), 3.02 (s, 3H).

### Example 64

### 5-Bromomethyl-4-ethoxy-1-methyl-2-phenyl-1,2-dihydro-pyrazol-3-one

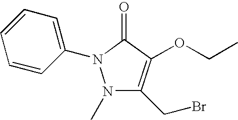

5-Bromomethyl-4-ethoxy-1-methyl-2-phenyl-1,2-dihydro-pyrazol-3-one was synthesized from 4-ethoxy-1,5-dimethyl-2-phenyl-1,2-dihydro-pyrazol-3-one (1.09, 4.70 mmol), and N-bromosuccinimide (1.00 g, 5.64 mmol) in carbon tetrachloride (20 mL). The product was isolated by column chromatography in 50% ethyl acetate and hexanes as a brown solid (0.940 g, 64%). ¹H NMR (300 MHz, CDCl₃): δ(ppm) 7.44-7.49 (m, 4H), 7.29-7.34 (m, 1H), 4.35-4.42 (s, 4H), 3.02 (s, 3H), 1.37 (t, 3H).

### Example 65

### 5-Bromomethyl-2-cyclohexyl-4-methoxy-1-methyl-1,2-dihydro-pyrazol-3-one

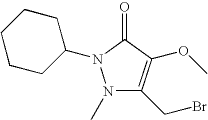

5-Bromomethyl-2-cyclohexyl-4-methoxy-1-methyl-1,2-dihydro-pyrazol-3-one was synthesized from 2-cyclohexyl-4-methoxy-1,5-dimethyl-1,2-dihydro-pyrazol-3-one (20 mg, 0.089 mmol), and N-bromosuccinimide (17 mg, 0.098 mmol) in carbon tetrachloride (2 mL). The product was isolated by column chromatography in 40% ethyl acetate and hexanes as a white (394.9 mg, 42%). ¹H NMR (300 MHz, CDCl₃): δ(ppm) 4.30 (s, 2H), 4.00 (s, 3H), 3.94-3.97 (m, 1H), 3.10 (s, 3H), 2.00 (qd, 3H), 1.85 (t, 4H), 1.67 (d, 1H), 1.23-1.38 (m, 3H).

### Example 66

### 5-Bromomethyl-2-cyclopentyl-4-methoxy-1-methyl-1,2-dihydro-pyrazol-3-one

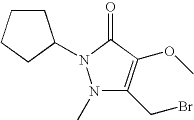

5-Bromomethyl-2-cyclopentyl-4-methoxy-1-methyl-1,2-dihydro-pyrazol-3-one was synthesized from 2-cyclopentyl-4-methoxy-1,5-dimethyl-1,2-dihydro-pyrazol-3-one (0.204 g, 0.970 mmol) and N-bromosuccinimide (0.2245 g, 1.26 mmol) in 5.0 mL of carbon tetrachloride. The crude product was purified by column chromatography in a solution of 10% acetone and dichloromethane to yield an orange oil (0.2044 g, 40.0%).

¹H NMR (300 MHz, CDCl₃) δ ppm: 1.60-1.57 (m, 2H), 2.03-1.85 (m, 6H) 3.09 (s, 3H), 3.95 (s, 2H), 4.23 (s, 2H), 4.48 (quintet, 1H).

### Example 67

### 5-Bromomethyl-4-difluoromethoxy-1-methyl-2-phenyl-1,2-dihydro-pyrazol-3-one

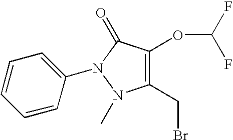

5-Bromomethyl-4-difluoromethoxy-1-methyl-2-phenyl-1,2-dihydro-pyrazol-3-one was synthesized from 4-Difluoromethoxy-1,5-dimethyl-2-phenyl-1,2-dihydro-pyrazol-3-one (135.1 mg, 0.53 mmol), and N-bromosuccinimide (104 mg, 0.58 mmol) in carbon tetrachloride (4 mL). The product was isolated by column chromatography in 30% ethyl acetate and hexanes as an off white solid (108.8 mg, 62%). ¹H NMR (300 MHz, CDCl₃): δ(ppm) 7.38-7.54 (m, 5H), 7.09 (t, 1H, CF₂—H), 4.39 (s, 2H), 3.16 (s, 3H).

### Dibromination

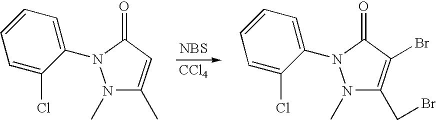

### General Procedure

Pyrazalone (1 equiv.), N-bromosuccinimide (2.3 equiv.) in carbon tetrachloride (15 mL) was refluxed for 1 h. The solid by-product was filtered and the filtrate is concentrated to give product. Proton NMR was used to identify the structure.

Intermediate compounds of Examples 68 through 72 were synthesized using a method analogue to the general procedure for bromination.

### Example 68

### 4-Bromo-5-bromomethyl-2-(2-chloro-phenyl)-1-methyl-1,2-dihydro-pyrazol-3-one

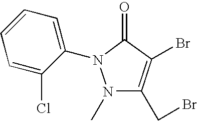

4-Bromo-5-bromomethyl-2-(2-chloro-phenyl)-1-methyl-1,2-dihydro-pyrazol-3-one was obtained from 2-(2-chloro-phenyl)-1,5-dimethyl-1,2-dihydro-pyrazol-3-one (1.36 mmol, 0.284 g), N-bromosuccinimide (3.12 mmol, 0.556 g) in carbon tetrachloride (15 mL). The product was purified using column chromatography (silicagel, 3:1 ethyl acetate:hexane) to give (0.093 g, 15%). ¹H NMR (300 MHz, CDCl₃): [symbol] (ppm) 7.36-7.63 (m, 4H), 4.41 (s, 2H), 3.04 (s, 3H).

### Example 69

### 4-Bromo-5-bromomethyl-2-(4-chloro-phenyl)-1-methyl-1,2-dihydro-pyrazol-3-one

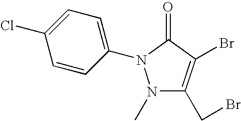

4-Bromo-5-bromomethyl-2-(4-chloro-phenyl)-1-methyl-1,2-dihydro-pyrazol-3-one was obtained from 2-(4-chloro-phenyl)-1,5-dimethyl-1,2-dihydro-pyrazol-3-one (1.15 mmol, 0.24 g), N-bromosuccinimide (2.7 mmol, 0.480 g) in carbon tetrachloride (15 mL). The product was purified using column chromatography (silicagel, 3:1 ethyl acetate:hexane) to give (0.146 g, 30%). ¹H NMR (300 MHz, CDCl₃): [symbol] (ppm) 7.47 (s, 2H), 7.35 (s, 2H), 4.38 (s, 2H), 3.21 (s, 3H).

### Example 70

### 4-Bromo-5-bromomethyl-2-(3-chloro-phenyl)-1-methyl-1,2-dihydro-pyrazol-3-one

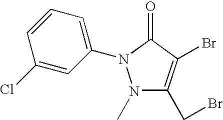

4-Bromo-5-bromomethyl-2-(3-chloro-phenyl)-1-methyl-1,2-dihydro-pyrazol-3-one was obtained from 2-(3-chloro-phenyl)-1,5-dimethyl-1,2-dihydro-pyrazol-3-one (0.92 mmol, 0.206 g), N-bromosuccinimide (1.9 mmol, 0.338 g) in carbon tetrachloride (10 mL). The crude product obtained (0.165 g, 47%) was used in next step. ¹H NMR (300 MHz, CDCl₃): [symbol] (ppm) 7.26-7.43 (m, 4H), 4.38 (s, 2H), 3.21 (s, 3H).

### Example 71

### 4-Bromo-5-bromomethyl-2-(4-methoxy-phenyl)-1-methyl-1,2-dihydro-pyrazol-3-one

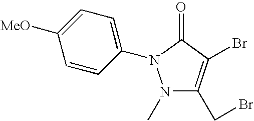

4-Bromo-5-bromomethyl-2-(4-methoxy-phenyl)-1-methyl-1,2-dihydro-pyrazol-3-one was obtained from 2-(4-methoxy-phenyl)-1,5-dimethyl-1,2-dihydro-pyrazol-3-one (0.81 mmol, 0.175 g), N-bromosuccinimide (1.7 mmol, 0.302 g) in carbon tetrachloride (10 mL). The crude product obtained (0.142 g, 47%) was used in next step. ¹H NMR (300 MHz, CDCl₃): δ(ppm) 7.28 (d, 2H), 7.04 (d, 2H), 4.38 (s, 2H), 3.83 (s, 3H), 3.21 (s, 3H).

### Example 72

### 4-Bromo-5-bromomethyl-2-(3-methoxy-phenyl)-1-methyl-1,2-dihydro-pyrazol-3-one

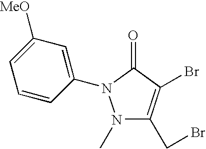

4-Bromo-5-bromomethyl-2-(3-methoxy-phenyl)-1-methyl-1,2-dihydro-pyrazol-3-one was obtained from 2-(3-methoxy-phenyl)-1,5-dimethyl-1,2-dihydro-pyrazol-3-one (0.81 mmol, 0.175 g), N-bromosuccinimide (1.7 mmol, 0.302 g) in carbon tetrachloride (10 mL). The crude product obtained (0.142 g, 47%) was used in next step.

An intermediate compound of Example 73 was synthesized as follows.

Methylation of 5-Fluoro-2-nitrophenol

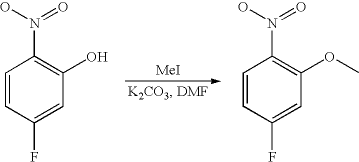

### Example 73

### 4-Fluoro-2-methoxy-1-nitro-benzene

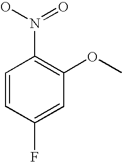

4-Fluoro-2-methoxy-1-nitro-benzene was synthesized by suspending 5-fluoro-2-nitrophenol (5.0 g, 31.8 mmol, 1.0 equiv.), potassium carbonate (6.59 g, 47.7 mmol, 1.5 equiv.), and Iodomethane (2.98 mL, 47.7 mmol, 1.5 equiv.) in DMF (50 mL) and allowing the resulting reaction mixture to stir overnight at 140° C. inside a sealed pressure flask. The reaction mixture was partitioned between ethyl acetate and distilled water three times. The organic layer was washed once with brine and dried over anhydrous sodium sulfate. The solvent was removed in vacuo. The product was isolated by column chromatography in 30% ethyl acetate and hexanes as a yellow solid (1.44 g, 26%). ¹H NMR (300 MHz, CDCl₃): δ(ppm) 7.98 (dd, 1H), 6.77 (m, 2H), 3.99 (s, 3H).

### Iron Reduction of Nitro to Produce Amine

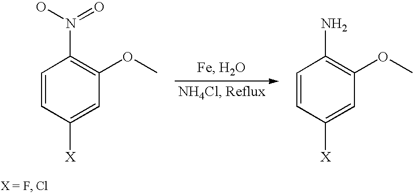

### General Procedure

A suspension of ferrum (5.0 equiv.), ammonium chloride (0.65 equiv.), and distilled water were refluxed for fifteen minutes. The nitro compound (1.0 equiv.) was added and the resulting reaction mixture was allowed to stir at reflux. When TLC showed that the reaction had stopped the mixture was neutralized by dropwise addition of a 5% aqueous solution of sodium bicarbonate and it was filtered through Celite. The filtrate was washed thrice with ethyl acetate. The combined organic layers were washed once with brine and once with a 5% aqueous solution of hydrochloric acid. The combined water layers were neutralized with 20% aqueous sodium hydroxide and extracted thrice with ethyl acetate. The organic layers were combined, dried over anhydrous sodium sulfate and the solvent removed in vacuo. The pure product was obtained using column chromatography in ethyl acetate and hexanes but sometimes the product was kept crude. The purity of the product was determined using 1H-NMR.

An intermediate compound of Example 74 was synthesized using a method analogous to the above general procedure for reduction of nitro to produce amine.

### Example 74

### 4-Chloro-2-methoxy-phenylamine

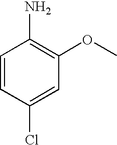

4-Chloro-2-methoxy-phenylamine was obtained from Ferrum (2.23 g, 40 mmol) ammonium chloride (278 mg, 5.2 mmol), water (48 mL) and 5-chloro-2-nitroanisole (1.5 g, 8.0 mmol) as a crude mixture, which was a dark purple oil (1.15 g, 91%). The reaction was complete in 1.5 hours. ¹H NMR (300 MHz, CDCl₃): δ(ppm) 6.78 (m, 2H), 6.65 (d, 1H), 3.86 (s, 3H).

4-Chloro-2-methoxy-phenylamine was obtained from Ferrum (4.47 g, 80.0 mmol) ammonium chloride (556 mg, 10.4 mmol), water (80 mL) and 5-chloro-2-nitroanisole (3.0 g, 16.0 mmol) as a crude mixture, which was a dark purple oil (2.35 g, 93%). The reaction was complete in 2 hours. No ¹H-NMR was performed.

### Example 75

### 4-Fluoro-2-methoxy-phenylamine

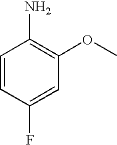

4-Fluoro-2-methoxy-phenylamine was obtained from Ferrum (2.35 g, 42.1 mmol) ammonium chloride (283 mg, 5.47 mmol), water (45 mL) and 4-Fluoro-2-methoxy-1-nitro-benzene (1.44 g, 8.42 mmol) after column chromatography as a dark oil (151.5 mg, 13%). ¹H NMR (300 MHz, CDCl₃): δ(ppm) 6.49-6.67 (m, 3H), 3.86 (s, 3H), 3.64 (broad s, 2H).

### Piperazine Synthesis

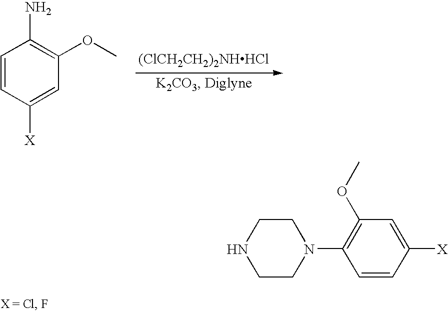

### General Procedures

### Absence of Sodium Iodide

In a sealed pressure flask a phenyl amine (1.0 equiv.), bis(2-chloroethyl)amine hydrochloride (1.5 equiv.), and potassium carbonate (1.5 equiv.) were suspended in diglyme. The resulting mixture was allowed to stir at 220° C. for 3.5 hours. The mixture was cooled to room temperature over two hours and further cooled to 0° C. It was then partitioned between dichloromethane and distilled water. The pH of the water layer was adjusted to basic pH (9-10) with 5% aqueous sodium hydroxide. The water phase was extracted thrice with dichloromethane. The combined organic layers were dried over anhydrous sodium sulfate and the solvent removed in vacuo. The product was purified by column chromatography in 2M ammonium/methanol and dichloromethane mixtures.

### Presence of Sodium Iodide

In a flask equipped with a water cooled condenser a phenyl amine (1.0 equiv.), bis(2-chloroethyl)amine hydrochloride (1.5 equiv.), potassium carbonate (1.5 equiv.) and sodium iodide (0.4 equiv.) were suspended in diglyme. The resulting reaction mixture was allowed to heat to reflux over a period of one hour and allowed to stir at reflux for an additional 2.5 hours. It was then partitioned between dichloromethane and distilled water. The pH of the water layer was adjusted to basic pH (9-10) with 5% aqueous sodium hydroxide. The water phase was extracted thrice with dichloromethane. The combined organic layers were washed once with 10% aqueous sodium thiosulfate to remove iodine, dried over anhydrous sodium sulfate and the solvent removed in vacuo. The product was purified by column chromatography in 2M ammonium/methanol and dichloromethane mixtures.

An intermediate compound of Example 76 was synthesized analogous to the general procedure for piperazine synthesis in the absence of sodium iodide.

### Example 76

### 1-(4-Chloro-2-methoxy-phenyl)-piperazine

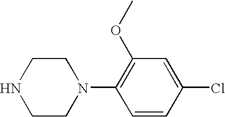

1-(4-Chloro-2-methoxy-phenyl)-piperazine was synthesized from 4-Chloro-2-methoxy-phenylamine (1.15 g, 7.30 mmol), bis(2-chloroethyl)amine hydrochloride (1.95 g, 10.95 mmol), and potassium carbonate (1.51 g, 10.95 mmol) in diglyme. Column chromatography 2.5% 2M ammonia/methanol in dichloromethane provided the product as a brown solid (187.9 mg, 11%). ¹H NMR (300 MHz, CDCl₃): δ(ppm) 6.93 (d, 1H), 6.84 (m, 2H), 3.87 (s, 3H), 3.12 (broad m, 4H), 2.88 (broad t, 4H).

Intermediate compounds of Examples 77 and 78 were synthesized in a manner analogous to the general procedure for piperazine synthesis in the presence of sodium iodide.

### Example 77

### 1-(4-Fluoro-2-methoxy-phenyl)-piperazine

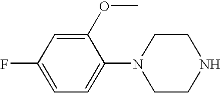

1-(4-Fluoro-2-methoxy-phenyl)-piperazine was synthesized from 4-Fluoro-2-methoxy-phenylamine (151.5 mg, 1.07 mmol), bis(2-chloroethyl)amine hydrochloride (287.4 mg, 1.61 mmol), potassium carbonate (222.5 mg, 1.61 mmol) and sodium iodide (64.5 mg, 0.43 mmol) in diglyme. Column chromatography 10% 2M ammonia/methanol in dichloromethane provided the product as a dark brown oil (89.8 mg, 40%). ¹H NMR (300 MHz, CDCl₃): δ(ppm) 6.78-6.90 (m, 1H), 6.57-6.65 (m, 2H), 3.86 (s, 3H), 3.14 (broad t, 2H), 3.05 (broad t, 4H), 2.95 (t, 1H), 2.72 (broad t, 2H).

### Example 78

### 1-(4-Chloro-2-methoxy-phenyl)-piperazine

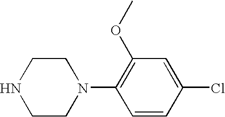

1-(4-Chloro-2-methoxy-phenyl)-piperazine was synthesized from 4-Chloro-2-methoxy-phenylamine (1.15 g, 7.30 mmol), bis(2-chloroethyl)amine hydrochloride (1.95 g, 10.95 mmol), potassium carbonate (1.51 g, 10.95 mmol) and sodium iodide (894.9 mg, 5.97 mmol) in diglyme. Column chromatography 10% 2M ammonia/methanol in dichloromethane provided the product as a brown solid (1.446 g, 43%). ¹H NMR (300 MHz, CDCl₃): δ(ppm) 6.70-6.84 (m, 3H), 3.87 (broad s, 1H), 3.83 (s, 3H), 3.00-3.13 (broad m, 4H), 2.70-2.84 (broad m, 4H).

### Procedure for Making Aryl Piperidines

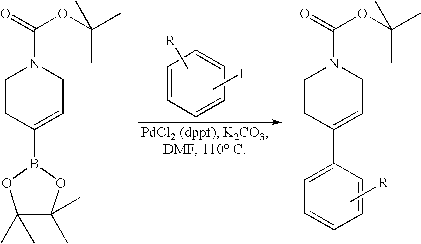

### General Procedure:

The boronate ester (1.0 equiv), iodo-benzene (1.0 equiv), palladium catalyst (0.1 equiv) and potassium carbonate (3.0 equiv) was added to a solution of deoxygenated DMF. The flask was flushed with argon for 15 minutes, fitted with a dry tube and run over night at 110° C. The reaction was poured onto water and extracted three times with ethyl acetate. The organic layers were washed with a brine solution, dried over anhydrous sodium sulfate. The reaction was purified through a 10 g SPE tube in a mixture of ethyl acetate and hexanes. ¹H NMR was used to confirm the purity of the product.

Intermediate compounds of Examples 79 through 82 were synthesized using a method analogous to the above general procedure for the coupling of a boronate ester to an iodo-phenyl group.

### Example 79

### 4-(5-Chloro-2-methyl-phenyl)-3,6-dihydro-2H pyridine-1-carboxylic acid tert-butyl ester

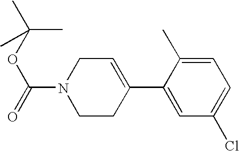

4-(5-Chloro-2-methyl-phenyl)-3,6-dihydro-2H pyridine-1-carboxylic acid tert-butyl ester was synthesized from 4-(4,4,5,5-tetramethyl-[1,3,2]dioxaborolan-2-yl)-3,6-dihydro-2H-pyridin-1-carboxylic acid tert-butyl ester (0.200 g, 0.647 mmol), 4-chloro-2-iodo-1-methyl-benzene (0.163 mg, 0.647 mmol), Pd Cl₂ (dppf) (0.053 g, 0.0647 mmol) and potassium carbonate (0.268 g, 1.94 mmol) in 20.0 mL of DMF. The reaction was purified by eluting through a 10 g SPE tube using a solution of 10% ethyl acetate and hexanes to yield a brown liquid (0.236 g, 124%). ¹H NMR (300 MHz, CDC₃) δ ppm: 1.54 (s, 9H), 2.02 (s, 2H), 2.39 (s, 3H), 3.66 (br, 2H), 4.15-4.06 (br, 2H), 5.52 (br, 1H), 7.78-7.07 (m, 3H).

### Example 80

### 4-(5-Chloro-2-methoxy-phenyl)-3,6-dihydro-2H-pyridine-1-carboxylic acid tert-butyl ester

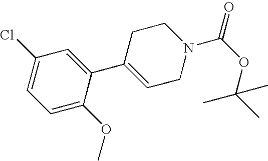

4-(5-Chloro-2-methoxy-phenyl)-3,6-dihydro-2H-pyridine-1-carboxylic acid tert-butyl ester was synthesized from 4-(4,4,5,-tetramethyl-[1,3]dioxolan-2-yl)-3,6-dihydro-2H-pyridine-1-carboxylic acid tert-butyl ester (0.884 g, 2.8 mmol), 4-chloro-2-iodo-1-methoxy-benzene (0.752 g, 2.8 mmol), Pd Cl₂ (dppf) (0.228 g, 0.28 mmol) and potassium carbonate (1.16 g, 8.4 mmol) in 30.0 mL of DMF. The crude reaction was purified by column chromatography in a solution of 12% ethyl acetate and hexanes to yield a yellow oil (0.434 g, 47.9%). ¹H NMR (300 MHz, CDCl₃) δ ppm: 1.49 (s, 9H), 2.45 (br, 2H), 3.57 (t, 2H), 4.03 (br, 2H), 5.8 (br, 1H), 6.78 (d, 1H), 7.11-7.18 (m, 2H).

### Example 81

### 4-(5-Chloro-2-difluoromethoxy-phenyl)-3,6-dihydro-2H-pyridine-1-carboxylic acid tert-butyl ester

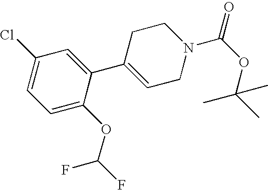

4-(5-Chloro-2-difluoromethoxy-phenyl)-3,6-dihydro-2H-pyridine-1-carboxylic acid tert-butyl ester was synthesized from 4-(4,4,5,-tetramethyl-[1,3]dioxolan-2-yl)-3,6-dihydro-2H-pyridine-1-carboxylic acid tert-butyl ester (0.300 g, 0.97 mmol), 4-chloro-2-iodo-1-difluoromethoxy-benzene (0.296 g, 0.97 mmol), Pd Cl₂ (dppf) (0.080 g, 0.097 mmol) and potassium carbonate (0.402 g, 2.92 mmol) in 30.0 mL of DMF. The crude reaction was purified by column chromatography in a solution of 12% ethyl acetate and hexanes to yield a yellow oil (0.201 g, 57.6%). ¹H NMR (300 MHz, CDCl₃) δ ppm: 7.17-7.25 (m, 2H), 7.05-7.08 (m, 1H), 6.42 (t, 1H), 5.84 (s, 1H), 4.06 (d, 2H), 3.60 (t, 2 h), 2.45 (s, 2H), 1.51 (s, 9H).

### Hydrogenation of Alkenes

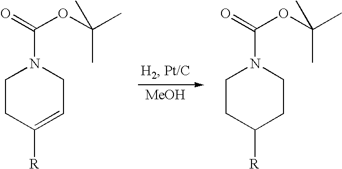

### General Procedure:

A round bottom flask was charged with the tert-butyl ester (1.0 equiv) and dissolved in methanol while being flushed with argon. A corresponding mass of platinum on activated carbon was added to the reaction. Lastly, the reaction was fitted with a balloon filled with hydrogen. The reaction was allowed to run overnight. The product was stirred with celite and run through a plug of celite. The product identity and purity was observed by ¹H NMR.

Intermediate compounds of Examples and 83 were synthesized using a method analogous to the above general procedure for hydrogenation of alkenes.

### Example 82

### 4-(5-Chloro-2-methyl-phenyl)-piperidine-1-carboxylic acid tert-butyl ester

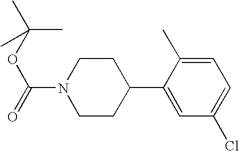

4-(5-Chloro-2-methyl-phenyl)-piperidine-1-carboxylic acid tert-butyl ester was synthesized from 4-(5-chloro-2-methyl-phenyl)-3,6-dihydro-2H-pyridine-1-carboxylic acid tert-butyl ester (50 mg, 0.170 mmol) and platinum on carbon (50 mg) in 5 mL of methanol. A balloon filled with hydrogen gas was then affixed to the reaction. The reaction yielded a colourless oil (48.2 mg, 95.8%). ¹H NMR (300 MHz, CDCl₃) δ ppm: 1.51 (s, 9H), 1.61 (d, 2H), 2.32 (s, 3H), 2.83 (td, 2H), 4.15 (br, 2H), 7.10 (s, 2H), 7.15 (s, 1H).

### Example 83

### 4-(5-Chloro-2-methoxy-phenyl)-piperidine-1-carboxylic acid tert-butyl ester

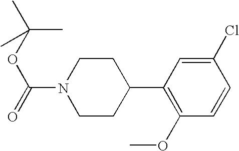

4-(5-Chloro-2-methoxy-phenyl)-piperidine-1-carboxylic acid tert-butyl ester was synthesized from 4-(2-methoxy-5-methyl-phenyl)-3,6-dihydro-2H-pyridine-1-carboxylic acid tert butyl ester (200 mg, 0.6176 mmol) and platinum on carbon (200 mg) in 20 mL of methanol. A balloon filled with hydrogen was then affixed to the reaction flask. The reaction yielded a colourless oil. ¹H NMR (300 MHz, CDCl₃) δ ppm: 1.51 (s, 9H), 1.76 (t, 2H), 2.0 (br, 2H), 2.86 (t, 2H), 3.21 (br, 2H), 4.27 (br, 1H) 6.77-6.80 (d, 1H), 7.20-7.17 (m, 2H).

### Procedure to Make Phenoxy-Ethyl Piperidine

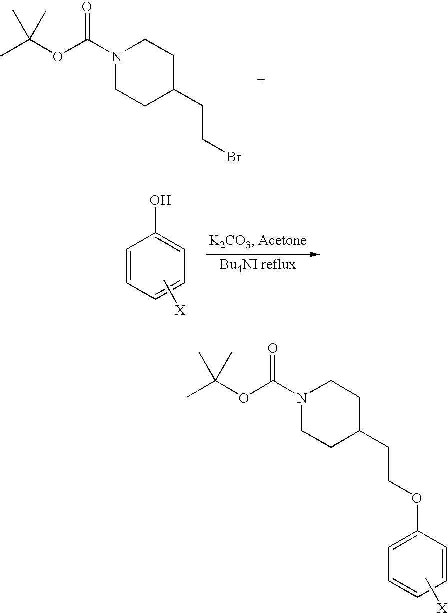

### General Procedure:

The phenol (1.0 equiv), tetrabutylammonium iodide (0.06 equiv), and potassium carbonate (2.0 equiv) was added to a solution of 4-(2-bromo-ethyl)-piperine-1-carboxylic acid tert-butyl ester (1.0 equiv) in acetone. The reaction mixture was refluxed overnight. After removing acetone, the residue was partitioned between ethyl acetate and water. The organic layer was washed with 1N sodium hydroxide aqueous solution, water, brine and dried over anhydrous sodium sulfate. The product was purified with flash chromatography on silica gel (20% ethyl acetate in hexanes). ¹H NMR was used to confirm the purity of the product.

Intermediate compounds 84 through 87 were synthesized using a method analogous to the above general procedure for making phenoxy-ethyl piperidine.

### Example 84

### 4-[2-(4-Fluoro-phenoxy)-ethyl]-piperidine-1-carboxylic acid tert-butyl ester

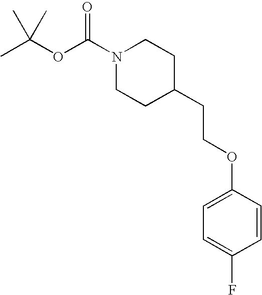

4-[2-(4-Fluoro-phenoxy)-ethyl]-piperidine-1-carboxylic acid tert-butyl ester was obtained from 4-fluoro-phenol (1.37 mmol, 0.153 g), tetrabutylammonium iodide (0.081 mmol, 0.03 g), 4-(2-bromo-ethyl)-piperine-1-carboxylic acid tert-butyl ester (1.37 mmol, 0.4 g) and potassium carbonate (2.74 mmol, 0.946 g) in acetone (10 ml) as a off white solid (0.423 g 95.8%). ¹H NMR (300 MHz, CDCl₃): δ(ppm) 6.88-6.94 (m, 2H), 6.75-6.79 (m, 2H), 4.01-4.06 (m, 2H), 3.90 (t, 2H), 2.62 (t, 2H), 1.59-1.67 (m, 5H), 1.42 (s, 9H), 1.12-1.15 (m, 2H).

### Example 85

### 4-[2-(4-Chloro-phenoxy)-ethyl]-piperidine-1-carboxylic acid tert-butyl ester

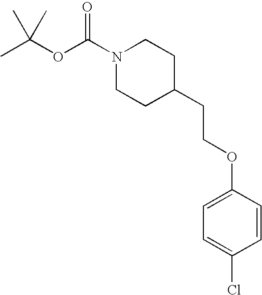

4-[2-(4-chloro-phenoxy)-ethyl]-piperidine-1-carboxylic acid tert-butyl ester was obtained from 4-chloro-phenol (1.37 mmol, 0.176 g), tetrabutylammonium iodide (0.081 mmol, 0.03 g), 4-(2-bromo-ethyl)-piperine-1-carboxylic acid tert-butyl ester (1.37 mmol, 0.4 g) and potassium carbonate (2.74 mmol, 0.946 g) in acetone (10 ml) as a off white solid (0.428 g 92%). ¹H NMR (300 MHz, CDCl3): δ(ppm) 7.19-7.22 (m, 2H), 6.78-6.82 (m, 2H), 4.02-4.06 (m, 2H), 3.95 (t, 2H), 2.65 (t, 2H), 1.68-1.72 (m, 5H), 1.46 (s, 9H), 1.06-1.10 (m, 2H).

### Example 86

### 4-[2-(3,4-Difluoro-phenoxy)-ethyl]-piperidine-1-carboxylic acid tert-butyl ester

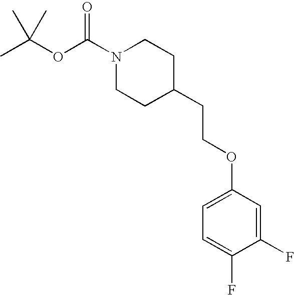

4-[2-(3,4-difluoro-phenoxy)-ethyl]-piperidine-1-carboxylic acid tert-butyl ester was obtained from 3,4-difluoro-phenol (1.03 mmol, 0.134 g), tetrabutylammonium iodide (0.061 mmol, 0.023 g), 4-(2-bromo-ethyl)-piperine-1-carboxylic acid tert-butyl ester (1.03 mmol, 0.3 g) and potassium carbonate (2.06 mmol, 0.285 g) in acetone (10 ml) as a off white solid (0.36 g 101%). ¹H NMR (300 MHz, CDCl3): δ(ppm) 6.69-7.05 (m, 1H), 6.63-6.69 (m, 1H), 6.52-6.57 (m, 1H), 4.05-4.12 (m, 2H), 3.91 (t, 2H), 2.68 (t, 2H), 1.66-1.75 (m, 5H), 1.43 (s, 9H), 1.08-1.15 (m, 2H).

### Example 87

### 4-[2-(3,4-Dichloro-phenoxy)-ethyl]-piperidine-1-carboxylic acid tert-butyl ester

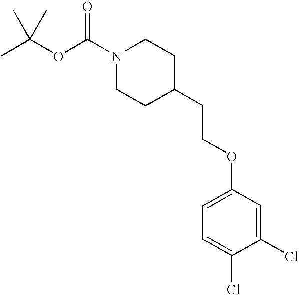

4-[2-(3,4-dichloro-phenoxy)-ethyl]-piperidine-1-carboxylic acid tert-butyl ester was obtained from 3,4-dichloro-phenol (1.03 mmol, 0.168 g), tetrabutylammonium iodide (0.061 mmol, 0.023 g), 4-(2-bromo-ethyl)-piperine-1-carboxylic acid tert-butyl ester (1.03 mmol, 0.3 g) and potassium carbonate (2.06 mmol, 0.285 g) in acetone (10 ml) as a off white solid (0.45 g 105%).

¹H NMR (300 MHz, CDCl3): δ(ppm) 7.18 (d, 1H), 6.95-6.96 (m, 1H), 6.69-6.73 (m, 1H), 4.05-4.12 (m, 2H), 3.94 (t, 2H), 2.69 (t, 2H), 1.67-1.71 (m, 5H), 1.45 (s, 9H), 1.08-1.17 (m, 2H).

Procedure to make Phenyl-Allyl Piperidine

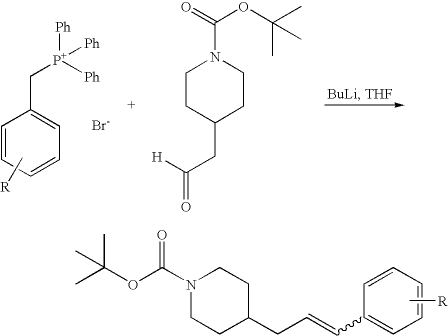

### General Procedure:

To a suspension of benzyl triphenyl phosphonium bromide (1.0 equiv) in dry THF, 2M butyllithium in pentane (1.35 equiv) was added at −10° C. After stirred for 30 min, the solution of piperidinyl acetaldehyde (1.05 equiv) in THF was added dropwise. The mixture was allowed to warm to room temperature and stirred for another 6 hours. After removing THF, the residue was partitioned between ether and water. The combined organic layers were washed with brine, dried over anhydrous sodium sulfate. The product was purified with flash chromatography on silica gel (30% ethyl acetate in hexanes). ¹H NMR was used to confirm the purity of the product.

Intermediate compounds 88 through 91 were synthesized using a method analogous to the above general procedure for making phenyl-allyl piperidine.

### Example 88

### 4-[2-(4-Fluoro-phenyl)-allyl]-piperidine-1-carboxylic acid tert-butyl ester

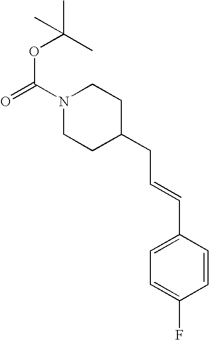

4-[2-(4-Fluoro-phenyl)-allyl]-piperidine-1-carboxylic acid tert-butyl ester was obtained from 4-fluoro-benzyl triphenyl phosphorium bromide (2.20 mmol, Ig), 2M butyllithium in pentane (2.98 mmol, 1.5 ml), 1-(2-oxo-ethyl)-piperine-4-carboxylic acid tert-butyl ester (2.30 mmol, 0.53 g) in THF (30 ml) as yellow foam (0.712 g 96.9%). ¹H NMR (300 MHz, CDCl3): δ(ppm) 7.11-7.26 (m, 2H), 6.87-6.97 (m, 2H), 6.20-6.38 (m, 1H), 5.96-6.06 and 5.56-5.62 (m, 1H), 4.00-4.08 (m, 2H), 2.61 (t, 2H), 2.04-2.16 (m, 2H), 1.57-1.64 (m, 3H), 1.41 (s, 9H), 1.08-1.17 (m, 2H).

### Example 89

### 4-(3-pyridin-4-yl-allyl)-piperidine-1-carboxylic acid tert-butyl ester

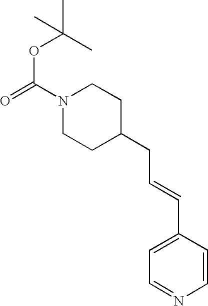

4-(3-pyridin-4-yl-allyl)-piperidine-1-carboxylic acid tert-butyl ester was obtained from triphenyl-pyridin-4-ylmethyl phosphorium bromide (2.13 mmol, 0.834 g), 2M butyllithium in pentane (2.87 mmol, 1.45 ml), 1-(2-oxo-ethyl)-piperine-4-carboxylic acid tert-butyl ester (2.23 mmol, 0.508 g) in THF (30 ml) as yellow foam (0.40 g 62%).

¹H NMR (300 MHz, CDCl₃): δ (ppm) 8.39-8.46 (m, 1H), 7.54-7.61 (m, 1H), 7.36-7.39 (m, 2H), 7.04-7.12 (m, 1H), 6.20-6.42 and 5.71-5.81 (m, 1H), 3.97-4.05 (m, 2H), 2.61 (t, 2H), 2.11-2.19 (m, 2H), 1.57-1.62 (m 3H), 1.37 (s, 9H), 1.02-1.12 (m, 2H).

### Example 90

### 4-(3-pyridin-3-yl-allyl)-piperidine-1-carboxylic acid tert-butyl ester

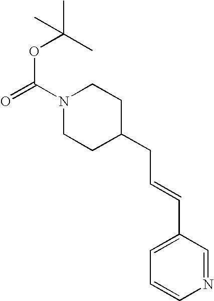

4-(3-pyridin-3-yl-allyl)-piperidine-1-carboxylic acid tert-butyl ester was obtained from triphenyl-pyridin-3-ylmethyl phosphorium bromide (0.33 mmol, 0.130 g), 2M butyllithium in pentane (0.45 mmol, 0.23 ml), 1-(2-oxo-ethyl)-piperine-4-carboxylic acid tert-butyl ester (0.35 mmol, 0.080 g) in THF (10 ml) as yellow foam (0.08 g 80%). ¹H NMR (300 MHz, CDCl₃): δ (ppm) 8.41-8.55 (m, 2H), 7.45-7.54 (m, 1H), 7.18-7.25 (m, 1H), 6.20-6.45 and 5.75-5.82 (m, 2H), 4.08-4.10 (m, 2H), 2.68 (t, 2H), 2.16-2.27 (m, 2H), 1.48-1.70 (m 3H), 1.44 (s, 9H), 1.11-1.17 (m, 2H).

### Example 91

### 4-(3-pyridin-2-yl-allyl)-piperidine-1-carboxylic acid tert-butyl ester

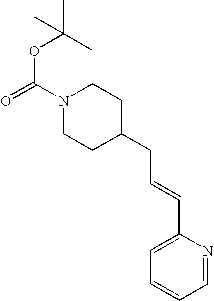

4-(3-pyridin-2-yl-allyl)-piperidine-1-carboxylic acid tert-butyl ester was obtained from triphenyl-pyridin-2-ylmethyl phosphorium bromide (3.29 mmol, 1.29 g), 2M butyllithium in pentane (4.44 mmol, 2.22 ml), 1-(2-oxo-ethyl)-piperine-4-carboxylic acid tert-butyl ester (3.45 mmol, 0.786 g) in THF (10 ml) as yellow foam (1.19 g 101%). ¹H NMR (300 MHz, CDCl₃): δ (ppm) 8.28-8.29 (m, 1H), 7.32-7.38 (m, 1H), 6.97-6.99 (m, 1H), 6.82-6.86 (m, 1H), 6.22-6.27 (m, 1H), 6.48-6.53 and 5.45-5.55 (m, 1H), 3.83-3.90 (m, 2H), 2.40 (t, 2H), 1.94-1.99 (m, 2H), 1.30-1.49 (m 3H), 1.22 (s, 9H), 0.89-1.09 (m, 2H).

Procedure to make Phenyl-Propyl Piperidine

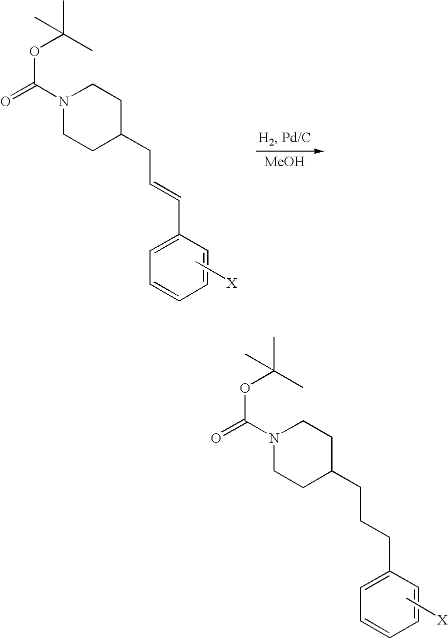

### General Procedure:

A round bottom flask was charged with the phenyl-allyl piperidine (1.0 equiv) and dissolved in methanol while being flushed with argon. A corresponding mass of platinum on activated carbon was added to the reaction. Lastly, the reaction was fitted with a balloon filled with hydrogen. The reaction was allowed to run overnight. The product was stirred with celite and run through a plug of celite. The product identity and purity was observed by ¹H NMR.

Intermediate compounds 92 through 95 were synthesized using a method analogous to the above general procedure for hydrogenation to make phenyl propyl piperidine.

### Example 92

### 4-[3-(4-Fluoro-phenyl)-propyl]-piperidine-1-carboxylic acid tert-butyl ester

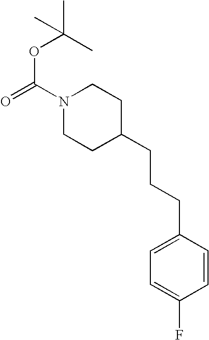

4-[3-(4-Fluoro-phenyl)-propyl]-piperidine-1-carboxylic acid tert-butyl ester was synthesized from 4-[2-(4-Fluoro-phenyl)-allyl]-piperidine-1-carboxylic acid tert-butyl ester (300 mg, 0.94 mmol) and platinum on carbon (150 mg) in 10 mL of methanol. A balloon filled with hydrogen gas was then affixed to the reaction. The reaction yielded yellow oil (250.7 mg, 82.9%). ¹H NMR (300 MHz, CDCL₃): δ (ppm) 7.08-7.13 (m, 2H), 6.91-6.97 (m, 2H), 4.00-4.08 (m, 2H), 2.52-2.61 (m, 4H), 1.59-1.65 (m, 4H), 1.45 (s, 9H), 1.24-1.27 (m, 3H), 0.95-1.05 (m, 2H).

### Example 93

### 4-(3-pyridin-4-yl-propyl)-piperidine-1-carboxylic acid tert-butyl ester

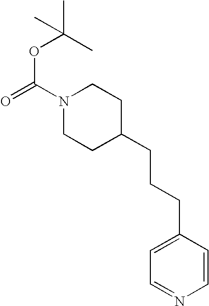

4-(3-pyridin-4-yl-propyl)-piperidine-1-carboxylic acid tert-butyl ester was synthesized from 4-(3-pyridin-4-yl-allyl)-piperidine-1-carboxylic acid tert-butyl ester (238 mg, 0.787 mmol) and platinum on carbon (140 mg) in 6 mL of methanol. A balloon filled with hydrogen gas was then affixed to the reaction. The reaction yielded yellow oil (230 mg, 96%). ¹H NMR (300 MHz, CDCL₃): δ (ppm) 8.40-8.48 (m, 1H), 7.56-7.63 (m, 1H), 7.32-7.37 (m, 2H), 3.01-4.09 (m, 2H), 2.85 (t, 2H), 2.13-2.65 (m, 2H), 1.45-1.81 (m 5H), 1.41 (s, 9H), 1.02-1.12 (m, 2H).

### Example 94

### 4-(3-pyridin-3-yl-propyl)-piperidine-1-carboxylic acid tert-butyl ester

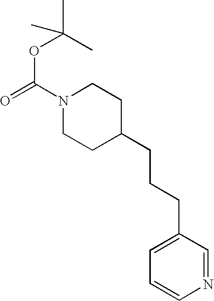

4-(3-pyridin-3-yl-propyl)-piperidine-1-carboxylic acid tert-butyl ester was synthesized from 4-(3-pyridin-3-yl-allyl)-piperidine-1-carboxylic acid tert-butyl ester (80 mg, 0.26 mmol) and platinum on carbon (40 mg) in 6 mL of methanol. A balloon filled with hydrogen gas was then affixed to the reaction. The reaction yielded yellow oil (75 mg, 95%). ¹H NMR (300 MHz, CDCl₃): δ(ppm) 8.45-8.55 (m, 2H), 7.45-7.54 (m, 1H), 7.18-7.25 (m, 1H), 4.08-4.10 (m, 2H), 2.68 (t, 2H), 2.16-2.27 (m, 2H), 1.48-1.70 (m 5H), 1.44 (s, 9H), 1.11-1.17 (m, 2H).

### Example 95

### 4-(3-pyridin-2-yl-propyl)-piperidine-1-carboxylic acid tert-butyl ester

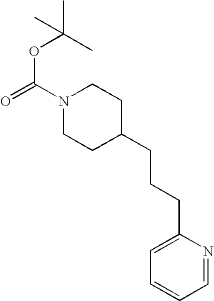

4-(3-pyridin-2-yl-propyl)-piperidine-1-carboxylic acid tert-butyl ester was synthesized from 4-(3-pyridin-2-yl-allyl)-piperidine-1-carboxylic acid tert-butyl ester (280 mg, 0.925 mmol) and platinum on carbon (140 mg) in 6 mL of methanol. A balloon filled with hydrogen gas was then affixed to the reaction. The reaction yielded yellow oil (265 mg, 94%). ¹H NMR (300 MHz, CDCl₃): δ (ppm) 8.45-8.47 (m, 1H), 7.60-7.61 (m, 1H), 7.11-7.14 (m, 2H), 3.83-3.90 (m, 2H), 2.74 (t, 2H), 2.57 (t, 2H), 1.54-1.69 (m 5H), 1.36 (s, 9H), 0.98-1.15 (m, 2H).

### Final Compounds and Further Intermediates

Coupling of Pyrazalones with piperazines, Piperidines, and Pyrrolidines

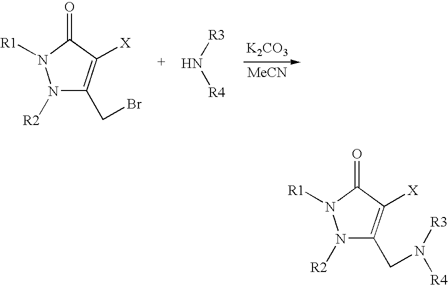

### General Procedure A

The amine (1.5 equiv.) was added to a mixture of potassium carbonate (5 equiv.) and 5-bromomethyl-4-chloro-1-methyl-2-phenylpyrazolidin-3-one (1 equiv.) in acetonitrile. It was left to stir overnight. The resulting reaction mixture was partitioned between water and dichloromethane. Solvent was removed from the organic layer. The resulting crude product was then purified using column chromatography with 50% hexanes and ethyl acetate.

Solvent was removed in vacuo. NMR was used to determine the purity of the isolated compounds.

Compounds of Examples 96 through 282 were synthesized using a method analogous to the above general procedure A for piperazine and pyrazolone coupling.

### Example 96

### 4-Chloro-5-[4-(4-chlorophenyl)piperazin-1-ylmethyl]-1-methyl-2-phenyl-1,2-dihydropyrazol-3-one

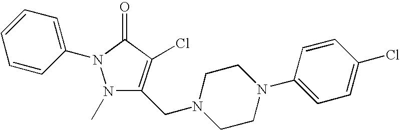

4-Chloro-5-[4-(4-chlorophenyl)piperazin-1-ylmethyl]-1-methyl-2-phenyl-1,2-dihydropyrazol-3-one was obtained from 5-bromomethyl-4-chloro-1-methyl-2-phenylpyrazolidin-3-one (30 mg, 0.1 mmol), 1-(4-chlorophenyl)piperazine dihydrochloride (40 mg, 0.15 mmol) and potassium carbonate (69 mg, 0.5 mmol) in acetonitrile (2 mL) as off white solid 38.4 mg (91%). ¹H NMR (300 MHz, CDCl₃): δ (ppm) 7.52-7.28 (m, 5H), 7.26-7.22 (d, 2H), 6.89-6.85 (d, 2H) 3.65 (s, 2H), 3.24 (s, 3H), 3.27-3.08 (br s, 4H), 2.74 (br s, 4H).

### Example 97

### 4-Chloro-1-methyl-2-phenyl-5-(4-o-tolylpiperazin-1-ylmethyl)-1,2-dihydropyrazol-3-one

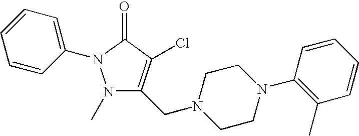

4-Chloro-1-methyl-2-phenyl-5-(4-o-tolylpiperazin-1-ylmethyl)-1,2-dihydropyrazol-3-one was obtained from 5-bromomethyl-4-chloro-1-methyl-2-phenylpyrazolidin-3-one (30 mg, 0.1 mmol), 1-(o-tolyl)piperazine hydrochloride (32 mg, 0.15 mmol) and potassium carbonate (49 mg, 0.35 mmol) in acetonitrile (2 mL) as an off-white solid 40 mg (65%). ¹H NMR (300 MHz, CDCl₃): δ (ppm) 7.51-7.41 (m, 5H), 7.28-7.20 (t, 2H), 7.06-7.02 (m, 2H), 3.68 (s, 2H), 3.28 (s, 3H), 2.99 (s, 4H), 2.76 (s, 4H), 2.33 (s, 3H).

### Example 98

### 4-Chloro-5-[4-(4-fluorophenyl)piperazin-1-ylmethyl]-1-methyl-2-phenyl-1,2-dihydropyrazol-3-one

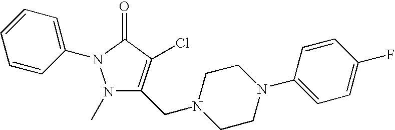

4-Chloro-5-[4-(4-fluorophenyl)piperazin-1-ylmethyl]-1-methyl-2-phenyl-1,2-dihydropyrazol-3-one was obtained from 5-bromomethyl-4-chloro-1-methyl-2-phenylpyrazolidin-3-one (30 mg, 0.1 mmol), 1-(4-fluorophenyl)piperazine (28 mg, 0.15 mmol) and potassium carbonate (49 mg, 0.35 mmol) in acetonitrile (2 mL) as white solid 27 mg (45%). ¹H NMR (300 MHz, CDCl₃): δ(ppm) 7.53-7.35 (m, 5H), 7.02-6.91 (m, 4H), 3.66 (s, 2H), 3.26 (s, 3H), 3.25-3.16 (br s, 4H), 2.76 (s, 4H).

### Example 99

### 5-[4-(4-Bromophenyl)piperazin-1-ylmethyl]-4-chloro-1-methyl-2-phenyl-1,2-dihydropyrazol-3-one

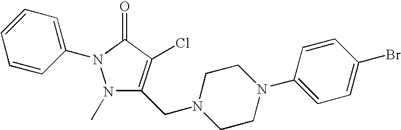

5-[4-(4-Bromophenyl)piperazin-1-ylmethyl]-4-chloro-1-methyl-2-phenyl-1,2-dihydropyrazol-3-one was obtained from 5-bromomethyl-4-chloro-1-methyl-2-phenylpyrazolidin-3-one (30 mg, 0.1 mmol), 1-(4-bromophenyl)piperazine hydrochloride (34 mg, 0.15 mmol) and potassium carbonate (49 mg, 0.35 mmol) in acetonitrile (2 mL) as an off-white solid 55 mg (79%). ¹H NMR (300 MHz, CDCl₃): δ(ppm) 7.52-7.47 (m, 2H), 7.42-7.35 (m, 5H), 6.83-6.80 (d, 2H), 3.64 (s, 2H), 3.27-3.20 (br s, 4H), 3.24 (s, 3H), 2.78-2.72 (br s, 4H).

### Example 100

### 4-Chloro-5-[4-(2-ethoxyphenyl)piperazin-1-ylmethyl]-1-methyl-2-phenyl-1,2-dihydropyrazol-3-one

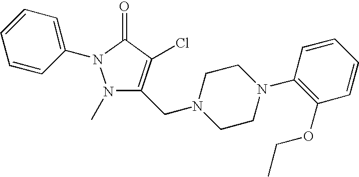

4-Chloro-5-[4-(2-ethoxyphenyl)piperazin-1-ylmethyl]-1-methyl-2-phenyl-1,2-dihydropyrazol-3-one was obtained from 5-bromomethyl-4-chloro-1-methyl-2-phenylpyrazolidin-3-one (30 mg, 0.1 mmol), 1-(2-ethoxyphenyl)piperazine monohydrochloride (31 mg, 0.15 mmol) and potassium carbonate (49 mg, 0.35 mmol) in acetonitrile (2 mL) as sticky yellow gum 64 mg (100%). ¹H NMR (300 MHz, CDCl₃): δ(ppm) 7.52-7.28 (m, 5H), 6.97-6.86 (m, 4H), 3.66 (s, 2H), 3.26 (s, 3H), 3.16-3.07 (br s, 4H), 2.79 (br s, 4H), 1.51-1.46 (t, 3H).

### Example 101

### 4-Chloro-5-[4-(2-ethylphenyl)piperazin-1-ylmethyl]-1-methyl-2-phenyl-1,2-dihydropyrazol-3-one

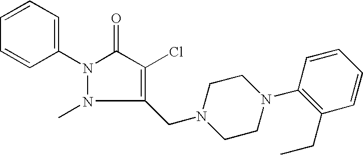

4-Chloro-5-[4-(2-ethylphenyl)piperazin-1-ylmethyl]-1-methyl-2-phenyl-1,2-dihydropyrazol-3-on was obtained from 5-bromomethyl-4-chloro-1-methyl-2-phenylpyrazolidin-3-one (30 mg, 0.1 mmol), 1-(5-chloro-2-methoxyphenyl)piperazine hydrochloride (27 mg, 0.15 mmol) and potassium carbonate (49 mg, 0.35 mmol) in acetonitrile (2 mL) as white solid 40 mg (64%). ¹H NMR (300 MHz, CDCl₃): δ(ppm) 7.53-7.41 (m, 5H), 7.38-7.10 (m, 4H), 3.67 (s, 2H), 3.28 (s, 3H), 2.97-2.96 (br s, 4H), 2.78-2.70 (br s, 4H), 2.75 (q, 2H), 1.28 (t, 3H).

### Example 102

### 4-Chloro-5-[4-(4-ethoxyphenyl)piperazin-1-ylmethyl]-1-methyl-2-phenyl-1,2-dihydropyrazol-3-one

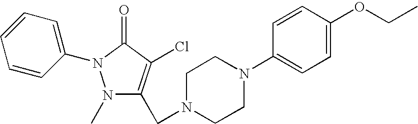

4-Chloro-5-[4-(4-ethoxyphenyl)piperazin-1-ylmethyl]-1-methyl-2-phenyl-1,2-dihydropyrazol-3-one was obtained from 5-bromomethyl-4-chloro-1-methyl-2-phenylpyrazolidin-3-one (30 mg, 0.1 mmol), 1-(4-ethoxyphenyl)piperazine hydrochloride (31 mg, 0.15 mmol) and potassium carbonate (49 mg, 0.35 mmol) in acetonitrile (2 mL) as white solid 72 mg (116%). ¹H NMR (300 MHz, CDCl₃): δ(ppm) 7.52-7.42 (m, 2H), 7.40-7.35 (m, 3H), 6.87 (q, 4H), 4.00 (q, 2H), 3.64 (s, 2H), 3.24 (s, 3H), 3.14 (s, 4H), 2.75 (s, 4H), 1.41 (t, 3H).

### Example 103

### 4-Chloro-5-[4-(5-chloro-2-methoxyphenyl)piperazin-1-ylmethyl]-1-methyl-2-phenyl-1,2-dihydropyrazol-3-one

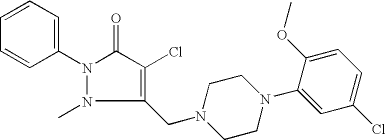

4-Chloro-5-[4-(5-chloro-2-methoxyphenyl)piperazin-1-ylmethyl]-1-methyl-2-phenyl-1,2-dihydropyrazol-3-one was obtained from 5-bromomethyl-4-chloro-1-methyl-2-phenylpyrazolidin-3-one (30 mg, 0.1 mmol), 1-(5-chloro-2-methoxyphenyl)piperazine hydrochloride (31 mg, 0.15 mmol) and potassium carbonate (49 mg, 0.35 mmol) in acetonitrile (2 mL) as white solid 52 mg (77%). ¹H NMR (300 MHz, CDCl₃): δ(ppm) 7.50-7.36 (m, 5H), 6.99-6.77 (m, 3H), 3.87 (s, 3H), 3.66 (s, 2H), 3.25 (s, 3H), 3.08 (s, 4H), 2.78 (s, 4H).

### Example 104

### 4-Chloro-5-[4-(2,4-difluorophenyl)piperazin-1-ylmethyl]-1-methyl-2-phenyl-1,2-dihydropyrazol-3-one

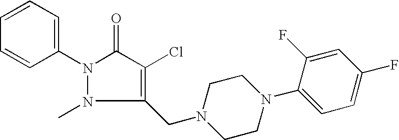

4-Chloro-5-[4-(2,4-difluorophenyl)piperazin-1-ylmethyl]-1-methyl-2-phenyl-1,2-dihydropyrazol-3-one product was obtained from 5-bromomethyl-4-chloro-1-methyl-2-phenylpyrazolidin-3-one (30 mg, 0.1 mmol), 1-(2,4-difluorophenyl)piperazine hydrochloride (29 mg, 0.15 mmol) and potassium carbonate (49 mg, 0.35 mmol) in acetonitrile (2 mL) as white solid 34 mg (54%). ¹H NMR (300 MHz, CDCl₃): δ(ppm) 7.53-7.35 (m, 5H), 6.92-6.80 (m, 3H), 3.66 (s, 2H), 3.25 (s, 3H), 3.10 (s, 4H), 2.77 (s, 4H).

### Example 105

### 4-Chloro-1-methyl-2-phenyl-5-[4-(2-trifluoromethylphenyl)piperazin-1-ylmethyl]-1,2-dihydropyrazol-3-one

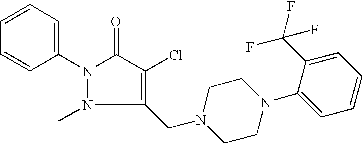

4-Chloro-1-methyl-2-phenyl-5-[4-(2-trifluoromethylphenyl)piperazin-1-ylmethyl]-1,2-dihydropyrazol-3-one was obtained from 5-bromomethyl-4-chloro-1-methyl-2-phenyl-pyrazolidin-3-one (30 mg, 0.1 mmol), 1-(2-trifluoromethylphenyl)piperazine (34 mg, 0.15 mmol) and potassium carbonate (49 mg, 0.35 mmol) in acetonitrile (2 mL) as off-white solid 60 mg (90%). ¹H NMR (300 MHz, CDCl₃): δ(ppm) 7.67 (d, 1H), 7.64-7.26 (m, 8H), 3.66 (s, 2H), 3.27 (s, 3H), 3.02-2.98 (br s, 4H), 2.74 (s, 4H).

### Example 106

### 4-Chloro-5-[4-(5-chloro-2-methylphenyl)piperazin-1-ylmethyl]-1-methyl-2-phenyl-1,2-dihydropyrazol-3-one

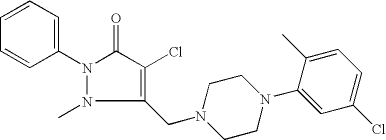

4-Chloro-5-[4-(5-chloro-2-methylphenyl)piperazin-1-ylmethyl]-1-methyl-2-phenyl-1,2-dihydropyrazol-3-one was obtained from 5-bromomethyl-4-chloro-1-methyl-2-phenylpyrazolidin-3-one (30 mg, 0.1 mmol), 1-(5-chloro-2-methylphenyl)piperazine (31 mg, 0.15 mmol) and potassium carbonate (49 mg, 0.35 mmol) in acetonitrile (2 mL) as a white fluffy solid 26 mg (40%). ¹H NMR (300 MHz, CDCl₃): δ(ppm) 7.53-7.36 (m, 5H), 7.12 (d, 1H), 6.99 (d, 2H), 3.66 (s, 2H), 3.28 (s, 3H), 2.95 (s, 4H), 2.75 (s, 4H), 2.28 (s, 3H).

### Example 107

### 4-Chloro-5-[4-(3,4-dimethoxyphenyl)piperazin-1-ylmethyl]-1-methyl-2-phenyl-1,2-dihydropyrazol-3-one

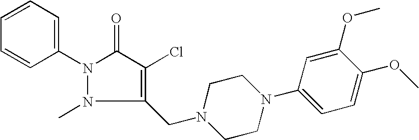

4-Chloro-5-[4-(3,4-dimethoxyphenyl)piperazin-1-ylmethyl]-1-methyl-2-phenyl-1,2-dihydropyrazol-3-one was obtained from 5-bromomethyl-4-chloro-1-methyl-2-phenylpyrazolidin-3-one (30 mg, 0.1 mmol), 1-(3,4-dimethoxyphenyl)piperazine hydrochloride (21 mg, 0.15 mmol) and potassium carbonate (49 mg, 0.35 mmol) in acetonitrile (2 mL) as a yellow-white solid 67 mg (124%). ¹H NMR (300 MHz, CDCl₃): δ(ppm) 7.53-7.35 (m, 7H), 6.62 (d, 1H), 3.86 (d, 6H), 3.66 (s, 2H), 3.25 (s, 3H), 3.16 (s, 4H), 2.76 (s, 4H).

### Example 108

### 5-(4-Benzothiazol-2-yl-piperazin-1-ylmethyl)-4-chloro-1-methyl-2-phenyl-1,2-dihydropyrazol-3-one

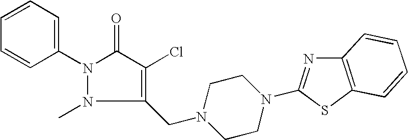

5-(4-Benzothiazol-2-yl-piperazin-1-ylmethyl)-4-chloro-1-methyl-2-phenyl-1,2-dihydropyrazol-3-one was obtained from 5-bromomethyl-4-chloro-1-methyl-2-phenyl-pyrazolidin-3-one (30 mg, 0.1 mmol), 2-piperazin-1-ylbenzothiazole (33 mg, 0.15 mmol) and potassium carbonate (49 mg, 0.35 mmol) in acetonitrile (2 mL) as yellow gum 81 mg (122%). ¹H NMR (300 MHz, CDCl₃): δ(ppm) 7.65-7.33 (m, 8H), 7.09 (m, 1H), 3.73-3.69 (br s, 4H), 3.65 (s, 2H), 3.25 (s, 3H), 2.75-2.72 (br s, 4H).

### Example 109

### 4-Chloro-5-[4-(3-chlorophenyl)piperazin-1-ylmethyl]-1-methyl-2-phenyl-1,2-dihydropyrazol-3-one

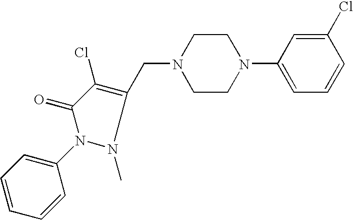

4-Chloro-5-[4-(3-chlorophenyl)piperazin-1-ylmethyl]-1-methyl-2-phenyl-1,2-dihydropyrazol-3-one was obtained from 5-bromomethyl-4-chloro-1-methyl-2-phenylpyrazolidin-3-one (30 mg, 0.1 mmol), 1-(3-chlorophenyl)piperazine (40 mg, 0.15 mmol) and potassium carbonate (49 mg, 0.35 mmol) in acetonitrile (2 mL) as an off-white solid 40 mg (55%). ¹H NMR (300 MHz, CDCl₃): δ(ppm) 7.50-7.35 (m, 5H), 7.22-7.17 (m, 1H), 6.90-6.83 (m, 3H), 3.65 (s, 2H), 3.25 (s, 7H), 2.31 (s, 4H).

### Example 110

### 4-Chloro-5-[4-(4-hydroxyphenyl)piperazin-1-ylmethyl]-1-methyl-2-phenyl-1,2-dihydropyrazol-3-one

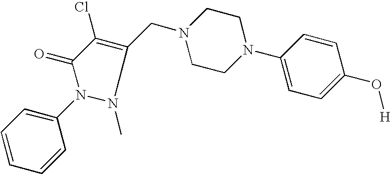

4-Chloro-5-[4-(4-hydroxyphenyl)piperazin-1-ylmethyl]-1-methyl-2-phenyl-1,2-dihydropyrazol-3-one was obtained from 5-bromomethyl-4-chloro-1-methyl-2-phenylpyrazolidin-3-one (30 mg, 0.1 mmol), 1-(4-hydroxyphenyl)piperazine (27 mg, 0.15 mmol) and potassium carbonate (49 mg, 0.35 mmol) in acetonitrile (2 mL) as an off-white solid 28 mg (47%). ¹H NMR (300 MHz, CDCl₃): δ(ppm) 7.61-7.41 (m, 5H), 6.92 (d, 2H), 6.73 (d, 2H), 3.74 (s, 2H), 3.33 (s, 3H), 3.12-3.08 (br s, 4H), 2.78-2.75 (br s, 4H).

### Example 111

### 4-Chloro-5-[4-(2,5-dimethylphenyl)piperazin-1-ylmethyl]-1-methyl-2-phenyl-1,2-dihydropyrazol-3-one

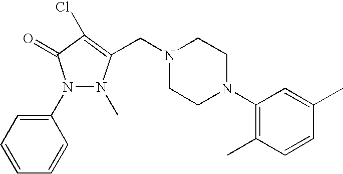

4-Chloro-5-[4-(2,5-dimethylphenyl)piperazin-1-ylmethyl]-1-methyl-2-phenyl-1,2-dihydropyrazol-3-one was obtained from 5-bromomethyl-4-chloro-1-methyl-2-phenylpyrazolidin-3-one (30 mg, 0.1 mmol), 1-(2,5-dimethylphenyl)piperazine (29 mg, 0.15 mmol) and potassium carbonate (49 mg, 0.35 mmol) in acetonitrile (2 mL) as an off-white gum 46 mg (75%). ¹H NMR (300 MHz, CDCl₃): δ(ppm) 7.54-7.33 (m, 5H), 7.36 (d, 1H), 6.86 (d, 2H), 3.67 (s, 2H), 3.28 (s, 3H), 2.96 (s, 4H), 2.69 (s, 4H), 2.32 (d, 6H).

### Example 112

### 4-Chloro-5-[5-(4-chlorophenyl)-2,5-diazabicyclo[2.2.1]hept-2-ylmethyl]-1-methyl-2-phenyl-1,2-dihydropyrazol-3-one

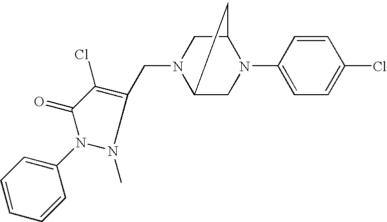

4-Chloro-5-[5-(4-chlorophenyl)-2,5-diazabicyclo[2.2.1]hept-2-ylmethyl]-1-methyl-2-phenyl-1,2-dihydropyrazol-3-one was obtained from 5-bromomethyl-4-chloro-1-methyl-2-phenylpyrazolidin-3-one (30 mg, 0.1 mmol), (1S,4S)-(−)-(4-chlorophenyl)-2-5-diazabicyclo[2.2.1]heptane hydrobromide (38 mg, 0.15 mmol) and potassium carbonate (49 mg, 0.35 mmol) in acetonitrile (2 mL) as white fluffy solid 47 mg (67%). ¹H NMR (300 MHz, CDCl₃): δ(ppm): 7.51-7.32 (s, 5H), 7.18 (d, 2H), 6.52 (d, 2H), 3.59 (q, 2H), 3.49 (s, 1H), 3.46 (d, 1H), 3.27-3.22 (m, 4H), 2.97-2.85 (q, 2H).

### Example 113

### 4-Chloro-1-methyl-2-phenyl-5-[4-(5-trifluoromethyl-pyrimidin-2-yl)-piperazin-1-ylmethyl]-1,2-dihydro-pyrazol-3-one

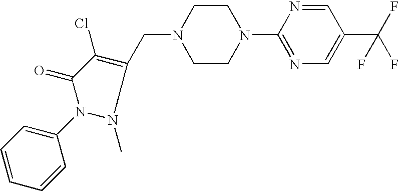

4-Chloro-1-methyl-2-phenyl-5-[4-(5-trifluoromethylpyrimidin-2-yl)-piperazin-1-ylmethyl]-1,2-dihydropyrazol-3-one was obtained from 5-bromomethyl-4-chloro-1-methyl-2-phenylpyrazolidin-3-one (30 mg, 0.1 mmol), 1-[4-(Trifluoromethyl)pyrimid-2-yl]piperazine (35 mg, 0.15 mmol) and potassium carbonate (49 mg, 0.35 mmol) in acetonitrile (2 mL) as an off-white solid 43 mg (63%). ¹H NMR (300 MHz, CDCl₃): δ(ppm): 8.52 (d, 1H), 7.53-7.33 (m, 5H), 6.80 (d, 1H), 3.93 (s, 4H), 3.64 (s, 2H), 3.26 (s, 3H), 2.65 (s, 4H).

### Example 114

### 4-Chloro-5-[4-(2,4-dimethyl-phenyl)-piperazin-1-ylmethyl]-1-methyl-2-phenyl-1,2-dihydropyrazol-3-one

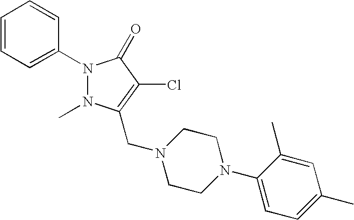

4-Chloro-5-[4-(2,4-dimethyl-phenyl)-piperazin-1-ylmethyl]-1-methyl-2-phenyl-1,2-dihydropyrazol-3-one was obtained from 5-bromomethyl-4-chloro-1-methyl-2-phenylpyrazolidin-3-one (30 mg, 0.1 mmol), 1-(2,4-dimethyl-phenyl)piperazine (29 mg, 0.15 mmol) and potassium carbonate (40 mg, 0.30 mmol) in acetonitrile (1.5 mL) as a solid 41 mg (100%). ¹H NMR (300 MHz, CDCl₃): δ(ppm): 7.38-7.53 (m, 5H), 6.96-7.04 (m, 3H), 3.66 (s, 2H), 3.27 (s, 3H), 2.95 (t, 4H), 2.74 (t, 4H), 2.36 (s, 3H), 2.30 (s, 3H).

### Example 115

### 4-Chloro-5-[4-(3,4-dimethylphenyl)piperazin-1-ylmethyl]-1-methyl-2-phenyl-1,2-dihydropyrazol-3-one

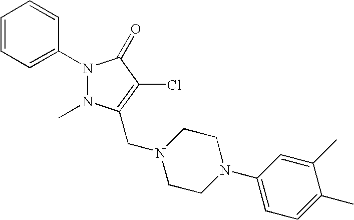

4-Chloro-5-[4-(3,4-dimethylphenyl)piperazin-1-ylmethyl]-1-methyl-2-phenyl-1,2-dihydropyrazol-3-one was obtained from 5-bromomethyl-4-chloro-1-methyl-2-phenylpyrazolidin-3-one (30 mg, 0.1 mmol), 1-(3,4-dimethylphenyl)piperazine (29 mg, 0.15 mmol) and potassium carbonate (40 mg, 0.30 mmol) in acetonitrile (1.5 mL) as a solid 37 mg (90%). ¹H NMR (300 MHz, CDCl₃): δ(ppm): 7.36-7.53 (m, 5H), 7.16 (d, 1H), 6.71-6.79 (m, 2H), 3.65 (s, 2H), 3.28 (s, 3H), 3.20 (t, 4H), 2.75 (t, 4H), 2.26 (s, 3H), 2.21 (s, 3H).

### Example 116

### 4-Chloro-5-[4-(2,4-dichlorophenyl)piperazin-1-ylmethyl]-1-methyl-2-phenyl-1,2-dihydropyrazol-3-one

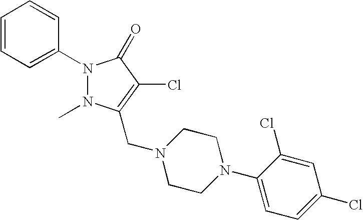

4-Chloro-5-[4-(2,4-dichlorophenyl)piperazin-1-ylmethyl]-1-methyl-2-phenyl-1,2-dihydropyrazol-3-one was obtained from 5-bromomethyl-4-chloro-1-methyl-2-phenylpyrazolidin-3-one (30 mg, 0.1 mmol), 1-(2,4-dichloro-phenyl)piperazine (40 mg, 0.15 mmol) and potassium carbonate (40 mg, 0.30 mmol) in acetonitrile (1.5 mL) as a solid 32 mg (65%). ¹H NMR (300 MHz, CDCl₃): δ(ppm): 7.37-7.49 (m, 6H), 7.19 (d, 1H), 6.97 (d, 1H), 3.66 (s, 2H), 3.24 (s, 3H), 3.08 (t, 4H), 2.77 (t, 4H).

### Example 117

### 4-Chloro-5-[4-(2,3-dimethyl-phenyl)-piperazin-1-ylmethyl]-1-methyl-2-phenyl-1,2-dihydropyrazol-3-one

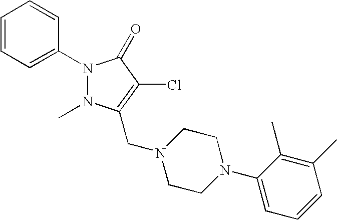

4-Chloro-5-[4-(2,3-dimethyl-phenyl)-piperazin-1-ylmethyl]-1-methyl-2-phenyl-1,2-dihydropyrazol-3-one was obtained from 5-bromomethyl-4-chloro-1-methyl-2-phenylpyrazolidin-3-one (30 mg, 0.1 mmol), 1-(2,3-dimethyl-phenyl)piperazine (29 mg, 0.15 mmol) and potassium carbonate (40 mg, 0.30 mmol) in acetonitrile (1.5 mL) as a solid 37 mg (90%). ¹H NMR (300 MHz, CDCl₃): δ(ppm): 7.35-7.53 (m, 5H), 7.10 (d, 1H), 6.94 (d, 2H), 3.67 (s, 2H), 3.27 (s, 3H), 2.96 (t, 4H), 2.77 (t, 4H), 2.31 (s, 3H), 2.26 (s, 3H).

### Example 118

### 4-Chloro-5-[4-(2,3-dichloro-phenyl)-piperazin-1-ylmethyl]-1-methyl-2-phenyl-1,2-dihydropyrazol-3-one

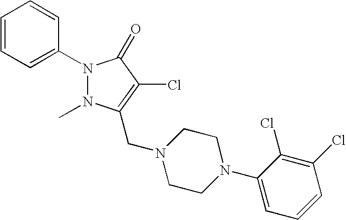

4-Chloro-5-[4-(2,3-dichloro-phenyl)-piperazin-1-ylmethyl]-1-methyl-2-phenyl-1,2-dihydropyrazol-3-one was obtained from 5-bromomethyl-4-chloro-1-methyl-2-phenylpyrazolidin-3-one (30 mg, 0.1 mmol), 1-(2,3-dichloro-phenyl)piperazine (40 mg, 0.15 mmol) and potassium carbonate (40 mg, 0.30 mmol) in acetonitrile (1.5 mL) as a solid 37 mg (90%). ¹H NMR (300 MHz, CDCl₃): δ(ppm): 7.34-7.49 (m, 5H), 7.19 (d, 2H), 6.96 (d, 1H), 3.66 (s, 2H), 3.25 (s, 3H), 3.11 (t, 4H), 2.78 (t, 4H).

### Example 119

### 4-Chloro-5-[4-(3,5-dimethyl-phenyl)-piperazin-1-ylmethyl]-1-methyl-2-phenyl-1,2-dihydropyrazol-3-one

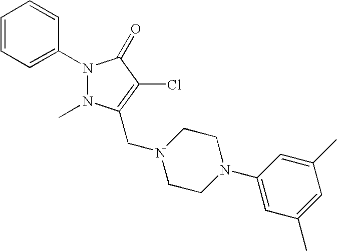

4-Chloro-5-[4-(3,5-dimethyl-phenyl)-piperazin-1-ylmethyl]-1-methyl-2-phenyl-1,2-dihydropyrazol-3-one was obtained from 5-bromomethyl-4-chloro-1-methyl-2-phenylpyrazolidin-3-one (30 mg, 0.1 mmol), 1-(3,5-dimethyl-phenyl)piperazine (29 mg, 0.15 mmol) and potassium carbonate (40 mg, 0.30 mmol) in acetonitrile (1.5 mL) as a solid 40 mg (100%). ¹H NMR (300 MHz, CDCl₃): δ(ppm): 7.35-7.53 (m, 5H), 6.59 (s, 2H), 6.57 (s, 1H), 3.66 (s, 2H), 3.25 (s, 3 h), 3.23 (t, 4H), 2.74 (t, 4H), 2.30 (s, 6H).

### Example 120

### 2-[4-(4-chloro-2-methyl-5-oxo-1-phenyl-2,5-dihydro-1H-pyrazol-3-ylmethyl)-piperazin-1-yl]-benzonitrile

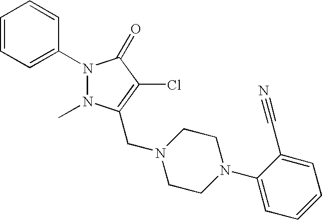

2-[4-(4-chloro-2-methyl-5-oxo-1-phenyl-2,5-dihydro-1H-pyrazol-3-ylmethyl)-piperazin-1-yl]-benzonitrile was obtained from 5-bromomethyl-4-chloro-1-methyl-2-phenylpyrazolidin-3-one (30 mg, 0.1 mmol), 2-piperazin-1-yl-benzonitrile (29 mg, 0.15 mmol) and potassium carbonate (40 mg, 0.30 mmol) in acetonitrile (1.5 mL) as a solid 40 mg (100%). ¹H NMR (300 MHz, CDCl₃): δ(ppm): 7.35-7.58 (m, 7H), 7.02-7.06 (m, 2H), 3.67 (s, 2H), 3.29 (t, 4 h), 3.25 (s, 3H), 2.82 (t, 4H).

### Example 121

### 4-Chloro-5-[4-(3-hydroxyphenyl)piperazin-1-ylmethyl]-1-methyl-2-phenyl-1,2-dihydropyrazol-3-one

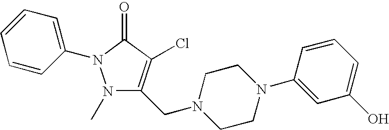

4-Chloro-5-[4-(3-hydroxyphenyl)piperazin-1-ylmethyl]-1-methyl-2-phenyl-1,2-dihydropyrazol-3-one was obtained from 5-bromomethyl-4-chloro-1-methyl-2-phenylpyrazolidin-3-one (30 mg, 0.1 mmol), 1-(3-hydroxyphenyl)piperazine (27 mg, 0.15 mmol) and potassium carbonate (41 mg, 0.30 mmol) in acetonitrile (1.5 mL) as a solid 39.9 mg (103%). ¹H NMR (300 MHz, MeOD): δ(ppm): 7.41-7.62 (m, 5H), 7.06 (t, 1H), 6.50 (dd, 1H), 6.43 (t, 1H), 6.34 (dd, 1H), 3.34 (s, 2H), 3.32 (t, 3H), 3.18 (t, 4H), 2.72 (t, 4H).

### Example 122

### 4-Chloro-1-methyl-5-(4-naphthalen-1-yl-piperazin-1-ylmethyl)-2-phenyl-1,2-dihydropyrazol-3-one

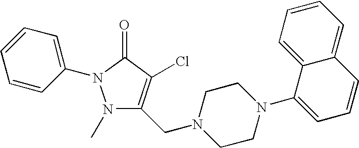

4-Chloro-1-methyl-5-(4-naphthalen-1-yl-piperazin-1-ylmethyl)-2-phenyl-1,2-dihydropyrazol-3-one was obtained from 5-bromomethyl-4-chloro-1-methyl-2-phenylpyrazolidin-3-one (30 mg, 0.1 mmol), 1-naphthalen-1-ylpiperazine (37 mg, 0.15 mmol) and potassium carbonate (41 mg, 0.30 mmol) in acetonitrile (1.5 mL) as a solid 43.3 mg (107%). ¹H NMR (300 MHz, CDCl₃): δ(ppm): 8.21-8.22 (m, 1H), 7.84-7.85 (m, 1H), 7.36-7.58 (m, 9H), 7.13 (dd, 1H), 3.73 (s, 2H), 3.29 (s, 3H), 3.12 (s, 4H), 2.90 (s, 4H).

### Example 123

### 4-Chloro-1-methyl-5-(3-methyl-4-m-tolyl-piperazin-1-ylmethyl)-2-phenyl-1,2-dihydropyrazol-3-one

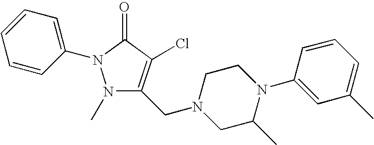

4-Chloro-1-methyl-5-(3-methyl-4-m-tolyl-piperazin-1-ylmethyl)-2-phenyl-1,2-dihydropyrazol-3-one was obtained from 5-bromomethyl-4-chloro-1-methyl-2-phenylpyrazolidin-3-one (30 mg, 0.1 mmol), 2-methyl-1-m-tolyl-piperazine (29 mg, 0.15 mmol) and potassium carbonate (41 mg, 0.30 mmol) in acetonitrile (1.5 mL) as a solid 41.1 mg (95%). ¹H NMR (300 MHz, CDCl₃): δ(ppm): 7.35-7.53 (m, 5H), 7.18 (t, 1H), 6.72 (t, 3H), 3.94-3.98 (m, 1H), 3.61 (s, 2H0, 3.30 (s, 3H), 3.18 (td, 2H), 2.94 (d, 1H), 2.64 (dd, 2H), 2.50 (td, 1H), 2.34 (s, 3H), 1.11 (d, 3H).

### Example 124

### 4-Chloro-1-methyl-5-(3-methyl-4-phenyl-piperazin-1-ylmethyl)-2-phenyl-1,2-dihydropyrazol-3-one

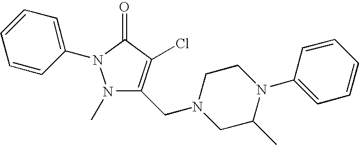

4-Chloro-1-methyl-5-(3-methyl-4-phenyl-piperazin-1-ylmethyl)-2-phenyl-1,2-dihydropyrazol-3-one was obtained from 5-bromomethyl-4-chloro-1-methyl-2-phenylpyrazolidin-3-one (30 mg, 0.1 mmol), 2-methyl-1-phenyl-piperazine (26 mg, 0.15 mmol) and potassium carbonate (41 mg, 0.30 mmol) in acetonitrile (1.5 mL) as a solid 39.7 mg (98%). ¹H NMR (300 MHz, CDCl₃): δ(ppm): 7.48-7.51 (m, 2H), 7.27-7.48 (m, 5H), 6.92-6.94 (m, 3H), 3.97-3.99 (m, 1H), 3.62 (s, 2H), 3.28 (s, 4H), 3.19 (td, 2H), 2.95 (d, 1H), 2.65 (dd, 2H), 2.51 (td, 1H), 1.11 (d, 3H).

### Example 125

### 5-(4-Biphenyl-4-yl-piperazin-1-ylmethyl)-4-chloro-2-phenyl-1,2-dihydropyrazol-3-one

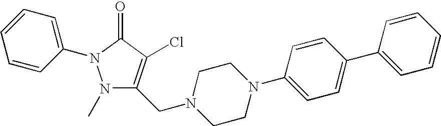

5-(4-Biphenyl-4-yl-piperazin-1-ylmethyl)-2-phenyl-1,2-dihydropyrazol-3-one was obtained from 5-bromomethyl-4-chloro-1-methyl-2-phenylpyrazolidin-3-one (30 mg, 0.1 mmol), 1-biphenyl-4-yl-piperazine (36 mg, 0.15 mmol) and potassium carbonate (41 mg, 0.30 mmol) in acetonitrile (1.5 mL) as a solid 45.9 mg (93%). ¹H NMR (300 MHz, CDCl₃): δ(ppm): 7.34-7.61 (m, 14H), 7.03 (d, 2H), 3.66 (s, 2H), 3.31 (t, 4H), 3.26 (s, 3H), 2.78 (t, 4H).

### Example 126

### 4-Chloro-1-methyl-2-phenyl-5-[4-(3-phenyl-[1,2,4]thiadiazol-5-yl)-piperazin-1-ylmethyl]-1,2-dihydropyrazol-3-one

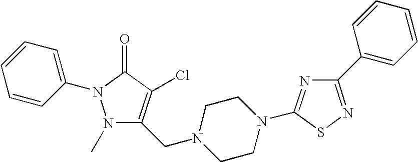

4-Chloro-1-methyl-2-phenyl-5-[4-(3-phenyl-[1,2,4]thiadiazol-5-yl)-piperazin-1-ylmethyl]-1,2-dihydropyrazol-3-one was obtained from 5-bromomethyl-4-chloro-1-methyl-2-phenylpyrazolidin-3-one (30 mg, 0.1 mmol), 1-(3-phenyl1[1,2,4]thiadiazol-5-yl)-piperazine (37 mg, 0.15 mmol) and potassium carbonate (41 mg, 0.30 mmol) in acetonitrile (1.5 mL) as a solid 46.7 mg (64%). ¹H NMR (300 MHz, CDCl₃): δ(ppm): 8.20-8.23 (m, 2H), 7.39-7.51 (m, 8H), 3.65-3.69 (m, 6H), 3.25 (s, 2H), 2.74 (t, 1H).

### Example 127

### 5-[4-(4-tert-Butyl-phenyl)-piperazin-1-ylmethyl]-4-chloro-1-methyl-2-phenyl-1,2-dihydropyrazol-3-one

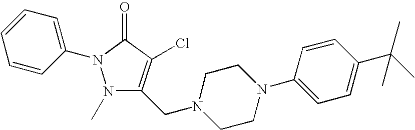

5-[4-(4-tert-Butyl-phenyl)-piperazin-1-ylmethyl]-4-chloro-1-methyl-2-phenyl-1,2-dihydropyrazol-3-one was obtained from 5-bromomethyl-4-chloro-1-methyl-2-phenylpyrazolidin-3-one (30 mg, 0.1 mmol), 1-(4-tert-butyl-phenyl)-piperazine (33 mg, 0.15 mmol) and potassium carbonate (41 mg, 0.30 mmol) in acetonitrile (1.5 mL) as a solid 43.9 mg (97%). ¹H NMR (300 MHz, CDCl₃): δ(ppm): 7.31-7.51 (m, 7H), 6.91 (d, 2H), 3.65 (s, 2H), 3.21-3.25 (m, 7H), 2.75 (t, 4H), 1.32 (s, 9H).

### Example 128

### 5-[4-(2-Acetyl-4-fluorophenyl)-piperazin-1-ylmethyl]-4-chloro-1-methyl-2-phenyl-1,2-dihydropyrazol-3-one

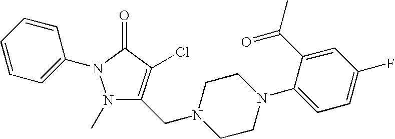

5-[4-(2-Acetyl-4-fluorophenyl)-piperazin-1-ylmethyl]-4-chloro-1-methyl-2-phenyl-1,2-dihydropyrazol-3-one was obtained from 5-bromomethyl-4-chloro-1-methyl-2-phenylpyrazolidin-3-one (30 mg, 0.1 mmol), 1-(5-fluoro-2-piperazin-1-yl-phenyl)ethanone (33 mg, 0.15 mmol) and potassium carbonate (41 mg, 0.30 mmol) in acetonitrile (2.0 mL) as a pale yellow solid 40.9 mg (92%). ¹H NMR (300 MHz, CDCl₃): δ(ppm): 7.48 (d, 2H), 7.35-7.41 (m, 3H), 7.10-7.15 (m, 3H), 3.65 (s, 2H), 3.23 (s, 3H), 3.00 (t, 4H), 2.70-2.78 (m, 7H).

### Example 129

### 4-Chloro-1-ethyl-5-[4-(4-fluoro-phenyl)-piperazin-1-ylmethyl]-2-phenyl-1,2-dihydro-pyrazol-3-one

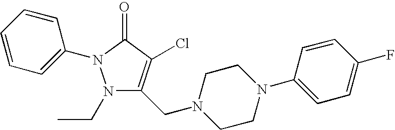

4-Chloro-1-ethyl-5-[4-(4-fluoro-phenyl)-piperazin-1-ylmethyl]-2-phenyl-1,2-dihydro-pyrazol-3-one was synthesized with general procedure #5 using 5-bromomethyl-4-chloro-1-ethyl-2-phenyl-1,2-dihydro-pyrazol-3-one (40 mg, 0.1267 mmol) and 1-(4-fluoro-phenyl)-piperazine (34.26 mg, 0.1901 mmol). The ¹H NMR (300 MHz, CDCl₃) δ (ppm): 7.47 (m, 5), 6.95 (m, 4H), 3.78 (q, 2H), 3.63 (s, 2H), 3.16 (t, 4H), 2.76 (t, 4H), 0.89 (t, 3H).

### Example 130

### 4-Chloro-5-[4-(2-ethoxy-phenyl)-piperazin-1-ylmethyl]-1-ethyl-2-phenyl-1,2-dihydro-pyrazol-3-one

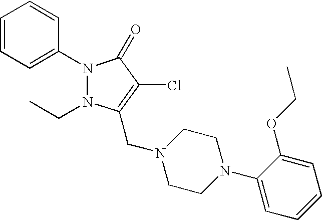

4-Chloro-5-[4-(2-ethoxy-phenyl)-piperazin-1-ylmethyl]-1-ethyl-2-phenyl-1,2-dihydro-pyrazol-3-one was synthesized with general procedure #5 using 5-bromomethyl-4-chloro-1-ethyl-2-phenyl-1,2-dihydro-pyrazol-3-one (40 mg, 0.1267 mmol) and 1-(2-Ethoxy-phenyl)-piperazine (38.84 mg, 0.1901 mmol). ¹H NMR (300 MHz, CDCl₃) δ (ppm): 7.45 (m, 5H), 6.93 (m, 4H), 4.10 (q, 2H), 3.81 (q, 2H), 3.64 (s, 2H), 3.16 (broad, 4H), 2.8 (broad, 4H), 1.49 (t, 3H), 0.88 (t, 3H).

### Example 131

### 5-[4-(4-Bromo-phenyl)-piperazin-1-ylmethyl]-4-chloro-1-ethyl-2-phenyl-1,2-dihydro-pyrazol-3-one

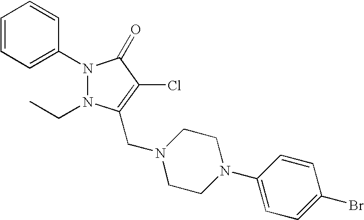

5-[4-(4-Bromo-phenyl)-piperazin-1-ylmethyl]-4-chloro-1-ethyl-2-phenyl-1,2-dihydro-pyrazol-3-one was synthesized using 5-bromomethyl-4-chloro-1-ethyl-2-phenyl-1,2-dihydro-pyrazol-3-one (40 mg, 0.1267 mmol) and 1-(4-bromo-phenyl)-piperazine (45.83 mg, 0.1901 mmol). ¹H NMR (300 MHz, CDCl₃) δ (ppm): 7.44 (m, 7H), 6.80 (m, 2H), 3.78 (q, 2H), 3.62 (s, 2H), 3.21 (t, 4H), 2.75 (t, 4H), 0.89 (s, 3H).

### Example 132

### 4-Chloro-1-ethyl-2-phenyl-5-(4-o-tolyl-piperazin-1-ylmethyl)-1,2-dihydro-pyrazol-3-one

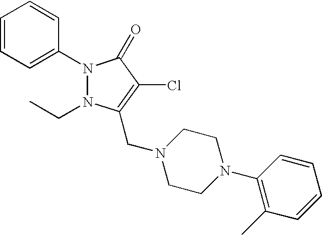

4-Chloro-1-ethyl-2-phenyl-5-(4-o-tolyl-piperazin-1-ylmethyl)-1,2-dihydro-pyrazol-3-one was synthesized using 5-bromomethyl-4-chloro-1-ethyl-2-phenyl-1,2-dihydro-pyrazol-3-one (40 mg, 0.1267 mmol) and 1-o-tolyl-piperazine (33.5 mg, 0.1901 mmol). ¹H NMR (300 MHz, CDCl₃) δ (ppm): 7.45 (m, 5H), 7.06 (m, 2H), 7.03 (m, 2H), 3.82 (q, 2H), 3.65 (s, 2H), 2.98 (broad, 4H), 2.77 (broad, 4H), 2.34 (s, 3H), 0.93 (t, 3H).

### Example 133

### 4-Chloro-ethyl-2-phenyl-5-[4-(3-phenyl-[1,2,4]thiadiazol-5-yl)-piperazin-1-ylmethyl]-1,2-dihydro-pyrazol-3-one

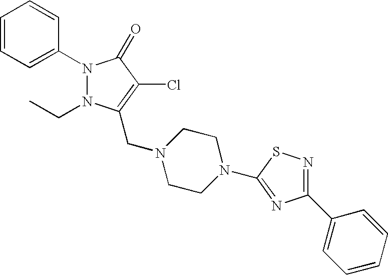

4-Chloro-ethyl-2-phenyl-5-[4-(3-phenyl-[1,2,4]thiadiazol-5-yl)-piperazin-1-ylmethyl]-1,2-dihydro-pyrazol-3-one synthesized with general procedure #5 with 5-bromomethyl-4-chloro-1-ethyl-2-phenyl-1,2-dihydro-pyrazol-3-one (40 mg, 0.1267 mmol) and 1-(3-phenyl-[1,2,4]thiadizol-5-yl)-piperazine (46.9 mg, 0.1901 mmol). ¹H NMR (300 MHz, CDCl₃) δ (ppm): 0.91 (t, 5H), 2.77 (t, 4H), 3.71 (m, 9H), 7.45 (m, 9H), 8.21 (m, 2H).

### Example 134

### 8-(4-Chloro-2-ethyl-5-oxo-1-phenyl-2,5-dihydro-1H-pyrazol-3-ylmethyl)-1-phenyl-1,3,8-triaza-spiro[4,5]decan-4-one

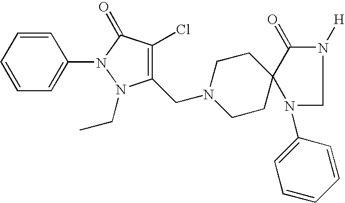

8-(4-Chloro-2-ethyl-5-oxo-1-phenyl-2,5-dihydro-1H-pyrazol-3-ylmethyl)-1-phenyl-1,3,8-triaza-spiro[4,5]decan-4-one synthesized with general procedure #5 using 5-bromomethyl-4-chloro-1-methyl-2-phenyl-1,2-dihydro-pyrazol-3-one (40 mg, 0.1267 mmol) and 1-phenyl-1,3,8-triaza-spiro[4,5]decan-4-one (43.5 mg, 0.1901 mmol). ¹H NMR (300 MHz, CDCl₃) δ (ppm): 7.43 (m, 7H), 6.86 (m, 3H), 4.78 (s, 2H), 3.87 (q, 2H), 3.67 (s, 2H), 3.06 (m, 2H), 2.91 (broad, 2H), 2.73 (m, 2H), 1.80 (d, 2H), 0.96 (m, 3H).

### Example 135

### 6-[4-(4-Chloro-2-methyl-5-oxo-1-phenyl-2,5-dihydro-1H-pyrazol-3-ylmethyl)-piperazin-1-yl]-nicotinonitrile

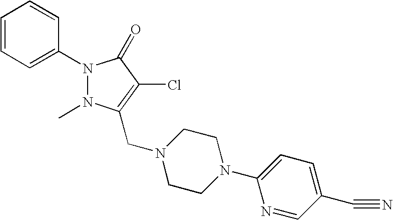

6-[4-(4-Chloro-2-methyl-5-oxo-1-phenyl-2,5-dihydro-1H-pyrazol-3-ylmethyl)-piperazin-1-yl]-nicotinonitrile synthesized using 5-bromomethyl-4-chloro-1-methyl-2-phenyl-1,2-dihydro-pyrazol-3-one (30 mg, 0.0995 mmol) and 6-piperazin-1-yl-nicotinonitrile (28.08 mg, 0.149 mmol). ¹H NMR (300 MHz, CDCl₃) δ (ppm): 8.43 (s, 1H), 7.63 (m, 1H), 7.45 (m, 5H), 6.63 (d, 1H), 3.74 (t, 4H), 3.64 (s, 2H), 3.25 (s, 2H), 2.68 (t, 4H).

### Example 136

### 4-Chloro-1-methyl-5-[4-(6-methyl-pyridin-2-yl)-piperazin-1-ylmethyl]-2-phenyl-1,2-dihydro-pyrazol-3-one

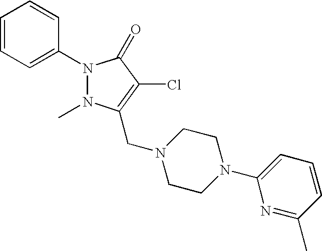

4-Chloro-1-methyl-5-[4-(6-methyl-pyridin-2-yl)-piperazin-1-ylmethyl]-2-phenyl-1,2-dihydro-pyrazol-3-one was synthesized using 5-bromomethyl-4-chloro-1-methyl-2-phenyl-1,2-dihydro-pyrazol-3-one (30 mg, 0.0995 mmol) and 6-piperazin-1-yl-nicotinonitrile (26.44 mg, 0.1492 mmol). ¹H NMR (300 MHz, CDCl₃) δ (ppm): 7.42 (m, 5H), 6.52 (m, 2H), 3.63 (s, 2H0, 3.59 (t, 4H), 3.26 (s, 3H), 2.69 (t, 4H), 2.45 (s, 3H).

### Example 137

### 4-Chloro-1-methyl-2-phenyl-5-[4-(3-trifluoromethyl-pyridin-2-yl)-piperazin-1-ylmethyl]-1,2-dihydro-pyrazol-3-one

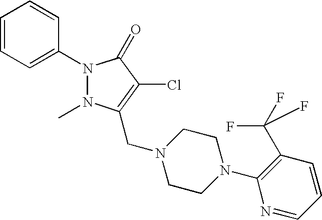

4-Chloro-1-methyl-2-phenyl-5-[4-(3-trifluoromethyl-pyridin-2-yl)-piperazin-1-ylmethyl]-1,2-dihydro-pyrazol-3-one was synthesized using 5-bromomethyl-4-chloro-1-methyl-2-phenyl-1,2-dihydro-pyrazol-3-one (30 mg, 0.0995 mmol) and 1-(3-trifluoromethyl-pyridin-2-yl)-piperazine (34.49 mg, 0.1492 mmol). ¹H NMR (300 MHz, CDCl₃) δ (ppm): 8.46 (d, 1H), 7.90 (q, 1H), 7.42 (m, 5H), 7.05 (m, 1H), 3.67 (s, 1H), 3.35 (t, 4H), 3.29 (s, 3H), 2.74 (s, 4H).

### Example 138

### 4-Chloro-1-methyl-2-phenyl-5-[4-(5-trifluoromethyl-pyridin-2-yl)-piperazin-1-ylmethyl]-1,2-dihydro-pyrazol-3-one

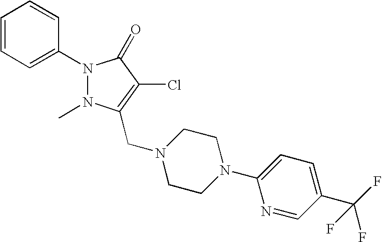

4-Chloro-1-methyl-2-phenyl-5-[4-(5-trifluoromethyl-pyridin-2-yl)-piperazin-1-ylmethyl]-1,2-dihydro-pyrazol-3-one was synthesized using 5-bromomethyl-4-chloro-1-methyl-2-phenyl-1,2-dihydro-pyrazol-3-one (30 mg, 0.0995 mmol) and 1-(5-trifluoromethyl-pyridin-2-yl)-piperazine (34.49 mg, 0.1492 mmol). ¹H NMR (300 MHz, CDCl₃) δ (ppm): 8.42 (m, 1H), 7.50 (q, 1H), 7.42 (m, 5H), 6.68 (d, 1H), 3.71 (t, 4H), 3.64 (s, 3H), 3.26 (s, 3H), 2.69 (t, 4H).

### Example 139

### 4-Chloro-1-methyl-5-[4(3-methyl-pyridin-2-yl)-piperazin-1-ylmethyl]-2-phenyl-1,2-dihydro-pyrazol-3-one

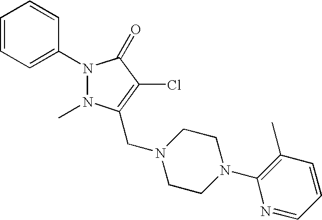

4-Chloro-1-methyl-5-[4(3-methyl-pyridin-2-yl)-piperazin-1-ylmethyl]-2-phenyl-1,2-dihydro-pyrazol-3-one was synthesized using 5-bromomethyl-4-chloro-1-methyl-2-phenyl-1,2-dihydro-pyrazol-3-one (30 mg, 0.0995 mmol) and 1-(3-methyl-pyridin-2-yl)-piperazine (26.44 mg, 0.1492 mmol). ¹H NMR (300 MHz, CDCl₃) δ (ppm): 8.17 (m, 1H), 7.41 (m, 6H), 6.90 (q, 1 h), 3.67 (s, 2H), 3.23 (m, 7H), 2.75 (t, 4H), 2.30 (s, 3H), 2.18 (s, 1H).

### Example 140

### 4-Chloro-5-[4-(3-chloro-5-trifluoromethyl-pyridin-2-yl)-piperazin-1-yl-methyl]-1-methyl-2-phenyl-1,2-dihydro-pyrazol-3-one

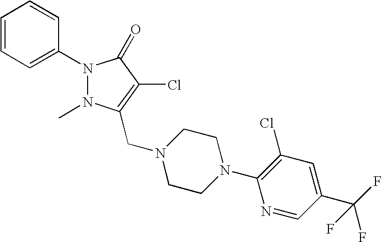

4-Chloro-5-[4-(3-chloro-5-trifluoromethyl-pyridin-2-yl)-piperazin-1-yl-methyl]-1-methyl-2-phenyl-1,2-dihydro-pyrazol-3-one was synthesized with general procedure using 5-bromomethyl-4-chloro-1-methyl-2-phenyl-1,2-dihydro-pyrazol-3-one (30 mg, 0.0995 mmol) and 1-(3-chloro-5-trifluoromethyl-pyridin-2-yl)-piperazine (39.63 mg, 0.1492 mmol). ¹H NMR (300 MHz, CDCl₃) δ (ppm): 8.42 (d, 1H), 7.79 (d, 1H), 7.43 (m, 5H), 3.67 (s, 2H), 3.58 (s, 4H), 3.29 (s, 3H), 2.76 (s, 4H).

### Example 141

### 2-[4-(4-Chloro-2-methyl-5-oxo-1-phenyl-2,5-dihydro-1H-pyrazol-3-ylmethyl)-piperazin-1-yl]-nicotinonitrile

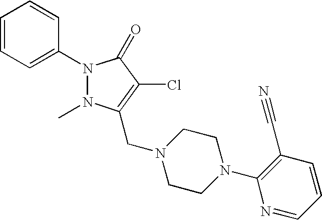

2-[4-(4-Chloro-2-methyl-5-oxo-1-phenyl-2,5-dihydro-1H-pyrazol-3-ylmethyl)-piperazin-1-yl]-nicotinonitrile was synthesized with general procedure using 5-bromomethyl-4-chloro-1-methyl-2-phenyl-1,2-dihydro-pyrazol-3-one (30 mg, 0.0995 mmol) and 2-piperazin-1-yl-nicotinonitrile (28.08 mg, 0.1492 mmol). ¹H NMR (300 MHz, CDCl₃) δ (ppm): 8.37 (q, 1H), 7.80 (q, 1H), 7.40 (m, 5H), 6.85 (q, 1H), 3.78 (t, 4H), 3.25 (s, 3H), 2.74 (s, 4H).

### Example 142

### 4-Chloro-1-methyl-5-[4-(4-methyl-pyridin-2-yl)-piperazin-1-ylmethyl]-2-phenyl-1,2-dihydro-pyrazol-3-one

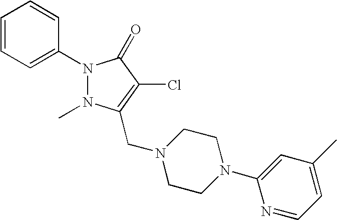

4-Chloro-1-methyl-5-[4-(4-methyl-pyridin-2-yl)-piperazin-1-ylmethyl]-2-phenyl-1,2-dihydro-pyrazol-3-one was synthesized with 5-bromomethyl-4-chloro-1-methyl-2-phenyl-1,2-dihydro-pyrazol-3-one (30 mg, 0.0995 mmol) and 1-(4-methyl-pyridin-2-yl)-piperazine (26.44 mg, 0.1492 mmol). ¹H NMR (300 MHz, CDCl₃) δ (ppm): 8.07 (q, 1H), 7.47 (m, 5H), 6.52 (q, 2H), 3.63 (s, 2H), 3.58 (t, 4H), 3.25 (s, 3H), 2.69 (t, 4H), 2.29 (s, 3H).

### Example 143

### 4-Chloro-1-methyl-2-phenyl-5-(4-m-tolyl-piperazin-1-ylmethyl)-1,2-dihydro-

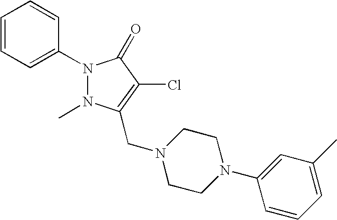

pyrazol-3-one

4-Chloro-1-methyl-2-phenyl-5-(4-m-tolyl-piperazin-1-ylmethyl)-1,2-dihydro-pyrazol-3-one was synthesized using 5-bromomethyl-4-chloro-1-methyl-2-phenyl-1,2-dihydro-pyrazol-3-one (30 mg, 0.0995 mmol) and 1-m-tolyl-piperazine (26.3 mg, 0.1492 mmol). ¹H NMR (300 MHz, CDCl₃) δ (ppm): 7.40 (m, 6H), 6.75 (q, 3H0, 3.65 (s, 2H), 2.83 (m, 7H), 2.75 (t, 4H), 2.35 (s, 3H).

### Example 144

### 4-Chloro-5-[4-(2-fluoro-phenyl)-piperazin-1-ylmethyl]-1-methyl-2-phenyl-1,2-dihydro-pyrazol-3-one

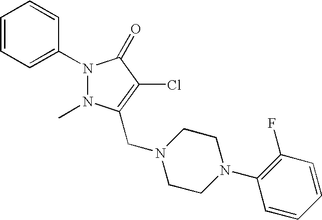

4-Chloro-5-[4-(2-fluoro-phenyl)-piperazin-1-ylmethyl]-1-methyl-2-phenyl-1,2-dihydro-pyrazol-3-one was synthesized using 5-bromomethyl-4-chloro-1-methyl-2-phenyl-1,2-dihydro-pyrazol-3-one (30 mg, 0.0995 mmol) and 1-(2-fluoro-phenyl)-4-methyl-piperazine (26.89 mg, 0.1492 mmol). ¹H NMR (300 MHz, CDCl₃) δ (ppm): 7.43 (m, 5H), 7.06 (m, 4H), 3.66 (s, 2H0, 3.26 (s, 3H), 3.16 (d, 4H), 2.79 (s, 4H).

### Example 145

### 4-Chloro-5-[4-(2-Chloro-phenyl)-piperazin-1-ylmethyl]-1-methyl-2-phenyl-1,2-dihydro-pyrazol-3-one

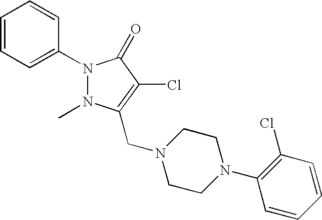

4-Chloro-5-[4-(2-Chloro-phenyl)-piperazin-1-ylmethyl]-1-methyl-2-phenyl-1,2-dihydro-pyrazol-3-one was synthesized with general procedure using 5-bromomethyl-4-chloro-1-methyl-2-phenyl-1,2-dihydro-pyrazol-3-one (30 mg, 0.0995 mmol) and 1-(2-chloro-phenyl)-4-methyl-piperazine (29.3 mg, 0.1492 mmol). ¹H NMR (300 MHz, CDCl₃) δ (ppm): 7.38 (m, 7H), 7.05 (m, 2H), 3.67 (s, 2H), 3.26 (s, 3H), 3.13 (s, 4H0, 2.79 (s, 4H).

### Example 146

### 4-Chloro-1-methyl-2-phenyl-5-(4-p-tolyl-piperazin-1-ylmethyl)-1,2-dihydro-pyrazol-3-one

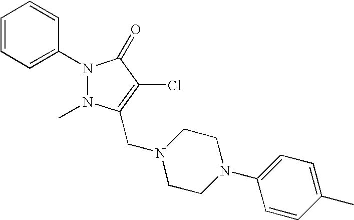

4-Chloro-1-methyl-2-phenyl-5-(4-p-tolyl-piperazin-1-ylmethyl)-1,2-dihydro-pyrazol-3-one was synthesized with general procedure using 5-bromomethyl-4-chloro-1-methyl-2-phenyl-1,2-dihydro-pyrazol-3-one (30 mg, 0.0995 mmol) and 1-p-tolyl-piperazine (26.3 mg, 0.1492 mmol). ¹H NMR (300 MHz, CDCl₃) δ (ppm): 7.42 (m, 5H), 7.11 (d, 2H), 6.89 (d, 2H), 3.65 (s, 2H), 3.25 (s, 3H), 3.20 (t, 4H), 2.76 (t, 4H), 2.30 (s, 3H).

### Example 147

### 8-(4-Chloro-5-oxo-1-phenyl-2-propyl-2,5-dihydro-1H-pyrazol-3-ylmethyl)-1-

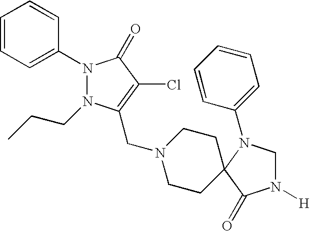

phenyl-1,3,8-triaza-spiro[4.5]decan-4-one

8-(4-Chloro-5-oxo-1-phenyl-2-propyl-2,5-dihydro-1H-pyrazol-3-ylmethyl)-1-phenyl-1,3,8-triaza-spiro[4.5]decan-4-one is made by following general procedure 5-bromomethyl-4-chloro-2-phenyl-1-propyl-1,2-dihydro-pyrazol-3-one (20 mg, 0.06 mmol), 1-phenyl-1,3,8-triaza-spiro[4.5]decan-4-one (21.04 mg, 0.091 mmols), K₂CO₃ (41.92 mg, 0.301 mmol), and 3 ml of acetonitrile was used to make 32 mg of product. ¹H NMR (300 MHz, CDCl₃) δ (ppm): 7.37 (m, 8H), 6.88 (t, 3H), 4.77 (s, 2H), 3.71 (q, 4H), 3.07 (t, 2H), 2.90 (d, 2H), 2.68 (m, 2H), 1.80 (d, 3H), 1.45 (m, 2H), 0.80 (t, 3H).

### Example 148

### 4-Chloro-5-[4-(5-chloro-2-methyl-phenyl)-piperazin-1-ylmethyl]-2-phenyl-1-propyl-1,2-dihydro-pyrazol-3-one

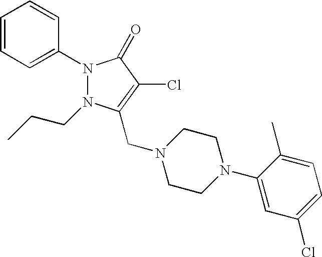

4-Chloro-5-[4-(5-chloro-2-methyl-phenyl)-piperazin-1-ylmethyl]-2-phenyl-1-propyl-1,2-dihydro-pyrazol-3-one was made with general procedure. 5-Bromomethyl-4-chloro-2-phenyl-1-propyl-1,2-dihydro-pyrazol-3-one (30 mg, 0.091 mmol), 1-(5-chloro-2-methyl-phenyl)-piperazine (28.8 mg, 0.1365 mmol), K₂CO₃ (62.9 mg, 0.455 mmol), and 4 ml of acetonitrile was used. ¹H NMR (300 MHz, CDCl₃) δ (ppm): 7.42 (m, 5H), 7.13 (d, 1H), 6.98 (t, 2H), 3.68 (m, 4H), 2.94 (t, 4H), 2.75 (s, 4H), 2.28 (s, 3H), 1.36 (m, 2H), 0.77 (m, 3H).

### Example 149

### 4-Chloro-5-[4-(5-chloro-2-methoxy-phenyl)-piperazin-1-ylmethyl]-2-phenyl-

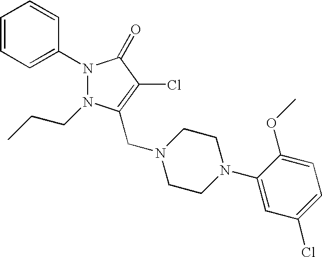

1-propyl-1,2-dihydro-pyrazol-3-one

4-Chloro-5-[4-(5-chloro-2-methoxy-phenyl)-piperazin-1-ylmethyl]-2-phenyl-1-propyl-1,2-dihydro-pyrazol-3-one was made with general procedure. 5-Bromomethyl-4-chloro-2-phenyl-1-propyl-1,2-dihydro-pyrazol-3-one (30 mg, 0.091 mmol), 1-(5-chloro-2-methoxy-phenyl)-piperazine (35.95 mg, 0.1365 mmol), K₂CO₃ (62.9 mg, 0.455 mmol), and 4 ml of acetonitrile was used. ¹H NMR (300 MHz, CDCl₃) δ (ppm): 7.38 (m, 5H), 6.96 (t, 1H), 6.88 (d, 1H), 6.78 (d, 1H), 3.87 (s, 3H), 3.67 (m, 4H), 3.09 (s, 4H), 2.78 (d, 4H), 1.30 (m, 2H), 0.75 (t, 3H).

### Example 150

### 5-(4-Acetyl-4-phenyl-piperidin-1-ylmethyl)-4-chloro-2-phenyl-1-propyl-1,2-dihydro-pyrazol-3-one

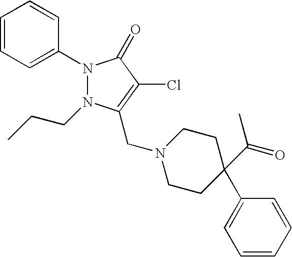

5-(4-Acetyl-4-phenyl-piperidin-1-ylmethyl)-4-chloro-2-phenyl-1-propyl-1,2-dihydro-pyrazol-3-one was made with general procedure. 5-Bromomethyl-4-chloro-2-phenyl-1-propyl-1,2-dihydro-pyrazol-3-one (30 mg, 0.091 mmol), 1-(4-phenyl-piperidin-4-yl)-ethanone (32.75 mg, 0.1365 mmol), K₂CO₃ (62.9 mg, 0.455 mmol), and 4 ml of acetonitrile was used. ¹H NMR (300 MHz, CDCl₃) δ (ppm): 7.36 (m, 10H), 3.63 (q, 2H), 3.52 (s, 2H), 2.78 (t, 2H), 2.46 (t, 4H), 2.09 (t, 2H), 1.94 (s, 3H), 1.29 (m, 2H), 0.74 (t, 3H).

### Example 151

### 4-Chloro-2-phenyl-5-(4-phenyl-4-propionyl-piperidin-1-ylmethyl)-1-propyl-1,2-dihydro-pyrazol-3-one

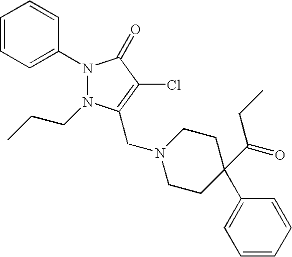

4-Chloro-2-phenyl-5-(4-phenyl-4-propionyl-piperidin-1-ylmethyl)-1-propyl-1,2-dihydro-pyrazol-3-one was made with general procedure. 5-Bromomethyl-4-chloro-2-phenyl-1-propyl-1,2-dihydro-pyrazol-3-one (30 mg, 0.091 mmol), 1-(4-phenyl-piperidin-4-yl)-propan-1-one (34.6 mg, 0.1365 mmol), K₂CO₃ (62.9 mg, 0.455 mmol) and 4 ml of acetonitrile was used. ¹H NMR (300 MHz, CDCl₃) δ (ppm): 7.37 (m, 10H), 3.60 (t, 2H), 3.49 (d, 2H), 2.77 (t, 2H), 2.48 (q, 4H), 2.27 (q, 2H), 2.09 (t, 2H), 1.29 (m, 2H), 0.91 (q, 3H), 0.71 (t, 3H).

### Example 152

### 5-(4-Butyryl-4-phenyl-piperidin-1-ylmethyl)-4-chloro-2-phenyl-1-propyl-1,2-dihydro-pyrazol-3-one

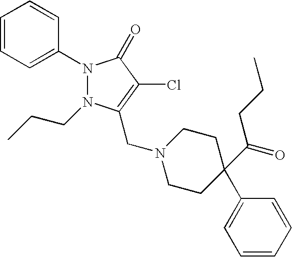

5-(4-Butyryl-4-phenyl-piperidin-1-ylmethyl)-4-chloro-2-phenyl-1-propyl-1,2-dihydro-pyrazol-3-one was made with general procedure. 5-Bromomethyl-4-chloro-2-phenyl-1-propyl-1,2-dihydro-pyrazol-3-one (30 mg, 0.091 mmol), 1-(4-phenyl-piperidin-4-yl)-butan-1-one (36.55 mg, 0.1365 mmol), K₂CO₃ (62.9 mg, 0.455 mmol), and 4 ml of acetonitrile was used. ¹H NMR (300 MHz, CDCl₃) δ (ppm): 7.38 (m, 10H), 3.63 (t, 2H), 3.49 (d, 2H), 2.76 (broad, 2H), 2.48 (d, 4H), 2.17 (broad, 4H), 1.44 (q, 2H), 1.28 (m, 2H), 0.69 (m, 6H).

### Example 153

### 1-(4-Chloro-5-oxo-1-phenyl-2-propyl-2,5-dihydro-1H-pyrazol-3-ylmethyl-4-phenyl-piperidine-4-carbonitrile

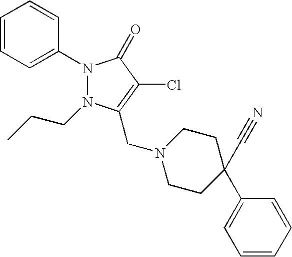

1-(4-Chloro-5-oxo-1-phenyl-2-propyl-2,5-dihydro-1H-pyrazol-3-ylmethyl-4-phenyl-piperidine-4-carbonitrile was made with general procedure. 5-Bromomethyl-4-chloro-2-phenyl-1-propyl-1,2-dihydro-pyrazol-3-one (30 mg, 0.091 mmol), 4-phenyl-piperidine-4-carbonitrile (30.40 mg, 0.14 mmol), K₂CO₃ (62.9 mg, 0.455 mmol), and 4 ml of acetonitrile was used. ¹H NMR (300 MHz, CDCl₃) δ (ppm): 7.46 (m, 10H), 3.65 (m, 4H), 3.09 (d. 2H), 2.74 (m, 2H), 2.11 (m, 4H), 1.29 (m, 2H), 0.76 (t, 3H).

### Example 154

### 4-Chloro-5-[4-(3,4-dimethyl-phenyl)-piperazin-1-ylmethyl]-1-ethyl-2-phenyl-1,2-dihydropyrazol-3-one

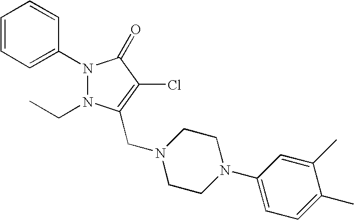

4-Chloro-5-[4-(3,4-dimethyl-phenyl)-piperazin-1-ylmethyl]-1-ethyl-2-phenyl-1,2-dihydropyrazol-3-one was obtained from 5-bromomethyl-4-chloro-1-ethyl-2-phenylpyrazolidin-3-one (31 mg, 0.1 mmol), 1-(3,4-dimethyl-phenyl)piperazine (29 mg, 0.15 mmol) and potassium carbonate (40 mg, 0.30 mmol) in acetonitrile (1.5 mL) as a solid 30 mg (70%). ¹H NMR (300 MHz, CDCl₃): δ (ppm) 7.35-7.51 (m, 5H), 7.06 (d, 1H), 6.80 (s, 1H), 6.78 (d, 1H), 4.13 (q, 2H), 3.63 (s, 2H), 3.19 (t, 4H), 2.77 (t, 4H), 2.26 (s, 3H), 2.21 (s, 3H), 0.90 (t, 3H).

### Example 155

### 4-Chloro-5-[4-(2,4-dichloro-phenyl)-piperazin-1-ylmethyl]-1-ethyl-2-phenyl-1,2-dihydropyrazol-3-one

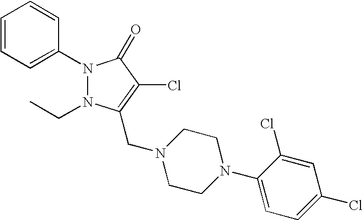

4-Chloro-5-[4-(2,4-dichloro-phenyl)-piperazin-1-ylmethyl]-1-ethyl-2-phenyl-1,2-dihydropyrazol-3-one was obtained from 5-bromomethyl-4-chloro-1-ethyl-2-phenylpyrazolidin-3-one (31 mg, 0.1 mmol), 1-(2,4-dichloro-phenyl)piperazine (40 mg, 0.15 mmol) and potassium carbonate (40 mg, 0.30 mmol) in acetonitrile (1.5 mL) as a solid 38 mg (83%). ¹H NMR (300 MHz, CDCl₃): δ (ppm) 7.35-7.50 (m, 6H), 7.20 (d, 1H), 6.97 (d, 1H), 3.81 (q, 2H), 3.65 (s, 2H), 3.08 (t, 4H), 2.79 (t, 4H), 0.91 (t, 3H).

### Example 156

### 4-Chloro-5-[4-(2,3-dimethyl-phenyl)-piperazin-1-ylmethyl]-1-ethyl-2-phenyl-1,2-dihydropyrazol-3-one

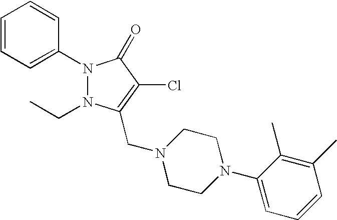

4-Chloro-5-[4-(2,3-dimethyl-phenyl)-piperazin-1-ylmethyl]-1-ethyl-2-phenyl-1,2-dihydropyrazol-3-one was obtained from 5-bromomethyl-4-chloro-1-ethyl-2-phenylpyrazolidin-3-one (31 mg, 0.1 mmol), 1-(2,3-dimethyl-phenyl)piperazine (29 mg, 0.15 mmol) and potassium carbonate (40 mg, 0.30 mmol) in acetonitrile (1.5 mL) as a solid 37 mg (88%). ¹H NMR (300 MHz, CDCl₃): δ (ppm) 7.35-7.51 (m, 5H), 7.04 (d, 1H), 6.72-6.80 (m, 2H), 3.80 (q, 2H), 3.63 (s, 2H), 3.20 (t, 4H), 2.77 (t, 4H), 2.26 (s, 3H), 2.07 (s, 3H), 0.89 (t, 3H).

### Example 157

### 4-Chloro-5-[4-(2,3-dichloro-phenyl)-piperazin-1-ylmethyl]-1-ethyl-2-phenyl-1,2-dihydropyrazol-3-one

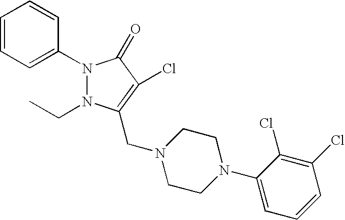

4-Chloro-5-[4-(2,3-dichloro-phenyl)-piperazin-1-ylmethyl]-1-ethyl-2-phenyl-1,2-dihydropyrazol-3-one was obtained from 5-bromomethyl-4-chloro-1-ethyl-2-phenylpyrazolidin-3-one (31 mg, 0.1 mmol), 1-(2,3-dichloro-phenyl)piperazine (40 mg, 0.15 mmol) and potassium carbonate (40 mg, 0.30 mmol) in acetonitrile (1.5 mL) as a solid 35 mg (76%). ¹H NMR (300 MHz, CDCl₃): δ (ppm) 7.36-7.50 (m, 5H), 7.16-7.19 (m, 2H), 6.97 (d, 1H), 3.80 (q, 2H), 3.66 (s, 3H), 3.11 (t, 4H), 2.81 (t, 4H), 0.91 (t, 3H).

### Example 158

### 4-Chloro-5-[4-(3,5-dichloro-phenyl)-piperazin-1-ylmethyl]-1-ethyl-2-phenyl-1,2-dihydropyrazol-3-one

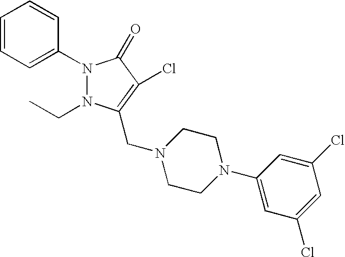

4-Chloro-5-[4-(3,5-dichloro-phenyl)-piperazin-1-ylmethyl]-1-ethyl-2-phenyl-1,2-dihydropyrazol-3-one was obtained from 5-bromomethyl-4-chloro-1-ethyl-2-phenylpyrazolidin-3-one (31 mg, 0.1 mmol), 1-(3,5-dichloro-phenyl)piperazine (40 mg, 0.15 mmol) and potassium carbonate (40 mg, 0.30 mmol) in acetonitrile (1.5 mL) as a solid 35 mg (76%). ¹H NMR (300 MHz, CDCl₃): δ (ppm) 7.36-7.53 (m, 5H), 6.88 (s, 1H), 6.77 (s, 2H), 4.13 (q, 2H), 3.63 (s, 2H), 3.23 (t, 4H), 2.74 (t, 4H), 0.90 (t, 3H).

### Example 159

### 4-Chloro-5-[4-(2,4-dimethyl-phenyl)-piperazin-1-ylmethyl]-1-ethyl-2-phenyl-1,2-dihydropyrazol-3-one

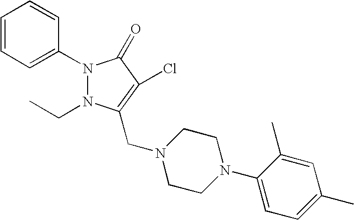

4-Chloro-5-[4-(2,4-dimethyl-phenyl)-piperazin-1-ylmethyl]-1-ethyl-2-phenyl-1,2-dihydropyrazol-3-one was obtained from 5-bromomethyl-4-chloro-1-ethyl-2-phenylpyrazolidin-3-one (31 mg, 0.1 mmol), 1-(2,4-dimethyl-phenyl)piperazine (29 mg, 0.15 mmol) and potassium carbonate (40 mg, 0.30 mmol) in acetonitrile (1.5 mL) as a solid 37.8 mg (88%). ¹H NMR (300 MHz, CDCl₃): δ (ppm) 7.35-7.53 (m, 5H), 6.94-7.04 (m, 3H), 4.13 (q, 2H), 3.65 (s, 2H), 2.95 (t, 4H), 2.76 (t, 4H), 2.35 (s, 3H), 2.30 (s, 3H), 0.91 (t, 3H).

### Example 160

### 4-Chloro-5-[4-(3,5-dimethyl-phenyl)-piperazin-1-ylmethyl]-1-ethyl-2-phenyl-1,2-dihydropyrazol-3-one

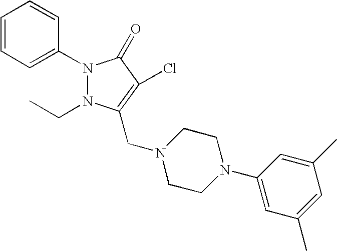

4-Chloro-5-[4-(3,5-dimethyl-phenyl)-piperazin-1-ylmethyl]-1-ethyl-2-phenyl-1,2-dihydropyrazol-3-one was obtained from 5-bromomethyl-4-chloro-1-ethyl-2-phenylpyrazolidin-3-one (31 mg, 0.1 mmol), 1-(3,5-dimethyl-phenyl)piperazine (29 mg, 0.15 mmol) and potassium carbonate (40 mg, 0.30 mmol) in acetonitrile (1.5 mL) as a solid 39.6 mg (92%). ¹H NMR (300 MHz, CDCl₃): δ(ppm) 7.33-7.53 (m, 5H), 6.60 (s, 2H), 6.57 (s, 1H), 3.79 (q, 2H), 3.63 (s, 2H), 3.23 (t, 4H), 2.75 (t, 4H), 2.30 (s, 6H), 0.91 (t, 3H).

### Example 161

### 2-[4-(4-chloro-2-ethyl-5-oxo-1-phenyl-2,5-dihydro-1H-pyrazol-3-ylmethyl)-piperazin-1-yl]-benzonitrile

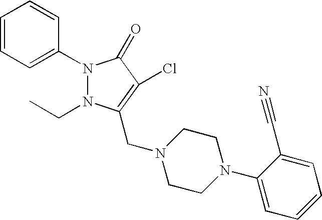

2-[4-(4-chloro-2-ethyl-5-oxo-1-phenyl-2,5-dihydro-1H-pyrazol-3-ylmethyl)-piperazin-1-yl]-benzonitrile was obtained from 5-bromomethyl-4-chloro-1-ethyl-2-phenylpyrazolidin-3-one (31 mg, 0.1 mmol), 2-piperazin-1-yl-benzonitrile (29 mg, 0.15 mmol) and potassium carbonate (40 mg, 0.30 mmol) in acetonitrile (1.5 mL) as a solid 35.2 mg (83%). ¹H NMR (300 MHz, CDCl₃): 10(ppm) 7.45-7.60 (m, 7H), 7.03-7.06 (m, 2H), 3.78 (q, 2H), 3.65 (s, 2H), 3.28 (t, 4H), 2.82 (t, 4H), 0.91 (t, 3H).

### Example 162

### 4-Chloro-1-ethyl-2-phenyl-5-[4-(2-trifluoromethylphenyl)piperazin 1-ylmethyl]-1,2-dihydropyrazol-3-one

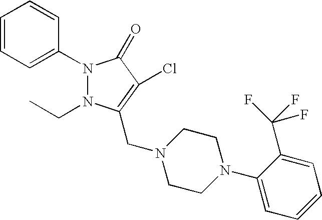

4-Chloro-1-ethyl-2-phenyl-5-[4-(2-trifluoromethylphenyl)piperazin-1-ylmethyl]-1,2-dihydropyrazol-3-one was obtained from 5-bromomethyl-4-chloro-1-ethyl-2-phenyl-pyrazolidin-3-one (31 mg, 0.1 mmol), 1-(2-trifluoromethylphenyl)piperazine (35 mg, 0.15 mmol) and potassium carbonate (40 mg, 0.30 mmol) in acetonitrile (1.5 mL) as a solid 46 mg (100%). ¹H NMR (300 MHz, CDCl₃): δ (ppm) 7.25-7.63 (m, 9H), 3.81 (q, 2H), 3.59 (s, 2H), 2.99 (t, 4H), 2.76 (t, 4H), 0.91 (t, 3H).

### Example 163

### 4-Chloro-1-ethyl-2-phenyl-5-[4-(4-trifluoromethylphenyl)piperazin-1-ylmethyl]-1,2-dihydropyrazol-3-one

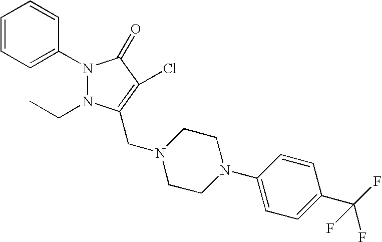

4-Chloro-1-ethyl-2-phenyl-5-[4-(4-trifluoromethylphenyl)piperazin-1-ylmethyl]-1,2-dihydropyrazol-3-one was obtained from 5-bromomethyl-4-chloro-1-ethyl-2-phenyl-pyrazolidin-3-one (31 mg, 0.1 mmol), 1-(4-trifluoromethylphenyl)piperazine (36 mg, 0.15 mmol) and potassium carbonate (40 mg, 0.30 mmol) in acetonitrile (1.5 mL) as a solid 36 mg (78%). ¹H NMR (300 MHz, CDCl₃): δ(ppm) 7.36-7.53 (m, 7H), 6.95 (d, 2H), 3.81 (q, 2H), 3.64 (s, 2H), 3.34 (t, 4H), 2.76 (t, 4H), 0.91 (t, 3H).

### Example 164

### 4-Chloro-5-[5-(4-chloro-2-methoxy-phenyl)-piperazin-1-ylmethyl]-1-ethyl-2-phenyl-1,2-dihydropyrazol-3-one

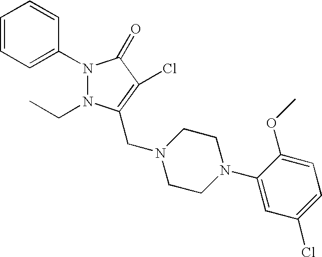

4-Chloro-5-[5-(4-chloro-2-methoxy-phenyl)-piperazin-1-ylmethyl]-1-ethyl-2-phenyl-1,2-dihydropyrazol-3-one was obtained from 5-bromomethyl-4-chloro-1-ethyl-2-phenylpyrazolidin-3-one (31 mg, 0.1 mmol), 1-(5-chloro-2-methoxy-phenyl)piperazine (38 mg, 0.15 mmol) and potassium carbonate (40 mg, 0.30 mmol) in acetonitrile (1.5 mL) as a solid 42 mg (85%). ¹H NMR (300 MHz, CDCl₃): δ(ppm) 7.35-7.49 (m, 5H), 6.98 (s, 1H), 6.89 (S, 1 h), 6.80 (d, 1H), 3.87 (s, 3H), 3.79 (q, 2H), 3.64 (s, 2H), 3.10 (t, 4H), 2.79 (t, 4H), 0.90 (t, 3H).

### Example 165

### 4-Chloro-1-ethyl-5-[4-(4-ethoxy-phenyl)-piperazin-1-ylmethyl]-2-phenyl-1,2-dihydropyrazol-3-one

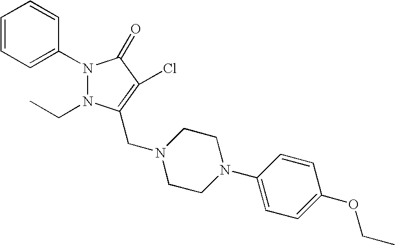

4-Chloro-1-ethyl-5-[4-(4-ethoxy-phenyl)-piperazin-1-ylmethyl]-2-phenyl-1,2-dihydropyrazol-3-one was obtained from 5-bromomethyl-4-chloro-1-ethyl-2-phenylpyrazolidin-3-one (31 mg, 0.1 mmol), 1-(4-ethoxy-phenyl)piperazine (31 mg, 0.15 mmol) and potassium carbonate (40 mg, 0.30 mmol) in acetonitrile (1.5 mL) as a solid (31 mg 70%). ¹H NMR (300 MHz, CDCl₃): δ (ppm) 7.33-7.52 (m, 5H), 6.84-6.94 (m, 4H), 3.99 (q, 2H0, 3.78 (q, 2H), 3.63 (s, 2H), 3.14 (t, 4H), 2.77 (t, 4H), 1.40 (t, 3H), 0.91 (t, 3H).

### Example 166

### 4-Chloro-1-ethyl-5-(4-hydroxy-4-phenyl-piperidin-1-ylmethyl)-2-phenyl-1,2-dihydropyrazol-3-one

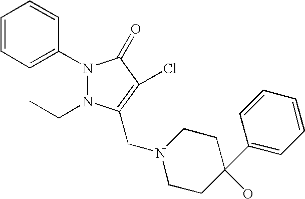

4-Chloro-1-ethyl-5-(4-hydroxy-4-phenyl-piperidin-1-ylmethyl)-2-phenyl-1,2-dihydropyrazol-3-one was obtained by the following procedure. A mixture of 5-bromomethyl-4-chloro-1-ethyl-2-phenylpyrazolidin-3-one (96 mg, 0.3 mmol), piperidine-4,4-diol hydrochloride (70 mg, 0.45 mmol), potassium carbonate (138 mg, 1 mmol) and acetonitrile (3 mL) was stirred at room temperature for 4 hours. The resulting mixture was directly subjected to silica gel column to afford intermediate 1-(4-chloro-2-ethyl-5-oxo-1-phenyl-2,5-dihydro-1H-pyrazol-3-ylmethyl)-piperidin-4-one (86 mg, 86%) as a solid. To this intermediate solution of THF (2 mL) was slowly added a THF solution of PhMgBr (1M, 0.6 mL) at 0° C. over 10 minutes and was stirred at room temperature overnight. After standard work-up the crude residue was purified on silica gel column to give the final product (42 mg, 40%). ¹H NMR (300 MHz, CDCl₃): δ (ppm) 7.26-7.54 (m, 10H), 3.80 (q, 2H), 3.63 (s, 2H), 2.71-2.86 (m, 4H), 2.13 (m, 2H), 1.83 (m, 2H), 0.92 (t, 3H).

### Example 167

### 4-Chloro-1-ethyl-2-phenyl-5-(4-phenyl-3,6-dihydro-2H-pyridin-1-ylmethyl)-1,2-dihydro-pyrazol-3-one

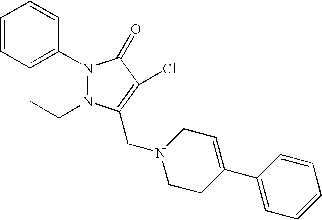

4-Chloro-1-ethyl-2-phenyl-5-(4-phenyl-3,6-dihydro-2H-pyridin-1-ylmethyl)-1,2-dihydro-pyrazol-3-one was obtained by the following procedure. A mixture of 4-Chloro-1-ethyl-5-(4-hydroxy-4-phenyl-piperidin-1-ylmethyl)-2-phenyl-1,2-dihydropyrazol-3-one (17 mg, 0.04 mmol), phosphorus pentaoxide (5 mg, 0.035 mmol) and toluene (1 mL) was heated to reflux for 4 hours. The resulting mixture was directly subjected to silica gel column to afford pure product (0.7 mg, 5%). ¹H NMR (300 MHz, CDCl₃): δ (ppm) 7.29-7.50 (m, 10H), 6.11 (d, 1H), 3.85 (q, 2H), 3.78 (s, 2H), 3.41 (d, 2H), 2.90 (t, 2H), 2.65 (t, 2H), 0.89 (t, 3H).

### Example 168

### 4-Chloro-1-ethyl-2-phenyl-5-(4-phenyl-piperidine-1-ylmethyl)-1,2-dihydro-pyrazol-3-one

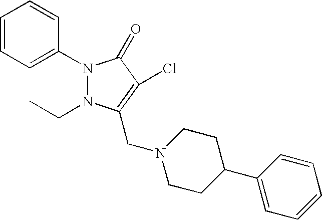

4-Chloro-1-ethyl-2-phenyl-5-(4-phenyl-piperidine-1-ylmethyl)-1,2-dihydro-pyrazol-3-one was obtained by the following procedure. To a solution of 1-benzyl-piperidin-4-one (114 mg, 0.6 mmol) in THF (1.5 mL) was added a THF solution of PhLi (1M, 1.5 mL) at −70° C. The reaction mixture was allowed to warm to room temperature over 2 hours and was kept stirring at r.t for an hour. After standard work-up the crude yellow solid was triturated in hexane to afford 1-benzyl-4-phenyl-piperidin-4-ol (110 mg, 64%), which was stirred with phosphorus pentaoxide (42 mg, 0.3 mmol) in toluene (2 mL) at 110° C. overnight. The resulting mixture was directly purified on silica gel column to give 1-benzyl-4-phenyl 1,2,3,6-tetrahydro-pyridine (27 mg, 28%). A mixture of 1-benzyl-4-phenyl 1,2,3,6-tetrahydro-pyridine (27 mg, 0.11 mmol), Pd/C (10%, 10 mg) and ethanol (1.5 mL) was stirred under hydrogen (1 atm) at room temperature for 20 hours. Ethanol was removed under reduced pressure and the remains were treated with 5-bromomethyl-4-chloro-1-ethyl-2-phenylpyrazolidin-3-one (25 mg, 0.08 mmol), potassium carbonate (20 mg, 0.14 mmol) in acetonitrile (3 mL) at room temperature overnight. The resulting mixture was directly subjected to silica gel column to give the final product (20 mg, 45% for 2 steps). ¹H NMR (300 MHz, CDCl₃): δ (ppm) 7.23-7.50 (m, 10H), 3.82 9q, 2H), 3.60 (s, 2H), 3.08 (m, 2H), 2.57 (m, 1H), 2.31 (m, 2H), 1.77-1.94 (m, 4H), 0.91 (t, 3H).

### Example 169

### 4-Bromo-5-[4-(5-chloro-2-methoxy-phenyl)-piperazin-1-ylmethyl]-1-ethyl-2-phenyl-1,2-dihydropyrazol-3-one

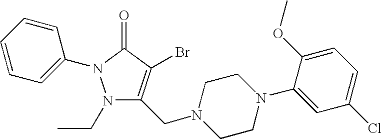

4-Bromo-5-[4-(5-chloro-2-methoxy-phenyl)-piperazin-1-ylmethyl]-1-ethyl-2-phenyl-1,2-dihydropyrazol-3-one was obtained from 4-bromo-5-bromomethyl-1-ethyl-2-phenylpyrazol-3-one (30 mg, 0.083 mmol), 1-(5-chloro-2-methoxyphenyl)piperazine (28 mg, 0.125 mmol) and potassium carbonate (34 mg, 0.249 mmol) in acetonitrile (2.0 mL) as an off-white solid (47 mg, 110%). ¹H NMR (300 MHz, CDCl₃): δ (ppm) 7.35-7.50 (m, 5H), 6.99 (dd, 1H), 6.89 (d, 1H), 6.79 (d, 1H), 3.88 (s, 3H), 3.83 (q, 2H), 3.64 (s, 2H), 3.11 (s, 4H), 2.79 (t, 4H), 0.92 (t, 3H).

### Example 170

### 4-Chloro-5-[4-(4-fluoro-2-methoxy-phenyl)-piperazin-1-ylmethyl]-1-methyl-2-phenyl-1,2-dihydro-pyrazol-3-one

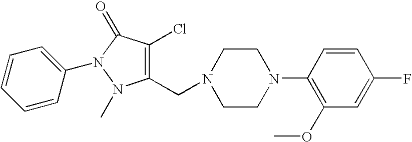

4-Chloro-5-[4-(4-fluoro-2-methoxy-phenyl)-piperazin-1-ylmethyl]-1-methyl-2-phenyl-1,2-dihydro-pyrazol-3-one was obtained from 1-(4-Fluoro-2-methoxy-phenyl)-piperazine (31.4 mg, 0.149 mmol), 5-Bromomethyl-4-chloro-1-methyl-2-phenyl-1,2-dihydro-pyrazol-3-one (30 mg, 0.099 mmol), and potassium carbonate (68.4 mg, 0.498 mmol) in acetonitrile (1.5 mL) as a white solid (29.2 mg, 68%). ¹H NMR (300 MHz, CDCl₃): δ (ppm) 7.50 (t, 2H), 7.42 (dd, 2H), 7.35 (t, 1H), 6.88 (t, 1H), 6.63 (d, 2H), 3.88 (s, 3H), 3.66 (s, 2H), 3.26 (s, 3H), 3.07 (broad s, 4H), 2.78 (broad t, 4H).

### Example 171

### 4-Chloro-1-ethyl-5-[4-(4-fluoro-2-methoxy-phenyl)-piperazin-1-ylmethyl]-2-phenyl-1,2-dihydro-pyrazol-3-one

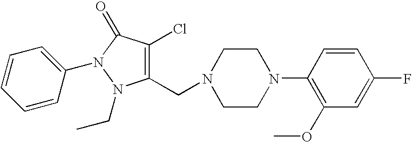

4-Chloro-1-ethyl-5-[4-(4-fluoro-2-methoxy-phenyl)-piperazin-1-ylmethyl]-2-phenyl-1,2-dihydro-pyrazol-3-one was obtained from 1-(4-Fluoro-2-methoxy-phenyl)-piperazine (30.1 mg, 0.143 mmol), 5-Bromomethyl-4-chloro-1-ethyl-2-phenyl-1,2-dihydro-pyrazol-3-one (30 mg, 0.095 mmol), and potassium carbonate (65.7 mg, 0.475 mmol) in acetonitrile (1.5 mL) as a white solid (33.4 mg, 79%). ¹H NMR (300 MHz, CDCl₃): δ (ppm) 7.50 (t, 2H), 7.42 (dd, 2H), 7.36 (t, 1H), 6.87 (t, 1H), 6.62 (d, 2H), 3.88 (s, 3H), 3.80 (q, 2H), 3.66 (s, 2H), 3.07 (broad s, 4H), 2.78 (broad t, 4H), 1.28 (t, 3H).

### Example 172

### 4-Chloro-5-[4-(4-chloro-2-methoxy-phenyl)-piperazin-1-ylmethyl]-1-methyl-2-phenyl-1,2-dihydro-pyrazol-3-one

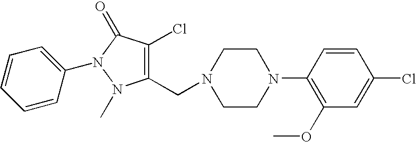

4-Chloro-5-[4-(4-chloro-2-methoxy-phenyl)-piperazin-1-ylmethyl]-1-methyl-2-phenyl-1,2-dihydro-pyrazol-3-one was obtained from 1-(4-Chloro-2-methoxy-phenyl)-piperazine (33.8 mg, 0.149 mmol), 5-Bromomethyl-4-chloro-1-methyl-2-phenyl-1,2-dihydro-pyrazol-3-one (30.0 mg, 0.099 mmol), and potassium carbonate (68.4, 0.495 mmol) in acetonitrile (1.5 mL) as a white film (5.1 mg, 12%). ¹H NMR (300 MHz, CDCl₃): δ (ppm): 7.42-7.52 (m, 4H), 7.38 (t, 1H), 6.91 (d, 1H), 6.85 (d, 2H), 3.89 (s, 3H), 3.79 (q, 2H), 3.63 (s, 2H), 3.09 (broad s, 4H), 2.80 (broad t, 4H), 0.91 (t, 3H).

### Example 173

### 4-Chloro-1-methyl-5-(3-methyl-3-phenyl-pyrrolidin-1-ylmethyl)-2-phenyl-1,2-dihydro-pyrazol-3-one

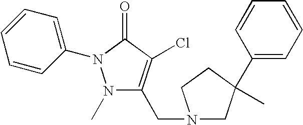

4-Chloro-1-methyl-5-(3-methyl-3-phenyl-pyrrolidin-1-ylmethyl)-2-phenyl-1,2-dihydro-pyrazol-3-one was obtained from 3-Methyl-3-phenyl-pyrrolidine (24.03 mg, 0.149 mmol), 5-Bromomethyl-4-chloro-1-methyl-2-phenyl-1,2-dihydro-pyrazol-3-one (30 mg, 0.099 mmol), and potassium carbonate (68.69 mg, 0.497 mmol) in acetonitrile (2 mL) as a pale yellow solid (37.4 mg, 99%) ¹H NMR (300 MHz, CDCl₃): δ (ppm) 7.34-7.52 (m, 9H), 7.23 (m, 1H), 3.76 (s, 2H), 3.25 (s, 3H), 2.99 (q, 2H), 2.81 (q, 2H), 2.31 (m, 1H), 2.05 (m, 1H), 1.48 (s, 3H).

### Example 174

### 4-Chloro-1-ethyl-5-(3-methyl-3-phenyl-pyrrolidin-1-ylmethyl)-2-phenyl-1,2-dihydro-pyrazol-3-one

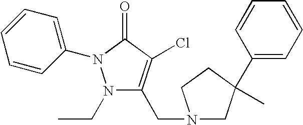

4-Chloro-1-ethyl-5-(3-methyl-3-phenyl-pyrrolidin-1-ylmethyl)-2-phenyl-1,2-dihydro-pyrazol-3-one was obtained from 3-Methyl-3-phenyl-pyrrolidine (23.06 mg, 0.143 mmol), 5-Bromomethyl-4-chloro-1-ethyl-2-phenyl-1,2-dihydro-pyrazol-3-one (30 mg, 0.095 mmol), and potassium carbonate (65.65 mg, 0.475 mmol) in acetonitrile (2 mL) as a colourless oil (33.0 mg, 88%) ¹H NMR (300 MHz, CDCl₃): δ (ppm) 7.23-7.52 (m, 10H), 3.84 (m, 1H), 3.74 (s, 3H), 3.01 (q, 2H), 2.83 (m, 2H), 2.29 (q, 1H), 2.04 (m, 1H), 1.48 (s, 3H), 0.89 (t, 3H).

### Example 175

### 1-[1-(4-Chloro-2-ethyl-5-oxo-1-phenyl-2,5-dihydro-1H-pyrazol-3-ylmethyl)-piperidin-4-yl]-1,3-dihydro-indol-2-one

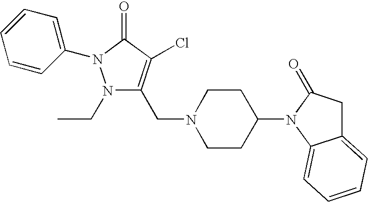

1-[1-(4-Chloro-2-ethyl-5-oxo-1-phenyl-2,5-dihydro-1H-pyrazol-3-ylmethyl)-piperidin-4-yl]-1,3-dihydro-indol-2-one was obtained from 1-Piperidin-4-yl-1,3-dihydro-indol-2-one (30.93 mg, 0.143 mmol), 5-Bromomethyl-4-chloro-1-ethyl-2-phenyl-1,2-dihydro-pyrazol-3-one (30 mg, 0.095 mmol), and potassium carbonate (65.65 mg, 0.475 mmol) in acetonitrile (2 mL) as a colourless oil (35.2 mg, 82%). ¹H NMR (300 MHz, CDCl₃): δ (ppm): 7.34-7.54 (m, 5H), 7.26 (d, 2H), 7.04 (m, 2H), 4.23 (tt, 1H), 3.84 (q, 2H), 3.62 (s, 2H), 3.54 (s, 2H), 3.13 (d, 2H), 2.53 (qd, 2H), 2.35 (t, 2H), 1.78 (d, 2H), 0.94 (t, 3H).

### Example 176

### Spiro[Indan-N-4-Chloro-2-methyl-5-oxo-1-phenyl-2,5-dihydro-1H-pyrazol-3-ylmethyl pyrrolidine]

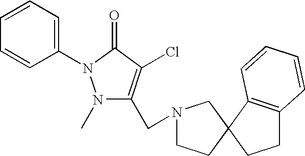

Spiro[Indan-N-4-Chloro-2-methyl-5-oxo-1-phenyl-2,5-dihydro-1H-pyrazol-3-ylmethyl pyrrolidine] was obtained from Spiro[Indanepyrrolidine] (43.14 mg, 0.249 mmol), 5-Bromomethyl-4-chloro-1-methyl-2-phenyl-1,2-dihydro-pyrazol-3-one (50.0 mg, 0.166 mmol), and potassium carbonate (114.7 mg, 0.83 mmol) in acetonitrile (2 mL) as an off-white solid (63.8 mg, 98%). ¹H NMR (300 MHz, CDCl₃): δ (ppm) 7.49 (m, 2H), 7.37 (dd, 2H), 7.32 (dd, 2H), 7.22 (m, 3H), 3.76 (s, 2H), 3.29 (s, 3H), 2.92 (m, 4H), 2.77 (m, 2H), 2.06-2.21 (m, 4H).

### Example 177

### Spiro[Indan-N-4-Chloro-2-ethyl-5-oxo-1-phenyl-2,5-dihydro-1H-pyrazol-3-ylmethyl pyrrolidine]

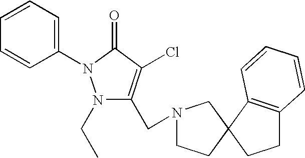

Spiro[Indan-N-4-Chloro-2-ethyl-5-oxo-1-phenyl-2,5-dihydro-1H-pyrazol-3-ylmethyl pyrrolidine] was obtained from Spiro[Indanepyrrolidine] (41.1 mg, 0.24 mmol), 5-Bromomethyl-4-chloro-1-ethyl-2-phenyl-1,2-dihydro-pyrazol-3-one (50.0 mg, 0.158 mmol), and potassium carbonate 109.2 mg, 0.790 mmol) in acetonitrile (2 mL) as a yellow solid (62.0 mg, 96%). ¹H NMR (300 MHz, CDCl₃): δ (ppm) 7.49 (m, 2H), 7.39 (m, 2H), 7.32 (m, 2H), 7.22 (m, 3H), 3.86 (m, 2H), 3.74 (s, 2H), 2.90 (m, 4H), 2.79 (q, 2H), 2.06-2.21 (m, 4H), 0.92 (t, 3H).

### Example 178

### 4-Chloro-2-(4-fluorophenyl)-1-methyl-5-(4-o-tolylpiperazin-1-ylmethyl)-1,2-dihydropyrazol-3-one

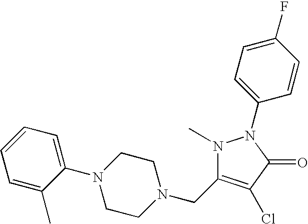

4-Chloro-2-(4-fluorophenyl)-1-methyl-5-(4-o-tolylpiperazin-1-ylmethyl)-1,2-dihydropyrazol-3-one was obtained from 5-bromomethyl-4-chloro-2-(4-fluorophenyl)-1-methyl-1,2-dihydropyrazol-3-one (30 mg, 0.093 mmol), 1-(o-tolyl)piperazine hydrochloride (30 mg, 0.14 mmol) and potassium carbonate (45 mg, 0.327 mmol) in acetonitrile (2 mL) as white solid 22 mg (39%). ¹H NMR (300 MHz, CDCl₃): δ (ppm) 7.38 (p, 2H), 7.20 (q, 4H), 7.03 (q, 2H), 3.66 (s, 2H), 3.53 (s, 3H), 2.98 (s, 4H), 2.78 (s, 4H), 2.34 (s, 3H).

### Example 179

### 4-Chloro-5-[4-(2-chlorophenyl)piperazin-1-ylmethyl]-2-(4-fluorophenyl)-1-methyl-1,2-dihydropyrazol-3-one

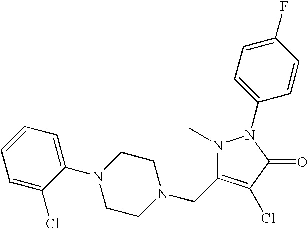

4-Chloro-5-[4-(2-chlorophenyl)piperazin-1-ylmethyl]-2-(4-fluorophenyl)-1-methyl-1,2-dihydropyrazol-3-one was obtained from 5-bromomethyl-4-chloro-2-(4-fluorophenyl)-1-methyl-1,2-dihydropyrazol-3-one (30 mg, 0.093 mmol), 1-(2-chlorophenyl)piperazine (38 mg, 0.14 mmol) and potassium carbonate (45 mg, 0.33 mmol) in acetonitrile (2 mL) as an off-white solid 45 mg (73%). ¹H NMR (300 MHz, CDCl₃): δ (ppm) 7.38 (m, 3H), 7.19 (m, 3H), 7.04 (q, 2H), 3.66 (s, 2H), 3.24 (s, 3H), 3.12 (s, 4H), 2.78 (s, 4H).

### Example 180

### 4-Chloro-5-[4-(5-chloro-2-methoxyphenyl)piperazin-1-ylmethyl]-2-(4-fluorophenyl)-1-methyl-1,2-dihydropyrazol-3-one

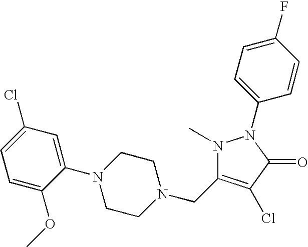

4-Chloro-5-[4-(5-chloro-2-methoxyphenyl)piperazin-1-ylmethyl]-2-(4-fluorophenyl)-1-methyl-1,2-dihydropyrazol-3-one was obtained from 5-bromomethyl-4-chloro-2-(4-fluorophenyl)-1-methyl-1,2-dihydropyrazol-3-one (30 mg, 0.0935 mmol), 1-(5-chloro-2-methoxyphenyl)piperazine hydrochloride (29 mg, 0.14 mmol) and potassium carbonate (45 mg, 0.327 mmol) in acetonitrile (2 mL) as white solid 26 mg (41%). ¹H NMR (300 MHz, CDCl₃): δ (ppm) 7.39 (m, 2H), 7.19 (t, 2H), 6.99 (d, 1H), 6.96 (s, 1H), 6.80 (d, 1H), 3.88 (s, 3H), 3.65 (s, 2H), 3.24 (s, 3H), 3.06 (s, 4H), 2.78 (s, 4H).

### Example 181

### 4-Chloro-5-[4-(3-ethoxyphenyl)piperazin-1-ylmethyl]-2-(4-fluorophenyl)-1-methyl-1,2-dihydropyrazol-3-one

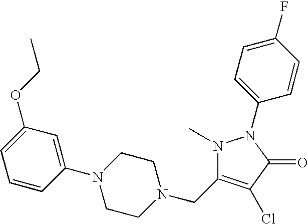

4-Chloro-5-[4-(3-ethoxyphenyl)piperazin-1-ylmethyl]-2-(4-fluorophenyl)-1-methyl-1,2-dihydropyrazol-3-one was obtained from 5-bromomethyl-4-chloro-2-(4-fluorophenyl)-1-methyl-1,2-dihydropyrazol-3-one (30 mg, 0.093 mmol), 1-(2-ethoxyphenyl)piperazine monohydrochloride (34 mg, 0.14 mmol) and potassium carbonate (45 mg, 0.327 mmol) in acetonitrile (2 mL) as red oil 39 mg (65%). ¹H NMR (300 MHz, CDCl₃): □(ppm) 7.39 (1m, 2H), 7.19 (t, 2H), 6.93 (m, 4H), 4.10 (q, 2H), 3.66 (s, 2H), 3.25 (s, 3H), 3.16 (s, 4H), 2.78 (s, 4H), 1.27 (m, 3H).

### Example 182

### 4-Chloro-5-[4-(5-chloro-2-methylphenyl)piperazin-1-ylmethyl]-2-(4-fluorophenyl)-1-methyl-1,2-dihydropyrazol-3-one

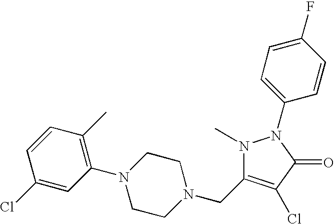

4-Chloro-5-[4-(5-chloro-2-methylphenyl)piperazin-1-ylmethyl]-2-(4-fluorophenyl)-1-methyl-1,2-dihydropyrazol-3-one was obtained from 5-bromomethyl-4-chloro-2-(4-fluorophenyl)-1-methyl-1,2-dihydropyrazol-3-one (30 mg, 0.093 mmol), 1-(5-chloro-2-methylphenyl)piperazine (30 mg, 0.14 mmol) and potassium carbonate (45 mg, 0.327 mmol) in acetonitrile (2 mL) as an off-white solid 35 mg (56%). ¹H NMR (300 MHz, CDCl₃): δ (ppm) 7.39 (m, 2H), 7.19 (t, 2H), 7.10 (d, 1H), 6.98 (d, 2H), 3.61 (s, 2H), 3.26 (s, 3H), 2.97 (s, 4H), 2.77 (s, 4H), 2.28 (s, 3H).

### Example 183

### 4-Chloro-5-[4-(2,4-dimethylphenyl)piperazin-1-ylmethyl]-2-(4-fluorophenyl)-1-methyl-1,2-dihydropyrazol-3-one

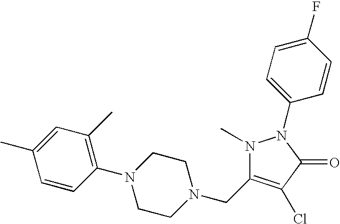

4-Chloro-5-[4-(2,4-dimethylphenyl)piperazin-1-ylmethyl]-2-(4-fluorophenyl)-1-methyl-1,2-dihydropyrazol-3-one was obtained from 5-bromomethyl-4-chloro-2-(4-fluorophenyl)-1-methyl-1,2-dihydropyrazol-3-one (30 mg, 0.93 mmol), 1-(2,4-dimethylphenyl)piperazine (27 mg, 0.14 mmol) and potassium carbonate (45 mg, 0.327 mmol) in acetonitrile (2 mL) as white solid 31 mg (52%). ¹H NMR (300 MHz, CDCl₃): δ (ppm) 7.40 (m, 2H), 7.20 (q, 2H), 6.98 (p, 3H), 3.63 (s, 2H), 3.26 (s, 3H), 2.95 (s, 4H), 2.74 (s, 4H), 2.30 (s, 6H).

### Example 184

### 4-Chloro-5-[4-(3,5-dichloropyridin-4-yl)-piperazin-1-ylmethyl]-2-(4-fluorophenyl)-1-methyl-1,2-dihydropyrazol-3-one

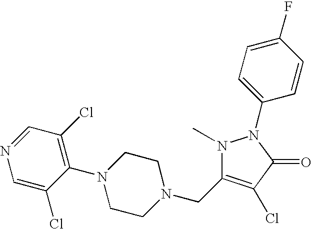

4-Chloro-5-[4-(3,5-dichloropyridin-4-yl)-piperazin-1-ylmethyl]-2-(4-fluorophenyl)-1-methyl-1,2-dihydropyrazol-3-one was obtained from 5-bromomethyl-4-chloro-2-(4-fluorophenyl)-1-methyl-1,2-dihydropyrazol-3-one (30 mg, 0.093 mmol), 1-(3,5-dichloropyridin-4-yl)piperazine (32 mg, 0.14 mmol) and potassium carbonate (45 mg, 0.32 mmol) in acetonitrile (2 mL) as a white solid 45 mg (64%). ¹H NMR (300 MHz, CDCl₃): δ (ppm) 8.22 (s, 2H), 7.38 (p, 2H), 7.19 (t, 2H), 3.66 (s, 3H), 3.42 (s, 4H), 3.28 (s, 3H), 2.73 (s, 4H).

### Example 185

### 8-[4-Chloro-1-(4-fluorophenyl)-2-methyl-5-oxo-2,5-dihydro-1H-pyrazol-3-ylmethyl]-1-phenyl-1,3,8-triazaspiro[4.5]decan-4-one

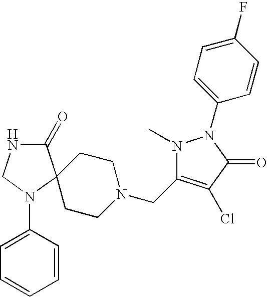

8-[4-Chloro-1-(4-fluorophenyl)-2-methyl-5-oxo-2,5-dihydro-1H-pyrazol-3-ylmethyl]-1-phenyl-1,3,8-triazaspiro[4.5]decan-4-one was obtained from 5-bromomethyl-4-chloro-2-(4-fluorophenyl)-1-methyl-1,2-dihydropyrazol-3-one (30 mg, 0.0935 mmol), 1-phenyl-1,3,8-triazaspiro[4,5]decan-4-one (42 mg, 0.14 mmol) and potassium carbonate (45 mg, 0.32 mmol) in acetonitrile (2 mL) as an off-white solid 44 mg (58%). ¹H NMR (300 MHz, CDCl₃): δ (ppm) 7.40 (m, 2H), 7.28 (m, 2H), 7.19 (m, 2H), 6.91 (t, 4H), 4.78 (s, 2H), 3.69 (s, 2H), 3.29 (s, 3H), 3.05 (t, 2H), 2.92 (t, 2H), 2.68 (m, 2H), 1.28 (d, 2H).

### Example 186

### 1-[4-Chloro-1-(4-fluorophenyl)-2-methyl-5-oxo-2,5-dihydro-1H-pyrazol-3-ylmethyl]-4-phenylpiperidine-4-carbonitrile

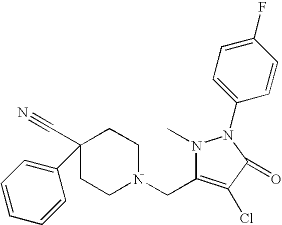

1-[4-Chloro-1-(4-fluorophenyl)-2-methyl-5-oxo-2,5-dihydro-1H-pyrazol-3-ylmethyl]-4-phenylpiperidine-4-carbonitrile was obtained from 5-bromomethyl-4-chloro-2-(4-fluorophenyl)-1-methyl-1,2-dihydropyrazol-3-one (30 mg, 0.0936 mmol), 4-cyano-4-phenylypiperidine hydrochloride (31 mg, 0.140 mmol) and potassium carbonate (45 mg, 0.328 mmol) in acetonitrile (2 mL) as yellow oil 42 mg (98%). ¹H NMR (300 MHz, CDCl₃): δ (ppm) 7.52 (d, 2H), 7.51-7.36 (m, 5H), 7.19 (t, 2H), 3.68 (s, 2H), 3.17 (s, 3H), 3.06 (d, 2H), 2.74 (t, 2H), 2.12 (q, 4H).

### Example 187

### 5-(4-Butyryl-4-phenylpiperidin-1-ylmethyl)-4-chloro-2-(4-fluorophenyl)-1-methyl-1,2-dihydropyrazol-3-one

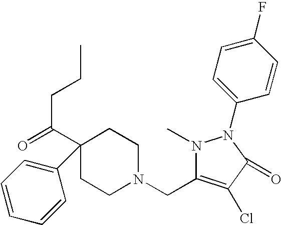

5-(4-Butyryl-4-phenylpiperidin-1-ylmethyl)-4-chloro-2-(4-fluorophenyl)-1-methyl-1,2-dihydropyrazol-3-one was obtained from 5-bromomethyl-4-chloro-2-(4-fluorophenyl)-1-methyl-1,2-dihydropyrazol-3-one (30 mg, 0.0936 mmol), 4-phenyl-4-propionylpiperidine hydrochloride (36 mg, 0.140 mmol) and potassium carbonate (45 mg, 0.328 mmol) in acetonitrile (2 mL) as a white solid 20 mg (45%). ¹H NMR (300 MHz, CDCl₃): δ (ppm) 7.38-7.29 (m, 8H), 7.20 (t, 1H), 3.51 (s, 2H), 3.15 (s, 3H), 2.74 (s, 2H), 2.42 (q, 4H), 2.27 (q, 2H), 2.10 (m, 2H), 0.90 (t, 3H).

### Example 188

### 4-Chloro-2-(4-fluoro-phenyl)-1-methyl-5-(4-phenyl-4-propionyl-piperidin-1-ylmethyl)-1,2-dihydro-pyrazol-3-one

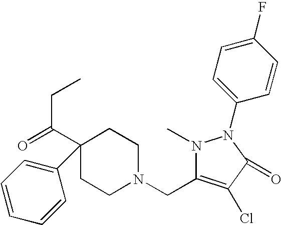

4-Chloro-2-(4-fluorophenyl)-1-methyl-5-(4-phenyl-4-propionylpiperidin-1-ylmethyl)-1,2-dihydropyrazol-3-one was obtained from 5-bromomethyl-4-chloro-2-(4-fluorophenyl)-1-methyl-1,2-dihydropyrazol-3-one (30 mg, 0.0936 mmol), 4-butyl-4-phenylpiperidine hydrochloride (38 mg, 0.140 mmol) and potassium carbonate (45 mg, 0.328 mmol) in acetonitrile (2 mL) as a white solid 12 mg (27%). ¹H NMR (300 MHz, CDCl₃): δ (ppm) 7.38-7.29 (m, 7H), 7.17 (t, 2H), 3.52 (s, 2H), 3.18 (s, 3H), 2.74 (s, 2H), 2.48 (q, 4H), 2.14 (m, 4H), 1.44 (q, 4H), 0.68 (t, 3H).

### Example 189

### 4-Chloro-2-(4-fluoro-phenyl)-1-methyl-5-[4-(3-phenyl-propyl)-piperidin-1-ylmethyl]-1,2-dihydro-pyrazol-3-one

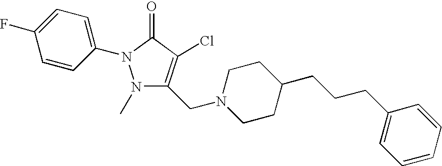

4-Chloro-2-(4-fluoro-phenyl)-1-methyl-5-[4-(3-phenyl-propyl)-piperidin-1-ylmethyl]-1,2-dihydro-pyrazol-3-one was synthesized from 5-bromomethyl-4-chloro-2-(4-fluoro-phenyl)-1-methyl-1,2-dihydro-pyrazol-3-one (0.040 g, 0.125 mmol), 4-(3-phenyl-propyl)-piperidine (0.038 g, 0.187 mmol) and potassium carbonate (0.052 g, 0.187 mmol) in 2.0 mL of acetonitrile. The crude material was purified by eluting through a 2 g SPE tube using a solution of 10% acetone and dichloromethane to yield a white solid (54.9 mg, 99.3%). ¹H NMR (300 MHz, CDCL₃): δ (ppm) 1.33-1.20 (m, 5H), 1.70 (m, 4H), 2.07 (t, 2H), 2.62 (t, 2H), 2.88 (d, 2H), 3.21 (s, 3H), 3.52 (s, 2H), 7.22-7.15 (m, 5H), 7.40-7.29 (m, 4H).

### Example 190

### 4-Chloro-5-[4-(5-chloro-2-methoxyphenyl)piperazin-1-ylmethyl]-1-methyl-2-(4-trifluoromethylphenyl)-1,2-dihydropyrazol-3-one

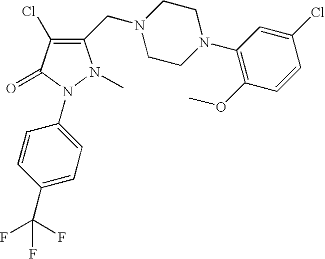

4-Chloro-5-[4-(5-chloro-2-methoxyphenyl)piperazin-1-ylmethyl]-1-methyl-2-(4-trifluoromethylphenyl)-1,2-dihydropyrazol-3-one was obtained from 5-bromomethyl-4-chloro-2-(4-trifluorophenyl)-1-methyl-1,2-dihydropyrazol-3-one (30 mg, 0.078 mmol), 1-(5-chloro-2-methoxyphenyl)piperazine hydrochloride (28 mg, 0.117 mmol) and potassium carbonate (38 mg, 0.274 mmol) in acetonitrile (2 mL) as yellow oil 44 mg (70%). ¹H NMR (300 MHz, CDCl₃): δ (ppm) 7.77 (d, 2H), 7.56 (d, 2H), 6.99 (d, 1H), 6.90 (s, 1H), 6.81 (d, 2H), 3.88 (s, 3H), 3.67 (s, 2H), 3.27 (s, 3H), 3.13 (s, 4H), 2.79 (s, 4H).

### Example 191

### 4-Chloro-5-[4-(2-ethoxyphenyl)piperazin-1-ylmethyl]-1-methyl-2-(4-trifluoromethylphenyl)-1,2-dihydropyrazol-3-one

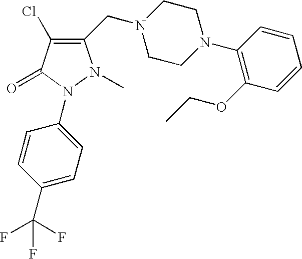

4-Chloro-5-[4-(2-ethoxyphenyl)piperazin-1-ylmethyl]-1-methyl-2-(4-trifluoromethylphenyl)-1,2-dihydropyrazol-3-one was obtained from 5-bromomethyl-4-chloro-2-(4-trifluorophenyl)-1-methyl-1,2-dihydropyrazol-3-one (30 mg, 0.078 mmol), 1-(2-ethoxyphenyl)piperazine monohydrochloride (29 mg, 0.117 mmol) and potassium carbonate (38 mg, 0.274 mmol) in acetonitrile (2 mL) as dark oil 32 mg (52%). ¹H NMR (300 MHz, CDCl₃): δ (ppm) 7.77 (d, 2H), 7.58 (d, 2H), 6.97 (m, 4H), 4.11 (m, 2H), 3.68 (s, 2H), 3.31 (s, 3H), 3.17 (s, 4H), 2.80 (s, 4H), 1.46 (m, 3H).

### Example 192

### 4-Chloro-5-[4-(3,5-dichloropyridin-4-yl)piperazin-1-ylmethyl]-1-methyl-2-(4-trifluoromethylphenyl)-1,2-dihydropyrazol-3-one

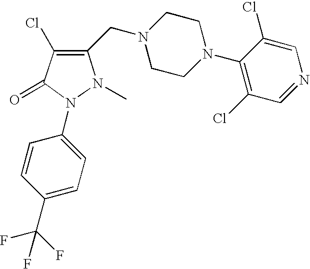

4-Chloro-5-[4-(3,5-dichloropyridin-4-yl)piperazin-1-ylmethyl]-1-methyl-2-(4-trifluoromethylphenyl)-1,2-dihydropyrazol-3-one was obtained from 5-bromomethyl-4-chloro-2-(4-trifluorophenyl)-1-methyl-1,2-dihydropyrazol-3-one (30 mg, 0.078 mmol), 1-(3,5-dichloropyridin-4-yl)piperazine (29 mg, 0.117 mmol) and potassium carbonate (38 mg, 0.274 mmol) in acetonitrile (2 mL) as an off-white solid 33 mg (50%). ¹H NMR (300 MHz, CDCl₃): δ (ppm) 8.37 (s, 2H), 7.76 (d, 2H), 7.58 (d, 2H), 3.68 (s, 2H), 3.44 (m, 4H), 3.29 (s, 3H), 2.75 (s, 4H).

### Example 193

### 8-[4-Chloro-2-methyl-5-oxo-1-(4-trifluoromethylphenyl)-2,5-dihydro-1H-pyrazol-3-ylmethyl]-1-phenyl-1,3,8-triazaspiro[4.5]decan-4-one

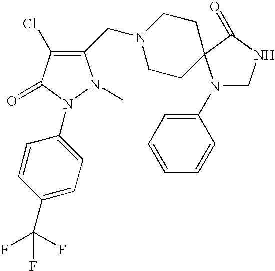

8-[4-Chloro-2-methyl-5-oxo-1-(4-trifluoromethylphenyl)-2,5-dihydro-1H-pyrazol-3-ylmethyl]-1-phenyl-1,3,8-triazaspiro[4.5]decan-4-one was obtained from 5-bromomethyl-4-chloro-2-(4-trifluorophenyl)-1-methyl-1,2-dihydropyrazol-3-one (30 mg, 0.078 mmol), 1-phenyl-1,3,8-triazaspiro[4,5]decan-4-one (35 mg, 0.117 mmol) and potassium carbonate (38 mg, 0.274 mmol) in acetonitrile (2 mL) as an off-white solid 23 mg (33%). ¹H NMR (300 MHz, CDCl₃): δ (ppm) 7.78 (d, 2H), 7.54 (t, 2H), 6.89 (t, 3H), 4.78 (s, 2H), 3.72 (s, 2H), 3.32 (s, 3H), 3.11-3.02 (t, 2H), 2.88 (d, 2H), 2.71 (t, 2H), 1.81 (d, 3H).

### Example 194

### 4-Chloro-5-[4-(2-methoxyphenyl)piperazin-1-ylmethyl]-1-methyl-2-(4-trifluoromethoxyphenyl)-1,2-dihydropyrazol-3-one

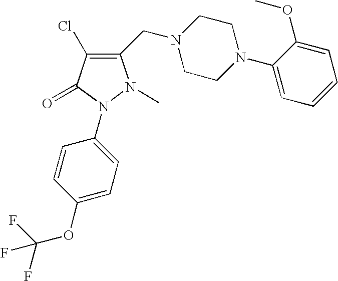

4-Chloro-5-[4-(2-methoxyphenyl)piperazin-1-ylmethyl]-1-methyl-2-(4-trifluoromethoxyphenyl)-1,2-dihydropyrazol-3-one was obtained from 5-bromomethyl-4-chloro-1-methyl-2-(4-trifluoromethoxyphenyl)-1,2-dihydropyrazol-3-one (20 mg, 0.052 mmol), 2-methoxyphenyl piperazine (15 mg, 0.0782 mmol) and potassium carbonate (25 mg, 0.183 mmol) in acetonitrile (2 mL) as yellow gum 20 mg (77%). ¹H NMR (300 MHz, CDCl₃): δ (ppm) 7.48-7.45 (d, 2H), 7.34 (d, 2H), 6.94 (t, 3H), 3.90 (s, 3H), 3.66 (s, 2H), 3.25 (s, 3H), 3.09 (s, 4H), 2.79 (s, 4H).

### Example 195

### 4-Chloro-5-[4-(2-chlorophenyl)piperazin-1-ylmethyl]-1-methyl-2-(4-trifluoromethoxyphenyl)-1,2-dihydropyrazol-3-one

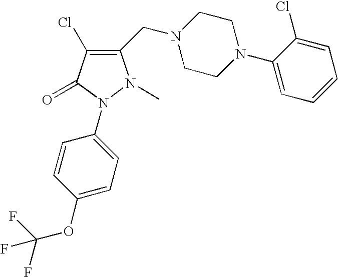

4-Chloro-5-[4-(2-chlorophenyl)piperazin-1-ylmethyl]-1-methyl-2-(4-trifluoromethoxyphenyl)-1,2-dihydropyrazol-3-one was obtained from 5-bromomethyl-4-chloro-1-methyl-2-(4-trifluoromethoxyphenyl)-1,2-dihydropyrazol-3-one (20 mg, 0.052 mmol), 1-(2-chlorophenyl)piperazine (17 mg, 0.0782 mmol) and potassium carbonate (25 mg, 0.183 mmol) in acetonitrile (2 mL) as yellow gum 17 mg (62%). ¹H NMR (300 MHz, CDCl₃): δ (ppm) 7.48 (d, 2H), 7.37 (t, 3H), 7.36 (t, 1H), 7.04 (q, 2H).

### Example 196

### 4-Chloro-5-[4-(5-chloro-2-methoxyphenyl)piperazin-1-ylmethyl]-1-methyl-2-(4-trifluoromethoxyphenyl)-1,2-dihydropyrazol-3-one

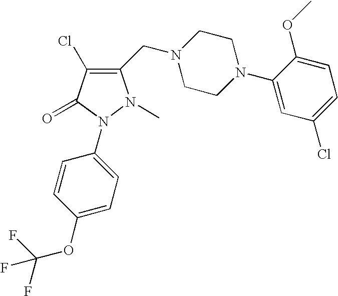

4-Chloro-5-[4-(5-chloro-2-methoxyphenyl)piperazin-1-ylmethyl]-1-methyl-2-(4-trifluoromethoxyphenyl)-1,2-dihydropyrazol-3-one was obtained from 5-bromomethyl-4-chloro-1-methyl-2-(4-trifluoromethoxyphenyl)-1,2-dihydropyrazol-3-one (20 mg, 0.052 mmol), 1-(5-chloro-2-methoxyphenyl)piperazine hydrochloride (16 mg, 0.0782 mmol) and potassium carbonate (25 mg, 0.183 mmol) in acetonitrile (2 mL) as yellow gum 22 mg (74%). ¹H NMR (300 MHz, CDCl₃): δ (ppm) 7.46 (d, 2H), 7.33 (d, 2H), 6.99 (d, 1H), 6.89 (s, 1H), 6.80 (d, 1H), 3.87 (s, 3H), 3.66 (s, 2H), 3.26 (s, 3H), 3.08 (s, 4H), 2.77 (s, 4H).

### Example 197

### 4-Chloro-5-[4-(2-ethoxyphenyl)piperazin-1-ylmethyl]-1-methyl-2-(4-trifluoromethoxyphenyl)-1,2-dihydropyrazol-3-one

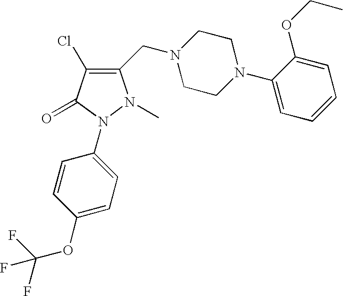

4-Chloro-5-[4-(2-ethoxyphenyl)piperazin-1-ylmethyl]-1-methyl-2-(4-trifluoromethoxyphenyl)-1,2-dihydropyrazol-3-one was obtained from 5-bromomethyl-4-chloro-1-methyl-2-(4-trifluoromethoxyphenyl)-1,2-dihydropyrazol-3-one (20 mg, 0.052 mmol), 1-(2-ethoxyphenyl)piperazine monohydrochloride (19 mg, 0.0782 mmol) and potassium carbonate (25 mg, 0.183 mmol) in acetonitrile (2 mL) as yellow gum 21 mg (75%). ¹H NMR (300 MHz, CDCl₃): δ (ppm) 7.46 (d, 2H), 7.33 (d, 2H), 7.02-6.87 (m, 4H), 4.11 (q, 2H), 3.66 (s, 2H), 3.25 (s, 3H), 3.11 (s, 4H), 2.78 (s, 4H), 1.48 (t, 3H).

### Example 198

### 4-Chloro-5-[4-(5-chloro-2-methylphenyl)piperazin-1-ylmethyl]-1-methyl-2-(4-trifluoromethoxyphenyl)-1,2-dihydropyrazol-3-one

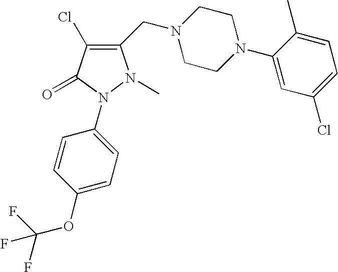

4-Chloro-5-[4-(5-chloro-2-methylphenyl)piperazin-1-ylmethyl]-1-methyl-2-(4-trifluoromethoxyphenyl)-1,2-dihydropyrazol-3-one was obtained from 5-bromomethyl-4-chloro-1-methyl-2-(4-trifluoromethoxyphenyl)-1,2-dihydropyrazol-3-one (20 mg, 0.052 mmol), 1-(5-chloro-2-methylphenyl)piperazine (18 mg, 0.0782 mmol) and potassium carbonate (25 mg, 0.183 mmol) in acetonitrile (2 mL) as an off-white solid 20 mg (72%). ¹H NMR (300 MHz, CDCl₃): δ (ppm) 7.46 (d, 2H), 7.39 (d, 2H), 7.13 (d, 1H), 6.98 (d, 2H), 3.66 (s, 2H), 3.26 (s, 3H), 2.89 (s, 4H), 2.74 (s, 4H), 2.27 (s, 3H).

### Example 199

### 4-Chloro-5-[4-(3,5-dichloropyridin-4-yl)piperazin-1-ylmethyl]-1-methyl-2-(4-trifluoromethoxyphenyl)-1,2-dihydropyrazol-3-one

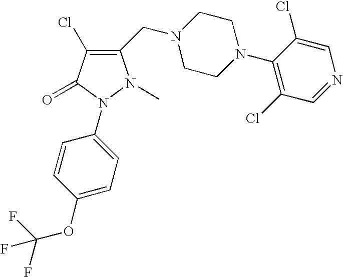

4-Chloro-5-[4-(3,5-dichloropyridin-4-yl)piperazin-1-ylmethyl]-1-methyl-2-(4-trifluoromethoxyphenyl)-1,2-dihydropyrazol-3-one was obtained from 5-bromomethyl-4-chloro-1-methyl-2-(4-trifluoromethoxyphenyl)-1,2-dihydropyrazol-3-one (20 mg, 0.052 mmol), 1-(3,5-dichloropyridin-4-yl)piperazine (19 mg, 0.0782 mmol) and potassium carbonate (25 mg, 0.183 mmol) in acetonitrile (2 mL) as yellow gum 31 mg (100%). ¹H NMR (300 MHz, CDCl₃): δ (ppm) 8.36 (s, 2H), 7.47 (d, 2H), 7.36 (d, 2H), 3.66 (s, 2H), 3.30 (s, 4H), 3.27 (s, 3H), 2.72 (s, 4H).

### Example 200

### 8-[4-Chloro-2-methyl-5-oxo-1-(4-trifluoromethoxyphenyl)-2,5-dihydro-1H-pyrazol-3-ylmethyl]-1-phenyl-1,3,8-triazaspiro[4.5]decan-4-one

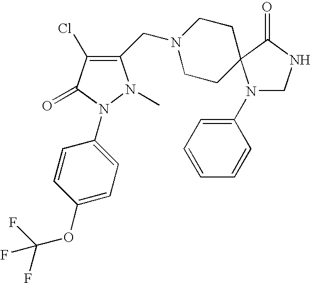

8-[4-Chloro-2-methyl-5-oxo-1-(4-trifluoromethoxyphenyl)-2,5-dihydro-1H-pyrazol-3-ylmethyl]-1-phenyl-1,3,8-triazaspiro[4.5]decan-4-one was obtained from 5-bromomethyl-4-chloro-1-methyl-2-(4-trifluoromethoxyphenyl)-1,2-dihydropyrazol-3-one (20 mg, 0.052 mmol), 1-phenyl-1,3,8-triazaspiro[4,5]decan-4-one (23 mg, 0.0782 mmol) and potassium carbonate (25 mg, 0.183 mmol) in acetonitrile (2 mL) as an off-white solid 27 mg (86%). ¹H NMR (300 MHz, CDCl₃): δ (ppm) 7.47 (d, 2H), 7.45-7.27 (m, 6H), 6.90 (t, 3H), 4.78 (s, 2H), 3.70 (s, 2H), 3.34 (s, 3H), 3.05 (t, 2H), 2.87 (d, 2H), 2.70 (t, 2H), 1.44 (d, 3H).

### Example 201

### 4-Chloro-1-methyl-5-[4-(3-phenyl-propyl)-piperidin-1-ylmethyl]-2-(4-trifluoromethoxy-phenyl)-1,2-dihydro-pyrazol-3-one

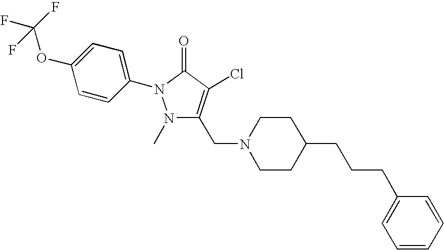

4-Chloro-1-methyl-5-[4-(3-phenyl-propyl)-piperidin-1-ylmethyl]-2-(4-trifluoromethoxy-phenyl)-1,2-dihydro-pyrazol-3-one was synthesized from 5-bromomethyl-4-chloro-1-methyl-2-(4-trifluoromethoxy-phenyl)-1,2-dihydro-pyrazol-3-one (30 mg, 0.078 mmol), 4-(3-phenyl-propyl)-piperidine (23.8 mg, 0.117 mmol) and potassium carbonate (31.78 mg, 0.23 mmol) in 3 mL of acetonitrile. The desired product was isolated by eluting the crude though a 2 g SPE tube in a solution of 15% acetone and hexanes (40.8 mg, 100.3%). ¹H NMR (300 MHz, CDCl₃): δ ppm 1.21-1.33 (m, 5H), 1.64-1.74 (m, 4H), 2.12 (t of d, 2H), 2.62 (t, 2H), 2.89 (d, 2H), 3.23 (s, 3H), 3.54 (s, 2H), 7.18-7.21 (m, 3H), 7.30-7.44 (m, 4H), 7.45-7.47 (m, 2H).

### Example 202

### 4-Chloro-2-(3-chloro-4-fluorophenyl)-5-[4-(3,5-dichloro-pyridin-4-yl)-piperazin-1-yl-methyl]-1-methyl-2,4-dihydro-pyrazol-3-one

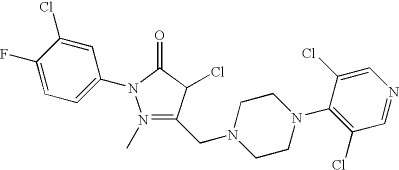

4-Chloro-2-(3-chloro-4-fluorophenyl)-5-[4-(3,5-dichloro-pyridin-4-yl)-piperazin-1-yl-methyl]-1-methyl-2,4-dihydro-pyrazol-3-one was obtained from 5-bromomethyl-4-chloro-2-(3-chloro-4-fluorophenyl)-1-methyl-1,2-dihydropyrazol-3-one (25 mg, 0.071 mmol), 1-(3,5-dichloro-pyridin-4-yl)-piperazine (25 mg, 0.110 mmol) and potassium carbonate (29 mg, 0.21 mmol) in acetonitrile (1.5 mL) as an off-white solid 34 mg (96%). ¹H NMR (300 MHz, CDCl₃): δ (ppm) 8.37 (s, 2H), 7.36-7.49 (m, 1H), 7.28-7.36 (m, 2H), 3.66 (s, 2H), 3.42 (t, 4H), 3.26 (s, 3H), 2.73 (t, 4H).

### Example 203

### 4-Chloro-2-(3-chloro-4-fluorophenyl)-5-[4-(5-chloro-2-methoxyphenyl)-piperazin-1-ylmethyl]-1-methyl-2,4-dihydro-pyrazol-3-one

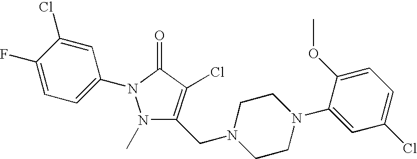

4-Chloro-2-(3-chloro-4-fluorophenyl)-5-[4-(5-chloro-2-methoxyphenyl)-piperazin-1-ylmethyl]-1-methyl-2,4-dihydro-pyrazol-3-one was obtained from 5-bromomethyl-4-chloro-2-(3-chloro-4-fluorophenyl)-1-methyl-1,2-dihydropyrazol-3-one (25 mg, 0.071 mmol), 1-(5-chloro-2-methoxyphenyl)-piperazine (29 mg, 0.110 mmol) and potassium carbonate (29 mg, 0.21 mmol) in acetonitrile (1.5 mL) as an amber oil 34 mg (97%). ¹H NMR (300 MHz, CDCl₃): δ (ppm) 7.48-7.49 (m, 1H), 7.24-7.47 (m, 2H), 6.98 (dd, 1H), 6.89 (d, 1H), 6.79 (d, 1H), 3.87 (s, 3H), 3.67 (s, 2H), 3.25 (t, 3H), 3.14 (s, 4H), 2.80 (s, 4H).

### Example 204

### 4-Chloro-2-(3-chloro-4-fluorophenyl)-1-methyl-5-[4-(3-phenyl-[1,2,4]thiadiazol-5-yl)-piperazin-1-ylmethyl]-1,2-dihydro-pyrazol-3-one

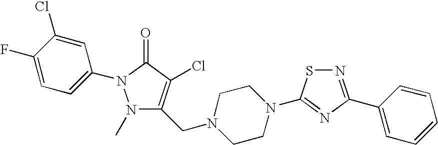

4-Chloro-2-(3-chloro-4-fluorophenyl)-1-methyl-5-[4-(3-phenyl-[1,2,4]thiadiazol-5-yl)-piperazin-1-ylmethyl]-1,2-dihydro-pyrazol-3-one was obtained from 5-bromomethyl-4-chloro-2-(3-chloro-4-fluorophenyl)-1-methyl-1,2-dihydropyrazol-3-one (25 mg, 0.071 mmol), 1-(3-phenyl-[1,2,4]thiadiazol-5-yl)piperazine (27 mg, 0.110 mmol) and potassium carbonate (29 mg, 0.21 mmol) in acetonitrile (1.5 mL) as an pale yellow oil, 33 mg (90%). ¹H NMR (300 MHz, CDCl₃): δ (ppm) 8.18-8.22 (m, 2H), 7.43-7.49 (m, 4H), 7.28-7.34 (m, 2H), 3.68 (t, 6H), 3.23 (s, 3H), 2.75 (t, 4H).

### Example 205

### 8-[4-Chloro-1-(3-chloro-4-fluorophenyl)-2-methyl-5-oxo-2,5-dihydro-1H-pyrazol-3-ylmethyl]-1-phenyl-1,3-8-triazaspiro[4.5]decan-4-one

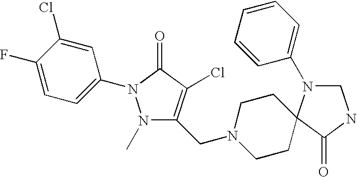

8-[4-Chloro-1-(3-chloro-4-fluorophenyl)-2-methyl-5-oxo-2,5-dihydro-1H-pyrazol-3-ylmethyl]-1-phenyl-1,3-8-triazaspiro[4.5]decan-4-one was obtained from 5-bromomethyl-4-chloro-2-(3-chloro-4-fluorophenyl)-1-methyl-1,2-dihydropyrazol-3-one (25 mg, 0.071 mmol), 1-phenyl-1,3,8-triaza-spiro[4.5]decan-4-one (25 mg, 0.110 mmol) and potassium carbonate (29 mg, 0.21 mmol) in acetonitrile (1.5 mL) as an off-white solid, 34 mg (96%). ¹H NMR (300 MHz, CDCl₃): δ (ppm) 7.44 (d, 1H), 7.21-7.28 (m, 4H), 6.81-6.86 (m, 3H), 4.68 (s, 2H), 3.62 (s, 2H), 3.26 (d, 6H), 2.98 (t, 2H), 2.80 (d, 2H), 2.56 (td, 2H), 1.73 (d, 2H).

### Example 206

### 4-Chloro-2-(3-chloro-4-fluorophenyl)-5-[4-(2-methoxyphenyl)-piperazin-1-ylmethyl]-1-methyl-1,2-dihydro-pyrazol-3-one

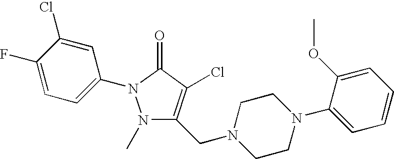

4-Chloro-2-(3-chloro-4-fluorophenyl)-5-[4-(2-methoxyphenyl)-piperazin-1-ylmethyl]-1-methyl-1,2-dihydro-pyrazol-3-one was obtained from 5-bromomethyl-4-chloro-2-(3-chloro-4-fluorophenyl)-1-methyl-1,2-dihydropyrazol-3-one (33 mg, 0.093 mmol), 1-(2-methoxyphenyl)-piperazine (27 mg, 0.140 mmol) and potassium carbonate (39 mg, 0.28 mmol) in acetonitrile (2.0 mL) as a colourless oil 15 mg (34%). ¹H NMR (300 MHz, CDCl₃): δ (ppm) 7.48 (dd, 1H), 7.27-7.34 (m, 3H), 6.91-7.02 (m, 4H), 3.90 (s, 3H), 3.66 (s, 2H), 3.25 (s, 3H), 3.08 (s, 4H), 2.79 (s, 4H).

### Example 207

### 4-Chloro-5-{1-[4-(5-chloro-2-methoxyphenyl)piperazin-1-yl]-ethyl}-1-methyl-2-phenyl-1,2-dihydro-pyrazol-3-one

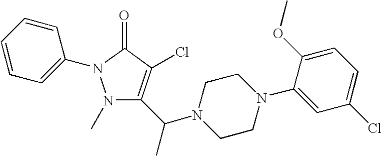

4-Chloro-5-{1-[4-(5-chloro-2-methoxyphenyl)piperazin-1-yl]-ethyl}-1-methyl-2-phenyl-1,2-dihydro-pyrazol-3-one was obtained from 5-(1-bromoethyl)-4-chloro-1-methyl-2-phenyl-1,2-dihydro-pyrazol-3-one (30 mg, 0.095 mmol), 1-(5-chloro-2-methoxyphenyl)piperazine (37 mg, 0.143 mmol) and potassium carbonate (53 mg, 0.38 mmol) in acetonitrile (2.0 mL) as a beige solid, 33.7 mg (51%). ¹H NMR (300 MHz, CDCl₃): δ (ppm) 7.49-7.50 (m, 3H), 7.34-7.47 (m, 2H), 6.95 (dd, 1H), 6.87 (d, 1H), 6.78 (d, 1H), 3.87 (s, 4H), 3.36 (s, 3H), 3.10 (s, 4H), 2.83 (s, 2H), 2.71 (d, 2H), 1.52 (d, 3H).

### Example 208

### 4-Chloro-5-{1-[4-(2-chloro-phenyl)piperazin-1-yl]-ethyl}-1-methyl-2-phenyl-1,2-dihydro-pyrazol-3-one

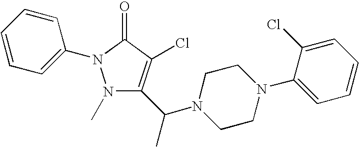

4-Chloro-5-{1-[4-(2-chloro-phenyl)piperazin-1-yl]-ethyl}-1-methyl-2-phenyl-1,2-dihydro-pyrazol-3-one was obtained from 5-(1-bromoethyl)-4-chloro-1-methyl-2-phenyl-1,2-dihydro-pyrazol-3-one (30 mg, 0.095 mmol), 1-(2-chlorophenyl)piperazine (33 mg, 0.143 mmol) and potassium carbonate (53 mg, 0.38 mmol) in acetonitrile (2.0 mL) as an oil, 43.0 mg (70%). ¹H NMR (300 MHz, CDCl₃): δ (ppm) 7.28-7.49 (m, 7H), 7.00-7.07 (m, 2H), 3.91 (q, 1H), 3.37 (s, 3H), 3.11 (s, 4H), 2.84 (s, 2H), 2.72 (d, 2H), 1.53 (d, 3H).

### Example 209

### 4-Chloro-5-{1-[4-(2-methoxyphenyl)piperazin-1-yl]-ethyl}-1-methyl-2-phenyl-1,2-dihydro-pyrazol-3-one

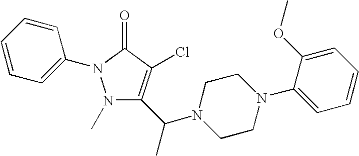

4-Chloro-5-{1-[4-(2-methoxyphenyl)piperazin-1-yl]-ethyl}-1-methyl-2-phenyl-1,2-dihydro-pyrazol-3-one was obtained from 5-(1-bromoethyl)-4-chloro-1-methyl-2-phenyl-1,2-dihydro-pyrazol-3-one (30 mg, 0.095 mmol), 1-(2-methoxyphenyl)piperazine (27 mg, 0.143 mmol) and potassium carbonate (53 mg, 0.38 mmol) in acetonitrile (2.0 mL) as an oil, 37.4 mg (61%). ¹H NMR (300 MHz, CDCl₃): δ (ppm) 7.34-7.49 (m, 5H), 6.88-7.00 (m, 4H), 3.89 (s, 4H), 3.37 (s, 3H), 3.12 (s, 4H), 2.86 (s, 2H), 2.72 (d, 2H), 1.52 (d, 3H).

### Example 210

### 4-Chloro-5-{1-[4-(5-chloro-2-methylphenyl)piperazin-1-yl]-ethyl}-1-methyl-2-phenyl-1,2-dihydro-pyrazol-3-one

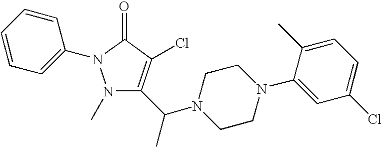

4-Chloro-5-{1-[4-(5-chloro-2-methylphenyl)piperazin-1-yl]-ethyl}-1-methyl-2-phenyl-1,2-dihydro-pyrazol-3-one was obtained from 5-(1-bromoethyl)-4-chloro-1-methyl-2-phenyl-1,2-dihydro-pyrazol-3-one (30 mg, 0.095 mmol), 1-(5-chloro-2-methylphenyl)piperazine (30 mg, 0.143 mmol) and potassium carbonate (53 mg, 0.38 mmol) in acetonitrile (2.0 mL) as an oil, 46.0 mg (72%). ¹H NMR (300 MHz, CDCl₃): δ (ppm) 7.49-7.52 (m, 2H), 7.35-7.47 (m, 3H), 7.09 (d, 1H), 6.97 (d, 2H), 3.89 (q, 4H), 3.37 (s, 3H), 2.95 (s, 4H), 2.80 (s, 2H), 2.69 (s, 2H), 1.53 (d, 3H).

### Example 211

### 4-Chloro-5-{1-[4-(2,4-dimethylphenyl)piperazin-1-yl]-ethyl}-1-methyl-2-phenyl-1,2-dihydro-pyrazol-3-one

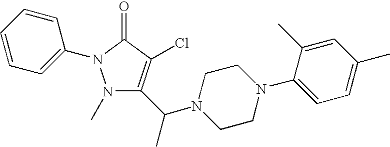

4-Chloro-5-{1-[4-(2,4-dimethylphenyl)piperazin-1-yl]-ethyl}-1-methyl-2-phenyl-1,2-dihydro-pyrazol-3-one was obtained from 5-(1-bromoethyl)-4-chloro-1-methyl-2-phenyl-1,2-dihydro-pyrazol-3-one (30 mg, 0.095 mmol), 1-(2,4-dimethylphenyl)piperazine (27 mg, 0.143 mmol) and potassium carbonate (53 mg, 0.38 mmol) in acetonitrile (2.0 mL) as a white solid, 39.0 mg (64%). ¹H NMR (300 MHz, CDCl₃): δ (ppm) 7.35-7.52 (m, 5H), 6.93-7.03 (d, 3H), 3.89 (q, 4H), 3.39 (s, 3H), 2.93 (s, 4H), 2.81 (s, 2H), 2.68 (s, 2H), 2.30 (s, 6 h), 1.53 (d, 3H).

### Example 212

### 4-Chloro-1-methyl-5-[1-(3-methyl-4-m-tolylpiperazin-1-yl)-ethyl]-2-phenyl-1,2-dihydro-pyrazol-3-one

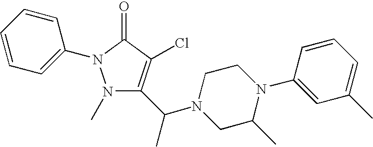

4-Chloro-1-methyl-5-[1-(3-methyl-4-m-tolylpiperazin-1-yl)-ethyl]-2-phenyl-1,2-dihydro-pyrazol-3-one was obtained from 5-(1-bromoethyl)-4-chloro-1-methyl-2-phenyl-1,2-dihydro-pyrazol-3-one (30 mg, 0.095 mmol), 2-methyl-1-m-tolylpiperazine (27 mg, 0.143 mmol) and potassium carbonate (53 mg, 0.38 mmol) in acetonitrile (2.0 mL) as an oil, 39.7 mg (65%). ¹H NMR (300 MHz, CDCl₃): δ (ppm) 7.36-7.51 (m, 5H), 7.23 (t, 1H), 6.71-6.75 (m, 3H), 4.13 (s, 1H), 3.81 (qu, 1H), 3.38 (s, 3H), 3.12-3.22 (m, 2H), 2.87-2.91 (m, 2H), 2.36-2.60 (m, 2H), 2.34 (s, 3H), 1.53 (t, 3H), 1.10 (dd, 3H).

### Example 213

### 1-[1-(4-chloro-2-methyl-5-oxo-1-phenyl-2,5-dihydro-1H-pyrazol-3-yl)-ethyl]-4-phenylpiperidine-4-carbonitrile

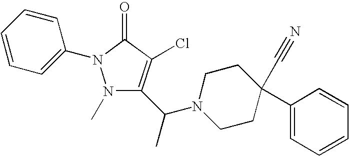

1-[1-(4-chloro-2-methyl-5-oxo-1-phenyl-2,5-dihydro-1H-pyrazol-3-yl)-ethyl]-4-phenylpiperidine-4-carbonitrile was obtained from 5-(1-bromoethyl)-4-chloro-1-methyl-2-phenyl-1,2-dihydro-pyrazol-3-one (32 mg, 0.095 mmol), 4-phenylpiperidine-4-carbonitrile (32 mg, 0.143 mmol) and potassium carbonate (53 mg, 0.38 mmol) in acetonitrile (2.0 mL) as an oil, 40.3 mg (67%). ¹H NMR (300 MHz, CDCl₃): δ (ppm) 7.36-7.50 (m, 10H), 3.94 (q, 1H), 3.30 (s, 4H), 3.06 (d, 1H), 2.65 (dd, 2H), 2.06-2.24 (m, 4H), 1.56 (d, 3H).

### Example 214

### 8-[1-(4-chloro-2-methyl-5-oxo-1-phenyl-2,5-dihydro-1H-pyrazol-3-yl)-ethyl]-1-phenyl-1,3,8-triazaspiro[4.5]decan-4-one

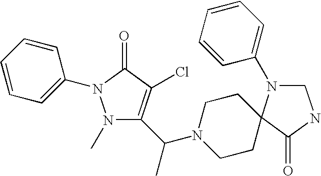

8-[1-(4-chloro-2-methyl-5-oxo-1-phenyl-2,5-dihydro-1H-pyrazol-3-yl)-ethyl]-1-phenyl-1,3,8-triazaspiro[4.5]decan-4-one was obtained from 5-(1-bromoethyl)-4-chloro-1-methyl-2-phenyl-1,2-dihydro-pyrazol-3-one (32 mg, 0.095 mmol), 1-phenyl-1,3,8-triazaspiro[4.5]decan-4-one (33 mg, 0.143 mmol) and potassium carbonate (53 mg, 0.38 mmol) in acetonitrile (2.0 mL) as a white solid, 49 mg. ¹H NMR (300 MHz, CDCl₃): δ (ppm) 7.81 (s, 1H), 7.24-7.49 (m, 7H), 6.85-6.89 (m, 3 h), 4.76 (s, 2H), 3.95 (q, 1H), 3.42 (s, 3H), 2.87-3.42 (m, 4H), 2.67-2.75 (m, 2H), 1.75 (q, 2H), 1.53 (d, 3H).

### Example 215

### 4-Chloro-5-{1-[4-(5-chloro-2-methoxy-phenyl)-piperazin-1-yl]-ethyl}-1-methyl-2-(4-trifluoromethoxy-phenyl)-1,2-dihydro-pyrazol-3-one

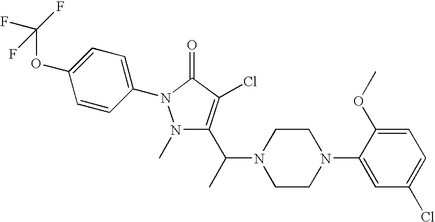

4-Chloro-5-{1-[4-(5-chloro-2-methoxy-phenyl)-piperazin-1-yl]-ethyl}-1-methyl-2-(4-trifluoromethoxy-phenyl)-1,2-dihydro-pyrazol-3-one was synthesized from 5-(1-bromo-ethyl)-4-chloro-1-methyl-2-(4-trifluoromethoxy-phenyl)-1,2-dihydro-pyrazol-3-one (30 mg, 0.0748 mmol), 1-(5-chloro-2-methoxy-phenyl)-piperazine (29.52 mg, 0.1122 mmol), and potassium carbonate (30.95 mg, 0.224 mmol) in 3 mL of acetonitrile. The crude product was purified by column chromatography using a solution of 40% ethyl acetate and hexanes to yield a pale yellow solid (41.3 mg, 101.1%). ¹H NMR (300 MHz, CDCl₃): δ ppm 1.53 (d, 3H), 2.73 (br, 2H), 2.85 (br, 2H), 3.12 (br, 4H), 3.37 (s, 3H), 3.87 (s, 2H), 3.90 (quartet, 1H), 6.80 (d, 1H), 6.89 (s, 1H), 6.99 (d of d, 1H), 7.33-7.45 (m, 2H).

### Example 216

### 4-Chloro-5-{1-[4-(5-chloro-phenyl)-piperazin-1-yl]-ethyl}-1-methyl-2-(4-trifluoromethoxy-phenyl)-1,2-dihydro-pyrazol-3-one

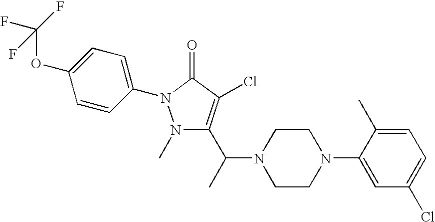

4-Chloro-5-{1-[4-(5-chloro-phenyl)-piperazin-1-yl]-ethyl}-1-methyl-2-(4-trifluoromethoxy-phenyl)-1,2-dihydro-pyrazol-3-one was synthesized from 5-(1-bromo-ethyl)-4-chloro-1-methyl-2-(4-trifluoromethoxy-phenyl)-1,2-dihydro-pyrazol-3-one (30 mg, 0.0748 mmol), 1-(5-chloro-2-methyl-phenyl)-piperazine (23.64 mg, 0.1122 mmol) and potassium carbonate (30.95 mg, 0.224 mmol) in 3 mL of acetonitrile. The crude product was purified by column chromatography using a solution of 40% ethyl acetate and hexanes to yield a pale yellow solid (39.2 mg, 101.5%). ¹H NMR (300 MHz, CDCl₃): δ ppm 1.53 (d, 3H), 2.28 (s, 3H), 2.69 (br, 2H), 2.91 (br, 4H), 3.91 (quartet, 1H), 6.99 (d, 2H), 7.13 (d, 1H), 7.34-7.46 (m, 4H).

### Example 217

### 4-Chloro-1-methyl-5-{1-[4-(3-phenyl-propyl)-piperidin-1-yl]-ethyl}-2-(4-trifluoromethoxy-phenyl)-1,2-dihydro-pyrazol-3-one

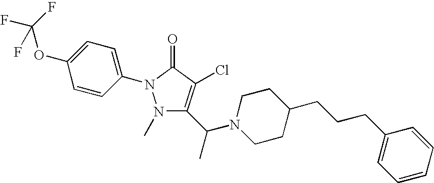

4-Chloro-1-methyl-5-{1-[4-(3-phenyl-propyl)-piperidin-1-yl]-ethyl}-2-(4-trifluoromethoxy-phenyl)-1,2-dihydro-pyrazol-3-one was synthesized from 5-(1-bromo-ethyl)-4-chloro-1-methyl-2-(4-trifluoromethoxy-phenyl)-1,2-dihydro-pyrazol-3-one (30 mg, 0.0748 mmol), 4-(3-phenyl-propyl)-piperidine (22.81 mg, 0.1122 mmol) and potassium carbonate (30.95 mg, 0.224 mmol) in 3 mL of acetonitrile. The crude product was purified by column chromatography using a solution of 40% ethyl acetate and hexanes to yield a pale yellow oil (31.1 mg, 79.7%). ¹H NMR (300 MHz, CDCl₃): δ ppm 1.18-1.32 (m, 6H), 1.44 (d, 3H), 1.64 (m, 5H), 2.00 (quintet, 2H), 2.62 (t, 2H), 2.62 (dd, 2H), 3.34 (s, 3H), 3.76 (quartet, 1H), 7.18-7.21 (m, 3H), 7.30-7.34 (m, 4H), 7.40-7.44 (m, 2H).

### Example 218

### 4-Chloro-5-[4-(5-chloro-2-methoxyphenyl)-piperazin-1-ylmethyl]-2-cyclohexyl-1-methyl-1,2-dihydro-pyrazol-3-one

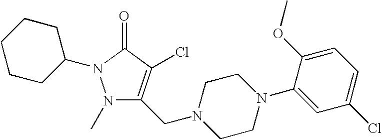

4-Chloro-5-[4-(5-chloro-2-methoxyphenyl)-piperazin-1-ylmethyl]-2-cyclohexyl-1-methyl-1,2-dihydro-pyrazol-3-one was synthesized using 5-bromomethyl-4-chloro-2-cyclohexyl-1-methyl-1,2-dihydro-pyrazol-3-one (40 mg, 0.13 mmol), 1-(5-chloro-2-methoxyphenyl)piperazine HCl (51 mg, 0.195 mmol) and potassium carbonate (72 mg, 0.520 mmol) in acetonitrile (2.0 mL) to yield 53.8 mg (91%). ¹H NMR (300 MHz, CDCl₃) δ (ppm): 6.96 (dd, 1H), 6.86 (s, 1H), 6.77 (d, 1H), 4.06-4.14 (m 1H), 3.86 (s, 3H), 3.52 (s, 2H), 3.38 (s, 3H), 3.06 (s, 4H), 2.68 (s, 4H), 1.96-2.05 (m, 2H), 1.85 (t, 4H), 1.70 (d, 1H), 1.25-1.40 (m, 3H).

### Example 219

### 4-Chloro-5-[4-(2-chlorophenyl)-piperazin-1-ylmethyl]-2-cyclohexyl-1-methyl-1,2-dihydro-pyrazol-3-one

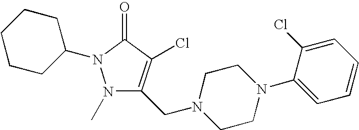

4-Chloro-5-[4-(2-chlorophenyl)-piperazin-1-ylmethyl]-2-cyclohexyl-1-methyl-1,2-dihydro-pyrazol-3-one was synthesized using 5-bromomethyl-4-chloro-2-cyclohexyl-1-methyl-1,2-dihydro-pyrazol-3-one (30 mg, 0.098 mmol), 1-(2-chlorophenyl)piperazine (35 mg, 0.146 mmol) and potassium carbonate (40 mg, 0.29 mmol) in acetonitrile (2.0 mL) to yield 33.3 mg (80%) of an off-white solid. ¹H NMR (300 MHz, CDCl₃) δ (ppm): 7.37 (dd, 1H), 7.20-7.28 (m, 1H), 6.98-7.05 (m, 2H), 4.07-4.12 (m, 1H), 3.53 (s, 2H), 3.38 (s, 3H), 3.07 (s, 4H), 2.70 (s, 4H), 1.96-2.06 (m, 2H), 1.84 (t, 4H), 1.70 (d, 1H), 1.20-1.40 (m, 3H).

### Example 220

### 4-Chloro-2-cyclohexyl-5-[4-(2-methoxyphenyl)-piperazin-1-ylmethyl]-1-methyl-1,2-dihydro-pyrazol-3-one

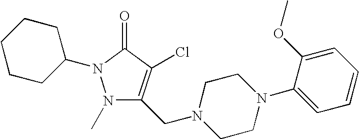

4-Chloro-2-cyclohexyl-5-[4-(2-methoxyphenyl)-piperazin-1-ylmethyl]-1-methyl-1,2-dihydro-pyrazol-3-one was synthesized using 5-bromomethyl-4-chloro-2-cyclohexyl-1-methyl-1,2-dihydro-pyrazol-3-one (30 mg, 0.098 mmol), 1-(2-methoxyphenyl)piperazine (28 mg, 0.146 mmol) and potassium carbonate (40 mg, 0.29 mmol) in acetonitrile (2.0 mL) to yield 38 mg (92%) of an off-white solid. ¹H NMR (300 MHz, CDCl₃) δ (ppm): 6.98-7.27 (m, 1H), 6.86-6.93 (m, 3H), 4.06-4.12 (m, 1H), 3.88 (s, 3H), 3.52 (s, 2H), 3.38 (s, 3H), 3.08 (s, 4H), 2.70 (s, 4H), 1.96-2.06 (m, 2H), 1.86 (t, 3H), 1.70 (d, 1H), 1.24-1.39 (m, 3H).

### Example 221

### 4-Chloro-5-[4-(5-chloro-2-methylphenyl)-piperazin-1-ylmethyl]-2-cyclohexyl-1-methyl-1,2-dihydro-pyrazol-3-one

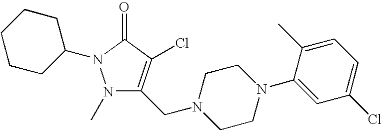

4-Chloro-5-[4-(5-chloro-2-methylphenyl)-piperazin-1-ylmethyl]-2-cyclohexyl-1-methyl-1,2-dihydro-pyrazol-3-one was synthesized using 5-bromomethyl-4-chloro-2-cyclohexyl-1-methyl-1,2-dihydro-pyrazol-3-one (40 mg, 0.130 mmol), 1-(5-chloro-2-methylphenyl)piperazine (41 mg, 0.195 mmol) and potassium carbonate (72 mg, 0.520 mmol) in acetonitrile (2.0 mL) to yield 58 mg (103%) of a solid. ¹H NMR (300 MHz, CDCl₃) δ (ppm): 7.09 (d, 1H), 6.94-6.98 (m, 2H), 4.07-4.14 (m, 1H), 3.53 (s, 2H), 3.39 (s, 3H), 2.90 (t, 4H), 2.65 (s, 4H), 2.25 (s, 3H), 1.97-2.06 (m, 2H), 1.86 (t, 4H), 1.71 (d, 1H), 1.25-1.40 (m, 3H).

### Example 222

### 4-Chloro-2-cyclohexyl-5-[4-(2-ethoxyphenyl)-piperazin-1-ylmethyl]-1-methyl-1,2-dihydro-pyrazol-3-one

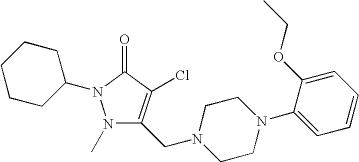

4-Chloro-2-cyclohexyl-5-[4-(2-ethoxyphenyl)-piperazin-1-ylmethyl]-1-methyl-1,2-dihydro-pyrazol-3-one was synthesized using 5-bromomethyl-4-chloro-2-cyclohexyl-1-methyl-1,2-dihydro-pyrazol-3-one (40 mg, 0.130 mmol), 1-(2-ethoxyphenyl)piperazine (47 mg, 0.195 mmol) and potassium carbonate (72 mg, 0.520 mmol) in acetonitrile (2.0 mL) to yield 52 mg (93%) of a solid. ¹H NMR (300 MHz, CDCl₃) δ (ppm): 6.84-7.01 (m, 4H), 4.04-4.12 (m, 1H), 3.52 (s, 2H), 3.39 (s, 3H), 2.70 (s, 4H), 2.68 (s, 4H), 1.97-2.06 (m, 2H), 1.86 (t, 4H), 1.70 (d, 1H), 1.46 (t, 2H), 1.24-1.39 (m, 3H).

### Example 223

### 4-Chloro-2-cyclohexyl-5-[4-(2,4-dimethylphenyl)-piperazin-1-ylmethyl]-1-methyl-1,2-dihydro-pyrazol-3-one

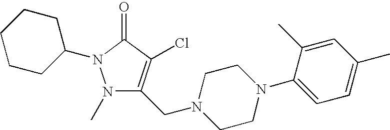

4-Chloro-2-cyclohexyl-5-[4-(2,4-dimethylphenyl)-piperazin-1-ylmethyl]-1-methyl-1,2-dihydro-pyrazol-3-one was synthesized using 5-bromomethyl-4-chloro-2-cyclohexyl-1-methyl-1,2-dihydro-pyrazol-3-one (40 mg, 0.130 mmol), 1-(2,4-dimethylphenyl)piperazine (37 mg, 0.195 mmol) and potassium carbonate (72 mg, 0.520 mmol) in acetonitrile (2.0 mL) to yield 50 mg (93%) of a solid. ¹H NMR (300 MHz, CDCl₃) δ (ppm): 6.91-7.02 (m, 3H), 4.05-4.13 (m, 1H), 3.53 (s, 2H), 3.40 (s, 3H), 2.90 (t, 4H), 2.65 (t, 4H), 2.90 (t, 4H), 2.28 (s, 6H), 1.97-2.07 (m, 2H), 1.87 (t, 4H), 1.70 (d, 1H), 1.26-1.40 (m, 3H).

### Example 224

### 4-Chloro-2-cyclohexyl-5-[4-(3,5-dichloropyridin-4-yl)-piperazin-1-ylmethyl]-1-methyl-1,2-dihydro-pyrazol-3-one

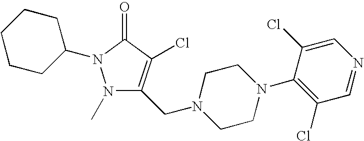

4-Chloro-2-cyclohexyl-5-[4-(3,5-dichloropyridin-4-yl)-piperazin-1-ylmethyl]-1-methyl-1,2-dihydro-pyrazol-3-one was synthesized using 5-bromomethyl-4-chloro-2-cyclohexyl-1-methyl-1,2-dihydro-pyrazol-3-one (30 mg, 0.0975 mmol), 1-(3,5-dichloropyridin-4-yl)piperazine (34 mg, 0.146 mmol) and potassium carbonate (40 mg, 0.290 mmol) in acetonitrile (2.0 mL) to yield 42.5 mg (95%) of a solid. ¹H NMR (300 MHz, CDCl₃) δ (ppm): 8.34 (s, 2H), 4.07-4.14 (m, 1H), 3.53-3.42 (m, 7H), 2.63 (t, 4H), 1.96-2.05 (m, 2H), 1.86 (t, 4H), 1.70 (d, 1H), 1.24-1.39 (m, 3H).

### Example 225

### 8-(4-Chloro-1-cyclohexyl-2-methyl-5-oxo-2,5-dihydro-1H-pyrazol-3-ylmethyl)-1-phenyl-1,3,8-triaza-spiro[4.5]decan-4-one

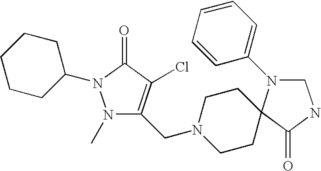

8-(4-Chloro-1-cyclohexyl-2-methyl-5-oxo-2,5-dihydro-1H-pyrazol-3-ylmethyl)-1-phenyl-1,3,8-triaza-spiro[4.5]decan-4-one was synthesized using 5-bromomethyl-4-chloro-2-cyclohexyl-1-methyl-1,2-dihydro-pyrazol-3-one (40 mg, 0.130 mmol), 1-phenyl-1,3,8-triaza-spiro[4.5]decan-4-one (45 mg, 0.195 mmol) and potassium carbonate (72 mg, 0.520 mmol) in acetonitrile (2.0 mL) to yield 47.6 mg (80%) of a solid. ¹H NMR (300 MHz, CDCl₃) δ (ppm): 7.58 (s, 1H), 7.28 (t, 2H), 6.86-6.91 (m, 2H), 4.77 (s, 2H), 4.07-4.14 (m, 1H), 3.55 (s, 2H), 3.44 (s, 3H), 2.77 (td, 2H), 2.70 (s, 2H), 2.63 (td, 2H), 1.73-2.05 (m, 7H), 1.25-1.40 (m, 3H).

### Example 226

### 1-(4-Chloro-1-cyclohexyl-2-methyl-5-oxo-2,5-dihydro-1H-pyrazol-3-ylmethyl)-4-phenylpiperidine-4-carbonitrile

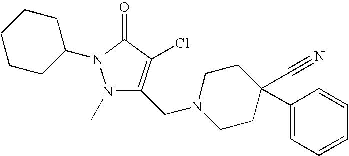

1-(4-Chloro-1-cyclohexyl-2-methyl-5-oxo-2,5-dihydro-1H-pyrazol-3-ylmethyl)-4-phenylpiperidine-4-carbonitrile was synthesized using 5-bromomethyl-4-chloro-2-cyclohexyl-1-methyl-1,2-dihydro-pyrazol-3-one (30 mg, 0.0975 mmol), 1-phenylpiperidine-4-carbonitrile (33 mg, 0.146 mmol) and potassium carbonate (54 mg, 0.390 mmol) in acetonitrile (2.0 mL) to yield 40.7 mg (101%) of a white solid. ¹H NMR (300 MHz, CDCl₃) δ (ppm): 7.28-7.52 (m, 5H), 4.06-4.09 (m, 1H), 3.57 (s, 2H), 3.37 (s, 3H), 2.98 (d, 2H), 2.70 (s, 2H), 2.66 (td, 2H), 1.59-2.00 (m, 11H), 1.24-1.39 (m, 3H).

### Example 227

### 4-Chloro-2-cyclohexyl-1-methyl-5-(4-phenyl-4-propionylpiperidin-1-ylmethyl)-1,2-dihydro-pyrazol-3-one

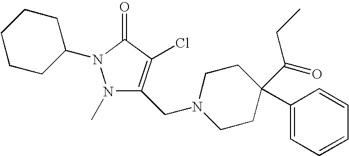

4-Chloro-2-cyclohexyl-1-methyl-5-(4-phenyl-4-propionylpiperidin-1-ylmethyl)-1,2-dihydro-pyrazol-3-one was synthesized using 5-bromomethyl-4-chloro-2-cyclohexyl-1-methyl-1,2-dihydro-pyrazol-3-one (30 mg, 0.0975 mmol), 1-(4-phenylpiperidin-4-yl)propan-1-one (37 mg, 0.146 mmol) and potassium carbonate (54 mg, 0.390 mmol) in acetonitrile (2.0 mL) to yield 40.2 mg (93%) of an off-white solid. ¹H NMR (300 MHz, CDCl₃) δ (ppm): 7.24-7.38 (m, 5H), 4.01-4.09 (m, 1H), 3.40 (s, 2H), 3.32 (s, 3H), 2.65 (d, 2H), 2.36-2.44 (m, 4H), 2.22 (q, 2H), 1.96-2.01 (m, 3H), 1.83 (t, 3H), 1.70 (d, 1H), 1.26-1.39 (m, 3H), 0.87 (t, 3H).

### Example 228

### 5-(4-Butyryl-4-phenylpiperidin-1-ylmethyl)-4-chloro-2-cyclohexyl-1-methyl-1,2-dihydro-pyrazol-3-one

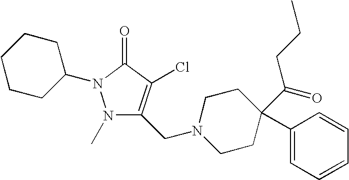

5-(4-Butyryl-4-phenylpiperidin-1-ylmethyl)-4-chloro-2-cyclohexyl-1-methyl-1,2-dihydro-pyrazol-3-one was synthesized using 5-bromomethyl-4-chloro-2-cyclohexyl-1-methyl-1,2-dihydro-pyrazol-3-one (40 mg, 0.130 mmol), 1-(4-phenylpiperidin-4-yl)butan-1-one (52 mg, 0.195 mmol) and potassium carbonate (72 mg, 0.520 mmol) in acetonitrile (2.0 mL) to yield 58 mg (97%) of a solid. ¹H NMR (300 MHz, CDCl₃) δ (ppm): 7.24-7.38 (m, 5H), 4.04-4.09 (m, 1H), 3.40 (s, 2H), 3.33 (s, 3H), 2.64 (s, 2H), 2.36-2.44 (m, 4H), 2.17 (t, 2H), 1.97-2.05 (m, 5H), 1.83 (t, 3H), 1.70 (d, 1H), 1.24-1.48 (m, 5 h), 0.66 (t, 3H).

### Example 229

### 4-Chloro-2-cyclohexyl-1-methyl-5-(3-methyl-4-m-tolyl-piperazin-1-ylmethyl)-1,2-dihydro-pyrazol-3-one

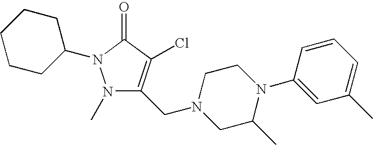

4-Chloro-2-cyclohexyl-1-methyl-5-(3-methyl-4-m-tolyl-piperazin-1-ylmethyl)-1,2-dihydro-pyrazol-3-one was synthesized using 5-bromomethyl-4-chloro-2-cyclohexyl-1-methyl-1,2-dihydro-pyrazol-3-one (30 mg, 0.0975 mmol), 2-methyl-1-m-tolyl-piperazine (28 mg, 0.146 mmol) and potassium carbonate (54 mg, 0.390 mmol) in acetonitrile (2.0 mL) to yield 35.9 mg (88%) of a yellow oil. ¹H NMR (300 MHz, CDCl₃) δ (ppm): 7.15 (t, 1H), 6.69 (t, 3H), 4.09-4.12 (m, 1H), 3.88-4.92 (m, 1H), 3.48 (s, 2H), 3.41 (s, 3H), 3.22 (dt, 1H), 3.10 (td, 1H), 2.82 (d, 1H), 2.58 (qd, 2H), 2.40 (td, 1H), 2.31 (s, 3H), 1.99-2.06 (m, 6H), 1.72 (d, 1H), 1.25-1.40 (m, 3H), 1.05 (d, 3H).

### Example 230

### 4-Chloro-5-[4-(5-chloro-2-methoxyphenyl)piperazine-1-ylmethyl]-2-cyclohexyl-1-ethyl-1,2-dihydro-pyrazol-3-one

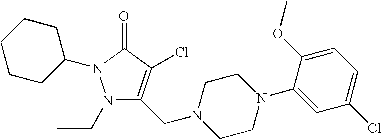

4-Chloro-5-[4-(5-chloro-2-methoxyphenyl)piperazine-1-ylmethyl]-2-cyclohexyl-1-ethyl-1,2-dihydro-pyrazol-3-one was synthesized using 5-bromomethyl-4-chloro-2-cyclohexyl-1-ethyl-1,2-dihydro-pyrazol-3-one (35 mg, 0.109 mmol), 1-(5-chloro-2-methoxyphenyl)piperazine (43 mg, 0.163 mmol) and potassium carbonate (60 mg, 0.436 mmol) in acetonitrile (2.0 mL) to yield 38 mg (75%) of an oil. ¹H NMR (300 MHz, CDCl₃) δ (ppm): 6.87-7.05 (m, 3H), 3.88 (s, 6H), 3.08 (s, 4H), 2.70 (s, 4H), 2.01-2.05 (m, 3H), 1.83 (s, 4H), 1.69 (d, 1H), 0.95-1.05 (m, 2H), 0.87 (t, 3H).

### Example 231

### 1-(4-Chloro-1-cyclohexyl-2-ethyl-5-oxo-2,5-dihydro-1H-pyrazol-3-ylmethyl)-4-phenylpiperidine-4-carbonitrile

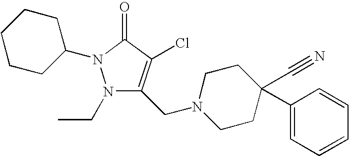

1-(4-Chloro-1-cyclohexyl-2-ethyl-5-oxo-2,5-dihydro-1H-pyrazol-3-ylmethyl)-4-phenylpiperidine-4-carbonitrile was synthesized using 5-bromomethyl-4-chloro-2-cyclohexyl-1-ethyl-1,2-dihydro-pyrazol-3-one (35 mg, 0.109 mmol), 4-phenylpiperidine-4-carbonitrile (38 mg, 0.163 mmol) and potassium carbonate (60 mg, 0.436 mmol) in acetonitrile (2.0 mL) to yield 31 mg of an oil. ¹H NMR (300 MHz, CDCl₃) δ (ppm): 7.35-7.52 (m, 5H), 3.89-4.01 (m, 1H), 3.85 (q, 2H), 3.54 (s, 2H), 3.00 (d, 2H), 2.66 (td, 2H), 2.04-2.14 (m, 6H), 1.89 (s, 4H), 1.85 (d, 1H), 1.25-1.37 (m, 3H), 1.02 (t, 3H).

### Example 232

### 4-Bromo-5-[4-(5-chloro-2-methoxyphenyl)-piperazin-1-ylmethyl]-2-cyclohexyl-1-methyl-1,2-dihydro-pyrazol-3-one

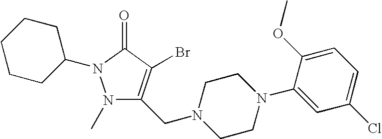

4-Bromo-5-[4-(5-chloro-2-methoxyphenyl)-piperazin-1-ylmethyl]-2-cyclohexyl-1-methyl-1,2-dihydro-pyrazol-3-one was synthesized using 4-bromo-5-bromomethyl-2-cyclohexyl-1-methyl-1,2-dihydro-pyrazol-3-one (30 mg, 0.085 mmol), 1-(5-chloro-2-methoxyphenyl)piperazine (34 mg, 0.128 mmol) and potassium carbonate (35 mg, 0.128 mmol) in acetonitrile (2.0 mL) to yield 44.3 mg of a beige solid. ¹H NMR (300 MHz, CDCl₃) δ (ppm): 6.95 (dd, 1H), 6.86 (d, 1H), 6.77 (d, 1H), 4.07-4.14 (m, 1H), 3.85 (s, 3H), 3.52 (s, 2H), 3.41 (s, 3H), 2.69 (s, 4H), 2.66 (t, 4H), 1.96-2.01 (m, 3H), 1.85 (t, 3H), 1.70 (d, 1H), 1.24-1.40 (m, 3H).

### Example 233

### 4-Bromo-5-[4-(5-chloro-2-methylphenyl)-piperazin-1-ylmethyl]-2-cyclohexyl-1-methyl-1,2-dihydro-pyrazol-3-one

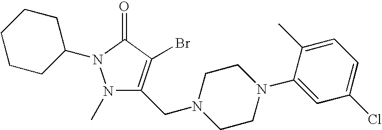

4-Bromo-5-[4-(5-chloro-2-methylphenyl)-piperazin-1-ylmethyl]-2-cyclohexyl-1-methyl-1,2-dihydro-pyrazol-3-one was synthesized using 4-bromo-5-bromomethyl-2-cyclohexyl-1-methyl-1,2-dihydro-pyrazol-3-one (30 mg, 0.085 mmol), 1-(5-chloro-2-methylphenyl)piperazine (27 mg, 0.128 mmol) and potassium carbonate (35 mg, 0.128 mmol) in acetonitrile (2.0 mL) to yield 42.3 mg of a yellow oil. ¹H NMR (300 MHz, CDCl₃) δ (ppm): 7.10 (d, 1H), 6.95-6.98 (m, 2H), 4.05-4.14 (m, 1H), 3.54 (s, 2H), 3.42 (s, 2H), 2.91 (t, 4H), 2.65 (s, 4H), 2.26 (s, 3H), 1.97-2.02 (m, 3H), 1.83 (t, 4H), 1.71 (d, 1H), 1.25-1.40 (m, 3H).

### Example 234

### 5-(4-Benzyl-piperidin-1-ylmethyl)-4-bromo-2-cyclohexyl-1-methyl-1,2-dihydro-pyrazol-3-one

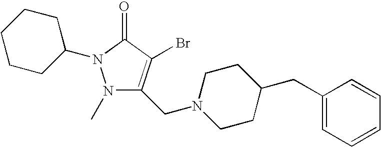

5-(4-Benzyl-piperidin-1-ylmethyl)-4-bromo-2-cyclohexyl-1-methyl-1,2-dihydro-pyrazol-3-one was synthesized using 4-bromo-5-bromomethyl-2-cyclohexyl-1-methyl-1,2-dihydro-pyrazol-3-one (30 mg, 0.085 mmol), 4-benzylpiperidine (22 mg, 0.128 mmol) and potassium carbonate (35 mg, 0.128 mmol) in acetonitrile (2.0 mL) to yield 37.2 mg of a beige solid. ¹H NMR (300 MHz, CDCl₃) δ (ppm): 7.13-7.31 (m, 5H), 4.05-4.11 (m, 1H), 3.39 (d, 5H), 2.81 (d, 2H), 2.53 (d, 2H), 2.01 (t, 4H), 1.89 (t, 4H), 1.54-1.72 (m, 4H), 1.18-1.39 (m, 5H).

### Example 235

### 4-Bromo-2-cyclohexyl-1-methyl-5-[4-(3-phenylpropyl)-piperidin-1-ylmethyl]-1,2-dihydro-pyrazol-3-one

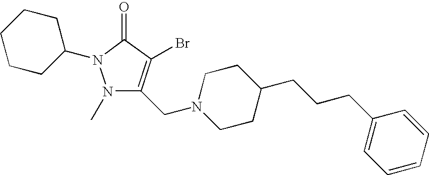

4-Bromo-2-cyclohexyl-1-methyl-5-[4-(3-phenylpropyl)-piperidin-1-ylmethyl]-1,2-dihydro-pyrazol-3-one was synthesized using 4-bromo-5-bromomethyl-2-cyclohexyl-1-methyl-1,2-dihydro-pyrazol-3-one (30 mg, 0.085 mmol), 4-(3-phenylpropyl)piperidine (25 mg, 0.128 mmol) and potassium carbonate (35 mg, 0.128 mmol) in acetonitrile (2.0 mL) to yield 30.1 mg of a beige solid. ¹H NMR (300 MHz, CDCl₃) δ (ppm): 7.26-7.32 (m, 2H), 7.17-7.21 (m, 3H), 4.06-4.11 (m, 1H), 3.40 (d, 5H), 2.81 (d, 2H), 2.60 (t, 2H), 2.00-2.07 (m, 4H), 1.85 (t, 5H), 1.60-1.69 (m, 5H), 1.19-1.39 (m, 7H).

### Example 236

### 5-[4-(4-Chloro-2-methoxyphenyl)-piperazin-1-ylmethyl]-2-cyclohexyl-4-methoxy-1-methyl-1,2-dihydro-pyrazol-3-one

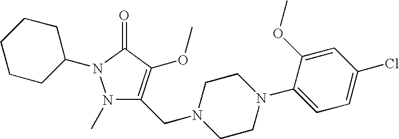

5-[4-(4-Chloro-2-methoxyphenyl)-piperazin-1-ylmethyl]-2-cyclohexyl-4-methoxy-1-methyl-1,2-dihydro-pyrazol-3-one was synthesized using 5-bromomethyl-2-cyclohexyl-1-methyl-1,2-dihydro-pyrazol-3-one (21 mg, 0.070 mmol), 1-(4-chloro-2-methoxyphenyl)piperazine (24 mg, 0.105 mmol) and potassium carbonate (44 mg, 0.315 mmol) in acetonitrile (2.0 mL) to yield 4.1 mg of a yellow oil. ¹H NMR (300 MHz, CDCl₃) δ (ppm): 6.82-6.93 (m, 3H), 3.96-4.05 (m, 1H), 3.91 (s, 3H), 3.87 (s, 3H), 3.44 (s, 2H), 3.17 (s, 3H), 3.05 (s, 4H), 2.66 (s, 4H), 2.20 (qd, 2H), 1.85 (t, 1H), 1.66 (s, 4H), 1.24-1.39 (m, 3H).

### Example 237

### 5-[4-(5-Chloro-2-methoxyphenyl)-piperazin-1-ylmethyl]-2-cyclohexyl-4-methoxy-1-methyl-1,2-dihydro-pyrazol-3-one

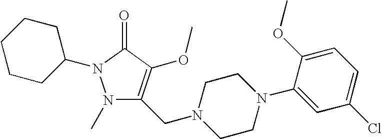

5-[4-(5-Chloro-2-methoxyphenyl)-piperazin-1-ylmethyl]-2-cyclohexyl-4-methoxy-1-methyl-1,2-dihydro-pyrazol-3-one was synthesized using 5-bromomethyl-2-cyclohexyl-1-methyl-1,2-dihydro-pyrazol-3-one (30 mg, 0.098 mmol), 1-(5-chloro-2-methoxyphenyl)piperazine (33 mg, 0.148 mmol) and potassium carbonate (41 mg, 297 mmol) in acetonitrile (2.0 mL) to yield 37.9 mg of a yellow oil. ¹H NMR (300 MHz, CDCl₃) δ (ppm): 6.95 (dd, 1H), 6.87 (d, 1H), 6.76 (d, 1H), 3.91-3.96 (m, 1H), 3.90 (s, 3H), 3.85 (s, 3H), 3.44 (s, 2H), 3.17 (s, 3H), 3.07 (s, 4H), 2.65 (s, 4H), 2.01 (qd, 2H), 1.83 (t, 4H), 1.67 (d, 1H), 1.24-1.37 (m, 3H).

### Example 238

### 2-Cyclohexyl-4-methoxy-1-methyl-5-[4-(3-phenylpropyl)-piperidin-1-ylmethyl]-1,2-dihydro-pyrazol-3-one

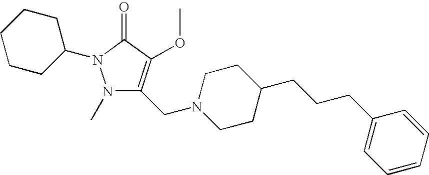

2-Cyclohexyl-4-methoxy-1-methyl-5-[4-(3-phenylpropyl)-piperidin-1-ylmethyl]-1,2-dihydro-pyrazol-3-one was synthesized using 5-bromomethyl-2-cyclohexyl-1-methyl-1,2-dihydro-pyrazol-3-one (30 mg, 0.098 mmol), 4-(3-phenylpropyl)piperidine (30 mg, 0.148 mmol) and potassium carbonate (41 mg, 297 mmol) in acetonitrile (2.0 mL) to yield 31.9 mg (76%) of a yellow oil. ¹H NMR (300 MHz, CDCl₃) δ (ppm): 7.27-7.31 (m, 2H), 7.17-7.27 (m, 3H), 3.91-3.95 (m, 1H), 3.88 (s, 3H), 3.32 (s, 2H), 3.13 (s, 3H), 2.82 (d, 2H), 2.60 (t, 2H), 1.97-2.01 (m, 4H), 1.88 (t, 5H), 1.61-1.68 (m, 5H), 1.19-1.33 (m, 7H).

### Example 239

### 5-(4-Acetyl-4-phenyl-piperidin-1-ylmethyl)-4-chloro-2-cyclohexyl-1-methyl-1,2-dihydro-pyrazol-3-one

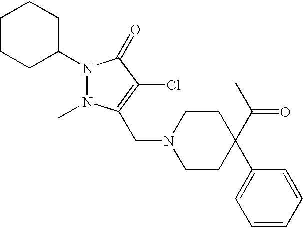

5-(4-Acetyl-4-phenyl-piperidin-1-ylmethyl)-4-chloro-2-cyclohexyl-1-methyl-1,2-dihydro-pyrazol-3-one was made with general procedure. 5-Bromomethyl-4-chloro-2-cyclohexyl-1-methyl-1,2-dihydro-pyrazol-3-one (30 mg, 0.0975 mmol), 1-(4-phenyl-piperidin-4-yl)-ethanone (35.09 mg, 0.1462 mmol), K₂CO₃ (67.37 mg, 0.4875 mmol), and 4 ml of acetonitrile was used. ¹H NMR (300 MHz, CDCl₃) δ (ppm): 7.31 (m, 5H), 4.04 (m, 1H), 3.41 (d, 2H), 3.35 (s, 3H), 2.65 (broad, 2H), 2.40 (m, 4H), 1.87 (broad, 15H), 1.33 (broad, 4H).

### Example 240

### 4-Chloro-5-[4-(5-chloro-2-methoxy-phenyl)-piperazin-1-ylmethyl]-2-cyclopentyl-1-methyl-1,2-dihydro-pyrazol-3-one

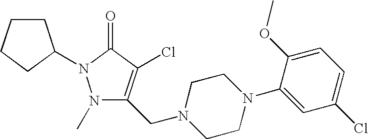

4-Chloro-5-[4-(5-chloro-2-methoxy-phenyl)-piperazin-1-ylmethyl]-2-cyclopentyl-1-methyl-1,2-dihydro-pyrazol-3-one was synthesized from 1-(5-chloro-2-methoxy-phenyl)-piperazine (40.3 mg, 0.153 mmol), 5-bromomethyl-4-chloro-2-cyclopentyl-1-methyl-1,2-dihydro-pyrazol-3-one (30 mg, 0.102 mmol) and potassium carbonate (42.3 mg, 0.306 mmol) in acetonitrile (2.0 mL). The crude material was purified by eluting through a 2 g SPE tube with a solution of 10% acetone and dichloromethane to form an orange gum (32.5 mg, 66.8%). ¹H NMR (300 MHz, CDCl₃): δ ppm 1.66-1.62 (m, 2H), 2.06-1.88 (m, 2H), 2.67 (br, 4H), 3.06 (br, 4H), 3.39 (s, 3H), 3.52 (s, 2H), 3.85 (s, 3H), 4.63 (quintet, 1H), 6.75 (d, 1H), 6.85 (d, 1H), 6.97 (d, 1H).

### Example 241

### 4-Chloro-5-[4-(chloro-2-methyl-phenyl)-piperazin-1-ylmethyl]-2-cyclopentyl-1-methyl-1,2dihydro-pyrazol-3-one

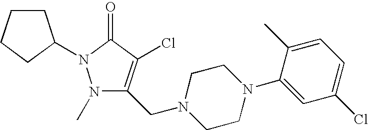

4-Chloro-5-[4-(chloro-2-methyl-phenyl)-piperazin-1-ylmethyl]-2-cyclopentyl-1-methyl-1,2dihydro-pyrazol-3-one was synthesized from 1-(5-chloro-2-methyl-phenyl)-piperazine (32.3 mg, 0.153 mmol), 5-bromomethyl-4-chloro-2-cyclopentyl-1-methyl-1,2-dihydro-pyrazol-3-one (30 mg, 0.102 mmol) and potassium carbonate (42.3 mg, 0.306 mmol) in acetonitrile (2.0 mL). The crude material was purified by eluting through a 2 g SPE tube using a solution of 10% acetone dichloromethane to form a colourless oil (18.9 mg, 43.7%). ¹H NMR (300 MHz, CDCl₃) δ ppm: 1.67-1.63 (m, 2H), 2.19-1.80 (m, 6H), 2.26 (s, 3H), 2.66 (br, 4H), 2.91 (br, 4H), 3.40 (s, 3H), 4.65 (quintet, 1H), 6.96 (br, 2H), 7.10 (br, 1H).

### Example 242

### 4-Chloro-2-cyclopentyl-1-methyl-5-[4-(3-phenyl-propyl)-piperazin-1-ylmethyl]-1,2-dihydro-pyrazol-3-one

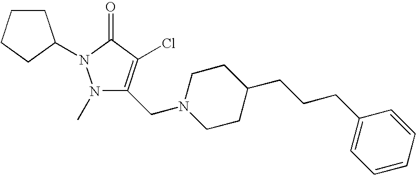

4-Chloro-2-cyclopentyl-1-methyl-5-[4-(3-phenyl-propyl)-piperazin-1-ylmethyl]-1,2-dihydro-pyrazol-3-one was synthesized from 4-(3-phenyl-propyl)-piperidine (31.1 mg, 0.153 mmol), 5-bromomethyl-4-chloro-2-cyclopentyl-1-methyl-1,2-dihydro-pyrazol-3-one (30 mg, 0.102 mmol) and potassium carbonate (42.3 mg, 0.306 mmol) in acetonitrile (2.0 mL). The crude material was purified by eluting through a 2 g SPE tube using a solution of 10% acetone and dichloromethane to form a colourless oil (19.6 mg, 46.1%.).

¹H NMR (300 MHz, CDC₃): δ ppm 1.26 (m, 6H), 1.66 (m, 6H), 1.91 (m, 6H), 2.03 (t, 2H), 2.07 (m, 1H), 3.36 (s, 3H), 2.60 (t, 2H), 2.84 (d, 2H), 3.39 (s, 2H), 4.63 (quintet, 1H), 7.32-7.17 (m, 5H).

### Example 243

### 5-[4-(5-Chloro-2-methoxy-phenyl)-piperazin-1-ylmethyl]-2-cyclopentyl-4-methoxy-1-methyl-1,2-dihydro-pyrazol-3-one

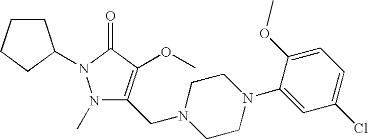

5-[4-(5-Chloro-2-methoxy-phenyl)-piperazin-1-ylmethyl]-2-cyclopentyl-4-methoxy-1-methyl-1,2-dihydro-pyrazol-3-one was synthesized from 1-(5-Chloro-2-methoxy-phenyl)-piperazine (47.7 mg, 0.1815 mmol), 5-Bromomethyl-2-cyclopentyl-4-methoxy-1-methyl-1,2-dihydro-pyrazol-3-one (35 mg, 0.121 mmol) and potassium carbonate (50.0 mg, 0.363 mmol) in acetonitrile (3.0 mL). The crude material was purified by eluting through a 2 g SPE tube using a solution of 12% acetone and dichloromethane to yield a yellow product (45 mg, 85.6%). ¹H NMR (300 MHz, CDCl₃): δ ppm 1.62-1.59 (m, 2H), 2.02-1.87 (m, 6H), 2.65 (br, 4H), 2.98 (br, 4H), 3.16 (s, 3H), 3.43 (s, 2H), 3.83 (s, 3H), 3.89 (s, 3H), 4.52 (quintet, 1H), 6.76 (d, 1H), 6.85 (d, 1H), 6.94 (dd, 1H).

### Example 244

### 5-[4-(Chloro-2-methyl-phenyl)-piperazin-1-ylmethyl]-2-cyclopentyl-4-methoxy-1-methyl-1,2,-dihydro-pyrazol-3-one

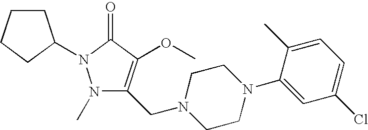

5-[4-(Chloro-2-methyl-phenyl)-piperazin-1-ylmethyl]-2-cyclopentyl-4-methoxy-1-methyl-1,2,-dihydro-pyrazol-3-one was synthesized from 1-(5-chloro-2-methyl-phenyl)-piperazine (38 mg, 0.1815 mmol), 5-bromomethyl-2-cyclopentyl-4-methoxy-1-methyl-1,2-dihydro-pyrazol-3-one (35 mg, 0.121 mmol), and potassium carbonate (50.0 mg, 0.363 mmol) in acetonitrile (3.0 mL). The crude material was purified by eluting through a 2 g SPE tube using a solution of 12% acetone and dichloromethane to yield a yellow product (36.1 mg, 71.2%). ¹H NMR (300 MHz, CDC₃): δ ppm 1.65-1.61 (m, 2H), 2.04-1.89 (m. 6H), 2.24 (s, 3H), 2.63 (4H), 2.90 (br, 4H), 3.18 (s, 3H), 3.87 (s, 2H), 3.91 (s, 3H), 4.538 (quintet, 1H), 6.94 (m, 2H), 7.07 (d, 1H).

### Example 245

### 2-Cyclopentyl-4-methoxy-1-methyl-5-[4-(3-phenyl-propyl)-piperidin-1-ylmethyl]-1,2-dihydro-pyrazol-3-one

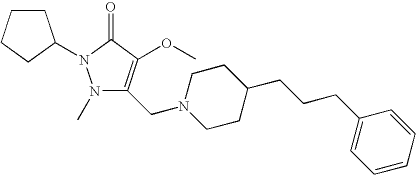

2-Cyclopentyl-4-methoxy-1-methyl-5-[4-(3-phenyl-propyl)-piperidin-1-ylmethyl]-1,2-dihydro-pyrazol-3-one was synthesized from 4-(3-phenyl-propyl)-piperidine (37 mg, 0.1815 mmol), 5-bromomethyl-2-cyclopentyl-4-methoxy-1-methyl-1,2-dihydro-pyrazol-3-one (35 mg, 0.121 mmol), and potassium carbonate (50.0 mg, 0.363 mmol) in acetonitrile (3.0 mL). The crude material was purified by eluting through a 2 g SPE tube using a solution of 12% acetone and dichloromethane to yield a yellow product (36.3 mg, 72.9%). ¹H NMR (300 MHz, CDCl₃): δ ppm 1.27-1.19 (m, 6H), 1.68-1.60 (m, 6H), 2.04-1.88 (m, 8H), 2.06 (m, 1H), 2.59 (t, 2H), 2.81 (d, 2H), 3.14 (s, 3H), 3.33 (s, 2H), 3.88 (s, 3H), 4.53 (quintet, 1H), 7.31-7.16 (m, 5H).

### Example 246

### 4-Chloro-5-[4-2(chloro-phenyl)-piperazin-1-ylmethyl]-2-(4-methoxy-phenyl)-1-methyl-1,2-dihydro-pyrazol-3-one

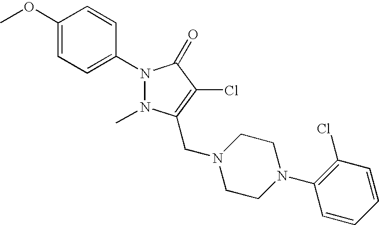

4-Chloro-5-[4-2(-chloro-phenyl)-piperazin-1-ylmethyl]-2-(4-methoxy-phenyl)-1-methyl-1,2-dihydro-pyrazol-3-one was synthesized using 5-bromomethyl-4-chloro-2-(4-methoxy-phenyl)-1-methyl-1,2-dihydro-pyrazol-3-one (20 mg, 0.0603 mmol) and 1-(2-chloro-phenyl)-piperazine (17.78 mg, 0.0904 mmol). ¹H NMR (300 MHz, CDCl₃) δ (ppm): 7.33 (m, 5H), 7.02 (m, 4H), 3.86 (d, 3H), 3.66 (s, 2H), 3.12 (s, 3H), 3.06 (s, 4H), 2.79 (s, 4H).

### Example 247

### 4-Chloro-5-[4-(2-hydroxy-phenyl)-piperazin-1-ylmethyl]-2-(4-methoxy-phenyl)-1-methyl-1,2-dihydro-pyrazol-3-one

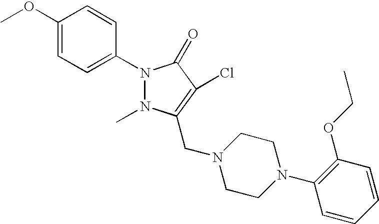

4-Chloro-5-[4-(2-hydroxy-phenyl)-piperazin-1-ylmethyl]-2-(4-methoxy-phenyl)-1-methyl-1,2-dihydro-pyrazol-3-one was synthesized with general procedure using 5-bromomethyl-4-chloro-2-(4-methoxy-phenyl)-1-methyl-1,2-dihydro-pyrazol-3-one (20 mg, 0.0603 mmol) and 1-(2-ethoxy-phenyl)-piperazine (18.65 mg, 0.090 mmol). ¹H NMR (300 MHz, CDCl₃) δ (ppm): 7.29 (m, 3H), 6.97 (m, 6H), 4.11 (q, 2H), 3.86 (t, 3H), 3.65 (s, 2H), 3.16 (s, 3H0, 3.05 (s, 4H), 2.78 (s, 4H), 1.27 (t, 3H).

### Example 248

### 4-Chloro-5-[4-(2-methoxy-phenyl)-piperazin-1-ylmethyl]-2-(4-methoxy-phenyl)-1-methyl-1,2-dihydro-pyrazol-3-one

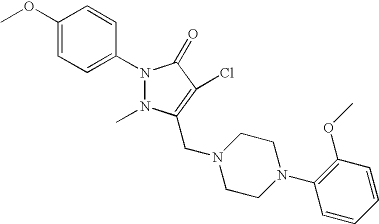

4-Chloro-5-[4-(2-methoxy-phenyl)-piperzin-1-ylmethyl]-2-(4-methoxy-phenyl)-1-methyl-1,2-dihydro-pyrazol-3-one was synthesized with general procedure using 5-bromomethyl-4-chloro-2-(4-methoxy-phenyl)-1-methyl-1,2-dihydro-pyrazol-3-one (20 mg, 0.0603 mmol) and 1-(2-methoxy-phenyl)-piperazine (20.49 mg, 0.0904 mmol). ¹H NMR (300 MHz, CDCl₃) δ (ppm): 7.30 (m, 3H), 7.00 (m, 3H), 6.89 (d, 1H0, 6.79 (d, 1H), 3.85 (t, 3H), 3.64 (s, 2H), 3.11 (s, 3H0, 3.06 (s, 4H), 2.77 (s, 4H).

### Example 249

### 8-[4-Chloro-1-(4-methoxy-phenyl)-2-methyl-5-oxo-2,5-dihydro-1H-pyrazol-3-ylmethyl]-1,3,8-triaza-spiro[4.5]decan-4-one

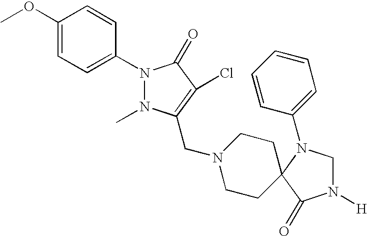

8-[4-Chloro-1-(4-methoxy-phenyl)-2-methyl-5-oxo-2,5-dihydro-1H-pyrazol-3-ylmethyl]-1,3,8-triaza-spiro[4.5]decan-4-one was synthesized with general procedure using 5-bromomethyl-4-chloro-2-(4-methoxy-phenyl)-1-methyl-1,2-dihydro-pyrazol-3-one (20 mg, 0.0603 mmol) and 1-phenyl-1,3,8-triaza-spiro[4.5]decan-4-one (20.90 mg, 0.0904 mmol). ¹H NMR (300 MHz, CDCl₃) δ (ppm): 7.31 (m, 5H), 7.01 (t, 2H), 6.88 (t, 3H), 4.76 (s, 2H), 3.85 (s, 3H), 3.68 (s, 2H), 3.00 (s, 3H), 2.88 (m, 2H), 2.74 (d, 2H), 2.69 (t, 2H), 1.79 (d, 2H).

### Example 250

### 4-Chloro-5-[4-(3,5-dichloro-pyridin-4-yl)-piperazin-1-ylmethyl]-2-(4-methoxy-phenyl)-1-methyl-1,2-dihydro-pyrazol-3-one

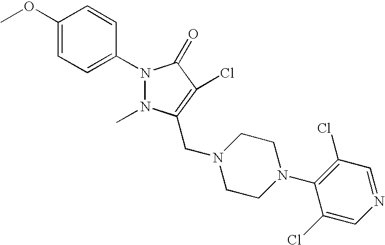

4-Chloro-5-[4-(3,5-dichloro-pyridin-4-yl)-piperazin-1-ylmethyl]-2-(4-methoxy-phenyl)-1-methyl-1,2-dihydro-pyrazol-3-one was synthesized with general procedure using 5-bromomethyl-4-chloro-2-(4-methoxy-phenyl)-1-methyl-1,2-dihydro-pyrazol-3-one (20 mg, 0.0603 mmol) and 1-(3,5-dichloro-pyridin-4-yl)-piperazine (20.89 mg, 0.0904 mmol). ¹H NMR (300 MHz, CDCl₃) δ (ppm): 8.38 (s, 2H), 7.30 (m, 3H), 7.00 (m, 2H), 3.85 (d, 3H), 3.66 (s, 2H), 3.42 (s, 4H), 3.25 (s, 3H), 2.74 (s, 4H).

### Example 251

### 4-Chloro-5-[4-(2,4-dimethyl-phenyl-piperazin-1-ylmethyl]-2-(4-methoxy-phenyl)-1-methyl-1,2-dihydro-pyrazol-3-one

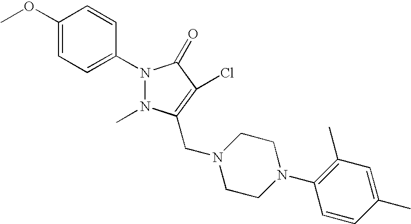

4-Chloro-5-[4-(2,4-dimethyl-phenyl-piperazin-1-ylmethyl]-2-(4-methoxy-phenyl)-1-methyl-1,2-dihydro-pyrazol-3-one was synthesized with general procedure using 5-bromomethyl-4-chloro-2-(4-methoxy-phenyl)-1-methyl-1,2-dihydro-pyrazol-3-one (20 mg, 0.0603 mmol) and 1-(2,4-dimethyl-phenyl)-piperazine (17.20 mg, 0.0904 mmol). ¹H NMR (300 MHz, CDCl₃) δ (ppm): 7.31 (q, 3H), 7.00 (m, 6H), 3.86 (s, 3H), 3.65 (s, 2H), 3.26 (d, 3H), 2.95 (s, 4H), 2.74 (s, 4H), 2.30 (s, 7H).

### Example 252

### 4-Chloro-2-(4-chloro-phenyl)-5-[4-(2-chloro-phenyl)-piperazin-1-ylmethyl]-1-methyl-1,2-dihydro-pyrazol-3-one

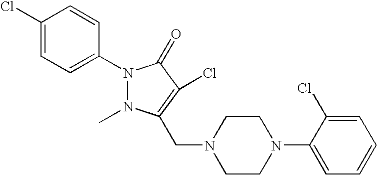

4-Chloro-2-(4-chloro-phenyl)-5-[4-(2-chloro-phenyl)-piperazin-1-ylmethyl]-1-methyl-1,2-dihydro-pyrazol-3-one is made by following general procedure 5-Bromomethyl-4-chloro-2-(4-chloro-phenyl)-1-methyl-1,2-dihydro-pyrazol-3-one (30 mg, 0.0898 mmol), 1-(2-chloro-phenyl)-piperazine (26.31 mg, 0.1338 mmols), K₂CO₃ (61.64 mg, 0.4459 mmol), and 4 ml of acetonitrile was used to obtain 60% yield of the product. ¹H NMR (300 MHz, CDCl₃) δ (ppm): 7.48 (m, 2H), 7.37 (m, 3H), 7.35 (m, 1H), 7.04 (m, 2H), 3.66 (s, 2H), 3.24 (s, 3H), 3.12 (s, 4H), 2.79 (d, 4H).

### Example 253

### 4-Chloro-2-(4-chloro-phenyl)-5-[4-(2-methoxy-phenyl)-piperazin-1-ylmethyl]-1 methyl-1,2-dihydro-pyrazol-3-one

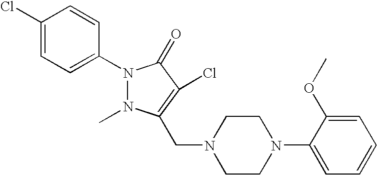

4-Chloro-2-(4-chloro-phenyl)-5-[4-(2-methoxy-phenyl)-piperazin-1-ylmethyl]-1 methyl-1,2-dihydro-pyrazol-3-one is made with general procedure 5-Bromomethyl-4-chloro-2-(4-chloro-phenyl)-1-methyl-1,2-dihydro-pyrazol-3-one (30 mg, 0.0898 mmol) 1-(2-methoxy-phenyl)-piperazine (25.72 mg, 0.1338 mmols), K₂CO₃ (61.64 mg, 0.4459 mmol), and 4 ml of acetonitrile was used to obtain 61.6% yield of the product. ¹H NMR (300 MHz, CDCl₃) δ (ppm): 7.46 (q, 2H), 7.36 (q, 2H), 6.94 (m, 4H), 3.89 (s, 3H), 3.65 (s, 3H), 3.24 (s, 3H), 3.13 (s, 4H), 2.78 (t, 4H).

### Example 254

### 4-Chloro-5-[4-(5-chloro-2-methoxy-phenyl)-piperazin-1-ylmethyl]-2-(4-chloro-phenyl)-1-methyl-1,2-dihydro-pyrazol-3-one

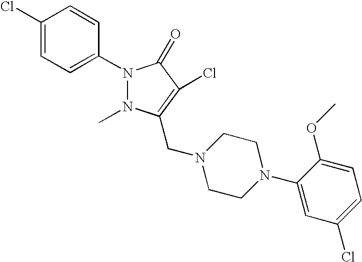

4-Chloro-5-[4-(5-chloro-2-methoxy-phenyl)-piperazin-1-ylmethyl]-2-(4-chloro-phenyl)-1-methyl-1,2-dihydro-pyrazol-3-one is made with general procedure 5-Bromomethyl-4-chloro-2-(4-chloro-phenyl)-1-methyl-1,2-dihydro-pyrazol-3-one (30 mg, 0.0898 mmol), 1-(5-chloro-2-methoxy-phenyl)-piperazine (30.33 mg, 0.1338 mmols), K₂CO₃ (61.64 mg, 0.4459 mmol), and 4 ml of acetonitrile was used to obtain 64.07% yield of the product. ¹H NMR (300 MHz, CDCl₃): 7.46 (q, 4H), 6.97 (q, 1H), 6.88 (d, 1H), 6.78 (d, 1H), 3.87 (s, 3H), 3.64 (s, 2H), 3.23 (s, 3H), 3.11 (s, 4H), 2.77 (d, 4H).

### Example 255

### 8-[4-Chloro-1-(4-chloro-phenyl)-2-methyl-5-oxo-2,5-dihydro-1H-pyrazol-3-ylmethyl]-1-phenyl-1,3,8-triaza-spiro[4.5]decan-4-one

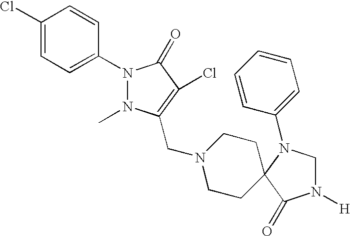

8-[4-Chloro-1-(4-chloro-phenyl)-2-methyl-5-oxo-2,5-dihydro-1H-pyrazol-3-ylmethyl]-1-phenyl-1,3,8-triaza-spiro[4.5]decan-4-one was made with general procedure. 5-Bromomethyl-4-chloro-2-(4-chloro-phenyl)-1-methyl-1,2-dihydro-pyrazol-3-one (30 mg, 0.090 mmol), 1-Phenyl-1,3,8-triaza-spiro[4.5]decan-4-one (30.94 mg, 0.14 mmol), K₂CO₃ (61.64 mg, 0.46 mmol), and 4 ml of acetonitrile was used to obtain 54.24% yield. ¹H NMR (300 MHz, CDCl₃) δ (ppm): 7.67 (s, 1H), 7.46 (m, 5H), 6.89 (m, 3H), 4.77 (s, 2H), 3.69 (s, 2H), 3.29 (s, 3H), 3.05 (m, 2H), 2.87 (t, 2H), 2.66 (s, 2H), 1.80 (d, 2H).

### Example 256

### 4-Chloro-2-(4-chloro-phenyl)-5-[4-(3,5-dichloro-pyridin-4-yl)-piperazin-1-ylmethyl]-1-methyl-1,2-dihydro-pyrazol-3-one

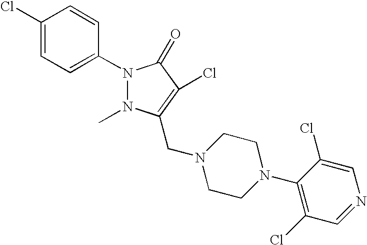

4-Chloro-2-(4-chloro-phenyl)-5-[4-(3,5-dichloro-pyridin-4-yl)-piperazin-1-ylmethyl]-1-methyl-1,2-dihydro-pyrazol-3-one was made with general procedure. 5-Bromomethyl-4-chloro-2-(4-chloro-phenyl)-1-methyl-1,2-dihydro-pyrazol-3-one (30 mg, 0.0898 mmol), 1-(3,5-Dichloro-pyridin-4-yl)-piperazine (30.92 mg, 0.1338 mmol), K₂CO₃ (61.64 mg, 0.4459 mmol), and 4 ml of acetonitrile was used to obtain 65.6% yield of the product. ¹H NMR (300 MHz, CDCl₃) δ (ppm): 8.36 (s, 2H), 7.47 (m, 2H), 7.37 (m, 2H), 4.07 (s, 2H), 3.41 (t, 4H), 3.25 (s, 3H), 2.72 (t, 4H).

### Example 257

### 4-Chloro-2-(4-chloro-phenyl)-5-[4-(2,4-dimethyl-phenyl)-piperazin-1-yl-methyl]-1-methyl-1,2-dihydro-pyrazol-3-one

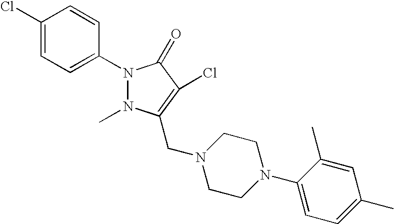

4-Chloro-2-(4-chloro-phenyl)-5-[4-(2,4-dimethyl-phenyl)-piperazin-1-yl-methyl]-1-methyl-1,2-dihydro-pyrazol-3-one was made with general procedure. 5-Bromomethyl-4-chloro-2-(4-chloro-phenyl)-1-methyl-1,2-dihydro-pyrazol-3-one (30 mg, 0.0898 mmol), 1-(2,4-dimethyl-phenyl)-piperazine (25.46 mg, 0.1338 mmol), K₂CO₃ (61.64 mg, 0.46 mmol), and 4 ml of acetonitrile was used to obtain 54.4% yield of the product. ¹H NMR (300 MHz, CDCl₃) δ (ppm): 7.48 (m, 2H), 7.38 (m, 2H), 6.99 (m, 3H), 3.65 (s, 2H), 3.26 (s, 3H), 2.94 (t, 4H), 2.73 (s, 4H), 2.30 (s, 6H).

### Example 258

### 4-Chloro-2-(4-chloro-phenyl)-5-[4-(2-ethoxy-phenyl)-piperazin-1-ylmethyl]1-1-methyl-1,2-dihydro-pyrazol-3-one

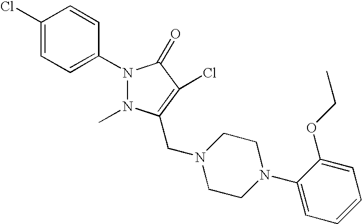

4-Chloro-2-(4-chloro-phenyl)-5-[4-(2-ethoxy-phenyl)-piperazin-1-ylmethyl]1-1-methyl-1,2-dihydro-pyrazol-3-one was made with general procedure 5-Bromomethyl-4-chloro-2-(4-chloro-phenyl)-1-methyl-1,2-dihydro-pyrazol-3-one (30 mg, 0.0898 mmol), 1-(2-Ethoxy-phenyl)-piperazine (27.6 mg, 0.1338 mmol), K₂CO₃ (61.64 mg, 0.4459 mmol), and 4 ml of acetonitrile was used to obtain 76.1% yield of the product. ¹H NMR (300 MHz, CDCl₃) δ (ppm): 7.47 (m, 2H), 7.36 (m, 2H), 6.92 (m, 4H), 4.09 (q, 2H), 3.65 (s, 2H), 3.25 (s, 3H), 3.16 (s, 4H), 2.78 (t, 4H), 1.48 (t, 3H).

### Example 259

### 4-Chloro-5-[4-(5-chloro-2-methyl-phenyl)-piperazin-1-ylmethyl]-2-(4-chloro-phenyl)-1-methy)-1-methyl-1,2-dihydro-pyrazol-3-one

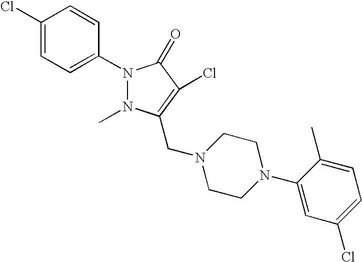

4-Chloro-5-[4-(5-chloro-2-methyl-phenyl)-piperazin-1-ylmethyl]-2-(4-chloro-phenyl)-1-methy)-1-methyl-1,2-dihydro-pyrazol-3-one was made with general procedure. 5-Bromomethyl-4-chloro-2-(4-chloro-phenyl)-1-methyl-1,2-dihydro-pyrazol-3-one (30 mg, 0.0898 mmol), 1-(5-Chloro-2-methyl-phenyl)-piperazine (30 mg, 0.1338 mmol), K₂CO₃ (61.64 mg, 0.4459 mmol), and 4 ml of acetonitrile was used to obtain 75.7% yield of the product. ¹H NMR (300 MHz, CDCl₃) δ (ppm): 7.47 (m, 2H), 7.38 (m, 2H), 7.11 (q, 1H), 6.965 (t, 2H), 0.65 (s, 2H), 3.25 (s, 3H), 2.95 (t, 4H), 2.73 (s, 4H), 2.27 (s, 3H).

### Example 260

### 1-[4-Chloro-1-(4-chloro-phenyl)-2-methyl-5-oxo-2,5-dihydro-1H-pyrazol-3-ylmethyl]-4-phenyl-piperidine-4-carbonitrile

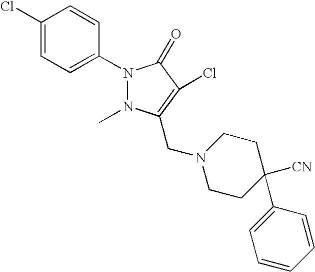

1-[4-Chloro-1-(4-chloro-phenyl)-2-methyl-5-oxo-2,5-dihydro-1H-pyrazol-3-ylmethyl]-4-phenyl-piperidine-4-carbonitrile was made with general procedure. 5-Bromomethyl-4-chloro-2-(4-chloro-phenyl)-1-methyl-1,2-dihydro-pyrazol-3-one (30 mg, 0.0898 mmol), 4-phenyl-piperidine-4-carbonitrile (29.79 mg, 0.1338 mmol), K₂CO₃ (61.64 mg, 0.4459 mmol), and 4 ml of acetonitrile was used to obtain 83% yield of the product. ¹H NMR (300 MHz, CDCl₃) δ (ppm): 7.45 (m, 9H), 3.69 (s, 2H), 3.21 (s, 3H), 3.08 (d, 2H), 2.73 (s, 2H), 2.14 (m, 4H).

### Example 261

### 4-Chloro-2-(4-chloro-phenyl)-1-methyl-5-(4-phenyl-4-propionyl-piperidin-1-ylmethyl)-1,2-dihydro-pyrazol-3-one

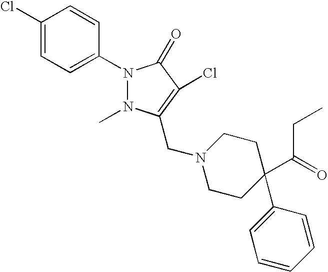

4-Chloro-2-(4-chloro-phenyl)-1-methyl-5-(4-phenyl-4-propionyl-piperidin-1-ylmethyl)-1,2-dihydro-pyrazol-3-one was made with general procedure. 5-Bromomethyl-4-chloro-2-(4-chloro-phenyl)-1-methyl-1,2-dihydro-pyrazol-3-one (30 mg, 0.0898 mmol), 1-(4-phenyl-piperidin-4-yl)-propan-1-one (33.95 mg, 0.1338 mmol), K₂CO₃ (61.64 mg, 0.4459 mmol), and 4 ml of acetonitrile was used to obtain 71.5% yield of the product. ¹H NMR (300 MHz, CDCl₃) δ (ppm): 7.35 (m, 9H), 3.53 (s, 2H), 2.77 (s, 3H), 2.75 (m, 2H), 2.48 (m, 4H), 2.25 (q, 2H), 2.11 (m, 2H), 0.92 (m, 3H).

### Example 262

### 5-(4-Butyryl-4-phenyl-piperidin-1-ylmethyl)-4-chloro-2-(4-chloro-phenyl)-1-methyl-1,2-dihydro-pyrazol-3-one

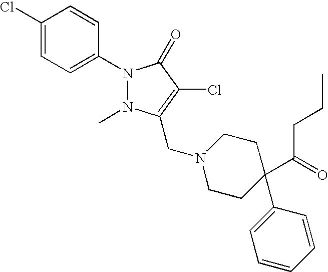

5-(4-Butyryl-4-phenyl-piperidin-1-ylmethyl)-4-chloro-2-(4-chloro-phenyl)-1-methyl-1,2-dihydro-pyrazol-3-one was made with general procedure. 5-Bromomethyl-4-chloro-2-(4-chloro-phenyl)-1-methyl-1,2-dihydro-pyrazol-3-one (30 mg, 0.0898 mmol), 1-(4-phenyl-piperidin-4-yl)-butan-1-one (35.83 mg, 0.1338 mmol), K₂CO₃ (61.64 mg, 0.4459 mmol) and 4 ml of acetonitrile was used to obtain 71.7% yield of the product. ¹H NMR (300 MHz, CDCl₃) δ (ppm): 7.34 (m, 9H), 3.52 (s, 2H), 3.18 (s, 3H), 2.73 (t, 2H), 2.48 (m, 4H), 2.14 (m, 4H), 1.45 (q, 2H), 0.68 (t, 3H).

### Example 263

### 4-Chloro-2-(4-chloro-phenyl)-1-methyl-5-(3-methyl-4-m-tolyl-piperazin-1-ylmethyl)-1,2-dihydro-pyrazol-3-one

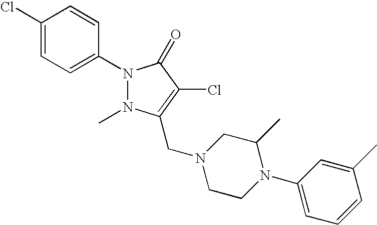

4-Chloro-2-(4-chloro-phenyl)-1-methyl-5-(3-methyl-4-m-tolyl-piperazin-1-ylmethyl)-1,2-dihydro-pyrazol-3-one was made with general procedure. 5-Bromomethyl-4-chloro-2-(4-chloro-phenyl)-1-methyl-1,2-dihydro-pyrazol-3-one (30 mg, 0.0898 mmol), 2-methyl-1-m-tolyl-piperazine (25.46 mg, 0.1338 mmol), K₂CO₃ (61.64 mg, 0.4459 mmol) and 4 ml of acetonitrile was used to obtain 71.7% yield of the product. ¹H NMR (300 MHz, CDCl₃) δ (ppm): 7.48 (m, 2H), 7.36 (m, 2H), 7.28 (t, 1H), 6.72 (t, 3H), 3.60 (d, 2H), 3.27 (s, 3H), 3.19 (m, 1H), 2.92 (d, 1H), 2.66 (m, 2H), 2.51 (m, 1H), 2.34 (s, 3H), 1.10 (d, 3H).

### Example 264

### 5-(4-Acetyl-4-phenyl-piperidin-1-ylmethyl)-4-chloro-2-(4-chloro-phenyl)-1-methyl-1,2-dihydro-pyrazol-3-one

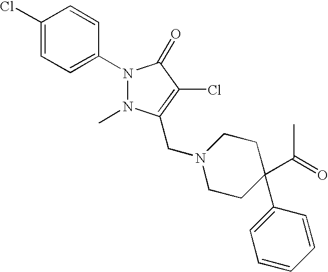

5-(4-Acetyl-4-phenyl-piperidin-1-ylmethyl)-4-chloro-2-(4-chloro-phenyl)-1-methyl-1,2-dihydro-pyrazol-3-one was made with general procedure. 5-Bromomethyl-4-chloro-2-(4-chloro-phenyl)-1-methyl-1,2-dihydro-pyrazol-3-one (30 mg, 0.0898 mmol), 1-(4-phenyl-piperidin-4-yl)-ethanone (32.29 mg, 0.1338 mmol), K₂CO₃ (61.64 mg, 0.4459 mmol) and 4 ml of acetonitrile was used to obtain 94.5% yield of the product. ¹H NMR (300 MHz, CDCl₃) δ (ppm): 7.34 (m, 10H), 3.53 (s, 2H), 3.18 (s, 3H), 2.76 (t, 2H), 2.48 (m, 4H), 2.07 (m, 2H), 1.93 (s, 3H).

### Example 265

### 4-Chloro-5-[4-(5-chloro-2-methoxy-phenyl)-piperazin-1-ylmethyl]-2-(4-chloro-phenyl)-1-ethyl-1,2-dihydro-pyrazol-3-one

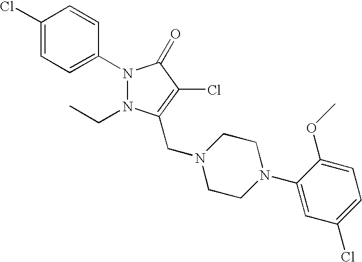

4-Chloro-5-[4-(5-chloro-2-methoxy-phenyl)-piperazin-1-ylmethyl]-2-(4-chloro-phenyl)-1-ethyl-1,2-dihydro-pyrazol-3-one was made with general procedure.

5-Bromomethyl-4-chloro-2-(4-chloro-phenyl)-1-methyl-1,2-dihydro-pyrazol-3-one (30 mg, 0.0857 mmol), 1-(5-chloro-2-methoxy-phenyl)-4-methyl-piperazine (33.83 mg, 0.1285 mmol), K₂CO₃ (59.22 mg, 0.4285 mmol) and 4 ml of acetonitrile was used. ¹H NMR (300 MHz, CDCl₃) δ (ppm): 7.44 (m, 4H), 6.97 (q, 1H), 6.88 (d, 1H), 6.78 (d, 1H), 3.87 (s, 3H), 3.80 (q, 2H), 3.62 (s, 2H), 3.10 (s, 4H), 2.78 (t, 4H), 0.89 (t, 3H).

### Example 266

### 4-Chloro-5-[4-(5-chloro-2-methyl-phenyl)-piperazin-1-ylmethyl]-2-(4-chloro-phenyl)-1-ethyl-1,2-dihydro-pyrazol-3-one

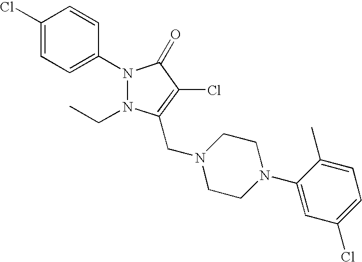

4-Chloro-5-[4-(5-chloro-2-methyl-phenyl)-piperazin-1-ylmethyl]-2-(4-chloro-phenyl)-1-ethyl-1,2-dihydro-pyrazol-3-one was made with general procedure.

5-Bromomethyl-4-chloro-2-(4-chloro-phenyl)-1-methyl-1,2-dihydro-pyrazol-3-one (30 mg, 0.0857 mmol), 1-(5-chloro-2-methyl-phenyl)-piperazine (27.09 mg, 0.1285 mmol), K₂CO₃ (59.22 mg, 0.4285 mmol), and 4 ml of acetonitrile was used. ¹H NMR (300 MHz, CDCl₃) δ (ppm): 7.45 (m, 4H), 7.11 (d, 1H), 6.97 (t, 2H), 3.80 (q, 2H), 3.63 (s, 2H), 2.95 (t, 4H), 2.75 (s, 4H), 2.28 (s, 3H), 0.90 (t, 3H).

### Example 267

### 1-[4-Chloro-1-(4-chloro-phenyl)-2-ethyl-5-oxo-2,5-dihydro-1H-pyrazol-3-ylmethyl]-4-phenyl-piperidine-4-carbonitrile

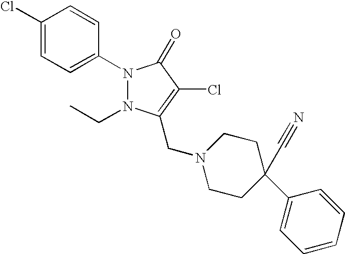

1-[4-Chloro-1-(4-chloro-phenyl)-2-ethyl-5-oxo-2,5-dihydro-1H-pyrazol-3-ylmethyl]-4-phenyl-piperidine-4-carbonitrile was made with general procedure.

5-Bromomethyl-4-chloro-2-(4-chloro-phenyl)-1-methyl-1,2-dihydro-pyrazol-3-one (30 mg, 0.0857 mmol), 4-phenyl-piperidine-4-carbonitrile (28.63 mg, 0.1285 mmol), K₂CO₃ (59.22 mg, 0.4285 mmol), and 4 ml of acetonitrile was used. ¹H NMR (300 MHz, CDCl₃) δ (ppm): 7.44 (m, 9H), 3.76 (q, 2H), 3.67 (s, 2H), 3.09 (d, 2H), 2.74 (m, 2H), 2.15 (m, 4H), 0.89 (t, 3H).

### Example 268

### 3-Amino-8-[4-chloro-1-(4-chloro-phenyl)-2-ethyl-5-oxo-2,5-dihydro-1-1H-pyrazol-3-ylmethyl]-1-phenyl-1,8-diaza-spiro[4.5]decan-4-one

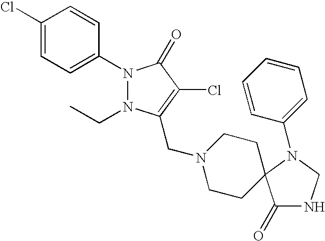

3-Amino-8-[4-chloro-1-(4-chloro-phenyl)-2-ethyl-5-oxo-2,5-dihydro-1-1H-pyrazol-3-ylmethyl]-1-phenyl-1,8-diaza-spiro[4.5]decan-4-one was made with general procedure. 5-Bromomethyl-4-chloro-2-(4-chloro-phenyl)-1-methyl-1,2-dihydro-pyrazol-3-one (30 mg, 0.0857 mmol), 3-Amino-1-phenyl-1,8-diaza-spiro[4.5]decan-4-one (34.42 mg, 0.1285 mmol), K₂CO₃ (59.22 mg, 0.4285 mmol), and 4 ml of acetonitrile was used. ¹H NMR (300 MHz, CDCl₃) δ (ppm): 7.45 (m, 7H), 6.87 (q, 3H), 4.76 (d, 2H), 3.85 (q, 2H), 3.66 (s, 2H), 3.05 (m, 2H), 2.89 (t, 2H), 2.71 (m, 2H), 1.79 (d, 2H), 0.95 (t, 3H).

### Example 269

### 8-(4-Chloro-1-isopropyl-2-methyl-5-oxo-2,5-dihydro-1H-pyrazol-3-ylmethyl)-1-phenyl-1,3,8-triaza-spiro[4.5]decan-4-one

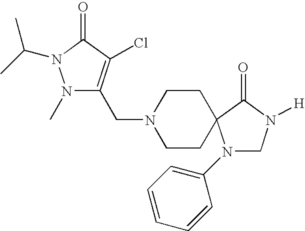

8-(4-Chloro-1-isopropyl-2-methyl-5-oxo-2,5-dihydro-1H-pyrazol-3-ylmethyl)-1-phenyl-1,3,8-triaza-spiro[4.5]decan-4-one was obtained from 1-Phenyl-1,3,8-triazaspiro[4.5]decan-4-one (39.3 mg, 0.17 mmol), 5-Bromomethyl-4-chloro-2-isopropyl-1-methyl-1,2-dihydro-pyrazol-3-one (30 mg, 0.113 mmol) and potassium carbonate (78.1 mg, 0.565 mmol) in acetonitrile (2 mL) as an off white solid (44.37 mg, 94%). ¹H NMR (300 MHz, CDCl₃): δ (ppm) 7.32 (d, 2H), 6.91 (m, 3H), 6.70 (s, 1H), 4.77 (s, 2H), 4.56 (m, 1H), 3.57 (s, 2H), 3.44 (s, 3H), 2.98 (m, 2H), 2.78 (m, 2H), 2.63 (m, 2H), 1.48 (d, 6H).

### Example 270

### 4-Chloro-5-[4-(5-chloro-2-methoxy-phenyl)-piperazin-1-ylmethyl]-2-isopropyl-1-methyl-1,2-dihydro-pyrazol-3-one

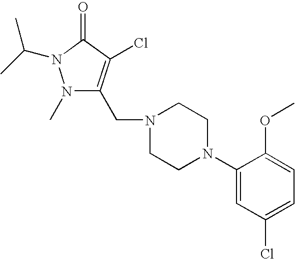

4-Chloro-5-[4-(5-chloro-2-methoxy-phenyl)-piperazin-1-ylmethyl]-2-isopropyl-1-methyl-1,2-dihydro-pyrazol-3-one was obtained from 1-(5-Chloro-2-methoxy-phenyl)-piperazine (38.54 mg, 0.170 mmol), 5-Bromomethyl-4-chloro-2-isopropyl-1-methyl-1,2-dihydro-pyrazol-3-one (30 mg, 0.113 mmol) and potassium carbonate (78.1 mg, 0.565 mmol) in acetonitrile (2 mL) as a white solid (44.4 mg, 95%). ¹H NMR (300 MHz, CDCl₃): δ (ppm) 6.97 (dd, 1H), 6.88 (d, 1H), 6.79 (d, 1H), 4.58 (m, 1H), 3.87 (s, 3H), 3.54 (s, 2H), 3.40 (s, 3H), 3.07 (broad s, 4H), 2.72 (broad t, 4H), 1.61 (s, 3H), 1.47 (d, 6H).

### Example 271

### 4-Chloro-5-[4-(5-chloro-2-methyl-phenyl)-piperazin-1-ylmethyl]-2-isopropyl-1-methyl-1,2-dihydro-pyrazol-3-one

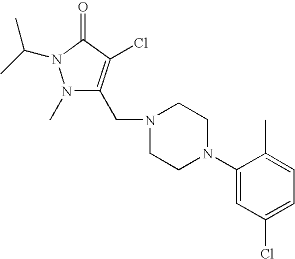

4-Chloro-5-[4-(5-chloro-2-methyl-phenyl)-piperazin-1-ylmethyl]-2-isopropyl-1-methyl-1,2-dihydro-pyrazol-3-one was obtained from 1-(5-Chloro-2-methyl-phenyl)-piperazine (35.82 mg, 0.170 mmol), 5-Bromomethyl-4-chloro-2-isopropyl-1-methyl-1,2-dihydro-pyrazol-3-one (30 mg, 0.113 mmol) and potassium carbonate (78.1 mg, 0.565 mmol) in acetonitrile (2 mL) as a white solid (46.5 mg, 104%). ¹H NMR (300 MHz, CDCl₃): δ (ppm) 7.11 (d, 1H), 6.98 (m, 2H), 4.58 (m, 1H), 3.55 (s, 2H), 3.42 (s, 3H), 2.92 (broad t, 4H), 2.66 (broad s, 4H), 2.26 (s, 3H), 1.47 (d, 6H).

### Example 272

### 5-[4-(5-Chloro-2-methoxy-phenyl)-piperazin-1-ylmethyl]-4-methoxy-1-methyl-2-phenyl-1,2-dihydro-pyrazol-3-one

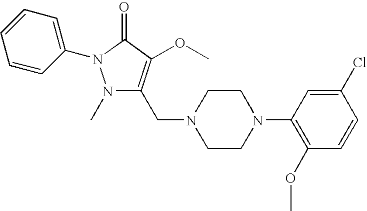

5-[4-(5-Chloro-2-methoxy-phenyl)-piperazin-1-ylmethyl]-4-methoxy-1-methyl-2-phenyl-1,2-dihydro-pyrazol-3-one was obtained from 1-(5-Chloro-2-methoxy-phenyl)-piperazine (57.13 mg, 0.252 mmol), 5-Bromomethyl-4-methoxy-1-methyl-2-phenyl-1,2-dihydro-pyrazol-3-one (50.0 mg, 0.168 mmol), and potassium carbonate (116.1 mg, 0.84 mmol) in acetonitrile (2 mL) as a white solid (75.1 mg, 101%). ¹H NMR (300 MHz, CDCl₃): δ (ppm) 7.46 (m, 4H), 7.28 (m, 1H), 6.97 (d, 1H), 6.91 (s, 1H), 6.79 (d, 1H), 3.97 (s, 3H), 3.87 (s, 3H), 3.58 (s, 2H), 3.12 (broad t, 4H), 3.08 (s, 3H), 2.76 (broad s, 4H).

### Example 273

### 5-[4-(5-Chloro-2-methyl-phenyl)-piperazin-1-ylmethyl]-4-methoxy-1-methyl-2-phenyl-1,2-dihydro-pyrazol-3-one

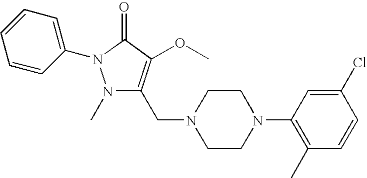

5-[4-(5-Chloro-2-methyl-phenyl)-piperazin-1-ylmethyl]-4-methoxy-1-methyl-2-phenyl-1,2-dihydro-pyrazol-3-one was obtained from 1-(5-chloro-2-methyl-phenyl)-piperazine (53.1 mg, 0.252 mmol), 5-bromomethyl-4-methoxy-1-methyl-2-phenyl-1,2-dihydro-pyrazol-3-one (50.0 mg, 0.168 mmol), and potassium carbonate (116.1 mg, 0.84 mmol) in acetonitrile (2 mL) as a colourless oil (72.0 mg, 100%). ¹H NMR (300 MHz, CDCl₃): δ (ppm) 7.47 (m, 4H), 7.29 (m, 1H), 7.12 (d, 1H), 7.00 (s, 1H), 6.98 (d, 1H), 3.99 (s, 3H), 3.59 (s, 2H), 3.09 (s, 3H), 2.96 (broad s, 4H), 2.73 (broad s, 4H), 2.28 (s, 3H).

### Example 274

### 2-(4-Fluoro-benzyl)-8-(4-methoxy-2-methyl-5-oxo-1-phenyl-2,5-dihydro-1H-pyrazol-3-ylmethyl)-4-phenyl-2,3,8-triaza-spiro[4,5]dec-3-en-1-one

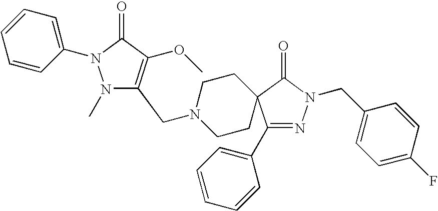

2-(4-Fluoro-benzyl)-8-(4-methoxy-2-methyl-5-oxo-1-phenyl-2,5-dihydro-1H-pyrazol-3-ylmethyl)-4-phenyl-2,3,8-triaza-spiro[4,5]dec-3-en-1-one was synthesized from 5-bromomethyl-4-methoxy-1-methyl-2-phenyl-1,2-dihydro-pyrazol-3-one (11.9 mg, 0.040 mmol), 2-(4-fluoro-benzyl)-4-phenyl-2,3,8-triaza-spiro[4,5]dec-3-en-1-one, (18.5 mg, 0.059 mmol) and potassium carbonate in 2.0 mL of acetonitrile. The desired product was isolated by eluting the crude though a 2 g SPE tube in a solution of 10% acetone and dichloromethane (21.6 mg, 97.6%). ¹H NMR (300 MHz, CDCl₃): δ ppm 1.79 (d, 2H), 2.48 (t of d, 2H), 2.85 (d, 2H), 3.08 (s, 3H), 3.15 (td, 2H), 3.63 (s, 2H), 4.05 (s, 3H), 4.90 (s, 2H), 7.04-7.07 (m, 2H), 7.28-7.47 (m, 10H), 7.80-7.83 (m, 2H).

### Example 275

### 8-(4-Ethoxy-2-methyl-5-oxo-1-phenyl-2,5-dihydro-1H-pyrazol-3-ylmethyl)-2-(4-fluoro-benzyl)-4-phenyl-2,3,8-triaza-spiro[4,5]dec-3-en-1-one

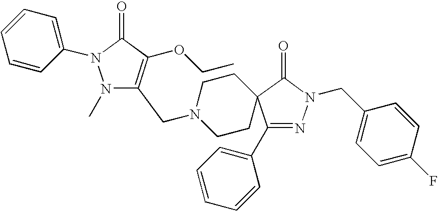

8-(4-Ethoxy-2-methyl-5-oxo-1-phenyl-2,5-dihydro-1H-pyrazol-3-ylmethyl)-2-(4-fluoro-benzyl)-4-phenyl-2,3,8-triaza-spiro[4,5]dec-3-en-1-one was synthesized from 5-bromomethyl-4-ethoxy-1-methyl-2-phenyl-1,2-dihydro-pyrazol-3-one (12.5 mg, 0.040 mmol), 2-(4-fluoro-benzyl)-4-phenyl-2,3,8-triaza-spiro[4,5]dec-3-en-1-one, (18.5 mg, 0.059 mmol) and potassium carbonate in 2.0 mL of acetonitrile. The desired product was isolated by eluting the crude though a 2 g SPE tube in a solution of 10% acetone and dichloromethane to yield a colourless oil (22.7 mg, 77.5%). ¹H NMR (300 MHz, CDCl₃): δ ppm 1.32 (t, 3H), 1.75 (d, br, 2H), 2.44 (td, 2H), 2.82 (d, br, 2H), 3.09 (s, 3H), 3.64 (s, 2H), 4.28 (dd, 2H), 4.91 (s, 2H), 7.02-7.07 (m. 2H), 7.29-7.48 (m, 10H), 7.80-7.83 (m, 2H).

### Example 276

### 5-[4-(5-Chloro-2-methyl-phenyl)-piperazin-1-ylmethyl]-4-ethoxy-1-methyl-2-phenyl-1,2-dihydro-pyrazol-3-one

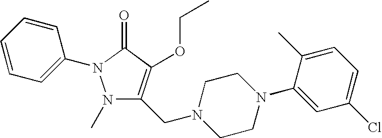

5-[4-(5-Chloro-2-methyl-phenyl)-piperazin-1-ylmethyl]-4-ethoxy-1-methyl-2-phenyl-1,2-dihydro-pyrazol-3-one was obtained from 1-(5-chloro-2-methyl-phenyl)-piperazine (30 mg, 0.144 mmol), 5-bromomethyl-4-ethoxy-1-methyl-2-phenyl-1,2-dihydro-pyrazol-3-one (30.0 mg, 0.096 mmol), and potassium carbonate (40 mg, 0.290 mmol) in acetonitrile (2 mL) as a yellow oil (42.0 mg, 93%). ¹H NMR (300 MHz, CDCl₃): δ (ppm) 7.46-7.50 (m, 4H), 7.27-7.31 (m, 1H), 7.11 (d, 1H), 6.95-7.00 (s, 2H), 4.28 (q, 2H), 3.58 (s, 2H), 3.09 (s, 3H), 2.96 (broad s, 4H), 2.72 (broad s, 4H), 2.28 (s, 3H), 1.36 (t, 3H).

### Example 277

### 5-[4-(5-Chloro-2-methoxyphenyl)-piperazin-1-ylmethyl]-4-ethoxy-1-methyl-2-phenyl-1,2-dihydro-pyrazol-3-one

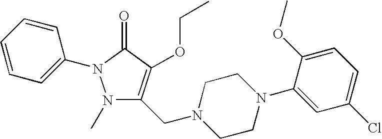

5-[4-(5-Chloro-2-methoxyphenyl)-piperazin-1-ylmethyl]-4-ethoxy-1-methyl-2-phenyl-1,2-dihydro-pyrazol-3-one was obtained from 1-(5-chloro-2-methoxyphenyl)-piperazine (33 mg, 0.144 mmol), 5-bromomethyl-4-ethoxy-1-methyl-2-phenyl-1,2-dihydro-pyrazol-3-one (30.0 mg, 0.096 mmol), and potassium carbonate (40 mg, 0.290 mmol) in acetonitrile (2 mL) as a yellow oil (41.0 mg, 93%). ¹H NMR (300 MHz, CDCl₃): δ (ppm) 7.44-7.47 (m, 4H), 7.27-7.31 (m, 1H), 6.97 (dd, 1H), 6.90 (d, 1H), 6.76 (d, 1H), 4.27 (q, 2H), 3.87 (s, 3H), 3.57 (s, 2H), 3.12 (broad s, 4H), 3.07 (s, 3H), 2.76 (broad s, 4H), 1.34 (t, 3H).

### Example 278

### 4-Ethoxy-1-methyl-2-phenyl-5-[4-(3-phenylpropyl)-piperidin-1-ylmethyl]-1,2-dihydro-pyrazol-3-one

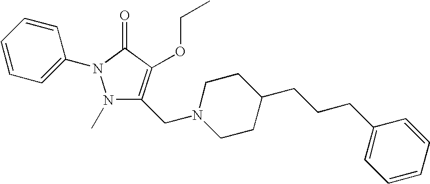

4-Ethoxy-1-methyl-2-phenyl-5-[4-(3-phenylpropyl)-piperidin-1-ylmethyl]-1,2-dihydro-pyrazol-3-one was obtained from 4-(3-phenylpropyl)-piperidine (29 mg, 0.144 mmol), 5-bromomethyl-4-ethoxy-1-methyl-2-phenyl-1,2-dihydro-pyrazol-3-one (30.0 mg, 0.096 mmol), and potassium carbonate (40 mg, 0.290 mmol) in acetonitrile (2 mL) as a yellow oil (37.7 mg, 94%). ¹H NMR (300 MHz, CDCl₃): δ (ppm) 7.45-7.47 (m, 4H), 7.26-7.33 (m, 3H), 7.19-7.22 (m, 3H), 4.25 (q, 2H), 3.47 (s, 2H), 3.05 (q, 3H), 2.93 (d, 2H), 2.62 (t, 2H), 2.06 (t, 2H), 1.64-1.73 (m, 4H), 1.22-1.36 (m, 8H).

### Example 279

### 5-[4-(5-Chloro-2-methoxy-phenyl)-piperazin-1-ylmethyl]-4-difluoromethoxy-1-methyl-2-phenyl-1,2-dihydro-pyrazol-3-one

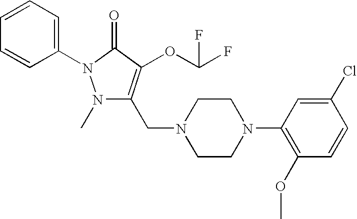

5-[4-(5-Chloro-2-methoxy-phenyl)-piperazin-1-ylmethyl]-4-difluoromethoxy-1-methyl-2-phenyl-1,2-dihydro-pyrazol-3-one was obtained from 1-(5-chloro-2-methoxy-phenyl)-piperazine (31.7 mg, 0.14 mmol), 5-bromomethyl-4-difluoromethoxy-1-methyl-2-phenyl-1,2-dihydro-pyrazol-3-one (30.0 mg, 0.093 mmol), and potassium carbonate (64.5 mg, 0.47 mmol) in acetonitrile (1.5 mL) as a white solid (39.8 mg, 89%). ¹H NMR (300 MHz, CDCl₃): δ (ppm) 7.51 (dd, 2H), 7.41 (dd, 2H), 7.36 (t, 1H), 7.03 (t, 1H), 6.97 (dd, 1H), 6.90 (d, 1H), 6.78 (s, 1H), 3.88 (s, 3H), 3.64 (s, 2H), 3.23 (s, 3H), 3.12 (broad s, 4H), 2.77 (broad t, 4H).

### Example 280

### 8-(4-Difluoromethoxy-2-methyl-5-oxo-1-phenyl-2,5-dihydro-1H-pyrazol-3-ylmethyl)-1-phenyl-1,3,8-triaza-spiro[4.5]decan-4-one

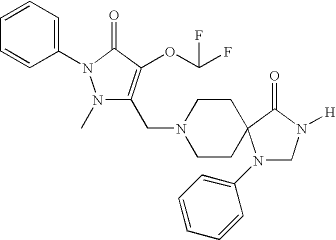

8-(4-Difluoromethoxy-2-methyl-5-oxo-1-phenyl-2,5-dihydro-1H-pyrazol-3-ylmethyl)-1-phenyl-1,3,8-triaza-spiro[4.5]decan-4-one was obtained from 1-Phenyl-1,3,8-triaza-spiro[4.5]decan-4-one (32.4 mg, 0.14 mmol), 5-Bromomethyl-4-difluoromethoxy-1-methyl-2-phenyl-1,2-dihydro-pyrazol-3-one (30.0 mg, 0.093 mmol), and potassium carbonate (64.5 mg, 0.47 mmol) in acetonitrile (1.5 mL) as a white solid (38.8 mg, 87%). ¹H NMR (300 MHz, CDCl₃): δ (ppm) 7.51 (m, 2H), 7.41 (d, 2H), 7.31 (m, 3H), 7.03 (t, 1H), 6.93 (m, 3H), 6.68 (broad s, 1H), 4.78 (s, 2H), 3.67 (s, 2H), 3.27 (s, 3H), 3.03 (td, 2H), 2.87 (broad d, 2H), 2.70 (td, 2H), 1.80 (broad d, 2H).

### Example 281

### 5-[4-(5-Chloro-2-methyl-phenyl)-piperazin-1-ylmethyl]-4-difluoromethoxy-1-methyl-2-phenyl-1,2-dihydro-pyrazol-3-one

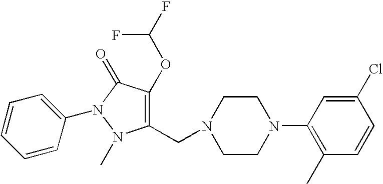

5-[4-(5-Chloro-2-methyl-phenyl)-piperazin-1-ylmethyl]-4-difluoromethoxy-1-methyl-2-phenyl-1,2-dihydro-pyrazol-3-one was obtained from 1-(5-Chloro-2-methyl-phenyl)-piperazine (53.5 mg, 0.254 mmol), 5-Bromomethyl-4-difluoromethoxy-1-methyl-2-phenyl-1,2-dihydro-pyrazol-3-one (30.0 mg, 0.170 mmol), and potassium carbonate (117.5 mg, 0.85 mmol) in acetonitrile (1.5 mL) as a white powder (32.3 mg, 41%). ¹H NMR (300 MHz, CDCl₃): δ (ppm) 7.52 (t, 2H), 7.42 (dd, 2H), 7.40 (t, 1H), 7.12 (d, 1H), 7.04 (t, 1H), 7.99 (d, 2H), 3.65 (s, 2H), 3.24 (s, 3H), 2.96 (broad t, 4H), 2.74 (broad s, 4H), 2.28 (s, 3H).

### Example 282

### 5-[4-(5-Chloro-2-methoxy-phenyl)-piperazin-1-ylmethyl]-1-methyl-2-phenyl-4-(2,2,2-trifluoro-ethoxy)-1,2-dihydro-pyrazol-3-one

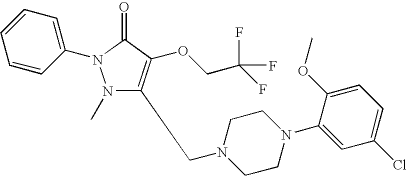

5-[4-(5-Chloro-2-methoxy-phenyl)-piperazin-1-ylmethyl]-1-methyl-2-phenyl-4-(2,2,2-trifluoro-ethoxy)-1,2-dihydro-pyrazol-3-one was synthesized from 1-(5-chloro-2-methoxy-phenyl)-piperazine (41.1 mg, 0.156 mmol), 5-bromomethyl-1-methyl-2-phenyl-4-(2,2,2-trifluoro-ethoxy)-1,2-dihydro-pyrazol-3-one (38 mg, 0.104 mmol) and potassium carbonate (43.1 mg, 0.312 mmol) in 2.0 mL of acetonitrile. The crude material was purified by eluting through a 2 g SPE tube using a solution of 20% acetone and hexanes to yield a colourless oil (41.2 mg, 78.8%). ¹H NMR (300 MHz, CDCl₃): δ ppm 2.77 (br, 4H), 3.13 (br, 4H), 3.13 (s, 3H), 3.60 (s, 2H), 3.87 (s, 3H), 4.75-4.66 (dd, 2H), 6.80-6.77 (d, 1H), 6.90-6.90 (d, 1H), 6.99-6.95 (dd, 1H), 7.35-7.32 (m, 1H), 7.52-7.41 (m, 4H).

### Deprotection of BOC Group and Coupling to Pyrazalone

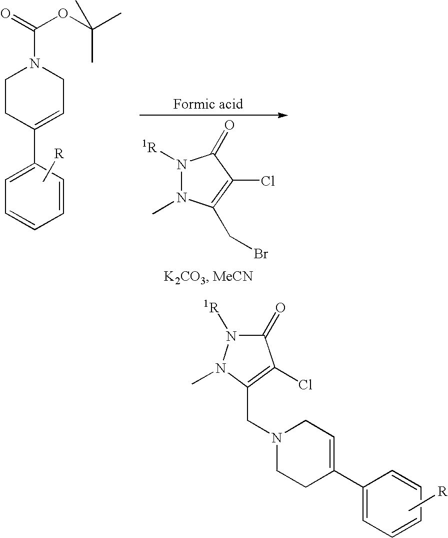

### General Procedure:

The tert-butyl ester (1.0 equiv) was allowed to stir in formic acid (2.0 mL) for 2 h. The formic acid was concentrated off and co-evaporated with dichloromethane. The deprotected piperidine (1.5 equiv) was reacted with the corresponding pyrazalone (1.0 equiv) and potassium carbonate (4.0 equiv) in 3.0 mL of acetonitrile for one day. The reaction was extracted three times with water and the product was purified by column chromatography using a 2 g SPE tube in a solution of acetone and dichloromethane. The identity of the product was verified by 1H NMR.

Compounds of Examples 283 through 296 were synthesized using a method analogous to the above general procedure for piperazine and pyrazalone coupling.

### Example 283

### 4-Chloro-5-[4-(5-chloro-2-methyl-phenyl)-3,6-dihydro-2H-pyridin-1-ylmethyl]-1-methyl-2-phenyl-1,2-dihydro-pyrazol-3-one

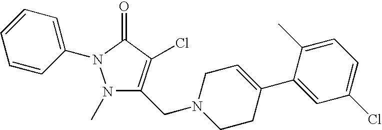

4-Chloro-5-[4-(5-chloro-2-methyl-phenyl)-3,6-dihydro-2H-pyridin-1-ylmethyl]-1-methyl-2-phenyl-1,2-dihydro-pyrazol-3-one was synthesized from 4-(5-chloro-2-methyl-phenyl)-3,6-dihydro-2H-pyridine-1-carboxylic acid tert-butyl ester (0.190 g, 0.647 mmol), 5-bromomethyl-4-chloro-1-methyl-2-phenyl-1,2-dihydro-pyrazol-3-one (87 mg, 0.287 mmol) and potassium carbonate (159 mg, 1.148 mmol) in 3.0 mL of acetonitrile. The crude product was purified by eluting through a 5 g SPE tube using a solution of 30% acetone and hexanes to yield a brown gum. (170 mg, 96.59%) ¹H NMR (300 MHz, CDCl₃): δ ppm 7.50-7.12 (m, 8H), 5.58 (br, 1H), 3.72 (s, 3H), 3.30 (s, 2H), 3.27 (br, 2H), 2.83 (br, 2H), 2.40 (br, 2H) 2.27 (br, 3H).

### Example 284

### 4-Chloro-5-[4-(5-chloro-2-methoxyphenyl)-3,6-dihydro-2H-pyridin-1-ylmethyl]-1-methyl-2-phenyl-1,2-dihydro-pyrazol-3-one

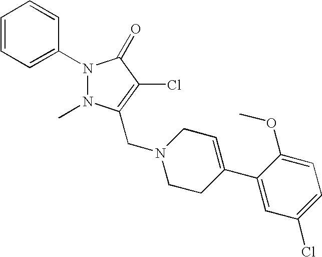

4-Chloro-5-[4-(5-chloro-2-methoxyphenyl)-3,6-dihydro-2H-pyridin-1-ylmethyl]-1-methyl-2-phenyl-1,2-dihydro-pyrazol-3-one was synthesized from 4-(5-chloro-2-methyl-phenyl)-3,6-dihydro-2H-pyridine-1-carboxylic acid tert-butyl ester (0.096 g, 0.43 mmol), 5-bromomethyl-4-chloro-1-methyl-2-phenyl-1,2-dihydro-pyrazol-3-one (87 mg, 0.290 mmol) and potassium carbonate (160 mg, 1.15 mmol) in 3.0 mL of acetonitrile. The product was purified by eluting through a 5 g SPE tube using a solution of 30% acetone and hexanes to yield a colourless solid (100 mg, 78%).

### Example 285

### 4-Chloro-5-[4-(5-chloro-2-methyl-phenyl)-3,6-dihydro-2H-pyridin-1-ylmethyl]-2-(4-fluoro-phenyl)-1-methyl-1,2-dihydro-pyrazol-3-one

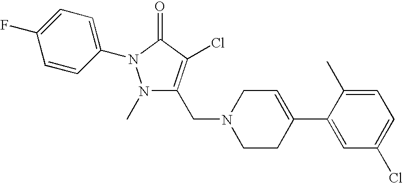

4-Chloro-5-[4-(5-chloro-2-methyl-phenyl)-3,6-dihydro-2H-pyridin-1-ylmethyl]-2-(4-fluoro-phenyl)-1-methyl-1,2-dihydro-pyrazol-3-one was synthesized from 5-bromomethyl-4-chloro-2-(4-fluoro-phenyl)-1-methyl-1,2-dihydro-pyrazol-3-one (29.15 mg, 0.096 mmol), 4-(5-chloro-2-methyl-phenyl)-3,6-dihydro-2H-pyridine-1-carboxylic acid tert-butyl ester (29.1 mg, 0.141 mmol) and potassium carbonate (38.8 mg, 0.281 mmol) in 3 mL of acetonitrile to yield a clear oil (31.6 mg, 75.2 mmol). ¹H NMR (300 MHz, CDCl₃): δ ppm 2.27 (s, 3H), 2.37-2.41 (br, 2H), 2.81-2.85 (t, 2H), 3.24-3.29 (br, 2H), 3.27 (s, 3H), 3.72 (s, 2H), 5.57-5.59 (br, 1H), 7.11-7.22 (m, 5H), 7.38-7.42 (m, 2H).

### Example 286

### 4-Chloro-5-[4-(5-chloro-2-methoxy-phenyl)-3,6-dihydro-2H-pyridin-1-ylmethyl]-2-(4-fluoro-phenyl)-1-methyl-1,2-dihydro-pyrazol-3-one

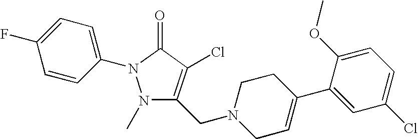

4-Chloro-5-[4-(5-chloro-2-methoxy-phenyl)-3,6-dihydro-2H-pyridin-1-ylmethyl]-2-(4-fluoro-phenyl)-1-methyl-1,2-dihydro-pyrazol-3-one was synthesized from 5-bromomethyl-4-chloro-2-(4-fluoro-phenyl)-1-methyl-1,2,-dihydro-pyrazol-3-one (43.9 mg, 0.137 mmol), 4-(5-chloro-2-methoxy-phenyl)-3,6-dihydro-2H-pyridine-1-carboxylic acid tert-butyl ester (66.6 mg, 0.21 mmol) and potassium carbonate (75.5 mg, 0.548 mmol) in 3.0 mL of acetonitrile. The crude reactions were purified by eluting through a 2 g SPE tube using a solution of 30% ethyl acetate and hexanes to yield a yellow gum (25.3 mg, 39.9%).

### Example 287

### 4-Chloro-5-[4-(5-chloro-2-methyl-phenyl)-3,6-dihydro-2H-pyridin-1-ylmethyl]-1-methyl-2-(4-trifluoromethoxy-phenyl)-1,2-dihydro-pyrazol-3-one

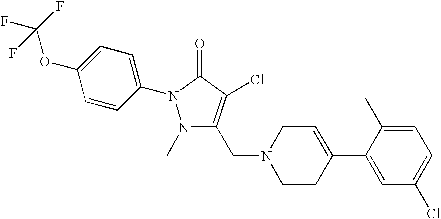

4-Chloro-5-[4-(5-chloro-2-methyl-phenyl)-3,6-dihydro-2H-pyridin-1-ylmethyl]-1-methyl-2-(4-trifluoromethoxy-phenyl)-1,2-dihydro-pyrazol-3-one was synthesized from 5-bromomethyl-4-chloro-1-methyl-2-(4-trifluoromethoxy-phenyl)-1,2-dihydro-pyrazol-3-one (30 mg, 0.078 mmol), 4-(5-chloro-2-methyl-phenyl)-3,6-dihydro-2H-pyridine-1-carboxylic acid tert-butyl ester (24.2 mg, 0.117 mmol) and potassium carbonate (31.78 mg, 0.23 mmol) in 3 mL of acetonitrile to yield a pale yellow solid (38.9 mg, 97.3%). ¹H NMR (300 MHz, CDCl₃): δ ppm 2.74 (s, 3H), 2.39 (br, 2H), 2.83 (t, 2H), 3.27 (t, 2H), 3.28 (s, 3H), 3.73 (s, 2H), 4.59 (br, 1H), 7.10-7.17 (m, 3H), 7.34-7.47 (m, 2H), 7.46-7.51 (dt, 2H).

### Example 288

### 4-Chloro-5-[4-(5-chloro-2-methoxy-phenyl)-3,6-dihydro-2H-pyridin-1-ylmethyl]-2-cyclopentyl-1-methyl-1,2-dihydro-pyrazol-3-one

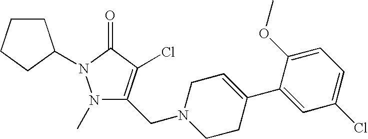

4-Chloro-5-[4-(5-chloro-2-methoxy-phenyl)-3,6-dihydro-2H-pyridin-1-ylmethyl]-2-cyclopentyl-1-methyl-1,2-dihydro-pyrazol-3-one was synthesized from 4-(5-chloro-2-methoxy-phenyl)-3,6-dihydro-2H-pyridine-1-carboxylic acid tert-butyl ester (66.6 mg, 0.21 mmol), 5-bromomethyl-4-chloro-2-cyclopentyl-1-methyl-1,2-dihydro-pyrazol-3-one 940.2 mg, 0.137 mmol) and potassium carbonate (75.5 mg, 0.548 mmol) in 3.0 mL of acetonitrile. The crude product was purified by eluting through a 2 g SPE tube using a solution of 30% ethyl acetate and hexanes to yield a yellow gum (35.6 mg, 75%). ¹H NMR (300 MHz, CDCl₃): δ ppm 1.89-1.62 (m, 2H), 2.03-1.89 (m, 6H), 2.50 (br, 2H), 2.70 (t, 2H), 3.18 (br, 2H), 3.43 (s, 3H), 3.56 (s, 2H), 3.79 (s, 3H), 4.60 (quintet, 1H), 5.79 (br, 1H), 6.79-6.76 (m, 1H), 7.19-7.12 (m, 2H).

### Example 289

### 5-[4-(5-Chloro-2-methoxy-phenyl)-3,6-dihydro-2H-pyridin-1-ylmethyl]-4-methoxy-1-methyl-2-phenyl-1,2-dihydro-pyrazol-3-one

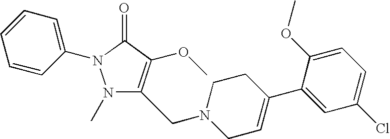

5-[4-(5-Chloro-2-methoxy-phenyl)-3,6-dihydro-2H-pyridin-1-ylmethyl]-4-methoxy-1-methyl-2-phenyl-1,2-dihydro-pyrazol-3-one was synthesized from 4-(5-chloro-2-methoxy-phenyl)-3,6-dihydro-2H-pyridine-1-carboxylic acid tert-butyl ester (66.6 mg, 0.21 mmol), 5-bromomethyl-4-methoxy-1-methyl-2-phenyl-1,2-dihydro-pyrazol-3-one (40.8 mg, 0.137 mmol) and potassium carbonate (75.5 mg, 0.548 mmol) in 3.0 mL of acetonitrile. The crude reactions were purified by eluting through a 2 g SPE tube using a solution of 30% ethyl acetate and hexanes to yield a yellow gum (39.5 mg, 65.5%). ¹H NMR (300 MHz, CDCl₃): δ ppm 2.56 (br, 2H), 2.77 (t, 2H), 3.07 (3H), 3.26 (br, 2H), 3.62 (s, 2H), 3.81 (s, 3H), 3.96 (s, 3H), 5.85 (br, 1H), 6.81-6.78 (m, 1H), 7.20-7.16 (m, 2H), 7.43-7.26 (m, 1H), 7.51-7.43 (m. 4H).

### Example 290

### 4-Chloro-5-[4-(chloro-2-methyl-phenyl)-piperidin-1-ylmethyl]-1-methyl-2-phenyl-1,2-dihydro-pyrazol-3-one

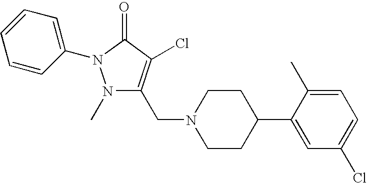

4-Chloro-5-[4-(chloro-2-methyl-phenyl)-piperidin-1-ylmethyl]-1-methyl-2-phenyl-1,2-dihydro-pyrazol-3-one was synthesized from 5-bromomethyl-4-chloro-1-methyl-2-phenyl-1,2-dihydro-pyrazol-3-one (33.5 mg, 0.111 mmol), 4-(5-chloro-2-methyl-phenyl)-piperidine-1-carboxylic acid tert-butyl ester (200 mg, 0.167 mmol) and potassium carbonate (46.16 mg, 0.334 mmol) in 3 mL of acetonitrile to yield a yellow gum (12.5 mg, 26.16%). ¹H NMR (300 MHz, CDCl₃): δ ppm 1.81-1.67 (m, 6H), 2.34-2.26 (m, 2H), 2.34 (s, 3H), 2.74 (quintet, 1H), 3.08 (d, (br), 2H), 3.28 (s, 3H), 3.62 (s, 2H), 7.10 (s, 2H), 7.22 (s, 1H), 7.36-7.54 (m, 5H).

### Example 291

### 4-Chloro-5-[4-(5-chloro-2-methyl-phenyl)-piperidin-1-ylmethyl]-2-(4-fluoro-phenyl)-1-methyl-1,2-dihydro-pyrazol-3-one

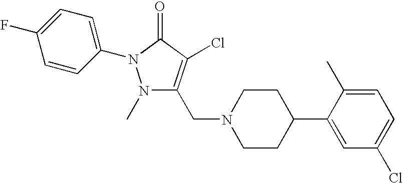

4-Chloro-5-[4-(5-chloro-2-methyl-phenyl)-piperidin-1-ylmethyl]-2-(4-fluoro-phenyl)-1-methyl-1,2-dihydro-pyrazol-3-one was synthesized from 5-bromomethyl-4-chloro-2-(4-fluoro-phenyl)-1-methyl-1,2-dihydro-pyrazol-3-one (29.15 mg, 0.096 mmol), 4-(5-chloro-2-methyl-phenyl)-piperidine-1-carboxylic acid tert-butyl ester (29.5 mg, 0.141 mmol) and potassium carbonate (38.8 mg, 0.281 mmol) in 3 mL of acetonitrile to yield a clear oil (10.3 mg, 25.0%). ¹H NMR (300 MHz, CDCl₃): δ ppm 1.77 (td, 2H) 1.81 (br, 2H), 2.321 (s, H), 2.29-2.34 (td, 2H), 2.34 (5, 1H), 2.74 (d (br), 2H), 3.06 (s, 3H), 3.61 (s, 2H), 7.09 (s, 2H), 7.10-7.23 (m, 3H), 7.39-7.43 (m, 2H).

### Example 292

### 4-Chloro-5-[4-(5-chloro-2-methoxy-phenyl)-piperidin-1-ylmethyl]-2-(4-fluoro-phenyl)-1-methyl-1,2-dihydro-phyrazol-3-one

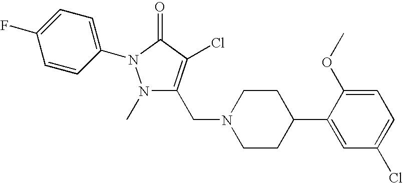

4-Chloro-5-[4-(5-chloro-2-methoxy-phenyl)-piperidin-1-ylmethyl]-2-(4-fluoro-phenyl)-1-methyl-1,2-dihydro-phyrazol-3-one was synthesized from 5-bromomethyl-4-chloro-2-(4-fluoro-phenyl)-1-methyl-1,2-dihydro-pyrazol-3-one (29.15 mg, 0.096 mmol), 4-(5-Chloro-2-methoxy-phenyl)-piperidine-1-carboxylic acid tert-butyl ester (34.0 mg, 0.414 mmol) and potassium carbonate (38.8 mg, 0.281 mmol) in 3 mL of acetonitrile to yield a clear oil (10.6 mg, 24.5%). ¹H NMR (300 MHz, CDCl₃): δ ppm 1.70 (td, 2H), 1.84 (d (br), 2H), 2.31 (td, 2H), 3.03 (d (br), 2H), 3.25 (s, 3H), 3.60 (s, 2H), 3.85 (s, 3H), 3.78 (d, 1H), 7.14-7.23 (m, 4H), 7.37-7.42 (m, 2H).

### Example 293

### 4-Chloro-5-[4-(5-chloro-2-methyl-phenyl)-cyclohexylmethyl]-1-methyl-2-(4-trifluoromethoxy-phenyl)-1,2-dihydro-pyrazol-3-one

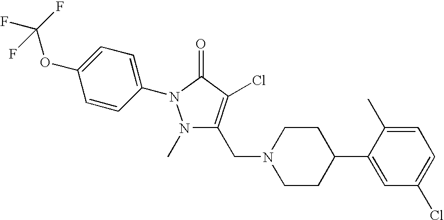

4-Chloro-5-[4-(5-chloro-2-methyl-phenyl)-cyclohexylmethyl]-1-methyl-2-(4-trifluoromethoxy-phenyl)-1,2-dihydro-pyrazol-3-one was synthesized from 5-bromomethyl-4-chloro-1-methyl-2-(4-trifluoromethoxy-phenyl)-1,2-dihydro-pyrazol-3-one (30 mg, 0.078 mmol), 4-(5-chloro-2-methyl-phenyl)-piperidine-1-carboxylic acid tert-butyl ester (24.5 mg, 0.117 mmol) and potassium carbonate (31.78 mg, 0.23 mmol) in 3 mL of acetonitrile to yield a pale yellow solid (40.6 mg, 100.1%). ¹H NMR (300 MHz, CDCl₃): δ ppm 1.73-1.84 (m, 2H), 2.26-2.35 (m, 2H), 2.32 (s, 3H), 2.74 (quintet, 1H), 3.07 (d, 2H), 3.28 (s, 3H), 3.62 (s, 2H), 7.09 (s, 2H), 7.10 (s, 1H), 7.38 (d, 2H), 7.45-7.49 (m, 2H).

### Example 294

### 4-Chloro-5-[4-(5-chloro-2-methyl-phenyl)-piperidin-1-ylmethyl]-2-cyclopentyl-1-methyl-1,2,-cyclopentyl-1-methyl-1,2-dihydro-pyrazol-3-one

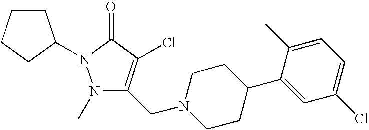

4-Chloro-5-[4-(5-chloro-2-methyl-phenyl)-piperidin-1-ylmethyl]-2-cyclopentyl-1-methyl-1,2,-cyclopentyl-1-methyl-1,2-dihydro-pyrazol-3-one was synthesized from 5-bromomethyl-4-chloro-2-cyclopentyl-1-methyl-1,2-dihydro-pyrazol-3-one (33.1 mg, 0.1126 mmol), 4-(5-chloro-2-methyl-phenyl)-piperidine-1-carboxylic acid tert-butyl ester (50 mg, 0.169 mmol) and potassium carbonate (46.71 mg, 0.338 mmol) in 3 mL of acetonitrile. The crude reactions were purified by eluting through a 2 g SPE tube using a solution of 30% ethyl acetate and hexanes to yield a yellow gum (37.8 mg, %). ¹H NMR (300 MHz, CDCl₃): δ ppm 1.63-1.75 (m, 6H), 2.01-2.18 (m, 8H), 2.33 (s, 3H), 2.96-3.00 (d, (br), 2H), 3.41 (s, H), 3.48 (s, 2H), 4.62-4.68 (quintet, 1H), 7.07-7.10 (m, 2H), 7.18 (s, 1H).

### Example 295

### 4-Chloro-5-{1-[4-(5-chloro-2-methoxy-phenyl)-3,6-dihydro-2H-pyridin-1-yl]-ethyl}-1-methyl-2-phenyl-1,2-dihydro-pyrazol-3-one

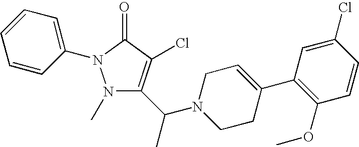

4-Chloro-5-{1-[4-(5-chloro-2-methoxy-phenyl)-3,6-dihydro-2H-pyridin-1-yl]-ethyl}-1-methyl-2-phenyl-1,2-dihydro-pyrazol-3-one was synthesized from 4-chloro-5-{1-[4-(5-chloro-2-methoxy-phenyl)-4-hydroxy-piperidin-1-yl]-ethyl}-1-methyl-2-phenyl-1,2-dihydro-pyrazol-3-one (21.2 mg, 0.044 mmol) and TFA (0.68 mL, 0.0088 mmol) in 2 mL of THF. The crude product was purified by column chromatography using a solution of 100% ethyl acetate and then switching to a solution of 100% acetone to yield a clear oil (3.2 mg, 15.9%). ¹H NMR (300 MHz, CDCl₃): δ ppm ˜1.6 (m, 1H) 1.99 (d, 3H), 2.24 (t, 2H), 2.62 (t, 2H), 3.27 (s, 3H), 3.54 (br, 3H) 3.59 (s, 3H), 4.99 (br, 1H), 6.91 (d, 1H), 7.28-7.31 (m, 2H), 7.31-7.51 (m, 3H), 7.46-7.56 (m, 2H).

### Example 296

### 4-Chloro-5-{1-[4-(5-chloro-2-methoxy-phenyl)-4-hydroxy-piperidin-1-yl]-ethyl}-1-methyl-2-phenyl-1,2-dihydro-pyrazol-3-one

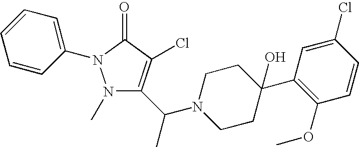

4-Chloro-5-{1-[4-(5-chloro-2-methoxy-phenyl)-4-hydroxy-piperidin-1-yl]-ethyl}-1-methyl-2-phenyl-1,2-dihydro-pyrazol-3-one was synthesized from 2-bromo-4-chloro-1-methoxy-benzene (0.188 g, 0.85 mmol) and magnesium (20.7 mg, 0.85 mmol) and 1-[1-(4-chloro-2-methyl-5-oxo-1-phenyl-2,5-dihydro-1H-pyrazol-3-yl)-ethyl]-piperidin-4-one (60 mg, 0.17 mmol in 4 mL of THF. The crude product was purified by eluting through a 2 g SPE tube using a solution of 80% ethyl acetate and hexanes to yield a clear oil. ¹H NMR (300 MHz, CDCl₃): δ ppm 11.53 (d, 3H), 2.10 (m, 4H), 2.75 (m, 4H), 3.36 (s, 3H), 3.83 (m, 1H), 3.96 (s, 3H), 6.82-7.55 (m, 8H).

### General Procedure B

The 1-(2-methoxy-phenyl)-piperazine (1.2 equiv.) was added to a mixture of potassium carbonate (2 equiv.) and 4-bromo-5-bromomethyl pyrazalones (1 equiv.) in acetone. It was left to stir overnight at 70° C. The resulting reaction mixture was partitioned between water and dichloromethane. Solvent was removed from the organic layer. The resulting crude product was then purified using column chromatography with 50% hexanes and ethyl acetate. Solvent was removed in vacuo. NMR was used to determine the purity of the isolated compounds.

Compounds of Examples 297 through 351 were synthesized using a method analogous to the above general procedure B for piperazine and pyrazolone coupling.

### Example 297

### 4-Bromo-2-(2-chloro-phenyl)-5-[4-(2-methoxy-phenyl)-piperazin-1-ylmethyl-1-methyl-1,2-dihydro-pyrazol-3-one

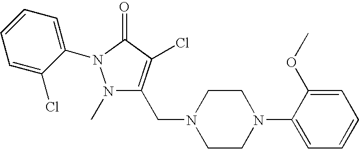

4-Bromo-2-(2-chloro-phenyl)-5-[4-(2-methoxy-phenyl)-piperazin-1-ylmethyl-1-methyl-1,2-dihydro-pyrazol-3-one was obtained from 1-(2-methoxy-phenyl)-piperazine (0.43 mmol, 0.083 g), 4-Bromo-5-bromomethyl-2-(2-chloro-phenyl)-1-methyl-1,2-dihydro-pyrazol-3-one (0.41 mmol, 0.155 g) and potassium carbonate (0.8 mmol, 0.111 g) in acetone (4 mL) as a off white solid (0.190 g, 95%).

¹H NMR (300 MHz, CDCl₃): δ (ppm) 7.40-7.58 (m, 4H), 6.92 (m, 4H), 3.88 (s, 3H), 3.64 (d, 2H), 3.25 (s, 3H), 3.10 (s, 4H), 2.78 (s, 4H).

### Example 298

### 4-Bromo-2-(4-chloro-phenyl)-5-[4-(2-methoxy-phenyl)-piperazin-1-ylmethyl-1-methyl-1,2-dihydro-pyrazol-3-one

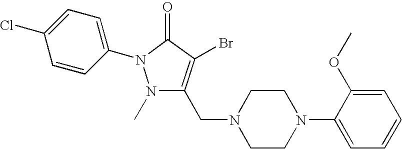

4-Bromo-2-(4-chloro-phenyl)-5-[4-(2-methoxy-phenyl)-piperazin-1-ylmethyl-1-methyl-1,2-dihydro-pyrazol-3-one was obtained from 1-(2-methoxy-phenyl)-piperazine (0.13 mmol, 0.025 g), 4-Bromo-5-bromomethyl-2-(4-chloro-phenyl)-1-methyl-1,2-dihydro-pyrazol-3-one (0.11 mmol, 0.040 g) and potassium carbonate (0.3 mmol, 0.041 g) in acetone (2 mL) as a off white solid (0.049 g, 92%).

¹H NMR (300 MHz, CDCl₃): δ (ppm) 7.44 (d, 2H), 7.34 (d, 2H0, 6.89-7.04 (m, 4H), 3.88 (s, 3H), 3.64 (s, 2H), 3.25 (s, 3H), 3.12 (s, 4H), 2.78 (s, 4H).

### Example 299

### 4-Bromo-2-(3-chloro-phenyl)-5-[4-(2-methoxy-phenyl)-piperazin-1-ylmethyl-1-methyl-1,2-dihydro-pyrazol-3-one

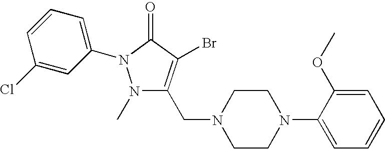

4-Bromo-2-(3-chloro-phenyl)-5-[4-(2-methoxy-phenyl)-piperazin-1-ylmethyl-1-methyl-1,2-dihydro-pyrazol-3-one was obtained from 1-(2-methoxy-phenyl)-piperazine (0.6 mmol, 0.115 g), 4-Bromo-5-bromomethyl-2-(3-chloro-phenyl)-1-methyl-1,2-dihydro-pyrazol-3-one (0.5 mmol, 0.190 g) and potassium carbonate (1.5 mmol, 0.207 g) in acetone (4 mL) as a off white solid (0.189 g, 77%).

¹H NMR (300 MHz, CDCl₃): δ (ppm) 7.28-7.42 (m, 4H), 6.89-7.04 (m, 4H), 3.88 (s, 3H0, 3.64 9d, 2H), 3.26 (s, 3H), 3.13 (s, 4 h), 2.79 (s, 4H).

### Example 300

### 4-Bromo-2-(4-methoxy-phenyl)-5-[4-(2-methoxy-phenyl)-piperazin-1-ylmethyl-1-methyl-1,2-dihydro-pyrazol-3-one

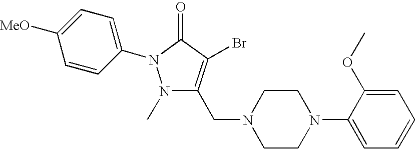

4-Bromo-2-(4-methoxy-phenyl)-5-[4-(2-methoxy-phenyl)-piperazin-1-ylmethyl-1-methyl-1,2-dihydro-pyrazol-3-one was obtained from 1-(2-methoxy-phenyl)-piperazine (0.42 mmol, 0.081 g), 4-Bromo-5-bromomethyl-2-(4-methoxy-phenyl)-1-methyl-1,2-dihydro-pyrazol-3-one (0.381 mmol, 0.142 g) and potassium carbonate (1.5 mmol, 0.207 g) in acetone (5 mL) as a off white solid (0.151 g, 82%).

¹H NMR (300 MHz, CDCl₃): δ (ppm) 7.24-7.30 (m, 2H), 6.87-7.04 (m, 6H), 3.88 9s, 3H), 3.84 (s, 3H0, 3.64 (d, 2H), 3.24 (s, 3H), 3.10 (s, 4H), 2.76 (s, 4H).

### Example 301

### 4-Bromo-2-(4-methoxy-phenyl)-5-[4-(2-methoxy-phenyl)-piperazin-1-ylmethyl-1-methyl-1,2-dihydro-pyrazol-3-one

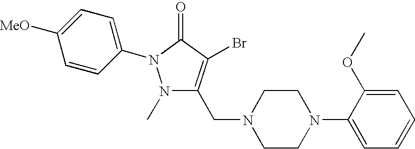

4-Bromo-2-(4-methoxy-phenyl)-5-[4-(2-methoxy-phenyl)-piperazin-1-ylmethyl-1-methyl-1,2-dihydro-pyrazol-3-one was obtained from 1-(2-methoxy-phenyl)-piperazine (0.42 mmol, 0.081 g), 4-Bromo-5-bromomethyl-2-(4-methoxy-phenyl)-1-methyl-1,2-dihydro-pyrazol-3-one (0.381 mmol, 0.142 g) and potassium carbonate (1.5 mmol, 0.207 g) in acetone (5 mL) as a off white solid (0.151 g, 82%).

¹H NMR (300 MHz, CDCl₃): δ (ppm) 7.24-7.30 (m, 2H), 6.87-7.04 (m, 6H), 3.88 9s, 3H), 3.84 (s, 3H0, 3.64 (d, 2H), 3.24 (s, 3H), 3.10 (s, 4H), 2.76 (s, 4H).

### Example 302

### 4-Chloro-5-{1-[4-(3-chloro-4-fluoro-phenyl)-piperazin-1-yl]-ethyl}-1-methyl-2-phenyldihydro-pyrazol-3-one

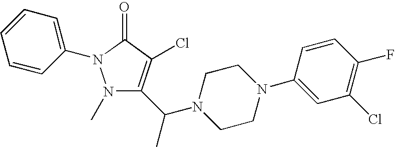

4-Chloro-5-{1-[4-(3-chloro-4-fluoro-phenyl)-piperazin-1-yl]-ethyl}-1-methyl-2-phenyldihydro-pyrazol-3-one was obtained from 1-(3-chloro-4-fluoro-phenyl)-piperazine (0.199 mmol, 0.051 g), 5-(1-bromo-ethyl)-4-chloro-1-methyl-2-phenyl-1,2-dihydro-pyrazol-3-one (0.138 mmol, 0.0040 g) and potassium carbonate (0.663 mmol, 0.092 g) in acetone (5 mL) as a off white solid (0.0527 g, 90%).

¹H NMR (300 MHz, CDCl₃): δ (ppm) 7.33-7.53 (m, 5H), 6.79-7.08 (m, 2H) 6.77-6.79 (m, 1H), 3.86 (q, 1H), 3.34 (s, 3H), 2.65-2.83 (m, 4H), 2.75 (d, 4H), 1.53 (d, 3H).

### Example 303

### 4-Chloro-5-[4-(3-chloro-4-fluoro-phenyl)piperazin-1-ylmethyl]-1-methyl-2-phenyl-1,2-dihydro-pyrazol-3-one

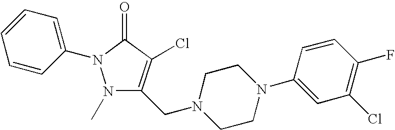

4-Chloro-5-[4-(3-chloro-4-fluoro-phenyl)piperazin-1-ylmethyl]-1-methyl-2-phenyl-1,2-dihydro-pyrazol-3-one was obtained from 1-(3-chloro-4-fluoro-phenyl)-piperazine (0.199 mmol, 0.051 g), 5-bromoethyl-4-chloro-1-methyl-2-phenyl-1,2-dihydro-pyrazol-3-one (0.133 mmol, 0.040 g) and potassium carbonate (0.663 mmol, 0.0916 g) in acetonitrile (3 mL) as a light yellow solid (0.0584 g, 98%).

¹H NMR (300 MHz, CDCl₃): δ (ppm) 7.36-7.52 (m, 5H), 6.95-7.09 (m, 2H), 6.65-6.81 (m, 1H), 3.65 (s, 2H), 3.24 (s, 3H), 3.15-3.19 (m, 4H), 2.72-2.76 (m, 4H).

### Example 304

### 4-Chloro-1-methyl-5-{1-[4-(6-methyl-pyridin-2-yl)-piperazin-1-yl]-ethyl}-2-phenyldihydro-pyrazol-3-one

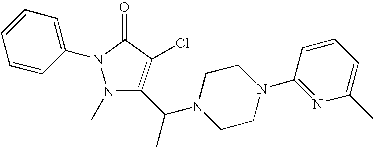

4-Chloro-1-methyl-5-{1-[4-(6-methyl-pyridin-2-yl)-piperazin-1-yl]-ethyl}-2-phenyldihydro-pyrazol-3-one was obtained from 1-(6-methyl-pyridin-2-yl)-piperazine (0.199 mmol, 0.035 g), 5-(1-bromo-ethyl)-4-chloro-1-methyl-2-phenyl-1,2-dihydro-pyrazol-3-one (0.138 mmol, 0.0.040 g) and potassium carbonate (0.663 mmol, 0.092 g) in acetone (5 mL) as a off white solid (0.0508 g, 94%).

¹H NMR (300 MHz, CDCl₃): δ (ppm) 7.33-7.52 (m, 6H), 6.46-6.55 (m, 2H), 3.86 (q, 1H), 3.37 (s, 3H), 2.62-2.79 (m, 4H), 2.42 (s, 3H), 1.53 (d, 3H).

### Example 305

### 4-Chloro-5-{1-[4-(2,5-dichloro-phenyl)-piperazin-1-yl]-ethyl}-1-methyl-2-phenyl-1,2-dihydro-pyrazol-3-one

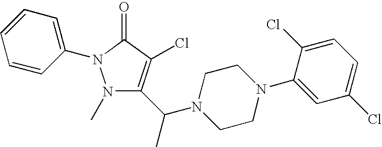

4-Chloro-5-{1-[4-(2,5-dichloro-phenyl-piperazin-1-yl]-ethyl}-1-methyl-2-phenyl-1,2-dihydro-pyrazol-3-one was obtained from 1-(2,5-dichloro-phenyl)-piperazine (0.20 mmol, 0.061 g), 5-(1-bromo-ethyl)-4-chloro-1-methyl-2-phenyl-1,2-dihydro-pyrazol-3-one (0.128 mmol, 0.040 g) and potassium carbonate (0.663 mmol, 0.0916 g) in acetonitrile (3 mL) as a light yellow solid (0.0593 g, 98%). ¹H NMR (300 MHz, CDCl₃): δ (ppm) 7.26-7.53 (m, 6H), 6.96-7.02 (m, 2H) 3.86-3.93 (q, 1H), 3.38 (s, 3H), 3.11 (s, 4H), 2.75 (dd, 4H), 1.54 (d, 3H).

### Example 306

### 4-Chloro-5-[4-(2,5-dichloro-phenyl)piperazine-1-ylmethyl]-1-methyl-2-phenyl-1,2-dihydro-pyrazol-3-one

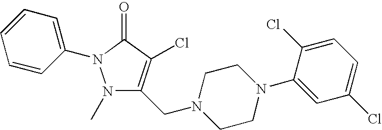

4-Chloro-5-[4-(2,5-dichloro-phenyl)piperazine-1-ylmethyl]-1-methyl-2-phenyl-1,2-dihydro-pyrazol-3-one was obtained from 1-(2,5-dichloro-phenyl)-piperazine (0.20 mmol, 0.061 g), 5-bromoethyl-4-chloro-1-methyl-2-phenyl-1,2-dihydro-pyrazol-3-one (0.133 mmol, 0.040 g) and potassium carbonate (0.663 mmol, 0.0916 g) in acetonitrile (3 mL) as a light yellow solid (0.0588 g, 98%). ¹H NMR (300 MHz, CDCl₃): δ (ppm) 7.31-7.52 (m, 6H), 6.96-7.02 (m, 2H), 3.72 (s, 2H), 3.25 (s, 3H), 3.11 (s, 4H), 2.79 (s, 4H).

### Example 307

### 4-Chloro-5-{1-[4-(5-chloro-2-methoxy-phenyl)-piperazin-1-yl]-ethyl}-2-(4-fluoro-phenyl)-1-methyl-1,2-dihydro-pyrazol-3-one

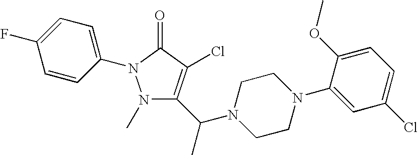

4-Chloro-5-{1-[4-(5-chloro-2-methoxy-phenyl)-piperazin-1-yl]-ethyl}-2-(4-fluoro-phenyl)-1-methyl-1,2-dihydro-pyrazol-3-one was obtained from 1-(5-chloro-2-methoxy-phenyl)-piperazine hydrochloride (0.14 mmol, 0.037 g), 5-(1-bromo-ethyl)-4-chloro-2(4-fluoro-phenyl)-1-methyl-1,2-dihydro-pyrazol-3-one (0.09 mmol, 0.030 g) and potassium carbonate (0.45 mmol, 0.062 g) in acetonitrile (3 mL) as a white foam (0.0405 g, 89%). ¹H NMR (300 MHz, CDCl₃): δ (ppm) 7.33-7.38 (m, 2H), 7.19 (t, 2H), 6.97 (dd, 1H), 6.88 (d, 1H), 6.75 (d, 1H), 4.13 (q, 1H), 3.86 (s, 3H), 3.34 (s, 3H), 3.11 (s, 4H), 2.84 (s, 2H), 2.69 (s, 2H), 1.52 (d, 3H).

### Example 308

### 4-Chloro-5-{1-[4-(5-chloro-2-methyl-phenyl)-piperazin-1-yl]-ethyl}-2-(4-fluoro-phenyl)-1-methyl-1,2-dihydro-pyrazol-3-one

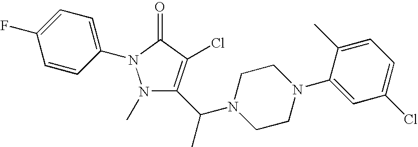

4-Chloro-5-{1-[4-(5-chloro-2-methyl-phenyl)-piperazin-1-yl]-ethyl}-2-(4-fluoro-phenyl)-1-methyl-1,2-dihydro-pyrazol-3-one was obtained from 1-(5-chloro-2-methyl-phenyl)-piperazine (0.14 mmol, 0.030 g), 5-(1-bromo-ethyl)-4-chloro-2(4-fluoro-phenyl)-1-methyl-1,2-dihydro-pyrazol-3-one (0.09 mmol, 0.030 g) and potassium carbonate (0.45 mmol, 0.062 g) in acetonitrile (3 mL) as a white foam (0.0432 g, 99%). ¹H NMR (300 MHz, CDCl₃): δ (ppm) 7.34-7.38 (m, 2H), 7.10-7.22 (m, 3H), 6.96-6.98 (m, 2H), 3.88 (q, 1H), 3.36 (s, 3H), 2.65-2.94 (m, 8H), 2.28 (s, 3H), 1.53 (d, 3H).

### Example 309

### 4-Chloro-5-{1-[4-(5-chloro-2-methoxy-phenyl)-3,6-dihydro-2H-pyridin-1-yl]-ethyl}-2-(4-fluoro-phenyl)-1-methyl-1,2-dihydro-pyrazol-3-one

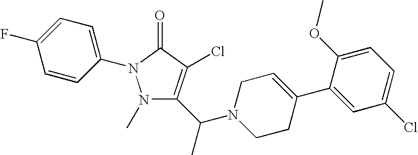

4-Chloro-5-{1-[4-(5-chloro-2-methoxy-phenyl)-3,6-dihydro-2H-pyridin-1-yl]-ethyl}-2-(4-fluoro-phenyl)-1-methyl-1,2-dihydro-pyrazol-3-one was obtained from 4-(5-chloro-2-methoxy-phenyl)-1,2,3,6-tetrahydro-pyridine (0.29 mmol, 0.065 g), 5-(1-bromo-ethyl)-4-chloro-2(4-fluoro-phenyl)-1-methyl-1,2-dihydro-pyrazol-3-one (0.19 mmol, 0.063 g) and potassium carbonate (0.95 mmol, 0.131 g) in acetonitrile (5 mL) as a brown oil (0.0529 g, 58%). ¹H NMR (300 MHz, CDCl₃): δ (ppm) 7.34-7.41 (m, 2H), 7.15-7.21 (m, 4H), 6.89 (m, 1H), 5.86 (s, 1H), 3.93 (q, 1H), 3.79 (S, 3H), 3.36-3.42 (m, 3H), 3.15-3.22 (m, 2H), 2.54-2.82 (m, 4H), 2.14 (d, 3H).

### Example 310

### 4-Chloro-5-{1-[4-(5-chloro-2-methyl-phenyl)-3,6-dihydro-2H-pyridin-1-yl]-ethyl}-2-(4-fluoro-phenyl)-1-methyl-1,2-dihydro-pyrazol-3-one

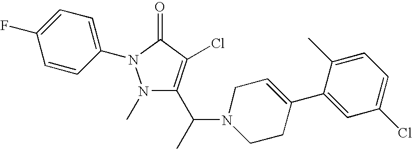

4-Chloro-5-{1-[4-(5-chloro-2-methyl-phenyl)-3,6-dihydro-2H-pyridin-1-yl]-ethyl}-2-(4-fluoro-phenyl)-1-methyl-1,2-dihydro-pyrazol-3-one was obtained from 4-(5-chloro-2-methyl-phenyl)-1,2,3,6-tetrahydro-pyridine (0.15 mmol, 0.110 g), 5-(1-bromo-ethyl)-4-chloro-2-(4-fluoro-phenyl)-1-methyl-1,2-dihydro-pyrazol-3-one (0.10 mmol, 0.040 g) and potassium carbonate (0.50 mmol, 0.069 g) in acetonitrile (3 mL) as a brown oil (0.0529 g, 91%). ¹H NMR (300 MHz, CDCl₃): δ (ppm) 7.35-7.39 (m, 4H), 7.10-7.22 (m, 3H), 5.58 (s, 1H), 3.96 (q, 1H), 3.38 (s, 3H), 3.10-3.23 (m, 2H), 2.77-2.86 (m, 2H), 2.32-2.41 (m, 2H), 2.31 (s, 3H), 1.56 (d, 3H).

### Example 311

### 4-Chloro-5-{1-[4-(5-chloro-2-methyl-phenyl)-3,6-dihydro-2H-pyridin-1-yl]-ethyl}-1-methyl-2-(4-trifluoromethoxy-phenyl)-1,2-dihydro-pyrazol-3-one

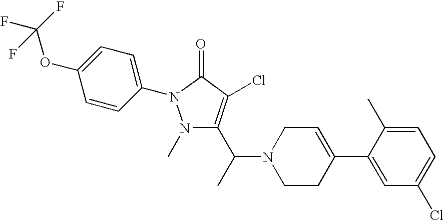

4-Chloro-5-{1-[4-(5-chloro-2-methyl-phenyl)-3,6-dihydro-2H-pyridin-1-yl]-ethyl}-1-methyl-2-(4-trifluoromethoxy-phenyl)-1,2-dihydro-pyrazol-3-one was obtained from 4-(5-chloro-2-methyl-phenyl)-1,2,3,6-tetrahydro-pyridine (0.153 mmol, 0.110 g), 5-(1-bromo-ethyl)-4-chloro-1-methyl-2-(4-trifluoromethoxy-phenyl)-1,2-dihydro-pyrazol-3-one (0.10 mmol, 0.040 g) and potassium carbonate (0.102 mmol, 0.0408 g) in acetonitrile (3 mL) as a yellow oil (0.0196 g, 37%). ¹H NMR (300 MHz, CDCl₃): δ (ppm) 7.34-7.51 (m, 4H), 7.10-7.14 (m, 3H), 5.60 (s, 1H), 3.97 (q, 1H), 3.41 (s, 3H), 3.22-3.28 (m, 2H), 2.77-2.85 (m, 2H), 2.33-2.50 (m, 2H), 2.27 (s, 3H), 1.57 (d, 3H).

### Example 312

### 4-Chloro-5-{1-[4-(5-chloro-2-methoxy-phenyl)-3,6-dihydro-2H-pyridin-1-yl]-ethyl}-1-methyl-2-(4-trifluoromethoxy-phenyl)-1,2-dihydro-pyrazol-3-one

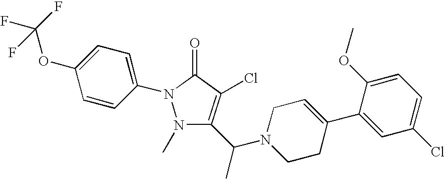

4-Chloro-5-{1-[4-(5-chloro-2-methoxy-phenyl)-3,6-dihydro-2H-pyridin-1-yl]-ethyl}-1-methyl-2-(4-trifluoromethoxy-phenyl)-1,2-dihydro-pyrazol-3-one was obtained from 4-(5-chloro-2-methoxy-phenyl)-1,2,3,6-tetrahydro-pyridine (0.167 mmol, 0.120 g), 5-(1-bromo-ethyl)-4-chloro-1-methyl-2-(4-trifluoromethoxy-phenyl)-1,2-dihydro-pyrazol-3-one (0.11 mmol, 0.044 g) and potassium carbonate (0.55 mmol, 0.076 g) in acetonitrile (3 mL) as a yellow oil (0.0348 g, 58%). ¹H NMR (300 MHz, CDCl₃): δ (ppm) 7.32-7.50 (m, 4H), 7.16-7.22 (m, 2H), 6.81 (d, 1H), 5.87 (s, 1H), 3.95 (q, 1H), 3.81 (s, 3H), 3.41 (s, 3H), 2.98-3.22 (m, 2H), 2.50-2.85 (m, 4H), 1.53 (d, 3H).

### Example 313

### 4-Chloro-5-[4-(5-chloro-2-methoxy-phenyl)-3,6-dihydro-2H-pyridin-1-yl-ethyl]-1-methyl-2-(4-trifluoromethoxy-phenyl)-1,2-dihydro-pyrazol-3-one

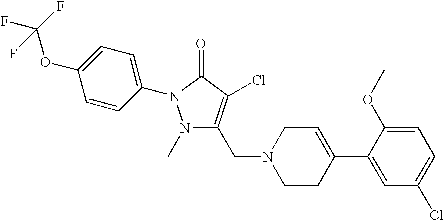

4-Chloro-5-[4-(5-chloro-2-methoxy-phenyl)-3,6-dihydro-2H-pyridin-1-yl-ethyl]-1-methyl-2-(4-trifluoromethoxy-phenyl)-1,2-dihydro-pyrazol-3-one was obtained from 4-(5-chloro-2-methoxy-phenyl)-1,2,3,6-tetrahydro-pyridine (0.167 mmol, 0.120 g), 5-bromomethyl-4-chloro-1-methyl-2-(4-trifluoromethoxy-phenyl)-1,2-dihydro-pyrazol-3-one (0.11 mmol, 0.043 g) and potassium carbonate (0.55 mmol, 0.076 g) in acetonitrile (3 mL) as a yellow oil (0.0459 g, 79%). ¹H NMR (300 MHz, CDCl₃): δ (ppm) 7.33-7.50 (m, 4H), 7.17-7.23 (m, 2H), 6.81 (d, 1H), 5.85 (s, 1H), 3.81 (s, 3H), 3.71 (s, 2H), 3.28-3.30 (m, 5H), 2.82 (t, 2H), 2.56 (s, 2H).

### Example 314

### 4-Chloro-1-methyl-2-phenyl-5-{1-[4-(3-phenyl-propyl)-piperidin-1-yl]-ethyl}-1,2-dihydro-pyrazol-3-one

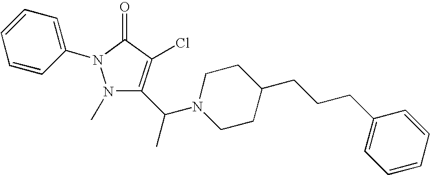

4-Chloro-1-methyl-2-phenyl-5-{1-[4-(3-phenyl-propyl)-piperidin-1-yl]-ethyl}-1,2-dihydro-pyrazol-3-one was synthesized from 5-(1-bromoethyl)-4-chloro-1-methyl-2-phenyl-1,2-dihydro-pyrazol-3-one (30.0 mg, 0.088 mmol), 4-(3-phenyl-propyl)-piperidine (26.9 mg, 0.132 mmol) and potassium carbonate (36.6 mg, 0.27 mmol) in 3 mL of acetonitrile. The desired product was isolated by eluting the crude though a 2 g SPE tube in a solution of 20% acetone and hexanes to yield a yellow oil (32.6 mg, 84.5%) ¹H NMR (300 MHz, CDCl₃): δ ppm 1.189-1.32 (m, 5H), 1.46 (d, 3H), 1.64-1.75 (m, 4H), 1.89-2.02 (quintet, 2H), 2.59 (t, 2H) 2.62 (d of d, 2H), 3.34 (s, 3H), 7.18-7.22 (m, 3H), 7.28-7.46 (m, 5H), 7.49-7.52 (m, 2H).

### Example 315

### 4-Chloro-2-(4-fluoro-phenyl)-5-{4-[3-(4-fluoro-phenyl)-allyl]-piperidin-1-ylmethyl}-1-methyl-1,2-dihydro-pyrazol-3-one

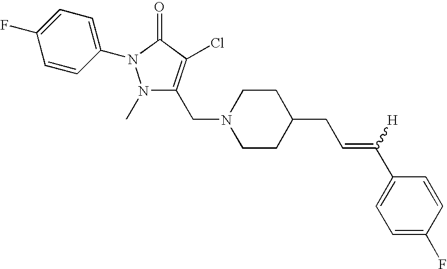

4-Chloro-2-(4-fluoro-phenyl)-5-{4-[3-(4-fluoro-phenyl)-allyl]-piperidin-1-ylmethyl}-1-methyl-1,2-dihydro-pyrazol-3-one was obtained from 4-[3-(4-fluoro-phenyl)-allyl]-piperidine (0.156 mmol, 0.034 g), 5-bromomethyl-4-chloro-2-(4-fluoro-phenyl)-1-methyl-1,2-dihydro-pyrazol-3-one (0.104 mmol, 0.033 g) and potassium carbonate (0.52 mmol, 0.072 g) in acetonitrile (3 mL) as a white solid [0.0364 g, 76% (cis: trans=3:1)]. ¹H NMR (300 MHz, CDCl₃): δ (ppm) 7.29-7.40 (m, 4H), 7.15-7.21 (m, 2H), 7.00-7.03 (m, 2H), 6.33-6.42 (m, 1H), 6.03-6.19 and 5.60-5.63 (m 1H), 3.52-3.54 (m, 2H), 3.19-3.21 (m, 3H), 2.89-2.93 (m, 2H), 2.06-2.29 (m, 4H), 1.70-1.79 (m, 2H), 1.25-1.35 (m, 3H).

### Example 316

### cis-4-Chloro-2-(4-fluoro-phenyl)-5-(1-{4-[3-(4-fluoro-phenyl)-allyl]-piperidin-1-yl}-ethyl)-1-methyl-1,2-dihydro-pyrazol-3-one

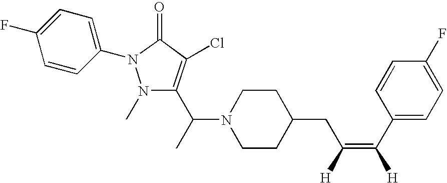

cis-4-Chloro-2-(4-fluoro-phenyl)-5-(1-{4-[3-(4-fluoro-phenyl)-allyl]-piperidin-1-yl}-ethyl)-1-methyl-1,2-dihydro-pyrazol-3-one was obtained from 4-[3-(4-fluoro-phenyl)-allyl]-piperidine (0.156 mmol, 0.034 g), 5-(1-bromo-ethyl)-4-chloro-2-(4-fluoro-phenyl)-1-methyl-1,2-dihydro-pyrazol-3-one (0.104 mmol, 0.035 g) and potassium carbonate (0.52 mmol, 0.072 g) in acetonitrile (3 mL) as a yellow oil [0.005 g, 11% (100% cis)]. ¹H NMR (300 MHz, CDCl₃): δ (ppm) 7.31-7.35 (m, 4H), 7.15-7.22 (m, 2H), 7.00-7.03 (m, 2H), 6.43-6.47 (m, 1H), 5.62-5.69 (m 1H), 3.76 (q, 1H), 3.35 (s, 3H), 3.16-3.19 (m, 1H), 2.82-2.91 (m, 1H), 1.99-2.25 (m, 4H), 1.68-1.82 (m, 2H), 1.47 (d, 3H), 1.24-1.30 (m, 3H).

### Example 317

### cis-4-Chloro-5-{4-[3-(4-fluoro-phenyl)-allyl]-piperidin-1-ylmethyl}-1-methyl-2-(4-trifluoromethoxy-phenyl)-1,2-dihydro-pyrazol-3-one

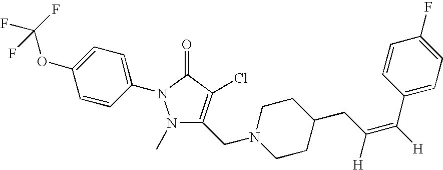

cis-4-Chloro-5-{4-[3-(4-fluoro-phenyl)-allyl]-piperidin-1-ylmethyl}-1-methyl-2-(4-trifluoromethoxy-phenyl)-1,2-dihydro-pyrazol-3-one was obtained from 4-[3-(4-fluoro-phenyl)-allyl]-piperidine (0.156 mmol, 0.034 g), 5-bromomethyl-4-chloro-1-methyl-2-(4-trifluoromethoxy-phenyl)-1,2-dihydro-pyrazol-3-one (0.104 mmol, 0.040 g) and potassium carbonate (0.52 mmol, 0.072 g) in acetonitrile (3 mL) as a white solid (0.004 g, 9%, 100% cis). ¹H NMR (300 MHz, CDCl₃): δ (ppm) 7.43-7.47 (m, 2H), 7.22-7.35 (m, 4H), 6.98-7.07 (m, 2H), 6.44-6.48 (m, 1H), 5.63-5.72 (m 1H), 3.54-3.55 (s, 2H), 3.21 (s, 3H), 2.90-2.93 (m, 2H), 2.10-2.29 (m, 4H), 1.75-1.79 (m, 2H), 1.22-1.30 (m, 3H).

### Example 318

### 4-Chloro-5-(1-{4-[3-(4-fluoro-phenyl)-allyl]-piperidin-1-yl}-ethyl)-1-methyl-2-(4-trifluoromethoxy-phenyl)-1,2-dihydro-pyrazol-3-one

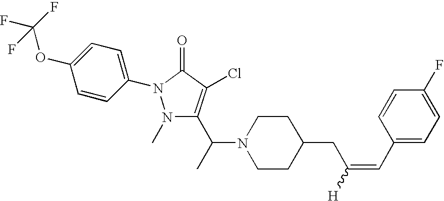

4-Chloro-5-(1-{4-[3-(4-fluoro-phenyl)-allyl]-piperidin-1-yl}-ethyl)-1-methyl-2-(4-trifluoromethoxy-phenyl)-1,2-dihydro-pyrazol-3-one was obtained from 4-[3-(4-fluoro-phenyl)-allyl]-piperidine (0.156 mmol, 0.042 g), 5-(1-bromo-ethyl)-4-chloro-1-methyl-2-(4-trifluoromethoxy-phenyl)-1,2-dihydro-pyrazol-3-one (0.104 mmol, 0.042 g) and potassium carbonate (0.52 mmol, 0.072 g) in acetonitrile (3 mL) as a yellow oil [0.051 g, 91%, (cis:trans=3:7)]. ¹H NMR (300 MHz, CDCl₃): δ (ppm) 7.16-7.47 (m, 6H), 6.97-7.05 (m, 2H), 6.33-6.43 (m, 1H), 6.08-6.18 and 5.62-5.69 (m 1H), 3.78 (m, 1H), 3.32-3.39 (m, 3H), 3.08-3.19 (m, 1H), 2.82-2.91 (m, 1H), 1.98-2.29 (m, 4H), 1.68-1.88 (m, 2H), 1.44-1.49 (m, 3H), 1.24-1.30 (m, 3H).

### Example 319

### 4-Chloro-2-(4-fluoro-phenyl)-5-{4-[3-(4-fluoro-phenyl)-propyl]-piperidin-1-ylmethyl}-1-methyl-1,2-dihydro-pyrazol-3-one

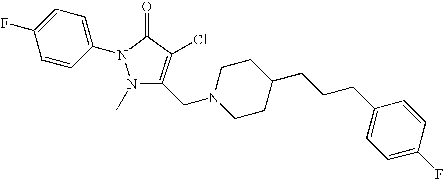

4-Chloro-2-(4-fluoro-phenyl)-5-{4-[3-(4-fluoro-phenyl)-propyl]-piperidin-1-ylmethyl}-1-methyl-1,2-dihydro-pyrazol-3-one was obtained from 4-[3-(4-fluoro-phenyl)-propyl]-piperidine (0.195 mmol, 0.053 g), 5-bromomethyl-4-chloro-2-(4-fluoro-phenyl)-1-methyl-1,2-dihydro-pyrazol-3-one (0.13 mmol, 0.042 g) and potassium carbonate (0.65 mmol, 0.090 g) in acetonitrile (3 mL) as a white solid (0.0364 g, 78%). ¹H NMR (300 MHz, CDCl₃): δ (ppm) 7.34-7.39 (m, 2H), 7.11-7.21 (m, 4H), 6.94-6.99 (m, 2H), 3.52 (s, 2H), 3.20 (s, 3H), 2.88-2.92 (m, 2H), 2.58 (t, 2H), 2.06-2.14 (m, 2H), 1.60-1.73 (m, 4H), 1.19-1.27 (m, 5H).

### Example 320

### 4-Chloro-2-(4-fluoro-phenyl)-5-(1-{4-[3-(4-fluoro-phenyl)-propyl]-piperidin-1-yl}-ethyl)-1-methyl-1,2-dihydro-pyrazol-3-one

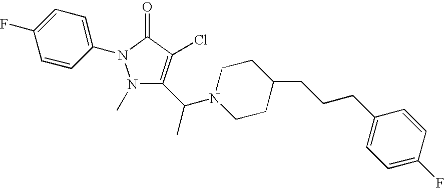

4-Chloro-2-(4-fluoro-phenyl)-5-(1-{4-[3-(4-fluoro-phenyl)-propyl]-piperidin-1-yl}-ethyl)-1-methyl-1,2-dihydro-pyrazol-3-one was obtained from 4-[3-(4-fluoro-phenyl)-propyl]-piperidine (0.195 mmol, 0.053 g), 5-(1-bromo-ethyl)-4-chloro-2-(4-fluoro-phenyl)-1-methyl-1,2-dihydro-pyrazol-3-one (0.13 mmol, 0.044 g) and potassium carbonate (0.65 mmol, 0.090 g) in acetonitrile (3 mL) as a yellow oil (0.0525 g, 85%). ¹H NMR (300 MHz, CDCl₃): δ (ppm) 7.32-7.36 (m, 2H), 7.10-7.20 (m, 4H), 6.94-6.99 (m, 2H), 3.77 (q, 1H), 3.32 (s, 3H), 3.08-3.18 (m, 1H), 2.89-2.92 (m, 1H), 2.57 (t, 2H), 1.93-2.19 (m, 2H), 1.55-1.80 (m, 4H), 1.45 (d, 3H), 1.09-1.29 (m, 5H).

### Example 321

### 4-Chloro-5-{4-[3-(4-fluoro-phenyl)-propyl]-piperidin-1-ylmethyl}-methyl-2-(4-trifluoromethoxy-phenyl)-1,2-dihydro-pyrazol-3-one

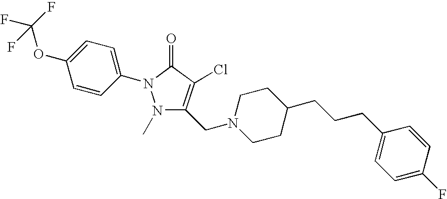

4-Chloro-5-{4-[3-(4-fluoro-phenyl)-propyl]-piperidin-1-ylmethyl}-1-methyl-2-(4-trifluoromethoxy-phenyl)-1,2-dihydro-pyrazol-3-one was obtained from 4-[3-(4-fluoro-phenyl)-propyl]-piperidine (0.195 mmol, 0.053 g), 5-bromomethyl-4-chloro-1-methyl-2-(4-trifluoromethoxy-phenyl)-1,2-dihydro-pyrazol-3-one (0.13 mmol, 0.050 g) and potassium carbonate (0.65 mmol, 0.090 g) in acetonitrile (3 mL) as a yellow oil (0.0455 g, 67%). ¹H NMR (300 MHz, CDCl₃): δ (ppm) 7.43-7.46 (m, 2H), 7.32-7.35 (m, 2H), 7.11-7.16 (m, 2H), 6.94-7.00 (m, 2H), 3.53 (s, 2H), 3.22 (s, 3H), 2.88-2.92 (m, 2H), 2.56 (t, 2H), 2.06-2.15 (m, 2H), 1.60-1.73 (m, 4H), 1.19-1.30 (m, 5H).

### Example 322

### 4-Chloro-5-(1-{4-[3-(4-fluoro-phenyl)-propyl]-piperidin-1-yl}-ethyl)-1-methyl-2-(4-trifluoromethoxy-phenyl)-1,2-dihydro-pyrazol-3-one

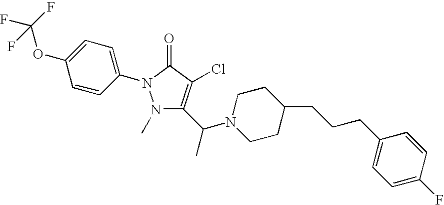

4-Chloro-5-(1-{4-[3-(4-fluoro-phenyl)-propyl]-piperidin-1-yl}-ethyl)-1-methyl-2-(4-trifluoromethoxy-phenyl)-1,2-dihydro-pyrazol-3-one was obtained from 4-[3-(4-fluoro-phenyl)-allyl]-piperidine (0.195 mmol, 0.053 g), 5-(1-bromo-ethyl)-4-chloro-1-methyl-2-(4-trifluoromethoxy-phenyl)-1,2-dihydro-pyrazol-3-one (0.13 mmol, 0.051 g) and potassium carbonate (0.65 mmol, 0.090 g) in acetonitrile (3 mL) as a yellow oil (0.0658 g, 94%). ¹H NMR (300 MHz, CDCl₃): δ (ppm) 7.32-7.44 (m, 4H), 7.11-7.16 (m, 2H), 6.95-7.00 (m, 2H), 3.79 (q, 1H), 3.36 (s, 3H), 3.11-3.19 (m, 1H), 2.86-2.93 (m, 1H), 2.58 (t, 2H), 2.00-2.14 (m, 2H), 1.58-1.88 (m, 4H), 1.47 (d, 3H), 1.17-1.30 (m, 5H).

### Example 323

### 4-Chloro-5-{4-[2-(4-fluoro-phenoxy)-ethyl]-piperidin-1-ylmethyl}-2-(4-fluoro-phenyl)-1-methyl-1,2-dihydro-pyrazol-3-one

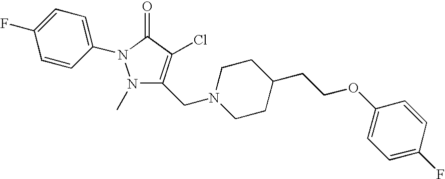

4-Chloro-5-{4-[2-(4-fluoro-phenoxy)-ethyl]-piperidin-1-ylmethyl}-2-(4-fluoro-phenyl)-1-methyl-1,2-dihydro-pyrazol-3-one was obtained from 4-[2-(4-fluoro-phenoxy)-ethyl]-piperidine (0.198 mmol, 0.056 g), 5-bromomethyl-4-chloro-2-(4-fluoro-phenyl)-1-methyl-1,2-dihydro-pyrazol-3-one (0.13 mmol, 0.042 g) and potassium carbonate (0.65 mmol, 0.090 g) in acetonitrile (3 mL) as a white solid (0.0332 g, 54%). ¹H NMR (300 MHz, CDCl₃): δ (ppm) 7.35-7.40 (m, 2H), 7.15-7.21 (m, 2H), 6.95-7.01 (m, 2H), 6.82-6.86 (m 2H), 3.98 (t, 2H), 3.54 (s, 2H), 3.21 (s, 3H), 2.91-2.95 (m, 2H), 2.11-2.19 (m 2H), 1.70-1.80 (m, 4H), 1.58-1.69 (m, 1H), 1.20-1.37 (m, 2H).

### Example 324

### 4-Chloro-5-(1-{4-[2-(4-fluoro-phenoxy)-ethyl]-piperidin-1-yl}-ethyl)-2-(4-fluoro-phenyl)-ethyl)-1-methyl-1,2-dihydro-pyrazol-3-one

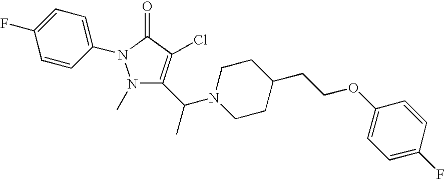

4-Chloro-5-(1-{4-[2-(4-fluoro-phenoxy)-ethyl]-piperidin-1-yl}-ethyl)-2-(4-fluoro-phenyl)-ethyl)-1-methyl-1,2-dihydro-pyrazol-3-one was obtained from 4-[2-(4-fluoro-phenoxy)-ethyl]-piperidine (0.198 mmol, 0.056 g), 5-(1-bromo-ethyl)-4-chloro-2-(4-fluoro-phenyl)-1-methyl-1,2-dihydro-pyrazol-3-one (0.13 mmol, 0.044 g) and potassium carbonate (0.65 mmol, 0.090 g) in acetonitrile (3 mL) as a yellow oil (0.0525 g, 85%). ¹H NMR (300 MHz, CDCl₃): δ (ppm) 7.32-7.37 (m, 2H), 7.15-7.21 (m, 2H), 6.98-7.01 (m, 2H), 6.81-6.86 (m 2H), 3.97 (t, 2H), 3.77 (q, 1H), 3.33 (s, 3H), 3.08-3.18 (m, 1H), 2.89-2.92 (m, 1H), 2.06-2.11 (m 2H), 1.65-1.85 (m, 4H), 1.55-1.63 (m, 1H), 1.47 (d, 3H), 1.15-1.37 (m, 2H).

### Example 325

### 4-Chloro-5-{4-[2-(4-fluoro-phenoxy)-ethyl]-piperidin-1-ylmethyl}-1-methyl-2-(4-trifluoromethoxy-phenyl)-1,2-dihydro-pyrazol-3-one

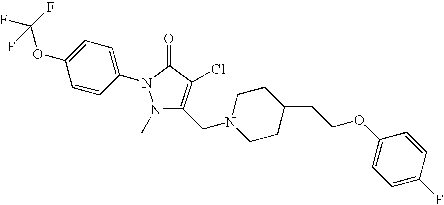

4-Chloro-5-{4-[2-(4-fluoro-phenoxy)-ethyl]-piperidin-1-ylmethyl}-1-methyl-2-(4-trifluoromethoxy-phenyl)-1,2-dihydro-pyrazol-3-one was obtained from 4-[2-(4-fluoro-phenoxy)-ethyl]-piperidine (0.198 mmol, 0.056 g), 5-bromomethyl-4-chloro-1-methyl-2-(4-trifluoromethoxy-phenyl)-1,2-dihydro-pyrazol-3-one (0.13 mmol, 0.050 g) and potassium carbonate (0.65 mmol, 0.090 g) in acetonitrile (3 mL) as a white solid (0.0398 g, 56%). ¹H NMR (300 MHz, CDCl₃): δ (ppm) 7.44-7.47 (m, 2H), 7.33-7.36 (m, 2H), 6.96-7.01 (m, 2H), 6.82-6.86 (m 2H), 3.98 (t, 2H), 3.55 (s, 2H), 3.25 (s, 3H), 2.58 (t, 2H), 2.13-2.20 (m, 2H), 1.72-1.86 (m, 4H), 1.50-1.67 (m, 1H), 1.27-1.38 (m, 2H).

### Example 326

### 4-Chloro-5-(1-{4-[2-(4-fluoro-phenoxy)-ethyl]-piperidin-1-yl}-ethyl)-1-methyl-2-(4-trifluoromethoxy-phenyl)-1,2-dihydro-pyrazol-3-one

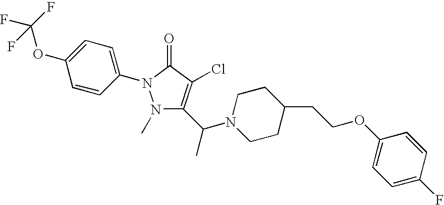

4-Chloro-5-(1-{4-[2-(4-fluoro-phenoxy)-ethyl]-piperidin-1-yl}-ethyl)-1-methyl-2-(4-trifluoromethoxy-phenyl)-1,2-dihydro-pyrazol-3-one was obtained from 4-[2-(4-fluoro-phenoxy)-ethyl]-piperidine (0.198 mmol, 0.056 g), 5-(1-bromo-ethyl)-4-chloro-1-methyl-2-(4-trifluoromethoxy-phenyl)-1,2-dihydro-pyrazol-3-one (0.13 mmol, 0.051 g) and potassium carbonate (0.65 mmol, 0.090 g) in acetonitrile (3 mL) as a yellow oil (0.0512 g, 73%). ¹H NMR (300 MHz, CDCl₃): δ (ppm) 7.40-7.44 (m, 2H), 7.32-7.36 (m, 2H), 6.96-7.01 (m, 2H), 6.82-6.86 (m 2H), 3.98 (t, 2H), 3.78 (q, 1H), 3.35 (s, 3H), 3.11-3.19 (m, 1H), 2.86-2.93 (m, 1H), 1.98-2.20 (m, 2H), 1.65-1.90 (m, 4H), 1.55-1.65 (m, 1H), 1.46 (d, 3H), 1.19-1.39 (m, 2H).

### Example 327

### 4-Chloro-5-{4-[2-(4-chloro-phenoxy)-ethyl]-piperidin-1-ylmethyl}-2-(4-fluoro-phenyl)-1-methyl-1,2-dihydro-pyrazol-3-one

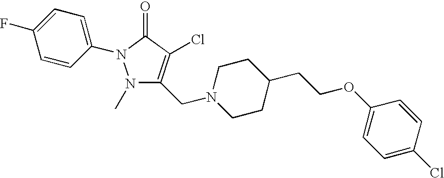

4-Chloro-5-{4-[2-(4-chloro-phenoxy)-ethyl]-piperidin-1-ylmethyl}-2-(4-fluoro-phenyl)-1-methyl-1,2-dihydro-pyrazol-3-one was obtained from 4-[2-(4-chloro-phenoxy)-ethyl]-piperidine (0.185 mmol, 0.044 g), 5-bromomethyl-4-chloro-2-(4-fluoro-phenyl)-1-methyl-1,2-dihydro-pyrazol-3-one (0.124 mmol, 0.041 g) and potassium carbonate (0.62 mmol, 0.085 g) in acetonitrile (3 mL) as a yellow oil (0.0508 g, 86%). ¹H NMR (300 MHz, CDCl₃): δ (ppm) 7.43-7.47 (m, 2H), 7.33-7.36 (m, 2H), 7.22-7.26 (m, 2H), 6.82-6.86 (m, 2H), 3.99 (t, 2H), 3.55 (s, 2H), 3.23 (s, 3H), 2.92-2.95 (m, 2H), 2.13-2.19 (m 2H), 1.72-1.80 (m, 4H), 1.58-1.69 (m, 1H), 1.21-1.39 (m, 2H).

### Example 328

### 4-Chloro-5-(1-{4-[2-(4-chloro-phenoxy)-ethyl]-piperidin-1-yl}-ethyl)-2-(4-fluoro-phenyl)-ethyl)-1-methyl-1,2-dihydro-pyrazol-3-one

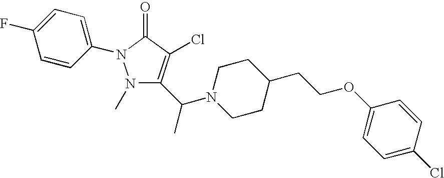

4-Chloro-5-(1-{4-[2-(4-chloro-phenoxy)-ethyl]-piperidin-1-yl}-ethyl-2-(4-fluoro-phenyl)-ethyl)-1-methyl-1,2-dihydro-pyrazol-3-one was obtained from 4-[2-(4-chloro-phenoxy)-ethyl]-piperidine (0.185 mmol, 0.044 g), 5-(1-bromo-ethyl)-4-chloro-2-(4-fluoro-phenyl)-1-methyl-1,2-dihydro-pyrazol-3-one (0.124 mmol, 0.040 g) and potassium carbonate (0.62 mmol, 0.085 g) in acetonitrile (3 mL) as a white solid (0.0525 g, 85%). ¹H NMR (300 MHz, CDCl₃): δ (ppm) 7.32-7.37 (m, 2H), 7.15-7.25 (m, 4H), 6.81-6.84 (m 2H), 3.98 (t, 2H), 3.76 (q, 1H), 3.33 (s, 3H), 3.08-3.16 (m, 1H), 2.89-2.92 (m, 1H), 2.03-2.11 (m 2H), 1.68-1.88 (m, 4H), 1.55-1.70 (m, 1H), 1.47 (d, 3H), 1.19-1.37 (m, 2H).

### Example 329

### 4-Chloro-5-(1-{4-[2-(4-chloro-phenoxy)-ethyl]-piperidin-1-yl}-ethyl)-1-methyl-2-(4-trifluoromethoxy-phenyl)-1,2-dihydro-pyrazol-3-one

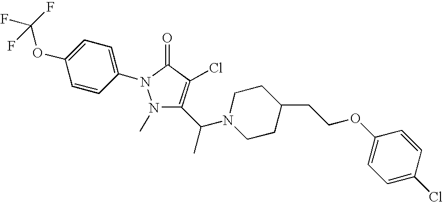

4-Chloro-5-(1-{4-[2-(4-chloro-phenoxy)-ethyl]-piperidin-1-yl}-ethyl)-1-methyl-2-(4-trifluoromethoxy-phenyl)-1,2-dihydro-pyrazol-3-one was obtained from 4-[2-(4-chloro-phenoxy)-ethyl]-piperidine (0.185 mmol, 0.044 g), 5-(1-bromo-ethyl)-4-chloro-1-methyl-2-(4-trifluoromethoxy-phenyl)-1,2-dihydro-pyrazol-3-one (0.124 mmol, 0.0496 g) and potassium carbonate (0.62 mmol, 0.085 g) in acetonitrile (3 mL) as a yellow oil (0.0653 g, 94%). ¹H NMR (300 MHz, CDCl₃): δ (ppm) 7.40-7.44 (m, 2H), 7.22-7.35 (m, 4H), 6.76-6.84 (m 2H), 3.98 (t, 2H), 3.78 (q, 1H), 3.35 (s, 3H), 3.16-3.22 (m, 1H), 2.88-2.92 (m, 1H), 1.98-2.20 (m, 2H), 1.63-1.90 (m, 4H), 1.55-1.62 (m, 1H), 1.48 (d, 3H), 1.19-1.39 (m, 2H).

### Example 330

### 4-Chloro-5-(1-{4-[2-(3,4-difluoro-phenoxy)-ethyl]-piperidin-1-yl}-ethyl)-2-(4-fluoro-phenyl)-1-methyl-1,2-dihydro-pyrazol-3-one

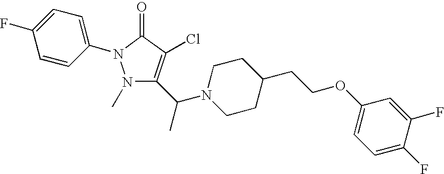

4-Chloro-5-(1-{4-[2-(3,4-difluoro-phenoxy)-ethyl]-piperidin-1-yl}-ethyl-2-(4-fluoro-phenyl)-ethyl)-1-methyl-1,2-dihydro-pyrazol-3-one was obtained from 4-[2-(3,4-fluoro-phenoxy)-ethyl]-piperidine (0.178 mmol, 0.043 g), 5-(1-bromo-ethyl)-4-chloro-2-(4-fluoro-phenyl)-1-methyl-1,2-dihydro-pyrazol-3-one (0.118 mmol, 0.039 g) and potassium carbonate (0.593 mmol, 0.082 g) in acetonitrile (3 mL) as a yellow oil (0.0496 g, 85%). ¹H NMR (300 MHz, CDCl₃): δ (ppm) 7.32-7.37 (m, 2H), 7.05-7.23 (m, 3H), 6.63-6.73 (m, 1H), 6.52-6.60 (m 1H), 3.96 (t, 2H), 3.78 (q, 1H), 3.33 (s, 3H), 3.14-3.17 (m, 1H), 2.88-2.92 (m, 1H), 2.04-2.11 (m 2H), 1.69-1.81 (m, 4H), 1.55-1.63 (m, 1H), 1.47 (d, 3H), 1.23-1.32 (m, 2H).

### Example 331

### 4-Chloro-5-{4-[2-3,(4-difluoro-phenoxy)-ethyl]-piperidin-1-ylmethyl}-1-methyl-2-(4-trifluoromethoxy-phenyl)-1,2-dihydro-pyrazol-3-one

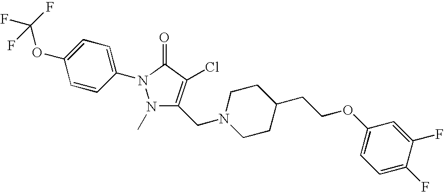

4-Chloro-5-{4-[2-(3,4-difluoro-phenoxy)-ethyl]-piperidin-1-ylmethyl}-1-methyl-2-(4-trifluoromethoxy-phenyl)-1,2-dihydro-pyrazol-3-one was obtained from 4-[2-(3,4-difluoro-phenoxy)-ethyl]-piperidine (0.178 mmol, 0.043 g), 5-bromomethyl-4-chloro-1-methyl-2-(4-trifluoromethoxy-phenyl)-1,2-dihydro-pyrazol-3-one (0.118 mmol, 0.046 g) and potassium carbonate (0.65 mmol, 0.090 g) in acetonitrile (3 mL) as a yellow oil (0.0507 g, 79%). ¹H NMR (300 MHz, CDCl₃): δ (ppm) 7.44-7.47 (m, 2H), 7.33-7.36 (m, 2H), 7.02-7.09 (m, 1H), 6.68-6.72 (m, 1H), 6.52-6.60 (m, 1H), 3.97 (t, 2H), 3.55 (s, 2H), 3.24 (s, 3H), 2.92-2.96 (m, 2H), 2.13-2.20 (m, 2H), 1.68-1.80 (m, 4H), 1.50-1.67 (m, 1H), 1.30-1.35 (m, 2H).

### Example 332

### 4-Chloro-5-(1-{4-[2-(3,4-difluoro-phenoxy)-ethyl]-piperidin-1-yl}-ethyl)-1-methyl-2-(4-trifluoromethoxy-phenyl)-1,2-dihydro-pyrazol-3-one

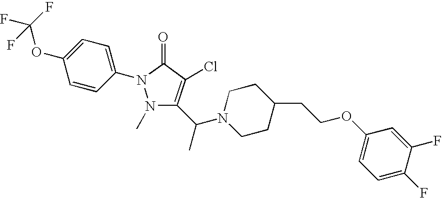

4-Chloro-5-(1-{4-[2-(3,4-difluoro-phenoxy)-ethyl]-piperidin-1-yl}-ethyl)-1-methyl-2-(4-trifluoromethoxy-phenyl)-1,2-dihydro-pyrazol-3-one was obtained from 4-[2-(3,4-difluoro-phenoxy)-ethyl]-piperidine (0.178 mmol, 0.043 g), 5-(1-bromo-ethyl)-4-chloro-1-methyl-2-(4-trifluoromethoxy-phenyl)-1,2-dihydro-pyrazol-3-one (0.118 mmol, 0.047 g) and potassium carbonate (0.593 mmol, 0.082 g) in acetonitrile (3 mL) as a yellow oil (0.0552 g, 83%). ¹H NMR (300 MHz, CDCl₃): δ (ppm) 7.40-7.44 (m, 2H), 7.31-7.35 (m, 2H), 7.01-7.08 (m, 1H), 6.68-6.72 (m, 1H), 6.52-6.60 (m, 1H), 3.96 (t, 2H), 3.78 (q, 1H), 3.36 (s, 3H), 3.11-3.19 (m, 1H), 2.86-2.93 (m, 1H), 1.98-2.20 (m, 2H), 1.65-1.90 (m, 4H), 1.55-1.65 (m, 1H), 1.48 (d, 3H), 1.19-1.39 (m, 2H).

### Example 333

### 4-Chloro-1-methyl-5-{1-[4-(3-pyridin-4-yl-propyl)-piperidin-1-yl]-ethyl}-2-(4-trifluoromethoxy-phenyl)-1,2-dihydro-pyrazol-3-one

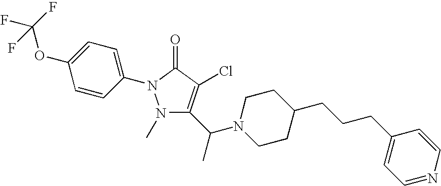

4-Chloro-1-methyl-5-{1-[4-(3-pyridin-4-yl-propyl)-piperidin-1-yl]-ethyl}-2-(4-trifluoromethoxy-phenyl)-1,2-dihydro-pyrazol-3-one was obtained from 4-(3-piperidin-4-yl-propyl)-pyridine (0.197 mmol, 0.040 g), 5-(1-bromo-ethyl)-4-chloro-1-methyl-2-(4-trifluoromethoxy-phenyl)-1,2-dihydro-pyrazol-3-one (0.131 mmol, 0.051 g) and potassium carbonate (0.92 mmol, 0.127 g) in acetonitrile (3 mL) as a yellow oil (0.0261 g, 38%). ¹H NMR (300 MHz, CDCl₃): δ (ppm) 7.65-7.71 (m, 2H), 7.41-7.55 (m, 4H), 7.35-7.40 (m, 2H), 3.77 (q, 1H), 3.34 (s, 3H), 3.13-3.21 (m, 1H), 2.90-2.98 (m, 1H), 2.15 (t, 2H), 1.58-1.88 (m, 5H), 1.47 (d, 3H), 1.25-1.30 (m, 6H).

### Example 334

### 4-Chloro-2-(4-fluoro-phenyl)-1-methyl-5-{1-[4-(3-pyridin-2-yl-propyl)-piperidin-1-yl]-ethyl}-1,2-dihydro-pyrazol-3-one

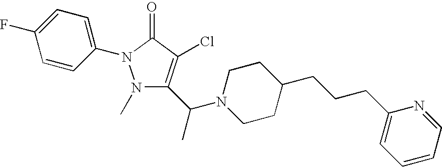

4-Chloro-2-(4-fluoro-phenyl)-1-methyl-5-{1-[4-(3-pyridin-2-yl-propyl)-piperidin-1-yl]-ethyl}-1,2-dihydro-pyrazol-3-one was obtained 2-(3-piperidin-4-yl-propyl)-pyridine (0.231 mmol, 0.047 g), 5-(1-bromo-ethyl)-4-chloro-2-(4-fluoro-phenyl)-1-methyl-1,2-dihydro-pyrazol-3-one (0.154 mmol, 0.051 g) and potassium carbonate (1.23 mmol, 0.170 g) in acetonitrile (3 mL) as a yellow oil (0.0550 g, 78%). ¹H NMR (300 MHz, CDCl₃): δ (ppm) 8.52-8.53 (m, 1H), 7.59-7.61 (m, 1H), 7.31-7.36 (m, 2H), 7.11-7.19 (m, 4H), 3.74 (q, 1H), 3.31 (s, 3H), 3.08-3.12 (m, 1H), 2.89-2.92 (m, 1H), 2.77 (t, 2H), 1.96-2.11 (m, 2H), 1.67-1.80 (m, 4H), 1.44 (d, 3H), 1.09-1.33 (m, 5H).

### Example 335

### 4-Chloro-1-methyl-5-[4-(3-pyridin-2-yl-propyl)-piperidin-1-ylmethyl]-2-(4-trifluoromethoxy-phenyl)-1,2-dihydro-pyrazol-3-one

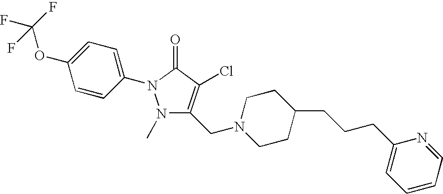

4-Chloro-1-methyl-5-[4-(3-pyridin-2-yl-propyl)-piperidin-1-ylmethyl]-2-(4-trifluoromethoxy-phenyl)-1,2-dihydro-pyrazol-3-one was obtained from 2-(3-piperidin-4-yl-propyl)-pyridine (0.231 mmol, 0.047 g), 5-bromomethyl-4-chloro-1-methyl-2-(4-trifluoromethoxy-phenyl)-1,2-dihydro-pyrazol-3-one (0.154 mmol, 0.059 g) and potassium carbonate (0.123 mmol, 0.170 g) in acetonitrile (3 mL) as a yellow oil (0.073 g, 93%). ¹H NMR (300 MHz, CDCl₃): δ (ppm) 8.53-8.55 (m, 1H), 7.58-7.64 (m, 1H), 7.42-7.46 (m, 2H), 7.32-7.35 (m, 2H), 7.12-7.17 (m, 2H), 3.53 (s, 2H), 3.22 (s, 3H), 2.88-2.92 (m, 2H), 2.79 (t, 2H), 2.07-2.14 (m, 2H), 1.72-1.77 (m, 4H), 1.21-1.36 (m, 5H).

### Example 336

### 4-Chloro-1-methyl-5-{1-[4-(3-pyridin-2-yl-propyl)-piperidin-1-yl]-ethyl}-2-(4-trifluoromethoxy-phenyl)-1,2-dihydro-pyrazol-3-one

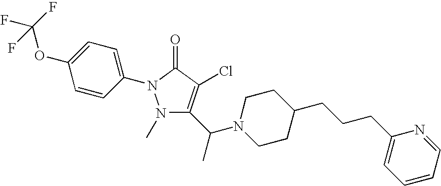

4-Chloro-1-methyl-5-{1-[4-(3-pyridin-2-yl-propyl)-piperidin-1-yl]-ethyl}-2-(4-trifluoromethoxy-phenyl)-1,2-dihydro-pyrazol-3-one was obtained from 2-(3-piperidin-4-yl-propyl)-pyridine (0.231 mmol, 0.047 g), 5-(1-bromo-ethyl)-4-chloro-1-methyl-2-(4-trifluoromethoxy-phenyl)-1,2-dihydro-pyrazol-3-one (0.154 mmol, 0.062 g) and potassium carbonate (0.123 mmol, 0.170 g) in acetonitrile (3 mL) as a yellow oil (0.0314 g, 39%). ¹H NMR (300 MHz, CDCl₃): δ (ppm) 8.53-8.54 (m, 1H), 7.57-7.61 (m, 1H), 7.31-7.43 (m, 4H), 7.09-7.17 (m, 2H), 3.76 (q, 1H), 3.33 (s, 3H), 3.11-3.15 (m, 1H), 2.82-2.89 (m, 1H), 2.77 (t, 2H), 1.98-2.11 (m, 2H), 1.62-1.84 (m, 4H), 1.44 (d, 3H), 1.18-1.34 (m, 5H).

### Example 337

### 4-Chloro-5-(1-{4-[2-(3,4-dichloro-phenoxy)-ethyl]-piperidin-1-yl}-ethyl)-2-(4-fluoro-phenyl)-1-methyl-1,2-dihydro-pyrazol-3-one

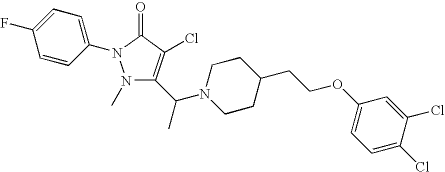

4-Chloro-5-(1-{4-[2-(3,4-dichloro-phenoxy)-ethyl]-piperidin-1-yl}-ethyl-2-(4-fluoro-phenyl)-1-methyl-1,2-dihydro-pyrazol-3-one was obtained from 4-[2-(3,4-chloro-phenoxy)-ethyl]-piperidine (0.146 mmol, 0.040 g), 5-(1-bromo-ethyl)-4-chloro-2-(4-fluoro-phenyl)-1-methyl-1,2-dihydro-pyrazol-3-one (0.097 mmol, 0.032 g) and potassium carbonate (0.487 mmol, 0.070 g) in acetonitrile (3 mL) as a yellow oil (0.0233 g, 46%). ¹H NMR (300 MHz, CDCl₃): δ (ppm) 7.31-7.37 (m, 3H), 7.15-7.21 (m, 2H), 6.78-6.99 (m, 1H), 6.74-6.78 (m 1H), 3.98 (t, 2H), 3.88 (q, 1H), 3.33 (s, 3H), 3.14-3.17 (m, 1H), 2.88-2.92 (m, 1H), 2.04-2.11 (m 2H), 1.69-1.81 (m, 4H), 1.55-1.63 (m, 1H), 1.47 (d, 3H), 1.24-1.33 (m, 2H).

### Example 338

### 4-Chloro-5-{4-[2-3,(4-dichloro-phenoxy)-ethyl]-piperidin-1-ylmethyl}-1-methyl-2-(4-trifluoromethoxy-phenyl)-1,2-dihydro-pyrazol-3-one

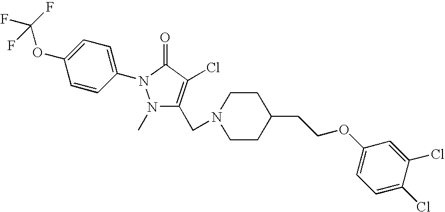

4-Chloro-5-{4-[2-(3,4-dichloro-phenoxy)-ethyl]-piperidin-1-ylmethyl}-1-methyl-2-(4-trifluoromethoxy-phenyl)-1,2-dihydro-pyrazol-3-one was obtained from 4-[2-(3,4-dichloro-phenoxy)-ethyl]-piperidine (0.146 mmol, 0.040 g), 5-bromomethyl-4-chloro-1-methyl-2-(4-trifluoromethoxy-phenyl)-1,2-dihydro-pyrazol-3-one (0.097 mmol, 0.037 g) and potassium carbonate (0.487 mmol, 0.070 g) in acetonitrile (3 mL) as a yellow solid (0.0446 g, 80%). ¹H NMR (300 MHz, CDCl₃): δ (ppm) 7.44-7.47 (m, 2H), 7.32-7.36 (m, 3H), 6.99-7.00 (m, 1H), 6.74-6.78 (m, 1H), 3.99 (t, 2H), 3.55 (s, 2H), 3.24 (s, 3H), 2.92-2.96 (m, 2H), 2.12-2.20 (m, 2H), 1.72-1.81 (m, 4H), 1.50-1.67 (m, 1H), 1.27-1.35 (m, 2H).

### Example 339

### 4-Chloro-5-(1-{4-[2-(3,4-dichloro-phenoxy)-ethyl]-piperidin-1-yl}-ethyl)-1-methyl-2-(4-trifluoromethoxy-phenyl)-1,2-dihydro-pyrazol-3-one

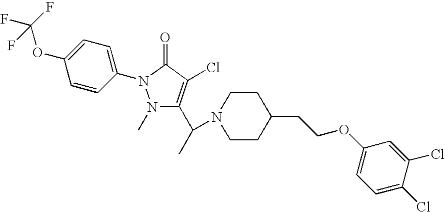

4-Chloro-5-(1-{4-[2-(3,4-difluoro-phenoxy)-ethyl]-piperidin-1-yl}-ethyl)-1-methyl-2-(4-trifluoromethoxy-phenyl)-1,2-dihydro-pyrazol-3-one was obtained from 4-[2-(3,4-dichloro-phenoxy)-ethyl]-piperidine (0.146 mmol, 0.040 g), 5-(1-bromo-ethyl)-4-chloro-1-methyl-2-(4-trifluoromethoxy-phenyl)-1,2-dihydro-pyrazol-3-one (0.097 mmol, 0.039 g) and potassium carbonate (0.487 mmol, 0.070 g) in acetonitrile (3 mL) as a yellow oil (0.0237 g, 41%). ¹H NMR (300 MHz, CDCl₃): δ (ppm) 7.41-7.44 (m, 2H), 7.32-7.35 (m, 3H), 6.99-7.00 (m, 1H), 6.74-6.78 (m, 1H), 3.98 (t, 2H), 3.78 (q, 1H), 3.35 (s, 3H), 3.11-3.19 (m, 1H), 2.89-2.94 (m, 1H), 2.04-2.12 (m, 2H), 1.67-1.90 (m, 4H), 1.55-1.65 (m, 1H), 1.48 (d, 3H), 1.27-1.45 (m, 2H).

### Example 340

### 4-Chloro-5-[4-(5-chloro-2-difluoromethoxy-phenyl)-3,6-dihydro-2H-pyridin-1-ylmethyl]-2-(4-fluoro-phenyl)-1-methyl-1,2-dihydro-pyrazol-3-one

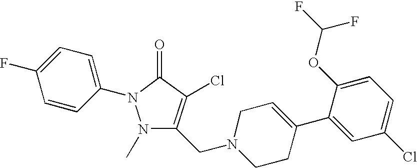

4-Chloro-5-[4-(5-chloro-2-difluoromethoxy-phenyl)-3,6-dihydro-2H-pyridin-1-ylmethyl]-2-(4-fluoro-phenyl)-1-methyl-1,2-dihydro-pyrazol-3-one was obtained from 4-(5-chloro-2-difluoromethoxy-phenyl)-1,2,3,6-tetrahydro-pyridine (0.104 mmol, 0.027 g), 5-bromomethyl-4-chloro-2-(4-fluoro-phenyl)-1-methyl-1,2-dihydro-pyrazol-3-one (0.07 mmol, 0.022 g) and potassium carbonate (0.35 mmol, 0.048 g) in acetonitrile (5 mL) as a brown oil (0.0349 g, 99%). ¹H NMR (300 MHz, CDCl₃): δ (ppm) 7.37-7.42 (m, 2H), 7.16-7.28 (m, 4H), 7.06-7.09 (m, 1H), 6.43 (t, 1H), 5.84 (s, 1H), 3.71 (s, 2H), 3.25-3.28 (m, 5H), 2.81 (t, 2H), 2.53 (s, 2H).

### Example 341

### 4-Chloro-5-[4-(5-chloro-2-difluoromethoxy-phenyl)-3,6-dihydro-2H-pyridin-1-ylmethyl]-1-methyl-2-(4-trifluoromethoxy-phenyl)-1,2-dihydro-pyrazol-3-one

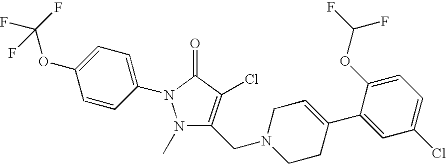

4-Chloro-5-[4-(5-chloro-2-difluoromethoxy-phenyl)-3,6-dihydro-2H-pyridin-1-ylmethyl]-1-methyl-2-(4-trifluoromethoxy-phenyl)-1,2-dihydro-pyrazol-3-one was obtained from 4-(5-chloro-2-difluoromethoxy-phenyl)-1,2,3,6-tetrahydro-pyridine (0.104 mmol, 0.027 g), 5-bromomethyl-4-chloro-1-methyl-2-(4-trifluoromethoxy-phenyl)-1,2-dihydro-pyrazol-3-one (0.07 mmol, 0.027 g) and potassium carbonate (0.55 mmol, 0.076 g) in acetonitrile (3 mL) as a yellow oil (0.0339 g, 85%). ¹H NMR (300 MHz, CDCl₃): δ (ppm) 7.09-7.50 (m, 6H), 7.091-7.093 (m, 1H), 6.41 (t, 1H), 5.85 (s, 1H), 3.73 (s, 2H), 3.27-3.30 (m, 5H), 2.82 (t, 2H), 2.53 (s, 2H).

### Example 342

### 4-Chloro-5-{1-[4-(5-chloro-2-difluoromethoxy-phenyl)-3,6-dihydro-2H-pyridin-1-yl]-ethyl}-1-methyl-2-(4-trifluoromethoxy-phenyl)-1,2-dihydro-pyrazol-3-one

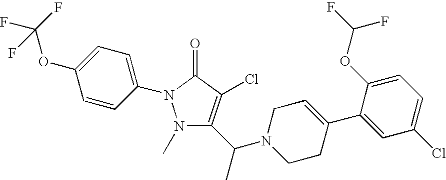

4-Chloro-5-{1-[4-(5-chloro-2-difluoromethoxy-phenyl)-3,6-dihydro-2H-pyridin-1-yl]-ethyl}-1-methyl-2-(4-trifluoromethoxy-phenyl)-1,2-dihydro-pyrazol-3-one was obtained from 4-(5-chloro-2-difluoromethoxy-phenyl)-1,2,3,6-tetrahydro-pyridine (0.104 mmol, 0.027 g), 5-(1-bromo-ethyl)-4-chloro-1-methyl-2-(4-trifluoromethoxy-phenyl)-1,2-dihydro-pyrazol-3-one (0.07 mmol, 0.028 g) and potassium carbonate (0.07 mmol, 0.027 g) in acetonitrile (3 mL) as a yellow oil (0.0169 g, 42%). ¹H NMR (300 MHz, CDCl₃): δ (ppm) 7.08-7.50 (m, 6H), 7.07-7.08 (m, 1H), 6.42 (t, 1H), 5.87 (s, 1H), 3.97 (q, 1H), 3.37 (s, 3H), 3.22-3.23 (m, 2H), 2.82 (t, 2H), 2.53 (s, 2H), 1.56 (d, 3H).

### Example 343

### 4-Chloro-2-(4-fluoro-phenyl)-1-methyl-5-[4-(3-pyridin-3-yl-propyl)-piperidin-1-ylmethyl]-1,2-dihydro-pyrazol-3-one

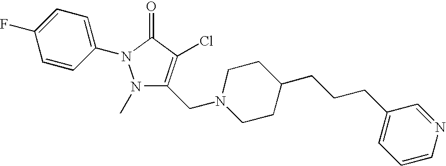

4-Chloro-2-(4-fluoro-phenyl)-1-methyl-5-[4-(3-pyridin-3-yl-propyl)-piperidin-1-ylmethyl]-1,2-dihydro-pyrazol-3-one was obtained 3-(3-piperidin-4-yl-propyl)-pyridine (0.065 mmol, 0.0133 g), 5-bromomethyl-4-chloro-2-(4-fluoro-phenyl)-1-methyl-1,2-dihydro-pyrazol-3-one (0.044 mmol, 0.014 g) and potassium carbonate (0.44 mmol, 0.061 g) in acetonitrile (3 mL) as a yellow oil (0.0096 g, 49%).

¹H NMR (300 MHz, CDCl₃): δ (ppm) 8.48 (s, 2H), 7.35-7.55 (m, 3H), 7.15-7.25 (m, 3H), 3.53 (s, 2H), 3.22 (s, 3H), 2.89-2.93 (m, 2H), 2.62 (t, 2H), 2.07-2.15 (m, 2H), 1.64-1.73 (m, 4H), 1.20-1.32 (m, 5H).

### Example 344

### 4-Chloro-2-(4-fluoro-phenyl)-1-methyl-5-{1-[4-(3-pyridin-3-yl-propyl)-piperidin-1-yl]-ethyl}-1,2-dihydro-pyrazol-3-one

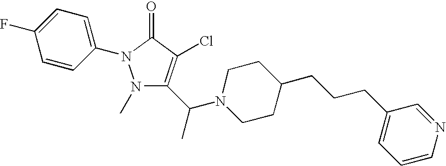

4-Chloro-2-(4-fluoro-phenyl)-1-methyl-5-{1-[4-(3-pyridin-3-yl-propyl)-piperidin-1-yl]-ethyl}-1,2-dihydro-pyrazol-3-one was obtained 3-(3-piperidin-4-yl-propyl)-pyridine (0.065 mmol, 0.0133 g), 5-(1-bromo-ethyl)-4-chloro-2-(4-fluoro-phenyl)-1-methyl-1,2-dihydro-pyrazol-3-one (0.044 mmol, 0.015 g) and potassium carbonate (0.44 mmol, 0.61 g) in acetonitrile (3 mL) as a white solid (0.0105 g, 52%). ¹H NMR (300 MHz, CDCl₃): δ (ppm) 8.46 (s, 2H), 7.49-7.52 (m, 1H), 7.32-7.37 (m, 2H), 7.15-7.25 (m, 3H), 3.74 (q, 1H), 3.32 (s, 3H), 3.08-3.12 (m, 1H), 2.89-2.92 (m, 1H), 2.62 (t, 2H), 1.99-2.06 (m, 2H), 1.63-1.78 (m, 4H), 1.46 (d, 3H), 1.17-1.29 (m, 5H).

### Example 345

### 4-Chloro-1-methyl-5-[4-(3-pyridin-3-yl-propyl)-piperidin-1-ylmethyl]-2-(4-trifluoromethoxy-phenyl)-1,2-dihydro-pyrazol-3-one

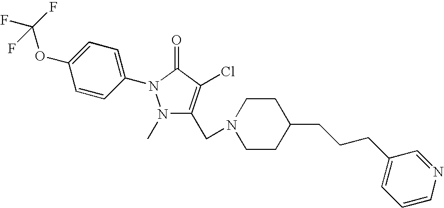

4-Chloro-1-methyl-5-[4-(3-pyridin-3-yl-propyl)-piperidin-1-ylmethyl]-2-(4-trifluoromethoxy-phenyl)-1,2-dihydro-pyrazol-3-one was obtained from 3-(3-piperidin-4-yl-propyl)-pyridine (0.065 mmol, 0.0133 g), 5-bromomethyl-4-chloro-1-methyl-2-(4-trifluoromethoxy-phenyl)-1,2-dihydro-pyrazol-3-one (0.044 mmol, 0.017 g) and potassium carbonate (0.44 mmol, 0.061 g) in acetonitrile (3 mL) as a yellow oil (0.0116 g, 52%). ¹H NMR (300 MHz, CDCl₃): δ (ppm) 8.48 (s, 2H), 7.44-7.53 (m, 3H), 7.33-7.36 (m, 3H), 3.54 (s, 2H), 3.22 (s, 3H), 2.89-2.32 (m, 2H), 2.63 (t, 2H), 2.07-2.16 (m, 2H), 1.62-1.74 (m, 4H), 1.21-1.33 (m, 5H).

### Example 346

### 4-Chloro-1-methyl-5-{1-[4-(3-pyridin-3-yl-propyl)-piperidin-1-yl]-ethyl}-2-(4-trifluoromethoxy-phenyl)-1,2-dihydro-pyrazol-3-one

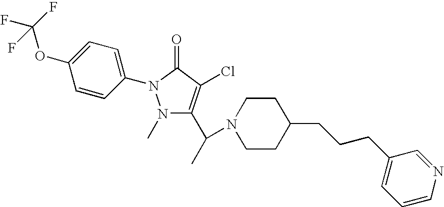

4-Chloro-1-methyl-5-{1-[4-(3-pyridin-3-yl-propyl)-piperidin-1-yl]-ethyl}-2-(4-trifluoromethoxy-phenyl)-1,2-dihydro-pyrazol-3-one was obtained from 3-(3-piperidin-4-yl-propyl)-pyridine (0.065 mmol, 0.0133 g), 5-(1-bromo-ethyl)-4-chloro-1-methyl-2-(4-trifluoromethoxy-phenyl)-1,2-dihydro-pyrazol-3-one (0.044 mmol, 0.018 g) and potassium carbonate (0.44 mmol, 0.061 g) in acetonitrile (3 mL) as a yellow oil (0.0091 g, 40%). ¹H NMR (300 MHz, CDCl₃): [symbol] (ppm) 8.46 (m, 2H), 7.40-7.52 (m, 3H), 7.32-7.35 (m, 3H), 3.77 (q, 1H), 3.34 (s, 3H), 3.11-3.15 (m, 1H), 2.82-2.89 (m, 1H), 2.62 (t, 2H), 2.00-2.17 (m, 2H), 1.62-1.79 (m, 4H), 1.47 (d, 3H), 1.18-1.32 (m, 5H).

### Example 347

### 4-Methoxy-1-methyl-2-phenyl-5-[4-(3-phenyl-propyl)-piperidin-1-ylmethyl]-1,2-dihydro-pyrazol-3-one

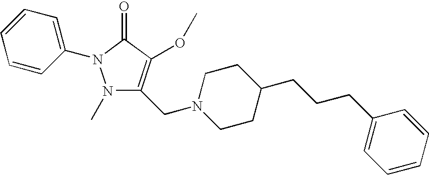

4-Methoxy-1-methyl-2-phenyl-5-[4-(3-phenyl-propyl)-piperidin-1-ylmethyl]-1,2-dihydro-pyrazol-3-one was obtained from 4-(3-Phenyl-propyl)-piperidine (29.1 μL, 0.152 mmol), 5-Bromomethyl-4-methoxy-1-methyl-2-phenyl-1,2-dihydro-pyrazol-3-one (30.0 mg, 0.101 mmol), and potassium carbonate (69.8 mg, 0.505 mmol) in anhydrous acetonitrile (1.5 mL) as a white solid (41.9 mg, 99%). ¹H NMR (300 MHz, CDCl₃): δ (ppm) 7.43-7.52 (m, 4H), 7.27-7.33 (m, 3H), 7.18-7.22 (m, 3H), 3.95 (s, 3H), 3.48 (s, 2H), 3.05 (s, 3H), 2.94 (broad d, 2H), 2.62 (t, 2H), 2.01-2.18 (m, 2H), 1.63-1.73 (m, 4H), 1.23-1.32 (m, 4H).

### Example 348

### 4-Chloro-1-methyl-2-phenyl-5-[4-(3-phenyl-propyl)-piperidin-1-ylmethyl]-1,2-dihydro-pyrazol-3-one

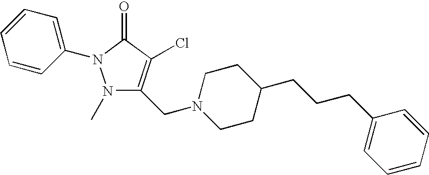

4-Chloro-1-methyl-2-phenyl-5-[4-(3-phenyl-propyl)-piperidin-1-ylmethyl]-1,2-dihydro-pyrazol-3-one was obtained from 4-(3-Phenyl-propyl)-piperidine (28.5 μL, 0.149 mmol), 5-Bromomethyl-4-chloro-1-methyl-2-phenyl-1,2-dihydro-pyrazol-3-one (30.0 mg, 0.099 mmol), and potassium carbonate (68.4 mg, 0.495 mmol) in anhydrous acetonitrile (1.5 mL) as a white solid (40.9 mg, 97%). ¹H NMR (300 MHz, CDCl₃): δ (ppm) 7.46-7.52 (m, 2H), 7.27-7.42 (m, 5H), 7.18-7.22 (m, 3H), 3.54 (s, 2H), 3.23 (s, 3H), 2.92 (broad m, 2H), 2.62 (t, 2H), 2.07-2.12 (broad m, 2H), 1.59-1.75 (m, 4H), 1.19-1.33 (m, 4H).

### Example 349

### 4-Chloro-1-ethyl-2-phenyl-5-[4-(3-phenyl-propyl)-piperidin-1-ylmethyl]-1,2-dihydro-pyrazol-3-one

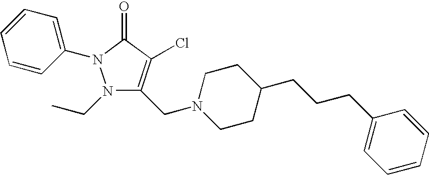

4-Chloro-1-ethyl-2-phenyl-5-[4-(3-phenyl-propyl)-piperidin-1-ylmethyl]-1,2-dihydro-pyrazol-3-one was obtained from 4-(3-Phenyl-propyl)-piperidine (27.4 μL, 0.143 mmol), 5-Bromomethyl-4-chloro-1-ethyl-2-phenyl-1,2-dihydro-pyrazol-3-one (30.0 mg, 0.095 mmol), and potassium carbonate (65.7 mg, 0.475 mmol) in anhydrous acetonitrile (1.5 mL) as a yellow, transparent oil (47.5 mg, 114%). ¹H NMR (300 MHz, CDCl₃): δ (ppm) 7.27-7.50 (m, 7H), 7.19 (m, 3H), 3.78 (q, 2H), 3.52 (s, 2H), 2.91-3.00 (broad m, 2H), 2.62 (t, 2H), 2.15 (m, 2H), 1.60-1.80 (m, 4H), 1.18-1.39 (m, 4H), 0.87 (t, 3H).

### Example 350

### 5-(4-Benzyl-piperidin-1-ylmethyl)-4-chloro-1-methyl-2-phenyl-1,2-dihydro-pyrazol-3-one

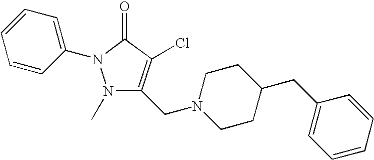

5-(4-Benzyl-piperidin-1-ylmethyl)-4-chloro-1-methyl-2-phenyl-1,2-dihydro-pyrazol-3-one was obtained from 4-benzylpiperidine (26.52 μL, 0.149 mmol), 5-Bromomethyl-4-chloro-1-methyl-2-phenyl-1,2-dihydro-pyrazol-3-one (30.0 mg, 0.099 mmol), and potassium carbonate (68.4 mg, 0.495 mmol) in anhydrous acetonitrile (1.5 mL) as a white solid (33.9 mg, 87%). 1H NMR (300 MHz, CDCl₃): δ (ppm) 7.45-7.55 (m, 2H), 7.29-7.42 (m, 5H), 7.15-7.22 (m, 3H), 3.54 (s, 2H), 3.22 (s, 3H, N—CH₃), 2.89-2.95 (broad m, 2H), 2.56 (d, 2H), 2.10 (m, 2H), 1.57-1.78 (m, 2H), 1.22-1.39 (m, 2H).

### Example 351

### 4-Chloro-5-[4-(4-chloro-2-methoxy-phenyl)-piperazin-1-ylmethyl]-1-methyl-2-phenyl-1,2-dihydro-pyrazol-3-one

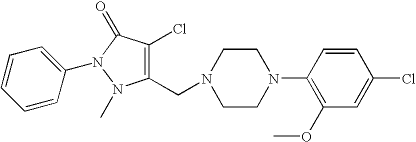

4-Chloro-5-[4-(4-chloro-2-methoxy-phenyl)-piperazin-1-ylmethyl]-1-methyl-2-phenyl-1,2-dihydro-pyrazol-3-one was obtained from 1-(4-Chloro-2-methoxy-phenyl)-piperazine (33.8 mg, 0.149 mmol), 5-Bromomethyl-4-chloro-1-methyl-2-phenyl-1,2-dihydro-pyrazol-3-one (30.0 mg, 0.099 mmol), and potassium carbonate (68.4, 0.495 mmol) in acetonitrile (1.5 mL) as a white film (5.1 mg, 12%). ¹H NMR (300 MHz, CDCl₃): δ (ppm) 7.42-7.52 (m, 4H), 7.38 (t, 1H), 6.91 (d, 1H), 6.85 (d, 2H), 3.89 (s, 3H), 3.79 (q, 2H), 3.63 (s, 2H), 3.09 (broad s, 4H), 2.80 (broad t, 4H), 0.91 (t, 3H).

The following experimental conditions for HPLC/MS plate analyses pertain to the characterization of compounds in the examples below.

Method A. The samples were dissolved in DMSO (0.5 ml) and diluted with 0.5 ml MeOH in 96 deep well plate format. They were analyzed by electrospray gradient LC/MS (METHOD A), in the positive ionization mode, using a Waters QTOF1 mass spectrometer and Agilent 1100 hplc. The following experimental conditions were employed:

### HPLC

| | |
|---|---|
| Column: | Supelco Discovery HS C18, |
| | 50 × 2.1 mm, 5 □m |
| Mobile Phase A: | Water/Acetonitrile/Formic acid |
| | (98:2:0.1% v/v) |
| Mobile Phase B: | Water/Acetonitrile/Formic acid |
| | (2:98:0.1% v/v) |
| Flow rate: | 0.5 ml/min |
| UV-DAD: | 210-330 nm |
| Column Temp: | 30′ C. |
| Inj. Vol.: | 1 □l |
| Gradient (Time in min(% B)): | Linear - 0(2); 4(95); 5(95); 5.2(2); 7(2) |

### QTOF1

| | |
|---|---|
| Mass range: | 130-800 Da |
| Scan Rate: | 0.5 s |
| Interscan delay: | 0.05 s |
| Cone voltage: | 35 v |
| Ionization mode: | ESP(+) |

Method B: Samples were run on a HP1100 HPLC equipped with an Agilent G1946A mass detector set to electrospray mode of ionization. LC conditions: Agilent C8-Symmetry® column (5 □m), 3.9×50 mm. Mobile phase: CH₃CN/H₂O. From 100% H₂O (containing 0.025% TFA) to 100% CH₃CN (containing 0.025% TFA) over 5 min.

Method C: APCI detection, Zorbax C8-stable bond column (50×2.1 mm). Mobile phase: CH₃CN/H₂O. From 98% H₂O (containing 0.1% formic acid) to 98% CH₃CN (containing 0.1% formic acid) over 5 min.

### Example 352

### 5-Methyl-2-phenyl-1,2-dihydropyrazol-3-one

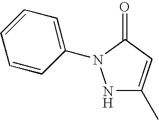

Phenylhydrazine (5.41 g, 50.0 mmol) in toluene (100 mL) was treated with ethyl acetoacetate (6.4 mL, 50.0 mmol) and refluxed for 24 hours. The mixture was concentrated and triturated with diethyl ether to give the product as an off-white solid (6.18 g, 71%).

### Example 353

### 1-Ethyl-5-methyl-2-phenyl-1,2-dihydropyrazol-3-one

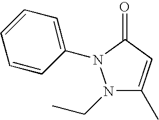

5-Methyl-2-phenyl-1,2-dihydro-pyrazol-3-one (1.0 g, 5.74 mmol) and iodoethane (5.0 mL, 62.5 mmol) were heated at 100° C. for 24 hours in a sealed tube. The mixture was concentrated and chromatographed with 5% 2.0M ammonia in methanol and dichloromethane to give the product as an amber oil (695 mg, 59%). ¹H NMR (300 MHz, d₆-DMSO): δ (ppm) 7.53-7.42 (m, 2H), 7.35-7.25 (m, 3H), 5.32 (s, 1H), 3.56 (q, 2H), 2.23 (s, 3H), 0.78 (t, 3H).

### Example 354

### 4-Bromo-5-bromomethyl-1-ethyl-2-phenyl-1,2-dihydro-pyrazol-3-one

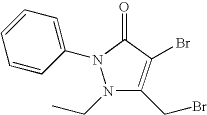

1-Ethyl-5-methyl-2-phenyl-1,2-dihydro-pyrazol-3-one (695 mg, 3.44 mmol) in carbon tetrachloride (35 mL) was treated with N-bromosuccinimide (1.23 g, 6.91 mmol) and heated at 50° C. for 2 hours. The mixture was diluted with dichloromethane and washed (1N NaOH, water, brine), dried (Na₂SO₄), and evaporated to a crude oil. The material was chromatographed with 20% acetonitrile in dichloromethane to give the product as an off-white solid (1.06 g, 85%). ¹H NMR (300 MHz, CDCl3): δ(ppm) 7.53-7.28 (m, 5H), 4.37 (s, 2H), 3.74 (q, 2H), 0.96 (s, 3H).

### Example 355

### 4-Bromo-5-[4-(3,5-dichloro-pyridin-4-yl)piperazin-1-ylmethyl]-1-ethyl-2-phenyl-1,2-dihydro-pyrazol-3-one

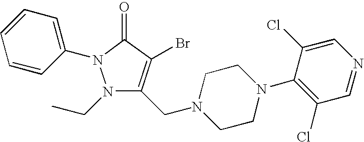

A mixture of 4-bromo-5-bromomethyl-1-ethyl-2-phenyl-1,2-dihydro-pyrazol-3-one (100 mg, 0.28 mmol), 1-(3,5-dichloro-4-pyridyl)piperazine (72 mg, 0.31 mmol), and triethylamine (100 μL, 0.72 mmol) in tetrahydrofuran (10 mL) was heated at 50° C. for 4.5 hours. Additional 1-(3,5-dichloro-4-pyridyl)piperazine (20 mg, 0.09 mmol) and acetonitrile (2 mL) were added and heating continued at 50° C. for 2 hours. The mixture was concentrated and the residue partitioned between water and dichloromethane. The organic portion was washed (water, brine), dried (Na₂SO₄), and concentrated to a crude oil that was chromatographed with 20% acetonitrile in dichloromethane and 35% acetonitrile in dichloromethane. The resulting solid was triturated with 19:1 hexane/ethyl acetate to give the product as a pale yellow solid (76 mg, 53%). ¹H NMR (300 MHz, CDCl₃): δ(ppm) 8.35 (s, 2H), 7.54-7.29 (m, 5H), 3.83 (q, 2H), 3.64 (s, 2H), 3.44-3.35 (m, 4H), 2.80-2.69 (m, 4H), 0.92 (t, 3H). LC/MS (METHOD A): 510 (M+H) at 4.63 min.

Compounds of Examples 356 through 361 were synthesized by a method analogous to the procedure of Example 355 using 4-bromo-5-bromomethyl-1-ethyl-2-phenyl-1,2-dihydro-pyrazol-3-one and the appropriate amine.

| | | | LC/MS | |
|---|---|---|---|---|
| | | | (METHOD | |
| | | | A) | m/z |
| Example | Structure | Name | (min.) | (M + H) |
| 356 | 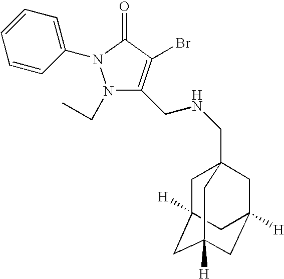 | 5-{[(adamantan-1-ylmethyl)-amino]-methyl}-e-bromo-1-ethyl-2-phenyl-1,2-dihydro-pyrazol-3-one | 3.89 | 444 |
| 357 | 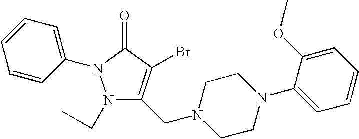 | 4-Bromo-1-ethyl-5-[4-(2-methoxy-phenyl)-piperazin-1-ylmethyl]-2-phenyl-1,2-dihydro-pyrazol-3-one | 3.94 | 471 |
| 358 | 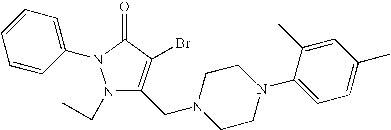 | 4-Bromo-5-[4-(2,4-dimethyl-phenyl)-piperazin-1-ylmethyl]-1-ethyl-2-phenyl-1,2-dihydro-pyrazol-3-one | 4.72 | 469 |
| 359 | 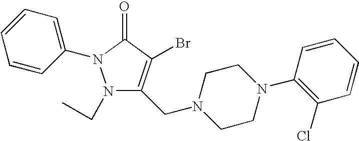 | 4-Bromo-5-[4-(2-chloro-phenyl)-piperazin-1-ylmethyl]-1-ethyl-2-phenyl-1,2-dihydro-pyrazol-3-one | 4.74 | 475 |
| 360 | 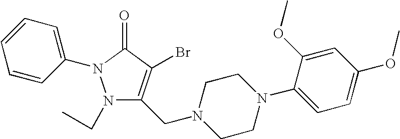 | 4-Bromo-5-[4-(2,4-dimethoxy-phenyl)-piperazin-1-ylmethyl]-1-ethyl-2-phe-nyl-1,2-dihydro-pyrazol-3-one | 3.80 | 501 |
| 361 | 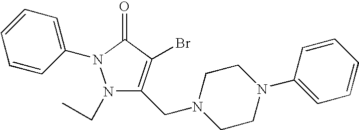 | 4-Bromo-1-ethyl-2-phenyl-5-(4-phenyl-piperazin-1-ylmethyl)-1,2-dihydro-pyrazol-3-one | 4.32 | 441 |

### Example 362

### 4-bromo-1-methyl-2-phenyl-5-piperazin-1-ylmethyl-1,2-dihydro-pyrazolo-3-one hydrochloride

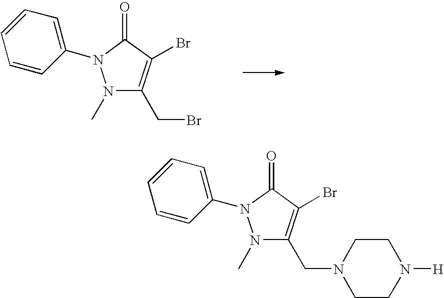

A solution of 4-bromo-5-bromomethyl-1-methyl-2-phenyl-1,2-dihydro-pyrazol-3-one (0.2 g, 0.58 mmol), 1-boc-piperazine 0.11 g, 0.58 mmol) and triethylamine (0.11 mL, 0.58 mmol) in acetonitrile (2 mL) was heated to 80° C. for 2 hours. The solution was diluted with ethyl acetate, washed with saturated NH₄Cl, the organic layer separated, dried (MgSO₄) and concentrated. The residue was dissolved in CH₂Cl₂ and treated with 4N HCl in dioxane at rt. After 12 hrs the solvent was evaporated and the residue recrystallized from CH₂Cl₂ to give 4-bromo-1-methyl-2-phenyl-5-piperazin-1-ylmethyl-1,2-dihydro-pyrazolo-3-one hydrochloride as a white solid (0.17 g, 85%). ¹H NMR (300 MHz, DMSO-d₆): [symbol] (ppm) 7.5 (m, 2H), 7.4 (m, 3H), 6.0 (bs, 1H), 3.9 (s, 2H), 3.2 (s, 3H), 3.1 (m, 4H), 2.8 (m, 4H).

### Example 363

### 4-Bromo-1-methyl-2-phenyl-5-[4-((1S,2S)-2-phenyl-cyclopropanecarbonyl)-piperazin-1-ylmethyl]-1,2-dihydro-pyrazol-3-one

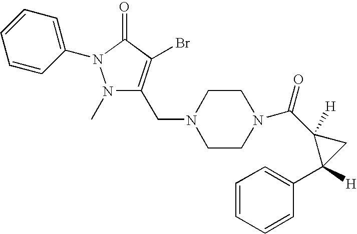

A solution of 4-bromo-1-methyl-2-phenyl-5-piperazin-1-ylmethyl-1,2-dihydro-pyrazolo-3-one hydrochloride (20 mg, 0.06 mmol), trans-2-phenyl-cylopronane carboxylic acid (14 mg, 0.085 mmol) and PS-carbodiimide (80 mg, 1.33 mmol/g, 0.11 mmol) in CH₂Cl₂ was stirred at rt for 12 hrs. The reaction was filtered and the solvent removed at reduced pressure. Chromatography (silica, 5% MeOH/CH₂Cl₂) gave 4-bromo-1-methyl-2-phenyl-5-[4-((1S,2S)-2-phenyl-cyclopropanecarbonyl)-piperazin-1-ylmethyl]-1,2-dihydro-pyrazol-3-one as a solid (21 mg, 75%). ¹H NMR (300 MHz, DMSO-d₆): [symbol] (ppm) 7.5-7.1 (m, 10H), 3.9 (s, 2H), 3.2 (s, 3H), 3.1 (m, 4H), 2.8 (m, 4H) 2.2 (m, 1H), 2.0 (m, 1H), 0.9 (m, 2H); LC/MS (METHOD A): 495 (M+H) at 4.36 min.

Compounds of Examples 365 through 367 were synthesized by a method analogous to the procedure of Example 364 using 4-bromo-1-methyl-2-phenyl-5-piperazin-1-ylmethyl-1,2-dihydro-pyrazolo-3-one hydrochloride and the appropriate carboxylic acid.

| | | | LC/MS | |
|---|---|---|---|---|
| | | | (METHOD | |
| | | | A) | m/z |
| Example | Structure | Name | (min) | (M + H) |
| 364 | 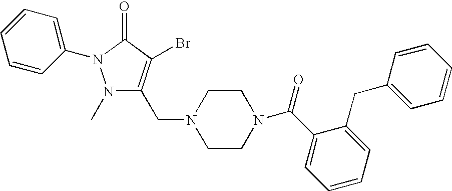 | 5-[4-(2-Benzyl-benzoyl)-piperazin-1-ylmethyl]-4-bromo-1-methyl-2-pheny-l-1,2-dihydro-pyrazol-3-one | 4.74 | 545 |
| 365 | 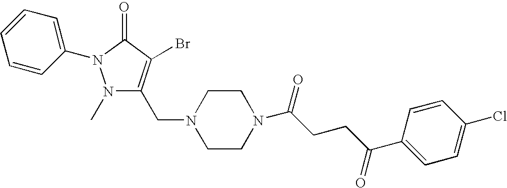 | 1-[4-(4-Bromo-2-methyl-5-oxo-1-phenyl-2,5-dihydro-1H-pyrazol-3-ylmethyl)-piperazin-1-yl]-4-(4-chloro-phenyl)-butane-1,4-dione | 4.40 | 546 |
| 366 | 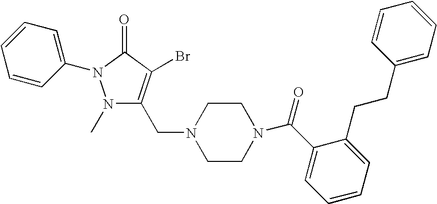 | 4-Bromo-1-methyl-5-[4-(2-phenethyl-benzoyl)-piperazin-1-ylmethyl]-2-phe-nyl-1,2-dihydro-pyrazol-3-one | 4.85 | 559 |
| 367 | 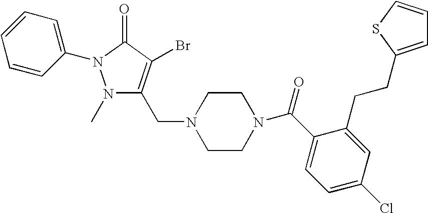 | 4-Bromo-5-{4-[4-chloro-2-(2-thiophen-2-yl-ethyl)-benzoyl]-piperazin-1-ylmethyl}-1-methyl-2-phenyl-1,2-di-hydro-pyrazol-3-one | 5.26 | 599 |

### Example 368

### 4-phenyl-2,3,8-triaza-spiro[4.5]dec-3-en-1-one

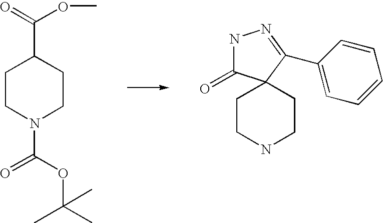

A solution of piperidine-1,4-dicarboxylic acid 1-tert-butyl ester 4-methyl ester (0.2 g, 0.82 mmol) in THF (1 mL) was treated with potassium hexamethyldisilazane (2.45 mL, 1.2 mmol) at rt via syringe. After 30 min benzoyl chloride (0.115 mL, 0.98 mmol) was added to the reaction. After 30 min the reaction was quenched with MeOH, diluted with ethyl acetate, washed with saturated NH₄Cl, the organic separated, dried (MgSO₄) and the solvent removed at reduced pressure. The residue was dissolved in n-BuOH (2 mL) and treated with hydrazine hydrate (0.14 mL, 2.46 mmol) and heated to 115° C. for 4 hrs. After cooling, the reaction was diluted with ethyl acetate, washed with 1N HCl, the organic separated, dried (MgSO₄) and the solvent removed at reduced pressure to afford 4-oxo-1-phenyl-2,3,8-triaza-spiro[4.5]dec-1-ene-8-carboxylic acid tert-butyl ester as an oil, which was used without further purification. The residue was dissolved in CH₂Cl₂ (1 mL) and treated with 4N HCl (2 mL) at rt. After 3 hrs the solvent was removed at reduced pressure and the residue recrystallized from ethyl acetate to give 4-phenyl-2,3,8-triaza-spiro[4.5]dec-3-en-1-one as a white solid (120 mg, 65%). ¹H NMR (300 MHz, DMSO-d₆): [symbol] (ppm) 11.8 (bs, 1H), 8.0 (m, 2H), 7.4 (m, 3H), 3.6 (m, 2H), 3.2 (m, 1H), 2.8 (m, 2H), 1.8 (m, 4H); LC/MS (METHOD A): 230 (M+H) at 0.89 min.

### Example 369

### 2-(4-Fluoro-benzyl)-4-phenyl-2,3,8-triaza-spiro[4.5]dec-3-en-1-one

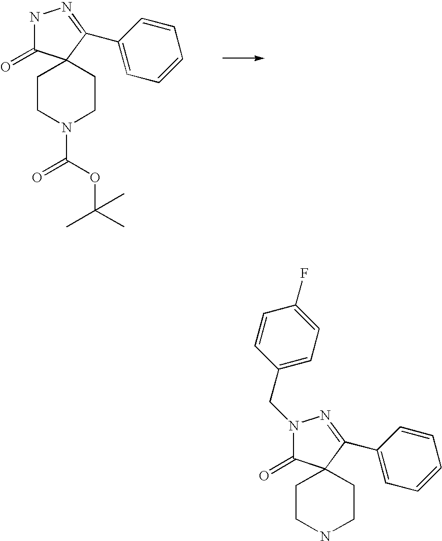

A solution of 4-oxo-1-phenyl-2,3,8-triaza-spiro[4.5]dec-1-ene-8-carboxylic acid tert-butyl ester (0.08 g, 0.24 mmol) in THF (1 mL) was treated with potassium hexamethyldisilazane (0.72 mL, 0.36 mmol) at rt via syringe. After 30 min p-fluorobenzyl bromide (0.04 mL, 0.3 mmol) was added to the reaction. After 30 min the reaction was quenched with MeOH, diluted with ethyl acetate, washed with saturated NH₄Cl, the organic separated, dried (MgSO₄) and the solvent removed at reduced pressure. The residue was dissolved in CH₂Cl₂ (1 mL) and treated with 4N HCl (2 mL) at rt. After 3 hrs the solvent was removed at reduced pressure and the residue recrystallized from ethyl acetate to give 2-(4-fluoro-benzyl)-4-phenyl-2,3,8-triaza-spiro[4.5]dec-3-en-1-one as a white solid (50 mg, 63%). ¹H NMR (300 MHz, DMSO-d₆): [symbol] (ppm) 8.0 (m, 2H), 7.4 (m, 5H), 7.1 (m, 2H), 4.8 (s, 2H), 3.6 (m, 2H), 3.2 (m, 1H), 2.8 (m, 2H), 1.8 (m, 4H); LC/MS (METHOD A): 338 (M+H) at 1.67 min

### Example 370

### 8-(4-Bromo-2-methyl-5-oxo-1-phenyl-2,5-dihydro-1H-pyrazol-3-ylmethyl)-2-(4-fluorobenzyl)-4-phenyl-2,3,8-triaza-spiro[4.5]dec-3-en-1-one

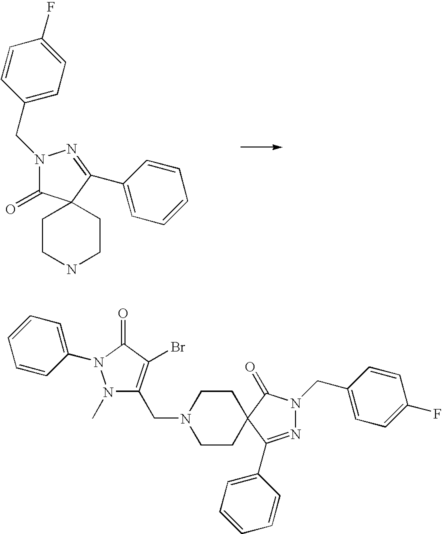

A solution of 4-bromo-5-bromomethyl-1-methyl-2-phenyl-1,2-dihydro-pyrazol-3-one (0.021 g, 0.06 mmol) and 2-(4-fluoro-benzyl)-4-phenyl-2,3,8-triaza-spiro[4.5]dec-3-en-1-one (0.02 g, 0.06 mmol) in acetonitrile (1.5 mL) was treated with triethylamine (0.016 mL, 0.06 mmol) and heated to reflux for 2 hrs. The reaction was cooled, diluted with ethyl acetate, washed with sat NH₄Cl solution, the organic phase separated, dried (MgSO₄) and the solvent removed at reduced pressure. Chromatography (silica, 5% MeOH in CH₂Cl₂) gave the product as a solid (0.031 g, 84%). ¹H NMR (300 MHz, CDCl₃): [symbol] (ppm) 7.5-7.1 (m, 12H), 6.8 (m, 2H), 4.8 (s, 2H), 3.6 (s, 2H), 3.2 (s, 3H), 3.1 (m, 2H), 2.8 (m, 1H), 2.3 (m, 1H), 1.8-1.6 (m, 4H); LC/MS (METHOD A): 603 (M+H) at 3.71 min.

Compounds of Examples 371 through 386 were synthesized by a method analogous to the procedure of Example 370 using either 4-bromo-5-bromomethyl-1-methyl-2-phenyl-1,2-dihydro-pyrazol-3-one, 4-bromo-5-bromomethyl-1-ethyl-2-phenyl-1,2-dihydro-pyrazol-3-one or 4-chloro-5-bromomethyl-1-ethyl-2-phenyl-1,2-dihydro-pyrazol-3-one and the appropriate amine.

| | | | LC/MS | |
|---|---|---|---|---|
| | | | (METHOD | |
| | | | A) | m/z |
| Example | Structure | Name | (min) | (M + H) |
| 371 | 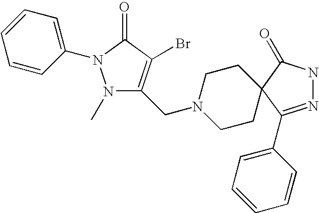 | 8-(4-Bromo-2-methyl-5-oxo-1-phenyl-2,5-dihydro-1H-pyrazol-3-ylmethyl)-4-phenyl-2,3,8-triaza-spiro[4.5]dec-3-en-1-one | 3.57 | 495 |
| 372 | 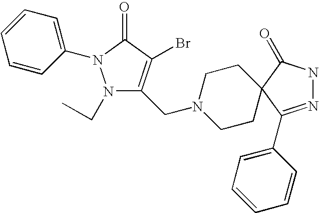 | 8-(4-Bromo-2-ethyl-5-oxo-1-phenyl-2,5-dihydro-1H-pyrazol-3-ylmethyl)-4-phenyl-2,3,8-triaza-spiro[4.5]dec-3-en-1-one | 3.59 | 509 |
| 373 | 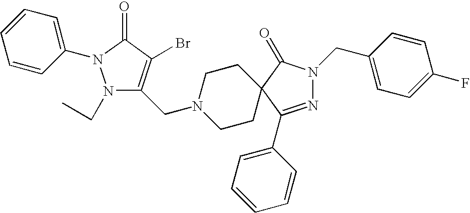 | 8-(4-Bromo-2-ethyl-5-oxo-1-phenyl-2,5-dihydro-1H-pyrazol-3-ylmethyl)-2-(4-fluoro-benzyl)-4-phenyl-2,3,8-triaza-spiro[4.5]dec-3-en-1-one | 3.74 | 616 |
| 374 | 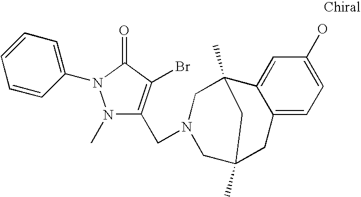 | 4-Bromo-5-((1R,9R)-4-hydroxy-1,9-dimethyl-11-aza-tricyclo[7.3.1.0*2,7*]tri-deca-2,4,6-trien-11-ylmethyl)-1-methyl-2-phenyl-1,2-dihydro-pyrazol-3-one | 3.48 | 483 |
| 375 | 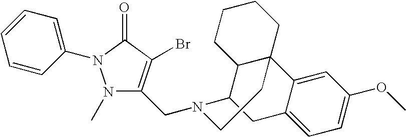 | 4-Bromo-5-((2S,6S,11R)-8-methoxy-6,11-cyclohexyl-1,2,5,6-tetrahydro-4H-2,6-methano-benzo[d]azocin-3-ylmethyl)-1-methyl-2-phenyl-1,2-dihydropyrazol-3-one | 3.44 | 523 |
| 376 | 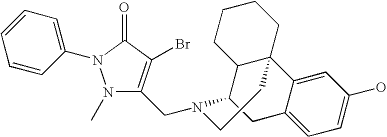 | 4-Bromo-5-((2S,6S,11R)-8-hydroxy-6,11-cyclohexyl-1,2,5,6-tetrahydro-4H-2,6-methano-benzo[d]azocin-3-ylmethyl)-1-methyl-2-phenyl-1,2-dihydropyrazol-3-one | 3.68 | 509 |
| 377 | 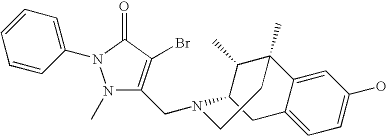 | 4-Bromo-5-((2S,6S,11R)-8-hydroxy-6,11-dimethyl-1,2,5,6-tetrahydro-4H-2,6-methano-benzo[d]azocin-3-ylmethyl)-1-methyl-2-phenyl-1,2-dihydropyrazol-3-one | 3.49 | 483 |
| 378 | 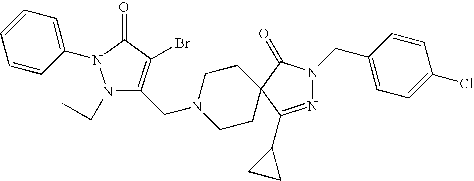 | 8-(4-Bromo-2-ethyl-5-oxo-1-phenyl-2,5-dihydro-1H-pyrazol-3-ylmethyl)-2-(4-chloro-benzyl)-4-cyclopropyl-2,3,8-triaza-spiro[4.5]dec-3-en-1-one | 4.45 | 597 |
| 379 | 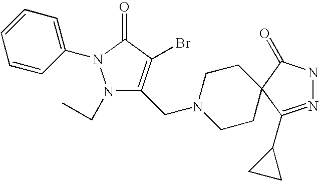 | 8-(4-Bromo-2-ethyl-5-oxo-1-phenyl-2,5-dihydro-1H-pyrazol-3-ylmethyl)-4-cyclopropyl-2,3,8-triaza-spiro[4.5]dec-3-en-1-one | 3.47 | 472 |
| 380 | 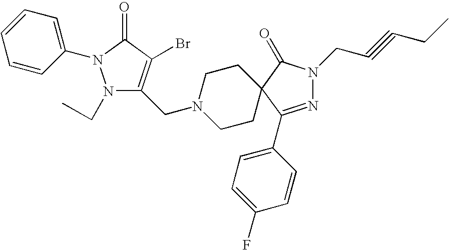 | 8-(4-Bromo-2-ethyl-5-oxo-1-phenyl-2,5-dihydro-1H-pyrazol-3-ylmethyl)-4-(4-fluoro-phenyl)-2-pent-2-ynyl-2,3,8-triaza-spiro[4.5]dec-3-en-1-one | 4.66 | 592 |
| 381 | 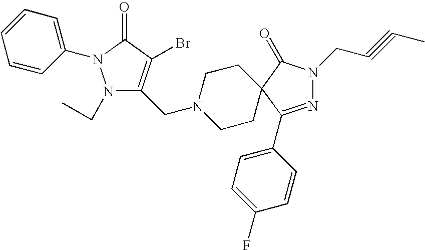 | 8-(4-Bromo-2-ethyl-5-oxo-1-phenyl-2,5-dihydro-1H-pyrazol-3-ylmethyl)-2-but-2-ynyl-4-(4-fluoro-phenyl)-2,3,8-triaza-spiro[4.5]dec-3-en-1-one | 4.44 | 578 |
| 382 | 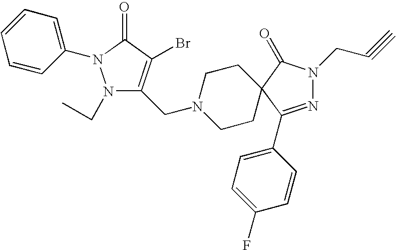 | 8-(4-Bromo-2-ethyl-5-oxo-1-phenyl-2,5-dihydro-1H-pyrazol-3-ylmethyl)-4-(4-fluoro-phenyl)-2-prop-2-ynyl-2,3,8-triaza-spiro[4.5]dec-3-en-1-one | 4.37 | 546 |
| 383 | 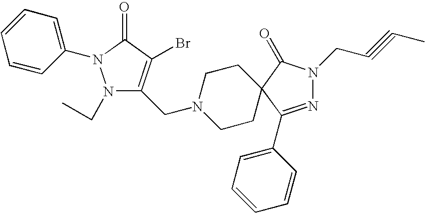 | 8-(4-Bromo-2-ethyl-5-oxo-1-phenyl-2,5-dihydro-1H-pyrazol-3-ylmethyl)-2-but-2-ynyl-4-phenyl-2,3,8-triaza-spiro[4.5]dec-3-en-1-one | 4.51 | 560 |
| 384 | 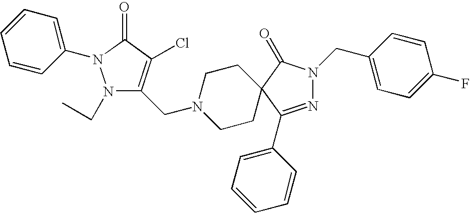 | 8-(4-Chloro-2-ethyl-5-oxo-1-phenyl-2,5-dihydro-1H-pyrazol-3-ylmethyl)-2-(4-fluoro-benzyl)-4-phenyl-2,3,8-triaza-spiro[4.5]dec-3-en-1-one | 4.63 | 572 |
| 385 | 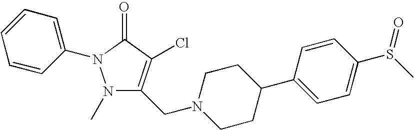 | 4-Chloro-5-[4-(4-methanesulfinyl-phe-nyl)-piperidin-1-ylmethyl]-1-methyl-2-phenyl-1,2-dihydro-pyrazol-3-one | 3.02 | 488 |
| 386 | 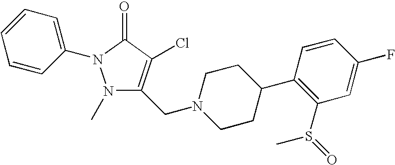 | 4-Chloro-5-[4-(4-fluoro-2-methanesul-finyl-phenyl)-piperidin-1-ylmethyl]-1-methyl-2-phenyl-1,2-dihydro-pyrazol-3-one | 3.20 | 507 |

### Example 387

### 4-Oxo-1-phenyl-1,3,8-triaza-spiro[4.5]decane-8-carboxylic acid tert-butyl ester

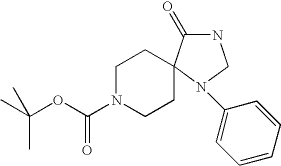

A mixture of 1-phenyl-1,3,8-triaza-spiro[4.5]decan-4-one (2.3 gm, 10 mmol), di-tert-butyldicarbonate (2.2 g, 10 mmol) and diisopropylethylamine (2.5 mL, 15 mmol) in tetrahydrofuran (150 mL) and acetonitrile (50 mL) was allowed to react at ambient temperature for 18 hours. The volatiles were evaporated and the residue was triturated with diethyl ether (30 mL) to give the product as a white solid (3.0 g, 91%). ¹H NMR (300 MHz, DMSO-d6): □ (ppm) 8.75 (s, 1H), 7.18 (t, J=8 Hz, 2H), 6.78-6.68 (m, 3H), 5.60 (s, 2H), 3.80-3.95 (m, 2H), 3.5-3.3 (m, 2H), 2.44-2.34 (m, 2H), 1.59 (d, J=13.8 Hz, 2H), 1.45 (s, 9H).

### Example 388

### 3-Benzyl-8-(4-bromo-2-methyl-5-oxo-1-phenyl-2,5-dihydro-1H-pyrazol-3-ylmethyl)-1-phenyl-1,3,8-triaza-spiro[4.5]decan-4-one

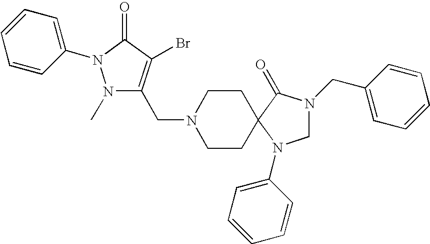

Sodium hydride (40 mg, 1 mmol) was added to a solution of 4-oxo-1-phenyl-1,3,8-triaza-spiro[4.5]decane-8-carboxylic acid tert-butyl ester (75 mg, 0.23 mmol) in NMP (4 mL). After 5 minutes benzyl bromide (36 uL, 0.3 mmol) was added. The mixture was stirred for 18 hours. The reaction was quenched by addition of water and extracted with ethyl acetate. The organic phase was washed with water then brine, evaporated and chromatographed on silica gel eluting with 0-100% ethyl acetate in methylene chloride. The Boc protecting group was removed by treatment with trifluoroacetic acid (1 ml) in THF (5 ml) then evaporated. The resulting amine intermediate (66 mg, 0.15 mmol) was mixed with 4-bromo-5-bromomethyl-1-methyl-2-phenyl-1,2-dihydro-pyrazol-3-one (52 mg, 0.15 mmol) and diisopropylethylamine (170 uL) in acetonitrile (3 mL). The reaction was heated in Emrys Optimizer microwave reactor to 150° C. for 10 minutes. The solvent was evaporated and the residue was chromatographed on 4 gram silica gel cartridge eluting with 0-100% ethyl acetate in methylene chloride. Obtained the title compound (30 mg, 22%) as an off-white solid. ¹H NMR (300 MHz, DMSO-d6): □ (ppm) 7.55-7.23 (m, 12H), 6.95-6.71 (m, 3H), 4.60 (s, 2H), 4.57 (s, 2H), 3.72 (s, 2H), 3.0-2.8 (m, 4H), 2.7-2.5 (m, 2H), 1.8-1.6 (m, 2H).

### Example 389

### 8-(4-Bromo-2-methyl-5-oxo-1-phenyl-2,5-dihydro-1H-pyrazol-3-ylmethyl)-3-(4-fluoro-benzyl)-1-phenyl-1,3,8-triaza-spiro[4.5]decan-4-one

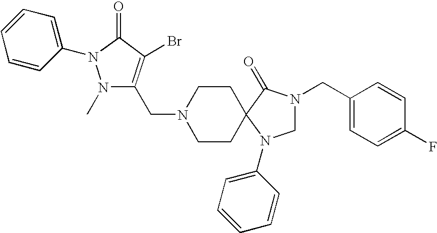

8-(4-Bromo-2-methyl-5-oxo-1-phenyl-2,5-dihydro-1H-pyrazol-3-ylmethyl)-1-phenyl-1,3,8-triaza-spiro[4.5]decan-4-one (200 mg, 0.4 mmol) was dissolved in hot NMP (6 mL). The solution was cooled to room temperature and sodium hydride (40 mg, 1 mmol) was added. After 15 minutes 1-bromomethyl-4-fluoro-benzene (50 uL, 0.4 mmol) was added and stirred 18 hours. Reaction was quenched by addition of water and extracted with ethyl acetate. The organic phase was washed 4 times with water then brine. The organic phase was evaporated and chromatographed on silica gel (4 gram column), eluting with 0-25% ethyl acetate in methylene chloride. Obtained the title compound (28 mg, 12%) as a yellow foam. ¹H NMR (300 MHz, DMSO-d6): □ (ppm) 7.54 (t, J=7.5 Hz, 2H), 7.43-7.34 (m, 5H), 7.25-7.18 (m, 4H), 6.83-6.74 (m, 3H), 4.59 (s, 2H), 4.54 (s, 2H), 3.70 (s, 2H), 2.97-2.85 (m, 4H), 2.63-2.53 (m, 2H), 1.70-1.63 (m, 2H).

Compounds of Examples 390 through 398 were synthesized by a method analogous to the procedure of Example 389 using 8-(4-bromo-2-methyl-5-oxo-1-phenyl-2,5-dihydro-1H-pyrazol-3-ylmethyl)-1-phenyl-1,3,8-triaza-spiro[4.5]decan-4-one and the appropriate alkylating reagent.

| | | | LC/MS | |
|---|---|---|---|---|
| | | | (METHOD | |
| | | | A) | M/z |
| Example | Structure | Name | (min.) | (m + H) |
| 390 | 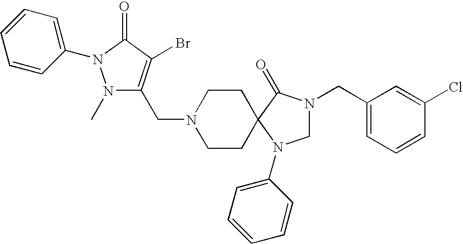 | 8-(4-Bromo-2-methyl-5-oxo-1-phenyl-2,5-dihydro-1H-pyrazol-3-ylmethyl)-3-(3-chloro-benzyl)-1-phenyl-1,3,8-triaza-spiro[4.5]decan-4-one | 4.52 | 620 |
| 391 | 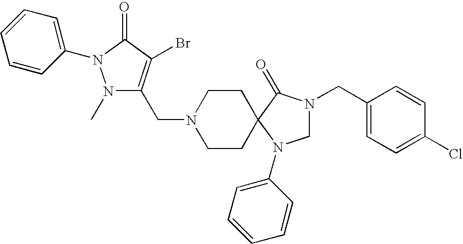 | 8-(4-Bromo-2-methyl-5-oxo-1-phenyl-2,5-dihydro-1H-pyrazol-3-ylmethyl)-3-(4-chloro-benzyl)-1-phenyl-1,3,8-triaza-spiro[4.5]decan-4-one | 4.49 | 620 |
| 392 | 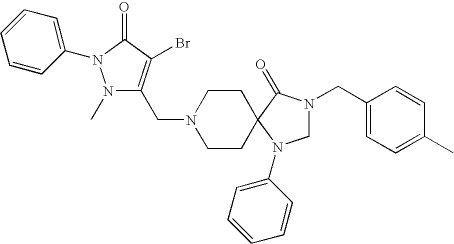 | 8-(4-Bromo-2-methyl-5-oxo-1-phenyl-2,5-dihydro-1H-pyrazol-3-ylmethyl)-1-phenyl-3-(4-methyl-benzyl)-1,3,8-triaza-spiro[4.5]decan-4-one | 4.46 | 600 |
| 393 | 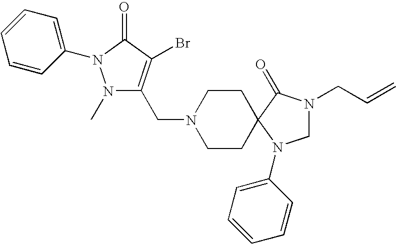 | 3-Allyl-8-(4-bromo-2-methyl-5-oxo-1-phenyl-2,5-dihydro-1H-pyrazol-3-ylmethyl)-1-phenyl-1,3,8-triaza-spiro[4.5]decan-4-one | 3.92 | 536 |
| 394 | 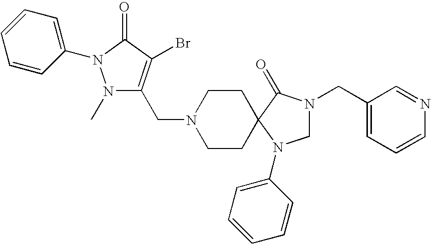 | 8-(4-Bromo-2-methyl-5-oxo-1-phenyl-2,5-dihydro-1H-pyrazol-3-ylmethyl)-1-phenyl-3-pyridin-3-ylmethyl-1,3,8-triaza-spiro[4.5]decan-4-one | 3.48 | 587 |
| 395 | 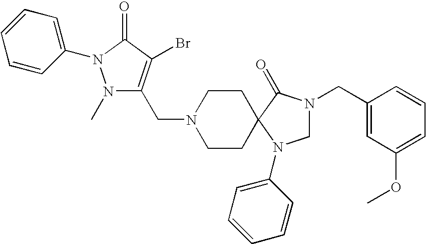 | 8-(4-Bromo-2-methyl-5-oxo-1-phenyl-2,5-dihydro-1H-pyrazol-3-ylmethyl)-3-(3-methoxy-benzyl)-1-phenyl-1,3,8-triaza-spiro[4.5]decan-4-one | 4.44 | 616 |
| 396 | 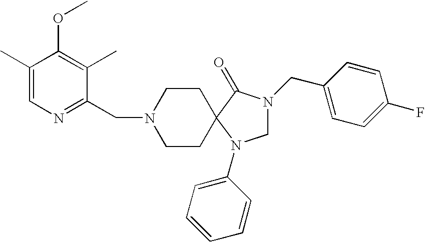 | 3-(4-Fluoro-benzyl)-8-(4-methoxy-3,5-dimethyl-pyridin-2-ylmethyl)-1-phenyl-1,3,8-triaza-spiro[4.5]decan-4-one | 4.15 | 489 |

| Example | Structure | Name | ¹HNMR |
|---|---|---|---|
| 397 | 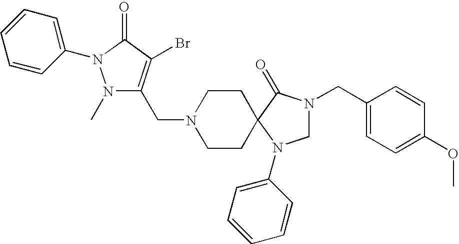 | 8-(4-Bromo-2-methyl-5-oxo-1-phenyl-2,5-dihydro-1H-pyrazol-3-ylmethyl)-3-(4-methoxy-benzyl)-1-phenyl-1,3,8-triaza-spiro[4.5]decan-4-one | (300 MHz,DMSO-d6): □(ppm) 7.57-7.51(m, 2 H),7.40-7.19(m,6 h), 6.89-6.85(m, 6 H), 4.59(s, 2 H), 4.53(s, 2 H), 3.75(s, 3 H), 3.70(s, 2 H), 3.00-2.80(m, 2 H),2.65-2.50(m,2 H), 1.68-1.63(m, 2 H) |
| 398 | 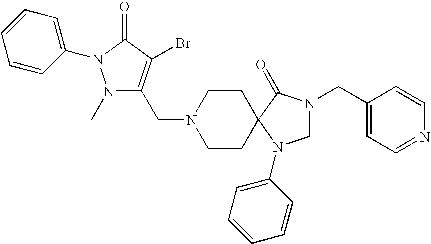 | 8-(4-Bromo-2-methyl-5-oxo-1-phenyl-2,5-dihydro-1H-pyrazol-3-ylmethyl)-1-phenyl-3-pyridin-4-ylmethyl-1,3,8-triaza-spiro[4.5]decan-4-one | (300 MHz,DMSO-d6): □(ppm) 8.56(d,J = 5.7 Hz,2 H), 7.57-7.53(m, 2 H), 7.43-7.21(m, 7 H),6.87(d, J = 8.1Hz, 2 H), 6.79(t, J = 7.2 Hz,1 H), 4.66(s, 2 H), 4.60(s,2 H), 3.70(s,2 H) |

### Example 399

### 1-(4-Bromo-2-methyl-5-oxo-1-phenyl-2,5-dihydro-1H-pyrazol-3-ylmethyl)-4-phenyl-piperidine-4-carboxylic acid 4-bromo-2-methyl-5-oxo-1-phenyl-2,5-dihydro-1H-pyrazol-3-ylmethyl ester

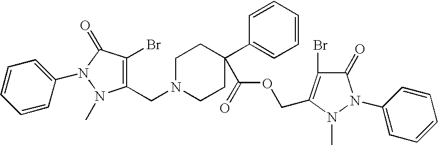

4-Bromo-5-bromomethyl-1-methyl-2-phenyl-1,2-dihydro-pyrazol-3-one (173 mg, 0.5 mmol), 4-phenyl-4-piperidinecarboxylic acid 4-methylbenzenesulfonate (189 mg, 0.5 mmol), and potassium carbonate (210 mg, 1.5 mmol) in acetonitrile (10 mL) were heated and stirred 18 hours, then partitioned between methylene chloride and water. The organic phase was evaporated and chromatographed on silica gel, eluting with 0-10% methanol in methylene chloride, to give the product as a white solid (130 mg, 35%). ¹H NMR (300 MHz, DMSO-d6): □ (ppm) 7.55-7.32 (m, 13H), 7.14 (d, J=7.5 Hz, 2H), 5.20 (s, 2H), 3.59 (s, 2H), 3.21 (s, 3H), 2.89-2.83 (m, 5H), 2.59-2.54 (m, 2H), 2.38-2.30 (m, 2H), 2.05-1.90 (m, 2H).

### Example 400

### 4-Phenyl-piperidine-1,4-dicarboxylic acid mono-tert-butyl ester

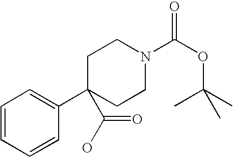

4-phenyl-4-piperidinecarboxylic acid 4-methylbenzenesulfonate (7.55 g, 20 mmol) was dissolved in a rapidly stirred mix of 1M NaOH (50 mL) and dioxane (25 mL). Di-t-butyldicarbonate (4.4 gm, 20 mmol) was added to the reaction. The reaction was stirred for 90 minutes. The reaction mix was transferred to a separatory funnel and washed with methylene chloride. The aqueous phase was made acidic by the addition of 1M hydrochloric acid (60 mL). Then the product was extracted from the aqueous phase with ethyl acetate and the resulting organic phase was evaporated to a colorless oil (4.3 g, 70%). ¹H NMR (300 MHz, DMSO-d6): □ (ppm) 12.66 (s, 1H), 7.41-7.24 (m, 5H), 3.82-3.77 (m, 2H), 3.05-2.90 (m, 2H), 2.38-2.33 (m, 2H), 1.76-1.66 (m, 2H), 1.39 (s, 9H).

### Example 401

### 4-Phenyl-piperidine-1,4-dicarboxylic acid 4-(4-bromo-2-methyl-5-oxo-1-phenyl-2,5-dihydro-1H-pyrazol-3-ylmethyl)ester 1-tert-butyl ester

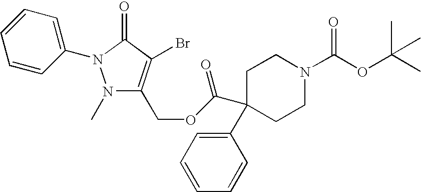

4-Bromo-5-bromomethyl-1-methyl-2-phenyl-1,2-dihydro-pyrazol-3-one (346 mg, 1 mmol), 4-phenyl-piperidine-1,4-dicarboxylic acid mono-tert-butyl ester (305 mg, 1 mmol), and diisopropylethylamine (0.18 mL, 1 mmol) in acetonitrile (3 mL) was microwaved 120° C. for 10 minutes. Partitioned the reaction between methylene chloride and saturated ammonium chloride. The organic phase was evaporated to a tan foam (560 mg, 98%). ¹H NMR (300 MHz, DMSO-d6): □ (ppm) 7.55-7.32 (m, 8H), 7.13 (d, J=7.5 Hz), 5.20 (s, 2H), 3.81-3.76 (m, 2H), 3.12-3.01 (m, 2H), 2.88 (s, 3H), 2.50-2.45 (m, 2H), 1.89-1.79 (m, 2H), 1.40 (s, 9H).

### Example 402

### 1-Benzyl-4-phenyl-piperidine-4-carboxylic acid 4-bromo-2-methyl-5-oxo-1-phenyl-2,5-dihydro-1H-pyrazol-3-ylmethyl ester

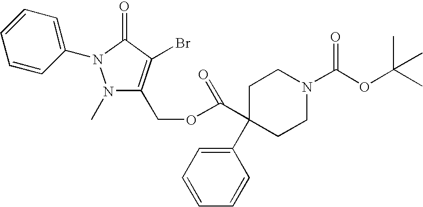

4-Phenyl-piperidine-1,4-dicarboxylic acid 4-(4-bromo-2-methyl-5-oxo-1-phenyl-2,5-dihydro-1H-pyrazol-3-ylmethyl)ester 1-tert-butyl ester (540 mg, 0.95 mmol) was dissolved in methylene chloride and trifluoroacetic acid (5 mL) and allowed to react for one hour. The volatiles were evaporated, and the residue was taken up in ether and crystals formed. Collected the tan solid by vacuum filtration (510 mg, 87%). A portion of this material (117 mg, 0.2 mmol) was dissolved in acetonitrile (4 mL) and diisopropylethylamine (0.18 mL, 1 mmol). Benzyl bromide (0.024 mL, 0.2 mmol) was added. After ten minutes the reaction was partitioned between ethyl acetate and water. The organic phase was evaporated. The residue was chromatographed on silica gel, eluting with 0-100% ethyl acetate in methylene chloride, to give the product (50 mg, 43%) as a white solid. ¹H NMR (300 MHz, DMSO-d6): □ (ppm) 7.55-7.14 (m, 15H), 5.17 (s, 2H), 3.43 (s, 2H), 2.87 (s, 3H), 2.75-2.70 (m, 2H), 2.6-2.5 (m, 2H), 2.20-2.12 (m, 2H), 1.99-1.92 (m, 2H).

### Example 403

### Piperidine-1,4-dicarboxylic acid 1-tert-butyl ester 4-ethyl ester

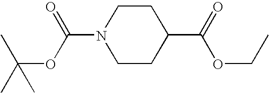

Piperidine-4-carboxylic acid ethyl ester (3.14 g, 20 mmol) was dissolved in acetonitrile (25 mL). Di-t-butyldicarbonate (5.23 g, 24 mmol) was added and the reaction was stirred for 30 minutes. Polyamine scavenger resin was added and reaction mix was allowed to stand for 18 hours. The resin was filtered away and the volatiles were evaporated. The residue was chromatographed on silica gel with 0-25% ethyl acetate in hexane. The title compound (4.88 g, 94%) was isolated as a colorless oil. ¹H NMR (300 MHz, DMSO-d6): □ (ppm) 4.06 (q, J=7.0 Hz, 2H), 3.85-3.80 (m, 2H), 2.86-2.78 (m, 2H), 2.54-2.46 (m, 2H), 1.80-1.76 (m, 2H), 1.39 (s, 9H), 1.18 (t, J=7.0 Hz, 3H).

### Example 404

### 4-Benzyl-piperidine-1,4-dicarboxylic acid 1-tert-butyl ester 4-ethyl ester

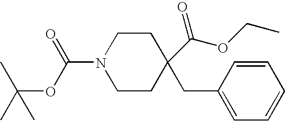

Piperidine-1,4-dicarboxylic acid 1-tert-butyl ester 4-ethyl ester (1.48 g, 5.76 mmol) was dissolved in dry THF (20 mL). The reaction was chilled to dry ice/acetone temperature. Potassium hexamethyldisilamide (6 mmol) was added dropwise. After 30 minutes benzyl bromide (1.5 mL, 12 mmol) was added. After 1 hour the cooling bath was removed and the reaction was stirred for three days. The reaction was partitioned between ethyl acetate and water. The organic phase was washed with dilute HCl and brine, then evaporated. The residue was chromatographed on silica gel, eluting with 0-25% ethyl acetate in hexane. Obtained the title compound (1.59 g, 80%) as a colorless oil. ¹H NMR (300 MHz, DMSO-d6): □ (ppm) 7.28-7.18 (m, 3H), 7.05 (d, J=6.8 Hz, 2H), 4.04 (q, J=7.1 Hz, 2H), 3.80-3.75 (m, 2H), 2.80-2.50 (m, 4H), 1.92-1.85 (m, 2H), 1.38 (s, 9H), 1.13 (t, J=7.0 Hz, 3H).

### Example 405

### 4-Benzyl-1-(4-bromo-2-methyl-5-oxo-1-phenyl-2,5-dihydro-1H-pyrazol-3-ylmethyl)-piperidine-4-carboxylic acid ethyl ester

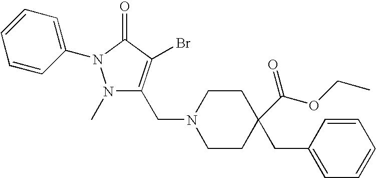

4-Benzyl-piperidine-1,4-dicarboxylic acid 1-tert-butyl ester 4-ethyl ester (135 mg, 0.39 mmol) was dissolved in methylene chloride (2 mL) and trifluoroacetic acid (1 mL). After 1 hour the volatiles were evaporated. The residue was dissolved in acetonitrile (2 mL) and diisopropylethylamine (0.5 mL). Added 4-bromo-5-bromomethyl-1-methyl-2-phenyl-1,2-dihydro-pyrazol-3-one (118 mg, 0.34 mmol). Microwaved the mixture at 160° C. for 10 minutes. Partitioned the reaction mix between methylene chloride and water. The organics phase was evaporated, and the residue was chromatographed on silica gel, eluting with 0-100% ethyl acetate in methylene chloride. Obtained the title compound (30 mg, 17%) as an off-white solid. ¹H NMR (300 MHz, DMSO-d6): □ (ppm) 7.56-7.51 (m, 2H), 7.42-7.21 (m, 6H), 7.05 (d, J=6.2 Hz, 2H), 4.05 (q, J=7.1 Hz, 2H), 3.53 (s, 2H), 3.19 (s, 3H), 2.70-2.85 (m, 2H), 2.12-1.96 (m, 4H), 1.60-1.50 (m, 2H), 1.14 (t, J=7.1 Hz, 3H).

### Example 406

### 4-Benzyl-piperidine-1,4-dicarboxylic acid 4-benzyl ester 1-tert-butyl ester

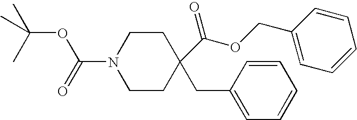

4-Benzyl-piperidine-1,4-dicarboxylic acid 1-tert-butyl ester 4-ethyl ester (500 mg, 1.44 mmol) was hydrolysed by suspending it in 6M sodium hydroxide (2 mL, 12 mmol) and methanol (1 mL) and was microwaved at 130° C. for 10 minutes. The resulting solution was partitioned between ethyl acetate and 1M HCl (25 mL, 25 mmol). The organic phase was evaporated and dried under vacuum. A portion of the resulting carboxylic acid (53 mg, 0.17 mmol) was dissolved in acetonitrile (10 mL) and diisopropylethylamine (90 ul, 0.5 mmol). Benzyl bromide (21 ul, 0.17 mmol) was added and the reaction was heated 70° C. for 2 hours, then at room temperature for 18 hours. Excess benzyl bromide was removed by stirring with polyamine resin for 3 hours. The resin was filtered off and the solvent was evaporated. Obtained a colorless oil (0.34 g, 85%). ¹H NMR (300 MHz, DMSO-d6): □ (ppm) 7.39-7.18 (m, 8H), 7.02-6.98 (m, 2H), 5.08 (s, 2H), 3.78-3.73 (m, 2H), 2.80-2.70 (m, 4H), 1.95-1.89 (m, 2H), 1.37 (s, 9H), 1.50-1.45 (m, 2H).

### Example 407

### 4-Benzyl-1-(4-bromo-2-methyl-5-oxo-1-phenyl-2,5-dihydro-1H-pyrazol-3-ylmethyl)-piperidine-4-carboxylic acid benzyl ester

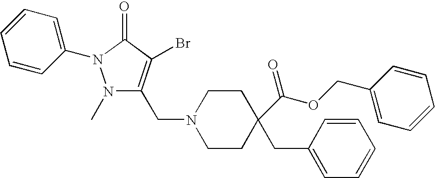

The Boc protecting group was removed by stirring in TFA (2 mL) in methylene chloride (5 mL) for 1 hour. The reaction was evaporated. The resulting residue (77 mg, 0.18 mmol) was mixed with 4-bromo-5-bromomethyl-1-methyl-2-phenyl-1,2-dihydro-pyrazol-3-one (63 mg, 0.18 mmol) and diisopropylethylamine (90 ul, 0.5 mmol) in acetonitrile (1 mL). This reaction was microwaved 150° C. for 10 minutes. The solvent was evaporated and the residue was chromatographed on silica gel, eluting with 0-100% ethyl acetate in methylene chloride. Obtained the product as a white solid (60 mg, 60%). ¹H NMR (300 MHz, DMSO-d6): □ (ppm) 7.55-7.50 (m, 2H), 7.42-7.20 (m, 1H), 7.10-7.00 (m, 2H), 5.08 (s, 2H), 3.50 (s, 2H), 2.82-2.75 (m, 5H), 2.10-1.97 (m, 4H), 1.65-1.50 (m, 2H).

### Example 408

### 4-Benzyl-1-(4-bromo-2-methyl-5-oxo-1-phenyl-2,5-dihydro-1H-pyrazol-3-ylmethyl)-piperidine-4-carboxylic acid phenyl ester

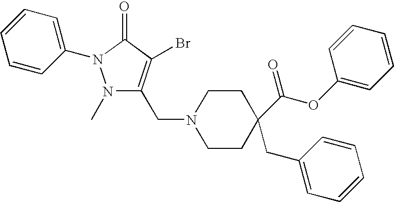

This compound was made in a method analogous to Example 407 using 4-phenyl-piperidine-1,4-dicarboxylic acid mono-tert-butyl ester. LC/MS (METHOD A): 560 (M+H) at 4.55 minutes.

### Example 409

### 4-Benzylcarbamoyl-4-phenyl-piperidine-1-carboxylic acid tert-butyl ester

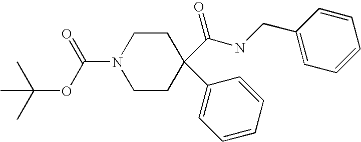

4-Phenyl-piperidine-1,4-dicarboxylic acid mono-tert-butyl ester (310 mg, 1 mmol) was dissolved in methylene chloride (10 mL) and diisopropylethylamine (350 ul, 2 mmol). The stirred reaction was chilled to ice bath temperature and thionyl chloride (88 uL, 1.2 mmol) was added. After 30 minutes benzyl amine (142 uL, 1.3 mmol) was added. The reaction was allowed to warm over 18 hours. Partitioned the reaction between ethyl acetate and 1M HCl. Evaporated the organic phase and chromatographed the residue on silica gel, eluting with 0-100% ethyl acetate in methylene chloride. Obtained the title compound (0.32 g, 82%) as a yellow foam. ¹H NMR (300 MHz, DMSO-d6): □ (ppm) 8.15 (t, J=5.8 Hz, 1H), 7.39-7.15 (m, 8H), 7.02 (d, J=6.2 Hz, 2H), 4.24 (d, J=5.8 Hz, 2H), 3.73-3.68 (m, 2H), 3.10-2.90 (m, 2H), 2.49-2.44 (m, 2H), 1.80-1.71 (m, 2H), 1.39 (s, 9H).

### Example 410

### 1-(4-Bromo-2-methyl-5-oxo-1-phenyl-2,5-dihydro-1H-pyrazol-3-ylmethyl)-4-phenyl-piperidine-4-carboxylic acid benzylamide

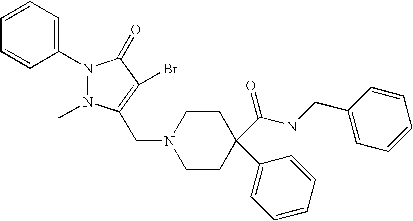

4-Benzylcarbamoyl-4-phenyl-piperidine-1-carboxylic acid tert-butyl ester (320 mg, 0.81 mmol) was dissolved in methylene chloride and TFA (3 mL). After 3 hours the volatiles were evaporated. A portion of this amine (82 mg, 0.2 mmol) was mixed with 4-bromo-5-bromomethyl-1-methyl-2-phenyl-1,2-dihydro-pyrazol-3-one (69 mg, 0.2 mmol) and diisopropylethylamine (90 uL, 0.5 mmol) in acetonitrile (1 mL). The reaction was microwaved 150° C. for 5 minutes. The volatiles were evaporated and the residue was chromatographed on silica gel, eluting with 0-100% ethyl acetate in methylene chloride. Obtained the title compound (32 mg, 29%) as an off-white foam. ¹H NMR (300 MHz, DMSO-d6): □ (ppm) 8.15 (m, 1H), 7.52-7.02 (m, 15H), 4.30-4.20 (m, 2H), 3.55 (s, 2H), 3.20 (s, 3H), 2.80-2.70 (m, 2H), 2.62-2.50 (m, 2H), 2.45-2.25 (m, 2H), 2.00-1.83 (m, 2H).

Compounds of Examples 411 and 412 were synthesized by a method analogous to the procedure of Example 410.

| | | | LC/MS | |
|---|---|---|---|---|
| | | | (METHOD | |
| | | | A) | M/z |
| Example | Structure | Name | (min.) | (M + H) |
| 411 | 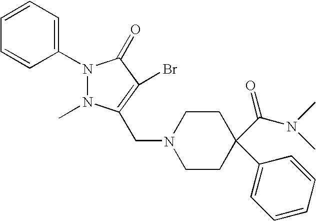 | 1-(4-Bromo-2-methyl-5-oxo-1-phenyl-2,5-dihydro-1H-pyrazol-3-ylmethyl)-4-phenyl-piperidine-4-carboxylic aciddimethylamide | 3.51 | 497 |
| 412 | 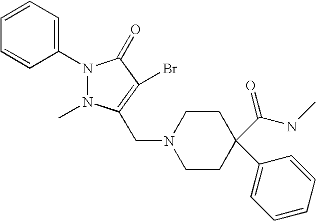 | 1-(4-Bromo-2-methyl-5-oxo-1-phenyl-2,5-dihydro-1H-pyrazol-3-ylmethyl)-4-phenyl-piperidine-4-carboxylic acidmethylamide | 3.24 | 483 |

### Example 413

### 1-(4-Bromo-2-methyl-5-oxo-1-phenyl-2,5-dihydro-1H-pyrazol-3-ylmethyl)-4-phenyl-piperidine-4-carbonitrile

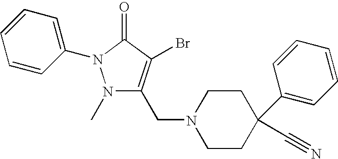

4-Bromo-5-bromomethyl-1-methyl-2-phenyl-1,2-dihydro-pyrazol-3-one (173 mg, 0.5 mmol), 4-cyano-4-phenylpiperidine hydrochloride (112 mg, 0.5 mmol), and diisopropylethylamine (0.5 mL, 2.8 mmol) in acetonitrile (2 mL) was microwaved at 170° C. for 10 minutes. The volatiles were evaporated, and the residue was chromatographed on silica gel, eluting with 0-100% ethyl acetate in methylene chloride. Obtained the title compound (170 mg, 74%) as a yellow foam. ¹H NMR (300 MHz, DMSO-d6): □ (ppm) 7.57-7.35 (m, 10H), 3.73 (s, 2H), 3.23 (s, 3H), 3.11-3.06 (m, 2H), 2.53-2.46 (m, 2H), 2.21-1.97 (m, 4H).

Compounds of Examples 414 through 421 were synthesized by a method analogous to the procedure of Example 413.

| | | | LC/MS | |
|---|---|---|---|---|
| | | | (METHOD | |
| | | | A) | M/z |
| Example | Structure | Name | (min.) | (M + H) |
| 414 | 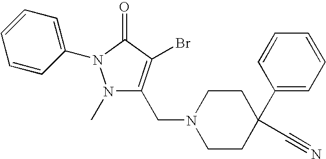 | 1-(4-Bromo-2-methyl-5-oxo-1-phenyl-2,5-dihydro-1H-pyrazol-3-ylmethyl)-4-phenyl-piperidine-4-carbonitrile | 3.6 | 509 |
| 415 | 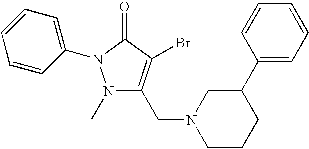 | 4-Bromo-1-methyl-2-phenyl-5-(3-phenyl-piperidin-1-ylmethyl)-1,2-dihydro-pyrazol-3-one | 3.69 | 426 |
| 416a | 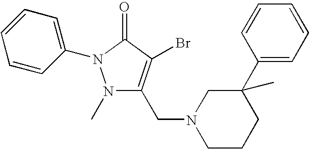 | 4-Bromo-1-methyl-5-(3-methyl-3-phenyl-piperidin-1-ylmethyl)-2-phenyl-1,2-dihydro-pyrazol-3-one | 4.57 | 440 |
| 417 | 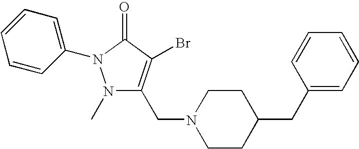 | 5-(4-Benzyl-piperidin-1-ylmethyl)-4-bromo-1-methyl-2-phenyl-1,2-dihydro-pyrazol-3-one | 3.72 | 440 |
| 418 | 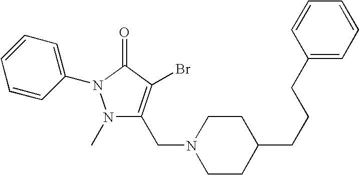 | 4-Bromo-1-methyl-2-phenyl-5-[4-(3-phenyl-propyl)-piperidin-1-ylmethyl]-1,2-dihydro-pyrazol-3-one | 4.03 | 468 |
| 419 | 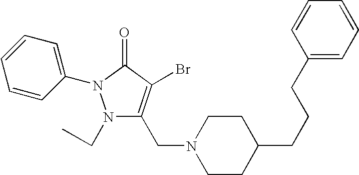 | 4-Bromo-1-ethyl-2-phenyl-5-[4-(3-phenyl-propyl)-piperidin-1-ylmethyl]-1,2-dihydro-pyrazol-3-one | 4.06 | 482 |
| 420 | 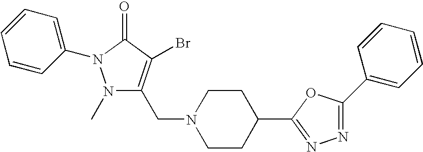 | 4-Bromo-1-methyl-2-phenyl-5-[4-(5-phenyl-[1,3,4]oxadiazol-2-yl)-piperidin-1-ylmethyl]-1,2-dihydro-pyrazol-3-one | 3.63 | 494 |
| 421 | 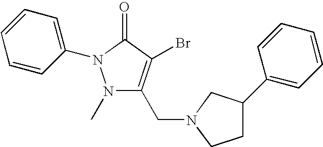 | 4-Bromo-1-methyl-2-phenyl-5-(3-phenyl-pyrrolidin-1-ylmethyl)-1,2-dihydro-pyrazol-3-one | 3.43 | 412 |

### Example 416b

### Resolution of 4-Bromo-1-methyl-5-(3-methyl-3-phenyl-piperidin-1-ylmethyl)-2-phenyl-1,2-dihydro-pyrazol-3-one

4-Bromo-1-methyl-5-(3-methyl-3-phenyl-piperidin-1-ylmethyl)-2-phenyl-1,2-dihydro-pyrazol-3-one (50 mg) was dissolved in isopropanol (0.75 mL) and diluted with hexane (1.5 mL). The solution was separated on a 1″ Chiracel OD column, equilibrated and eluted with 40% isopropanol in hexane with a flow rate of 4.5 mL/min. Obtained a baseline separation of the 2 enantiomers. The solvents were evaporated and the resulting oils were dissolved in ether. Scratched to form crystals then evaporated. The first eluting enantiomer was labelled (10 mg). The second eluting enantiomer was labelled (10 mg). No rotation was run on these enantiomers. Each had LC/MS (METHOD A) (m+H) 440 at 4.57 min.

### Example 422

### 1-(4-Bromo-2-methyl-5-oxo-1-phenyl-2,5-dihydro-1H-pyrazol-3-ylmethyl)-4-phenyl-piperidine-4-carboxylic acid amide

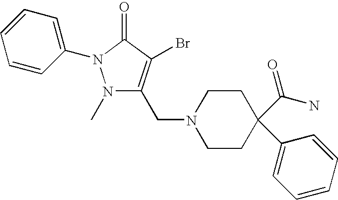

1-(4-Bromo-2-methyl-5-oxo-1-phenyl-2,5-dihydro-1H-pyrazol-3-ylmethyl)-4-phenyl-piperidine-4-carbonitrile (96 mg, 0.21 mmol) was dissolved in concentrated sulfuric acid (10 mL) and heated to 55° C. for 18 hours. Partitioned between methylene chloride and 1M sodium hydroxide. The organic phase was evaporated and the residue was chromatographed on silica gel, eluting with 0-10% methanol in methylene chloride. Obtained the title compound (80 mg, 80%) as a white solid. ¹H NMR (300 MHz, DMSO-d6): □ (ppm) 7.55-7.50 (m, 2H), 7.41-7.31 (m, 8H), 7.24-7.20 (m, 1H), 7.14 (s, 1H), 6.93 (s, 1H), 3.56 (s, 2H), 3.21 (s, 3H), 3.82-3.75 (m, 2H), 2.55-2.45 (m, 2H), 2.37-2.30 (m, 2H), 1.90-1.79 (m, 2H).

### Example 423

### Piperidine-1,4-dicarboxylic acid 4-benzyl ester 1-tert-butyl ester

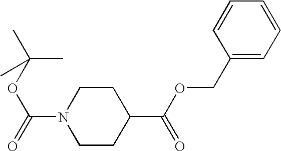

Piperidine-4-carboxylic acid (12.9 g, 100 mmol) was dissolved in a rapidly stirred mixture of dioxane (100 mL) and 1M sodium hydroxide (300 mmol). Di-t-butyldicarbonate (22 g, 100 mmol) was added. After 18 hours the volatiles were evaporated. The aqueous residue was acidified with 1M hydrochloric acid and extracted with methylene chloride. The organic phase was evaporated to give the intermediate piperidine-1,4-dicarboxylic acid mono-tert-butyl ester as a white solid (19.6 g, 85%). ¹H NMR (300 MHz, DMSO-d6): □ (ppm) 12.20 (s, 1H), 3.85-3.80 (m, 2H), 2.85-2.77 (m, 2H), 2.44-2.35 (m, 2H), 1.80-1.75 (m, 2H), 1.44-1.31 (m, 11H). A portion of this intermediate (2.29 g, 10 mmol) was mixed with potassium carbonate (1.7 g, 12 mmol) and benzyl bromide (1.2 mL, 10 mmol) in acetonitrile (20 mL). Heated the reaction to 60° C. for 18 hours. The reaction was partitioned between ethyl acetate and water. The organic phase was washed with water and brine, then dried over magnesium sulfate and evaporated. The residue was chromatographed on silica gel with 0-25% ethyl acetate in methylene chloride. Obtained the title compound (2.3 g, 72%) as a colorless oil. ¹H NMR (300 MHz, DMSO-d6): □ (ppm): 7.41-7.30 (m, 5H), 5.10 (s, 2H), 3.86-3.81 (m, 2H), 2.87-2.78 (m, 2H), 2.64-2.55 (m, 1H), 1.85-1.80 (m, 2H), 1.49-1.38 (m, 11H).

### Example 424

### 1-(4-Bromo-2-methyl-5-oxo-1-phenyl-2,5-dihydro-1H-pyrazol-3-ylmethyl)-piperidine-4-carboxylic acid benzyl ester

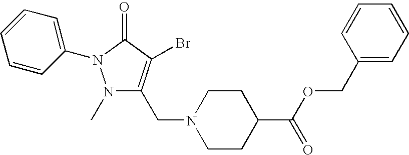

Piperidine-1,4-dicarboxylic acid 4-benzyl ester 1-tert-butyl ester (200 mg, 0.63 mmol) was dissolved in methylene chloride (5 mL) and treated with trifluoroacetic acid (2 mL). After three hours the volatiles were evaporated. The residue was partioned between methylene chloride and 1M sodium hydroxide. The organic phase was evaporated and the residue was chromatographed on silica gel, eluting with 0-10% methanol in methylene chloride. The resulting intermediate (69 mg, 0.32 mmol), was mixed with 4-bromo-5-bromomethyl-1-methyl-2-phenyl-1,2-dihydro-pyrazol-3-one (109 mg, 0.32 mmol) and diisopropylethylamine (170 uL, 1 mmol) in acetonitrile (1 mL). The reaction was microwaved at 150° C. for 5 minutes. The volatiles were evaporated and the residue was chromatographed on silica gel, eluting with 0-25% ethyl acetate in methylene chloride.

The product was recrystallized from ether (15 mL) to give the title compound (58 mg, 19%) as a white solid. ¹H NMR (300 MHz, DMSO-d6): □ (ppm): 7.55-7.50 (m, 2H), 7.41-7.33 (m, 8H), 5.11 (s, 2H), 3.58 (s, 2H), 3.20 (s, 3H), 2.88-2.84 (m, 2H), 2.50-2.40 (m, 1H), 2.20-2.13 (m, 2H), 1.89-1.85 (m, 2H), 2.70-2.55 (m, 2H).

### Example 425

### 1-(4-Bromo-2-methyl-5-oxo-1-phenyl-2,5-dihydro-1H-pyrazol-3-ylmethyl)-piperidine-4-carboxylic acid phenyl ester

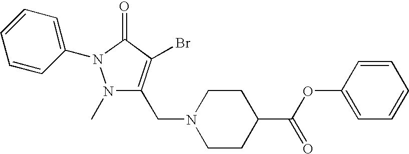

This compound was made in a method analogous to Example 424. LC/MS (METHOD A): 470 (m+H) at 3.76 min.

### Example 426

### 5-Bromomethyl-4-fluoro-1-methyl-2-phenyl-1,2-dihydro-pyrazol-3-one

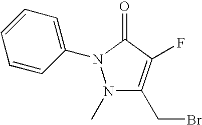

4-Fluoro-1,5-dimethyl-2-phenyl-1,2-dihydro-pyrazol-3-one (290 mg, 1.4 mmol) was dissolved in hot carbon tetrachloride (100 mL). N-bromosuccinamide (250 mg, 1.4 mmol) and benzoyl peroxide (50 mg) were added. The reaction was photolysed/heated with a tungsten lamp. After 15 minutes the solids were filtered off and the volatiles were evaporated. The residue was chromatographed on silica gel, eluting with 0-100% ethyl acetate in methylene chloride. The title product (250 mg, 63%) was obtained as an off-white solid. ¹H NMR (300 MHz, DMSO-d6): □ (ppm) 7.63-7.52 (m, 2H), 7.43-7.26 (m, 2H), 4.80 (s, 2H), 3.05 (s, 3H).

### Example 427

### 8-(4-Fluoro-2-methyl-5-oxo-1-phenyl-2,5-dihydro-1H-pyrazol-3-ylmethyl)-1-phenyl-1,3,8-triaza-spiro[4.5]decan-4-one

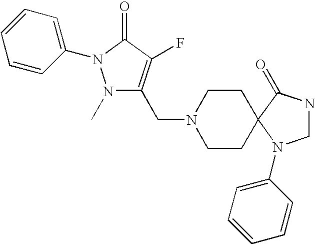

5-Bromomethyl-4-fluoro-1-methyl-2-phenyl-1,2-dihydro-pyrazol-3-one (350 mg, 0.1 mmol), 1-phenyl-1,3,8-triaza-spiro[4.5]decan-4-one (350 mg, 0.08 mmol) and diisopropylethylamine (120 uL, 0.7 mmol) in acetonitrile (1 mL) was microwaved at 150° C. for five minutes. The title compound crystallized as a tan solid on standing. The solid (110 mg, 73%) was collected by vacuum filtration and washed with acetonitrile (2 mL). ¹H NMR (300 MHz, DMSO-d6): □ (ppm) 8.63 (s, 1H), 7.56-7.51 (m, 2H), 7.40-7.36 (m, 3H), 7.23 (t, J=8.1 Hz, 2H), 6.86 (d, J=8.4 Hz, 2H), 6.76 (t, J=7.2 Hz, 1H), 4.57 (s, 2H), 3.69 (s, 2H), 3.10 (s, 3H), 2.95-2.80 (m, 4H), 2.62-2.52 (m, 2H), 1.65-1.61 (m, 2H).

### Example 428

### 4-(2-Phenoxyethyl)-piperidine trifluoroacetate

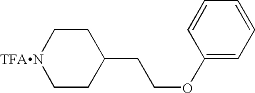

To a solution of 4-(2-hydroxyethyl)-piperidine-1-carboxylic acid tert-butyl ester (0.22 mL, 1 mmol), phenol (0.094 g, 1 mmol), and triphenylphosphine (0.26 g, 1 mmol) in dry THF (5 mL) was added dropwise diisopropylazodicarboxylate (0.2 mL, 1 mmol). The mixture was stirred 1 hour, then evaporated. The residue was chromatographed on silica gel eluting with 0-25% ethyl acetate in hexane. This intermediate was deprotected by treatment with trifluoroacetic acid (1 mL) in methylene chloride (5 mL) for 1 hour. The reaction was evaporated and the resulting solid was dried in vacuo (0.19 g, 59%). ¹H NMR (300 MHz, DMSO-d6): □(ppm) 8.5 (bs, 1H), 8.22 (bs, 1H), 7.28 (t, J=7.9 hz, 2H), 6.95-6.90 (m, 3H), 4.01 (t, J=6.2 hz, 2H), 3.28-3.23 (m, 2H), 2.90-2.85 (m, 2H), 1.90-1.67 (m, 5H), 1.40-1.25 (m, 2H).

### Example 429

### 4-Bromo-1-methyl-5-[4-(2-phenoxyethyl)piperidin-1-ylmethyl]-2-phenyl-1,2-dihydropyrazol-3-one

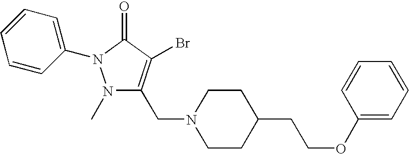

A mixture of 4-(2-phenoxyethyl)-piperidine trifluoroacetate (0.09 g, 0.28 mmol), 4-bromo-5-bromomethyl-1-methyl-2-phenyl-1,2-dihydro-pyrazol-3-one (0.1 g, 0.28 mmol), and diisopropylethylamine (0.18 mL, 1 mmol) in acetonitrile (1 mL) was microwaved at 150° C. for 3 minutes. The volatiles were evaporated and the residue was chromatographed on silica gel, eluting with 0-100% ethyl acetate in hexane. The title compound was isolated as a white foam (0.073 g, 56%). ¹H NMR (300 MHz, DMSO-d6): □(ppm) 7.54 (t, J=7.6 hz, 2H), 7.42-7.24 (m, 5H), 6.94-6.88 (m, 3H), 4.00 (t, J=6.4 hz, 2H), 3.57 (s, 2H), 3.21 (s, 3H), 2.93-2.88 (m, 2H), 2.07 (t, J=11 hz, 2H), 1.75-1.63 (m, 4H), 1.55-1.40 (m, 1H), 1.30-1.15 (m, 2H).

Compounds of Examples 430 through 444 were synthesized by a method analogous to the procedure of Example 429.

| | | | LC/MS | |
|---|---|---|---|---|
| | | | (METHOD | |
| | | | A) | M/z |
| Example | Structure | Name | (min.) | (m + H) |
| 430 | 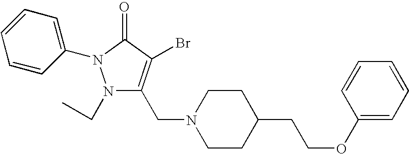 | 4-Bromo-1-ethyl-5-[4-(2-phenoxy-ethyl)-piperidin-1-ylmethyl]-2-phenyl-1,2-dihydro-pyrazol-3-one | 4.07 | 484 |
| 431 | 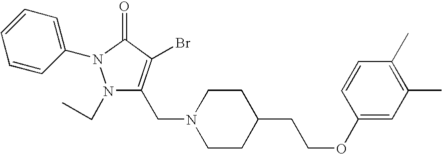 | 4-Bromo-5-{4-[2-(3,4-dimethylphenoxy)ethyl]piperidin-1-ylmethyl}-1-ethyl-2-phenyl-1,2-dihydro-pyrazol-3-one | 4.34 | 512 |
| 432 | 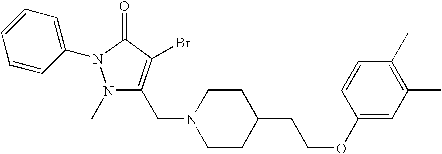 | 4-Bromo-5-{4-[2-(3,4-dimethyl-phenoxy)-ethyl]-piperidin-1-ylmethyl}-1-methyl-2-phenyl-1,2-dihydro-pyrazol-3-one | 4.25 | 498 |
| 433 | 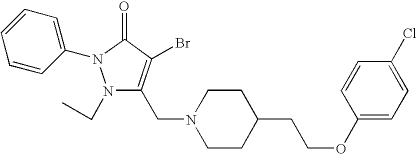 | 4-Bromo-5-{4-[2-(4-chlorophenoxy)-ethyl]piperidin-1-ylmethyl}-1-ethyl-2-phenyl-1,2-dihydropyrazol-3-one | 4.35 | 518 |
| 434 | 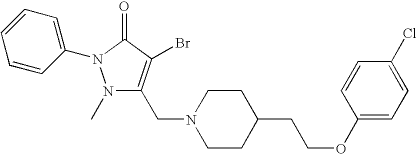 | 4-Bromo-5-{4-[2-(4-chlorophenoxy)-ethyl]piperidin-1-ylmethyl}-1-meth-yl-2-phenyl-1,2-dihydropyrazol-3-one | 4.22 | 504 |
| 435 | 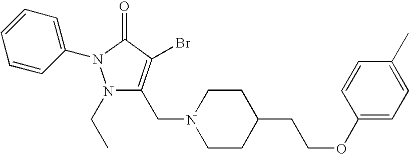 | 4-Bromo-1-ethyl-2-phenyl-5-[4-(2-p-tolyloxyethyl)-piperidin-1-ylmethyl]-1,2-dihydro-pyrazol-3-one | 4.05 | 498 |
| 436 | 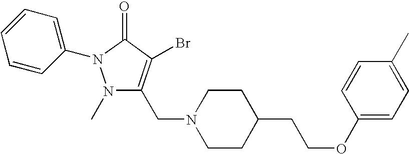 | 4-Bromo-1-methyl-2-phenyl-5-[4-(2-p-tolyloxyethyl)-piperidin-1-ylmethyl]-1,2-dihydro-pyrazol-3-one | 3.94 | 484 |
| 437 | 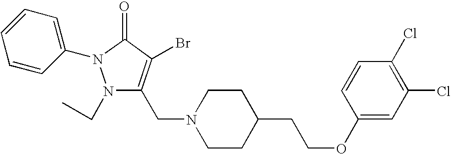 | 4-Bromo-5-{4-[2-(3,4-dichlorophenoxy)ethyl]-piperidin-1-ylmethyl}-1-ethyl-2-phenyl-1,2-dihydropyrazol-3-one | 4.33 | 552 |
| 438 | 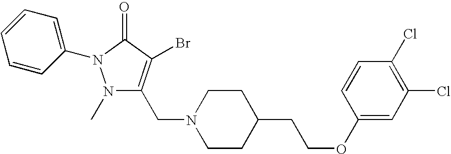 | 4-Bromo-5-{4-[2-(3,4-dichlorophenoxy)ethyl]piperidin-1-ylmethyl}-1-methyl-2-phenyl-1,2-dihydropyrazol-3-one | 4.23 | 538 |
| 439 | 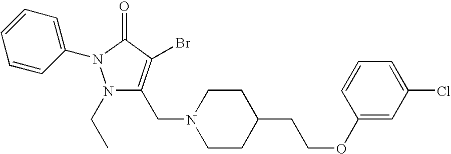 | 4-Bromo-5-{4-[2-(3-chlorophenoxy)-ethyl]piperidin-1-ylmethyl}-1-ethyl-2-phenyl-1,2-dihydropyrazol-3-one | 4.19 | 518 |
| 440 | 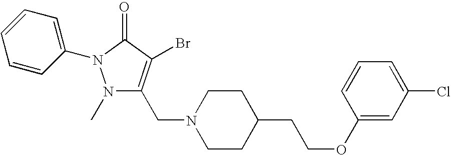 | 4-Bromo-5-{4-[2-(3-chlorophenoxy)-ethyl]-piperidin-1-ylmethyl}-1-methyl-2-phenyl-1,2-dihydropyrazol-3-one | 4.07 | 504 |
| 441 | 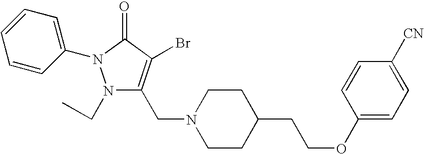 | 4-{2-[1-(4-Bromo-2-ethyl-5-oxo-1-phenyl-2,5-dihydro-1H-pyrazol-3-ylmeth-yl)piperidin-4-yl]-ethoxy}benzonitrile | 3.83 | 509 |
| 442 | 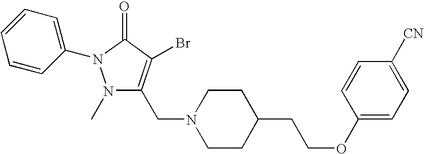 | 4-{2-[1-(4-Bromo-2-methyl-5-oxo-1-phenyl-2,5-dihydro-1H-pyrazol-3-ylmeth-yl)-piperidin-4-yl]-ethoxy}benzonitrile | 3.72 | 495 |
| 443 | 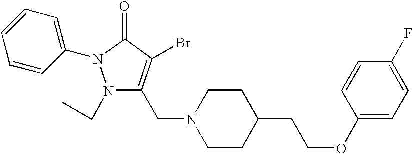 | 4-Bromo-1-ethyl-5-{4-[2-(4-fluoro-phenoxy)-ethyl]-piperidin-1-ylmethyl}-2-phenyl-1,2-dihydropyrazol-3-one | 4.02 | 502 |
| 444 | 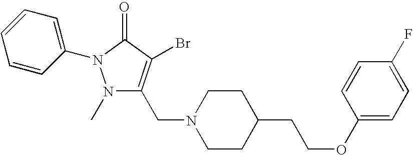 | 4-Bromo-5-{4-[2-(4-fluoro-phenoxy)-ethyl]-piperidin-1-ylmethyl}-1-methyl-2-phenyl-1,2-dihydropyrazol-3-one | 3.90 | 488 |

### Example 445

### 4-Hydroxymethylpiperidine-1-carboxylic acid tert-butyl ester

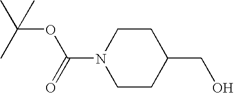

Piperidin-4-yl-methanol (1.15 g, 10 mmol) was dissolved in methylene chloride (20 ml) and diisopropylethylamine (1.8 mL, 10 mmol). Di-tert-butyldicarbonate (2.18 g, 10 mmol) was added and stirred for 1 hour. Volatiles were evaporated. The residue was partitioned between ethyl acetate and saturated ammonium chloride. The organic phase was washed with brine and evaporated to a colorless oil that crystallized on standing (2.11 g, 98%). ¹H NMR (300 MHz, DMSO-d6): □(ppm) 4.42 (t, J=5.3 hz, 1H), 4.00-3.90 (m, 2H), 3.27-3.16 (m, 4H), 3.75-3.60 (m, 2H), 1.63-1.59 (m, 2H), 1.55-1.45 (m, 1H), 1.38 (s, 9H), 1.03-0.98 (2H).

### Example 446

### 4-(4-Fluorobenzyloxymethyl)piperidine Trifluoroacetic Acid Salt

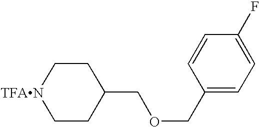

4-Hydroxymethylpiperidine-1-carboxylic acid tert-butyl ester (0.34 g, 1.58 mmol) was dissolved in NMP (5 mL). Sodium hydride (0.12 g, 3 mmol) was added and stirred for 10 minutes. 4-Fluorobenzyl bromide (0.24 mL, 2 mmol) was added and stirred for 3 hours. The reaction was quenched by addition of water. The mix was extracted with ethyl acetate and the organic phase was washed 5 times with brine and evaporated. Excess fluorobenzylbenzyl bromide was removed by dissolving the residue in methylene chloride and treating with poly-amine scavenger resin for 16 hours. The resulting crude product was further purified chromatography on silica gel eluting with 0-25% ethyl acetate in methylene chloride. The boc group was removed by treatment with trifluoroacetic acid (2 mL) in methylene chloride (5 mL) for 30 minutes. The reaction was evaporated and dried in vacuo to give a yellow oil (0.2 g, 38%).

### Example 447

### 4-Bromo-5-[4-(4-fluoro-benzyloxymethyl)-piperidin-1-ylmethyl]-1-methyl-2-phenyl-1,2-dihydro-pyrazol-3-one

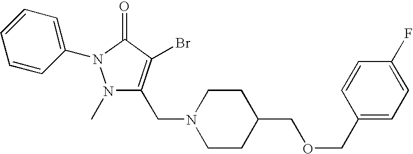

A mixture of 4-(4-fluorobenzyloxymethyl)piperidine trifluoroacetate (0.1 g, 0.3 mmol), 4-bromo-5-bromomethyl-1-methyl-2-phenyl-1,2-dihydro-pyrazol-3-one (0.11 g, 0.3 mmol), and diisopropylethylamine (0.18 mL, 1 mmol) in acetonitrile (1 mL) was microwaved at 150° C. for 3 minutes. The volatiles were evaporated and the residue was chromatographed on silica gel eluting with 0-100% ethyl acetate in methylene chloride. The title compound was obtained as a colorless oil (56 mg, 40%). ¹H NMR (300 MHz, DMSO-d6): □(ppm) 7.53 (t, J=7.6 hz, 2H), 7.41-7.30 (m, 5H), 7.16 (t, J=8.9 hz, 2H), 4.43 (s, 2H), 3.57 (s, 2H), 3.27 (obscured), 3.19 (s, 3H), 2.92-2.88 (m, 2H), 2.07 (t, J=19.6 hz, 2H), 1.70-1.50 (m, 3H), 1.35-1.20 (m, 2H). LC/MS (METHOD A) M/z (M+H) 485 at 3.68 min.

Compounds of Examples 448 through 451 were synthesized by a method analogous to the procedure of Example 447.

| | | | LC/MS | |
|---|---|---|---|---|
| | | | (METHOD | M/z |
| Example | Structure | Name | A) (min.) | (m + H) |
| 448 | 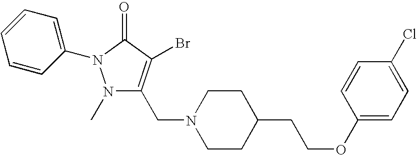 | 4-Bromo-5-[4-(4-chloro-benzyloxymeth-yl)-piperidin-1-ylmethyl]-1-methyl-2-phenyl-1,2-dihydro-pyrazol-3-one | 3.86 | 504 |
| 449 | 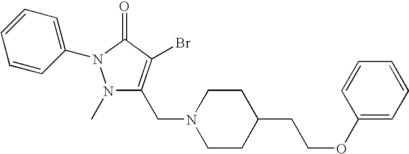 | 5-(4-Benzyloxy-methylpiperidin-1-ylmethyl)-4-bromo-1-methyl-2-phenyl-1,2-dihydro-pyrazol-3-one | 3.63 | 470 |
| 450 | 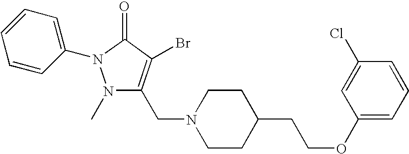 | 4-Bromo-5-[4-(3-chloro-benzyloxy-methyl)-piperidin-1-ylmethyl]-1-methyl-2-phenyl-1,2-dihydro-pyrazol-3-one | 3.86 | 504 |

### Example 451

### 4-(2-Iodo-ethyl)-piperidine-1-carboxylic acid tert-butyl ester

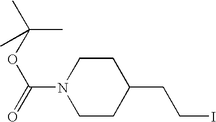

4-(2-Hydroxy-ethyl)-piperidine-1-carboxylic acid tert-butyl ester (14.3 g, 62.6 mmol), imidazole (4.35 g, 64 mmol), and triphenylphosphine (17.6 g, 67 mmol) were dissolved in acetonitrile (50 mL) and ether (50 mL). Iodine (17 g, 67 mmol) was added in small portions over 30 minutes. After 2 hours the reaction was diluted with ether (500 mL). The triphenylphosphine oxide byproduct precipitated and was filtered off. The filtrate was evaporated and the residue was dissolved/suspended in ether. The solids were filtered off and the filtrate was evaporated and the resulting oil was chromatographed on silica gel eluting with 0-25% ethyl acetate in hexane. The title compound was obtained as a yellow oil (15.3 g, 72%).

### Example 452

### 4-(2-Triphenylphosphonium-ethyl)-piperidine-1-carboxylic acid tert-butyl ester iodide

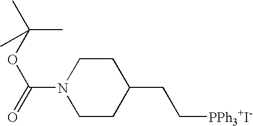

4-(2-Iodo-ethyl)-piperidine-1-carboxylic acid tert-butyl ester (15.3 g, 45.1 mmol) and triphenylphosphine (11.8 g, 45.1 mmol) were dissolved in acetonitrile (100 mL) and refluxed 16 hours. At that time the condenser was removed and the reaction was distilled to a white solid. The solid was washed with THF (25 mL) and dried in vacuo (23.2 g, 85%).

### Example 453

### 4-[3-(3-Fluoro-phenyl)-propyl]-piperidine

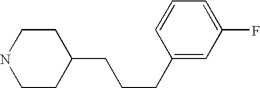

4-(2-Triphenylphosphonium-ethyl)-piperidine-1-carboxylic acid tert-butyl ester iodide (6 g, 10 mmol) was dissolved in dry THF. The solution was cooled to ice bath temperature. A 1.6 M solution of n-butyllithium (10 mL 16 mmol) was added over 5 minutes. The reaction was heated to reflux. 3-Fluorobenzaldehyde (1.17 mL, 10 mmol) was added. The reaction was refluxed for 5 hours. The reaction was evaporated and partitioned between saturated ammonium chloride and methylene chloride. The organic phase was washed with brine and dried over magnesium sulfate. Silica gel chromatography with 0-25% ethyl acetate in hexane afforded the olefin intermediate (2.5:1 E:Z ratio) as a yellow oil (1.6 g, 50%). A portion of this material (1 g, 3.1 mmol) was dissolved in ethanol (50 mL) and hydrogenated over Pd/C at 50 psi hydrogen. After one hour the catalyst was filtered off and the filtrate evaporated to a yellow oil (0.85 g, 85%). The oil was dissolved in methylene chloride (10 mL) and trifluoroacetic acid (3 mL). After one hour the reaction was evaporated. The residue was partitioned between 1M sodium hydroxide and methylene chloride. The organic phase was washed with brine and dried over magnesium sulfate. Evaporation gave the title compound as a yellow oil (0.54 g, 92%). ¹H NMR (300 MHz, CDCl₃): □ (ppm) 7.25-7.18 (m, 1H), 6.95-6.83 (m, 3H), 3.70-3.50 (m, 4H), 1.70-1.55 (m, 4H), 1.40-1.20 (m, 4H), 1.15-1.00 (m, 2H).

### Example 454

### 4-Bromo-5-{4-[3-(3-fluorophenyl)propyl]-piperidin-1-ylmethyl}-1-methyl-2-phenyl-1,2-dihydropyrazol-3-one

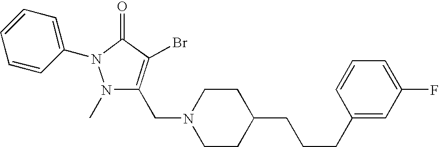

A mix of 4-[3-(3-fluoro-phenyl)-propyl]-piperidine (0.066 g, 0.3 mmol), 4-bromo-5-bromomethyl-1-methyl-2-phenyl-1,2-dihydro-pyrazol-3-one (0.104 g, 0.3 mmol) and diisopropylethylamine (0.74 mL, 1 mmol) in acetonitrile (1 mL) was microwaved 150° C. for 5 minutes. The volatiles were evaporated and the residue was chromatographed on silica gel eluting with 0-100% ethyl acetate in hexanes. Obtained the title compound as a colorless oil that crystallized on standing (0.09 g, 62%). ¹H NMR (300 MHz, CDCl₃): □ (ppm) 7.49-7.19 (m, 6H), 6.96-6.85 (m, 3H), 3.52 (s, 2H), 3.23 (s, 3H), 2.95-2.83 (m, 2H), 2.60 (t, J=7.5 Hz, 2H), 2.11 (t, J=10.2 Hz, 2H), 1.72-1.55 (m, 3H), 1.30-1.10 (m, 6H).

Compounds of Examples 455 through 466 were synthesized by a method analogous to the procedure of Example 454.

| | | | LC/MS | |
|---|---|---|---|---|
| | | | (METHOD | |
| | | | A) | M/z |
| Example | Structure | Name | (min.) | (m + H) |
| 455 | 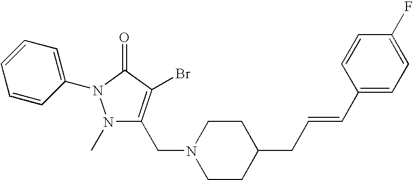 | 4-Bromo-5-{4-[(E)-3-(4-fluorophenyl)-allyl]-piperidin-1-ylmethyl}-1-methyl-2-phenyl-1,2-dihydro-pyrazol-3-one | 3.95 | 484 |
| 456 | 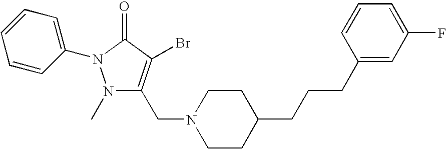 | 4-Bromo-5-{4-[3-(3-fluorophenyl)-propyl]-piperidin-1-ylmethyl}-1-methyl-2-phenyl-1,2-dihydropyrazol-3-one | 3.97 | 486 |
| 457 | 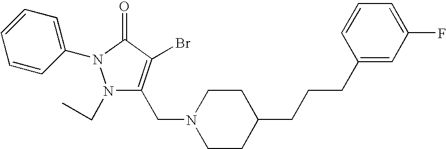 | 4-Bromo-1-ethyl-5-{4-[3-(3-fluoro-phenyl)propyl]-piperidin-1-ylmethyl}-2-phenyl-1,2-dihydro-pyrazol-3-one | 4.09 | 500 |
| 458 | 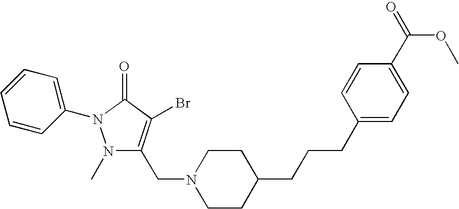 | 4-{3-[1-(4-Bromo-2-methyl-5-oxo-1-phenyl-2,5-dihydro-1H-pyrazol-3-ylmethyl)-piperidin-4-yl]-propyl}-benzoicacid methylester | 3.88 | 5.26 |
| 459 | 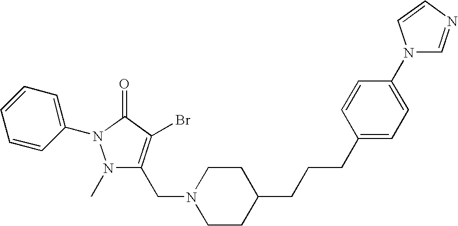 | 4-Bromo-5-{4-[3-(4-imidazol-1-yl-phenyl)-propyl]-piperidin-1-ylmethyl}-1-methyl-2-phenyl-1,2-dihydro-pyrazol-3-one | 3.05 | 534 |
| 460 | 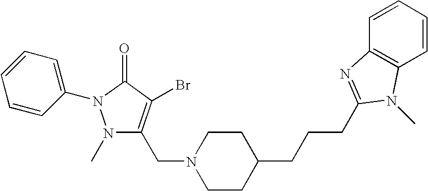 | 4-Bromo-1-methyl-5-{4-[3-(1-methyl-1H-benzoimidazol-2-yl)-propyl]-piperidin-1-ylmethyl}-2-phenyl-1,2-dihydropyrazol-3-one | 2.77 | 522 |
| 461 | 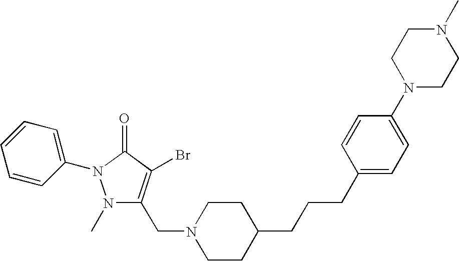 | 4-Bromo-1-methyl-5-(4-{3-[4-(4-methyl-piperazin-1-yl)-phenyl]-propyl}-piperidin-1-ylmethyl)-2-phenyl-1,2-dihydropyrazol-3-one | 3.13 | 566 |
| 462 | 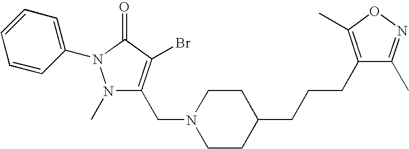 | 4-Bromo-5-{4-[3-(3,5-dimethyl-isoxazol-4-yl)-propyl]-piperidin-1-ylmethyl}-1-methyl-2-phenyl-1,2-dihydro-pyrazol-3-one | 3.50 | 487 |

| Example | Structure | Name | ¹H NMR |
|---|---|---|---|
| 459a | 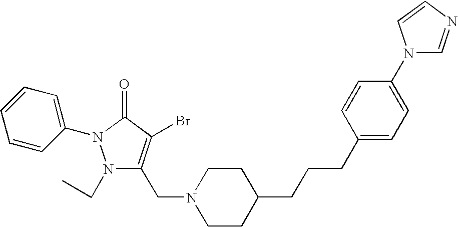 | 4-Bromo-5-{4-[3-(4-imidazol-1-yl-phenyl)-propyl]-piperidin-1-ylmethyl}-1-ethyl-2-phenyl-1,2-dihydro-pyrazol-3-one | ¹H NMR (300 MHz,CDCl₃) □ 7.83(s,1 H), 7.50-7.17(m,11 H), 3.78(q, J = 6.9Hz, 2 H), 3.51(s, 2 H),2.93(d, J = 11.3 Hz,2 H), 2.65(t, J = 7.6Hz, 2 H), 2.13(t, J =10.3 Hz, 2 H), 1.76-1.52(m, 4 H), 1.36-1.14(m, 5 H), 0.86(t,J = 6.9 Hz, 3 H). |
| 463 | 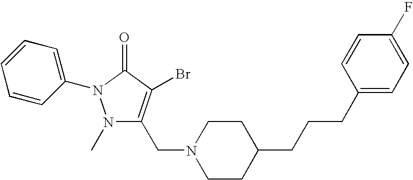 | 4-Bromo-5-{4-[3-(4-fluoro-phenyl)-propyl]-piperidin-1-ylmethyl}-1-methyl-2-phenyl-1,2-dihydro-pyrazol-3-one | (300 MHz, DMSO-d6): □ (ppm) 7.52(t,J = 7.5 hz, 2 H), 7.5-7.30(m, 3 H), 7.28-7.18(m,2 H), 7.20-7.05(t,J = 8.7 hz, 2 H), 3.55(s,2 H), 3.23(s, 2 H),2.90-2.80(m, 2 H),2.57-2.50(m, 1 H),2.10-2.00(m, 2 H),1.70-1.50(m, 4 H),1.22-1.07(m, 6 H). |
| 464 | 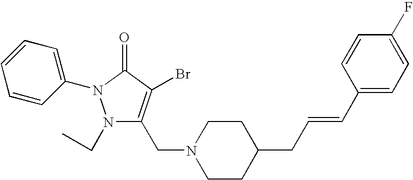 | 4-Bromo-1-ethyl-5-{4-[(E/Z)-3-(4-fluorophenyl)-allyl]-piperidin-1-ylmethyl}-2-phenyl-1,2-dihydro-pyrazol-3-one | (300 MHz, CDCl₃): □(ppm) 7.49-7.38(m,4 H), 7.35-7.18(m,3 H), 7.05-6.95(m,2 H), 6.45-6.32(m,1 H), 6.16-6.06(m,0.6 H), 5.70-5.61(m,0.4 H), 3.83-3.74(m,2 H), 3.52(s, 1.2 H),3.50(s, 0.8 H), 2.95-2.85(m, 2 H), 2.29-2.05(m, 4 H), 1.78-1.72(m, 2 H), 1.45-1.20(m, 3 H), 0.89-0.83(m, 3 H). |
| 465 | 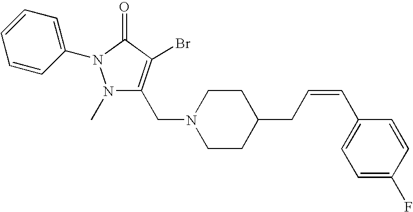 | 4-Bromo-5-{4-[(Z)-3-(4-fluorophenyl)-allyl]-piperidin-1-ylmethyl}-1-methyl-2-phenyl-1,2-dihydro-pyrazol-3-one | (300 MHz, CDCl₃): □(ppm) 7.46-7.21(m,7 H), 7.01(t, J = 8.6 hz,2 H), 6.44(d,J = 11.9 hz, 1 H), 5.70-5.60(m, 1 H), 3.22(s, 3 H), 2.85-2.95(m,2 H), 2.30-2.20(m,2 H), 2.20-2.05(m,2 H), 1.80-1.70(m,2 H), 1.50-1.40(m,1 H), 1.35-1.20(m,2 H). |
| 466 | 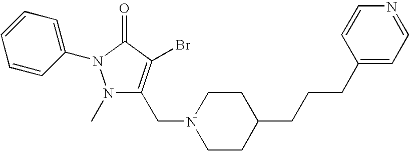 | 4-Bromo-1-methyl-2-phenyl-5-[4-(3-pyridin-4-yl-propyl)-piperidin-1-ylmethyl]-1,2-dihydro-pyrazol-3-one | (300 MHz, CDCl₃): □(ppm) 7.73-7.70(m,1 H), 7.47(t, J = 8.1 hz,2 H), 7.40-7.20(m,6 H), 3.52(s, 2 H), 3.23(s, 3 H), 2.93-2.85(m,4 H), 2.11(t, J = 9.9 H),1.95-1.86(m, 2 H),1.74(d, J = 12 hz, 2 H),1.49-1.23(m, 5 H). |

### Example 467

### 4-Bromo-1-methyl-2-phenyl-5(4-phenyl-piperidin-1-ylmethyl)-1,2-dihydro-pyrazol-3-one

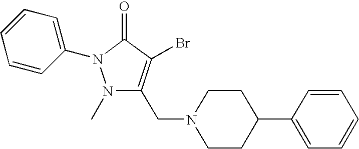

A mixture of 4-bromo-5-bromomethyl-1-methyl-2-phenyl-1,2-dihydro-pyrazol-3-one (200 mg, 0.57 mmol), 4-phenyl-piperidine (91 mg, 0.57 mmol), and triethylamine (79 □l, 0.057 mmol) in tetrahydrofuran (5 mL) was heated to 50° for several hours. The reaction was worked up by diluting with CH₂Cl₂ and washing several times with H₂O. The organics were dried over MgSO₄ then filtered. The filtrates were concentrated on the rotovap then placed on a SiO₂ column and eluted with 5% MeOH in CH₂Cl₂. A foamy white solid was obtained (229 mg, 94%). ¹H NMR (300 MHz, CDCl₃): [symbol] (ppm) 7.50-7.41 (m, 2H), 7.40-7.38 (d, 2H), 7.35-7.31 (m, 3H), 7.24-7.18 (m, 3H), 3.82 (s, 2H), 3.27 (s, 3H), 3.08-3.04 (d, 2H), 2.59-2.50 (m, 1H), 2.31-2.25 (t, 2H), 1.91-1.83 (m, 2H), 1.84-1.72 (m, 2H). LC/MS (METHOD A): 426 (M+H) at 3.63 min.

Compounds of Examples 468 through 487 were synthesized by a method analogous to the procedure of Example 467, using 4-bromo-5-bromomethyl-1-methyl-2-phenyl-1,2-dihydro-pyrazol-3-one and the appropriate amine.

| | | | LC/MS | |
|---|---|---|---|---|
| Exam- | | | (METHOD | M/z |
| ple | Structure | Name | A) (min) | (M + H) |
| | | | LC/MS | |
| 468 | 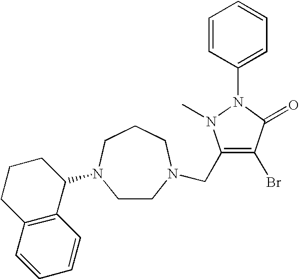 | 4-Bromo-1-methyl-2-phenyl-5-[(R)-4-(1,2,3,4-tetrahydro-naphthalen-1-yl)-[1,4]diazepan-1-ylmethyl]-1,2-dihydro-pyrazol-3-one | 3.63 | 495 |
| 469 | 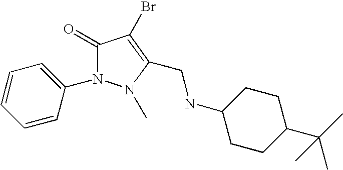 | 4-Bromo-5-[(4-tert-butyl-cyclohexylamino)-methyl]-1-methyl-2-phenyl-1,2-dihydro-pyrazol-3-one | 3.81 | 420 |
| 470 | 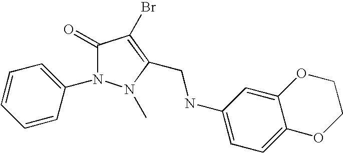 | 4-Bromo-5-[(2,3-dihydro-benzo[1,4]di-oxin-6-ylamino)-methyl]-1-methyl-2-phenyl-1,2-dihydro-pyrazol-3-one | 4.28 | 416 |
| 471 | 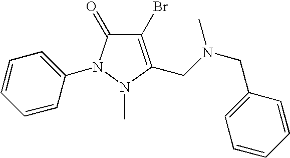 | 5-[(Benzyl-methyl-amino)-methyl]-4-bromo-1-methyl-2-phenyl-1,2-dihydro-pyrazol-3-one | 3.63 | 386 |
| 472 | 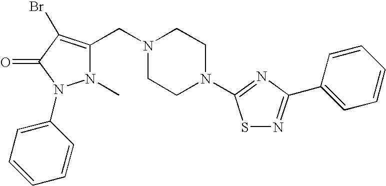 | 4-Bromo-1-methyl-2-phenyl-5-[4-(3-phe-nyl-[1,2,4]thiadiazol-5-yl)-piper-azin-1-ylmethyl]-1,2-dihydro-pyrazol-3-one | 5.19 | 511 |
| 473 | 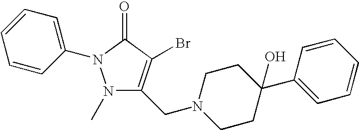 | 4-Bromo-5-(4-hydroxy-4-phenyl-piperidin-1-ylmethyl)-1-methyl-2-phenyl-1,2-dihydro-pyrazol-3-one | 3.21 | 442 |
| 474 | 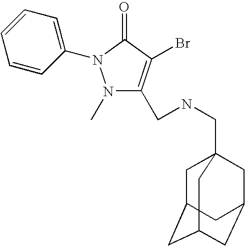 | 5-{[(Adamantan-1-ylmethyl)-amino]-meth-yl}-4-bromo-1-methyl-2-phenyl-1,2-dihydro-pyrazol-3-one | 3.79 | 430 |
| 475 | 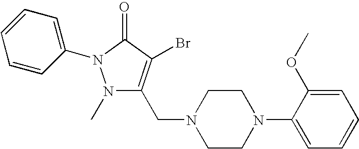 | 4-Bromo-5-[4-(2-methoxy-phenyl)-piper-azin-1-ylmethyl]-1-methyl-2-phenyl-1,2-dihydro-pyrazol-3-one | 3.74 | 457 |
| 476 | 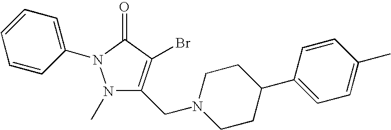 | 4-Bromo-1-methyl-2-phenyl-5-(4-p-tolyl-piperazin-1-ylmethyl)-1,2-dihydro-pyrazol-3-one | 4.18 | 441 |
| 477 | 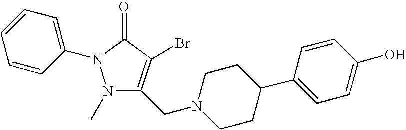 | 4-Bromo-5-[4-(4-hydroxy-phenyl)-piper-azin-1-ylmethyl]-1-methyl-2-phenyl-1,2-dihydro-pyrazol-3-one | 3.18 | 443 |
| 478 | 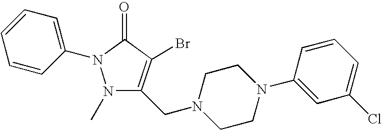 | 4-Bromo-5-[4-(3-chloro-phenyl)-piper-azin-1-ylmethyl]-1-methyl-2-phenyl-1,2-dihydro-pyrazol-3-one | 4.75 | 461 |
| 479 | 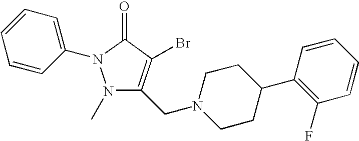 | 4-Bromo-5-[4-(2-fluoro-phenyl)-piperazin-1-ylmethyl]-1-methyl-2-phenyl-1,2-dihydro-pyrazol-3-one | 4.28 | 445 |
| 480 | 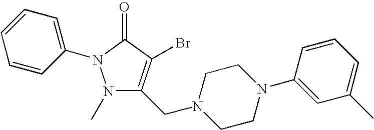 | 4-Bromo-1-methyl-2-phenyl-5-(4-m-tolyl-piperazin-1-ylmethyl)-1,2-dihydro-pyrazol-3-one | 4.23 | 441 |
| 481 | 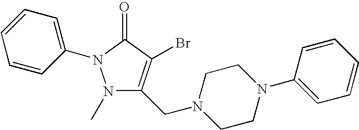 | 4-Bromo-1-methyl-2-phenyl-5-(4-phenyl-piperazin-1-ylmethyl)-1,2-dihydro-pyrazol-3-one | 4.05 | 427 |
| 482 | 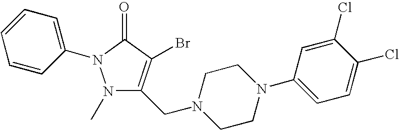 | 4-Bromo-5-[4-(3,4-dichloro-phenyl)-piperazin-1-ylmethyl]-1-methyl-2-phenyl-1,2-dihydro-pyrazol-3-one | 5.18 | 495 |
| 483 | 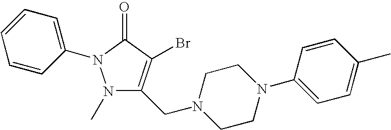 | 4-Bromo-5-[4-(4-chloro-phenyl)-piper-azin-1-ylmethyl]-1-methyl-2-phenyl-1,2-dihydro-pyrazol-3-one | 4.67 | 461 |
| 484 | 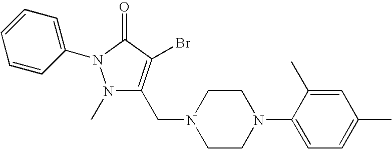 | 4-Bromo-5-[4-(2,4-dimethyl-phenyl)-piperazin-1-ylmethyl]-1-methyl-2-phenyl-1,2-dihydro-pyrazol-3-one | 4.49 | 455 |
| 485 | 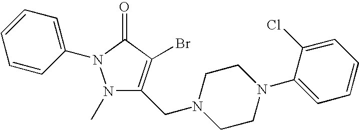 | 4-Bromo-5-[4-(chloro-phenyl)-piperazin-1-ylmethyl]-1-methyl-2-phenyl-1,2-di-hydro-pyrazol-3-one | 4.50 | 461 |

| | | | |
|---|---|---|---|
| 486 | 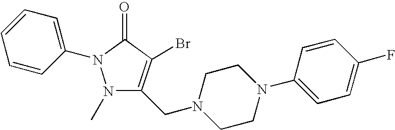 | 4-Bromo-5-[4-(4-fluoro-phenyl)-piper-azin-1-ylmethyl]-1-methyl-2-phenyl-1,2-dihydro-pyrazol-3-one | ¹H NMR (300 MHz,CDCl₃): [symbol] (ppm)7.50-7.47(m, 2 H),7.40-7.33(m, 3 H),7.00-6.86(m, 4 H),3.63(s, 2 H), 3.25(s,3 H), 3.17-3.13(m,4 H), 2.76-2.72(m,4 H) |
| 487 | 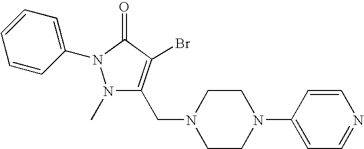 | 4-bromo-1-methyl-2-phenyl-5-(4-pyridin-4-yl-piperazin-1-ylmethyl)-1,2-dihydro-pyrazol-3-one | ¹H NMR (300 MHz,DMSO): [symbol] (ppm)8.27-8.24(m, 2 H),7.57-7.52(m, 2 H),7.43-7.35(m, 3 H),7.23-7.21(m, 2 H),3.74(m, 4 H), 3.72(s,2 H), 3.25(s, 3 H),2.69(m, 4 H) |

### Example 488

### 5-Bromomethyl-4-chloro-1-methyl-2-phenyl-1,2-dihydro-pyrazol-3-one

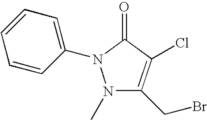

To a solution of antipyrine (1.0 g, 5.3 mmol) in CH₂Cl₂ (20 mL) was added N-chlorosuccinimide (709 mg, 5.3 mmol). The resultant mixture was stirred for 1 h then washed with 1N NaOH (1×40 mL), water (1×40 mL) and brine (1×40 mL) and dried over Na₂SO₄. Evaporation of the solvent afforded material that was chromatographed on silica gel using hexanes to 1:1 hexanes:ethyl acetate as eluant to afford a white solid, 4-chloro-1,5-dimethyl-2-phenyl-1,2-dihydro-pyrazol-3-one (971 mg, 4.36 mmol, 82%). This material was taken up in CCl₄ (15 mL) and N-bromosuccinimide (776 mg, 4.36 mmol) was added; the reaction was then heated to 50° C. for 1 h, at which time it was cooled to rt. It was then washed with 1N NaOH, water and brine then dried over Na₂SO₄. Filtration and concentration afforded a yellow liquid which was chromatographed on silica gel using hexanes to 1:1 hexanes:ethyl acetate as eluant to afford a white solid, 5-bromomethyl-4-chloro-1-methyl-2-phenyl-1,2-dihydro-pyrazol-3-one (621 mg, 2.05 mmol, 47%). ¹H NMR (300 MHz, CDCl₃): [symbol] (ppm) 7.51-7.46 (m, 2H), 7.41-7.35 (m, 3H), 4.38 (s, 2H), 3.17 (s, 3H).

### Example 489

### 4-Chloro-5-[4-(2-methoxy-phenyl)-piperazin-1-ylmethyl]-1-methyl-2-phenyl-1,2-dihydro-pyrazol-3-one

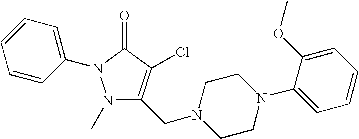

To a solution of 1-(2-methoxy-phenyl)-piperazine (64 mg, 0.33 mmol) in THF (2 mL) was added 5-bromomethyl-4-chloro-1-methyl-2-phenyl-1,2-dihydro-pyrazol-3-one (100 mg, 0.33 mmol) and triethylamine (46 μL, 0.33 mmol). This solution was heated to 50° C. for 2 h, at which time it was cooled to rt and water (5 mL) and CH₂Cl₂ (5 mL) were added. The layers were separated and the organic fraction evaporated to give a product that was purified by silical gel chromatography using CH₂Cl₂-5% 2M NH₃ in MeOH/CH₂Cl₂ as eluant to afford 4-chloro-5-[4-(2-methoxy-phenyl)-piperazin-1-ylmethyl]-1-methyl-2-phenyl-1,2-dihydro-pyrazol-3-one as a white solid. ¹H NMR (300 MHz, CDCl₃): [symbol] (ppm) 7.50-7.39 (m, 4H), 7.33 (t, 1H), 7.05-6.98 (m, 1H), 6.94-6.86 (m, 3H), 3.88 (s, 3H), 3.64 (s, 2H), 3.24 (s, 3H), 3.12 (m, 4H), 2.77 (m, 4H); LC/MS (METHOD A): 413 (M+H) at 3.68 min.

Compounds of Examples 490 through 493 were synthesized by a method analogous to the procedure of Example 489, using 5-bromomethyl-4-chloro-1-methyl-2-phenyl-1,2-dihydro-pyrazol-3-one and the appropriate amine.

| | | | LC/MS | |
|---|---|---|---|---|
| | | | (METHOD | M/z |
| Example | Structure | Name | A) (min) | (M + H) |
| 490 | 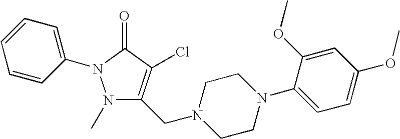 | 4-Chloro-5-[4-(2,4-dimethoxy-phenyl)-piperazin-1-ylmethyl]-1-methyl-2-phenyl-1,2-dihydro-pyraozl-3-one | 3.59 | 443 |
| 491 | 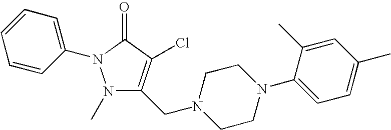 | 4-Chloro-5-[4-(2,4-dimethyl-phenyl)-piperazin-1-ylmethyl]-1-methyl-2-phenyl-1,2-dihydro-pyrazol-3-one | 4.37 | 411 |
| 492 | 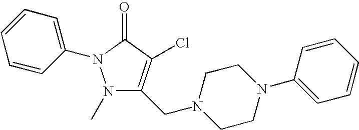 | 4-Chloro-1-methyl-2-phenyl-5-(4-phenyl-piperazin-1-ylmethyl)-1,2-dihydro-pyrazol-3-one | 3.93 | 383 |
| 493 | 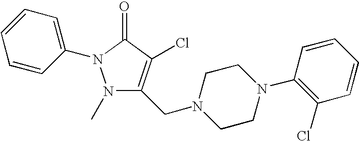 | 4-Chloro-5-[4-(2-chloro-phenyl)-piper-azin-1-ylmethyl]-1-methyl-2-phenyl-1,2-dihydro-pyrazol-3-one | 4.37 | 417 |

### Example 494

### 5-Methyl-2-phenyl-1-propyl-1,2-dihydro-pyrazol-3-one

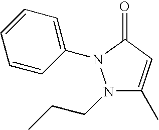

5-Methyl-2-phenyl-1,2-dihydro-pyrazol-3-one (1.0 g, 5.7 mmol) and iodopropane (7.0 mL, 71.8 mmol) were heated at 100° C. for 24 hours in a sealed tube. The mixture was concentrated and chromatographed with 5% 2.0M ammonia in methanol and dichloromethane to give the product as a pale yellow oil (326 mg, 26%). ¹H NMR (300 MHz, d₆-DMSO): δ(ppm) 7.52-7.41 (m, 2H), 7.35-7.25 (m, 3H), 5.25 (s, 1H), 3.51 (t, 2H), 2.25 (s, 3H), 1.33-1.17 (m, 2H), 0.67 (s, 3H).

### Example 495

### 4-Bromo-5-bromomethyl-2-phenyl-2-phenyl-1-propyl-1,2-dihydro-pyrazol-3-one

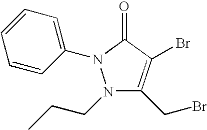

5-Methyl-2-phenyl-1-propyl-1,2-dihydro-pyrazol-3-one (326 mg, 1.5 mmol) in carbon tetrachloride (30 mL) was treated with N-bromosuccinimide (537 mg, 3.0 mmol) and heated at 50° C. for 2 hours. The mixture was diluted with dichloromethane and washed (1N NaOH, water, brine), dried (Na₂SO₄), and evaporated to a brown oil. The oil was chromatographed with 20% acetonitrile in dichloromethane to give the product as an off-white solid (491 mg, 87%). ¹H NMR (300 MHz, d₆-DMSO): 7.60-7.50 (m, 2H), 7.47-7.33 (m, 3H), 4.74 (s, 2H), 3.69 (t, 2H), 1.38-1.21 (m, 2H), 0.67 (s, 3H).

### Example 496

### 4-Bromo-5-[4-(3,5-dichloro-pyridin-4-yl)-piperazin-1-ylmethyl]-1-ethyl-2-phenyl-1,2-dihydro-pyrazol-3-one

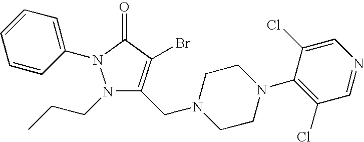

A mixture of 4-bromo-5-bromomethyl-2-phenyl-1-propyl-1,2-dihydro-pyrazol-3-one (80 mg, 0.21 mmol), 1-(3,5-dichloro-4-pyridyl)piperazine (55 mg, 0.24 mmol), and triethylamine (100 μL, 0.72 mmol) in tetrahydrofuran (10 mL) was heated at 50° C. for 2.5 hours. Additional 1-(3,5-dichloro-4-pyridyl)piperazine (20 mg, 0.09 mmol) and acetonitrile (2 mL) were added and heating was continued at 50° C. for 2 hours followed by 70° C. for one hour. The mixture was concentrated and the residue partitioned between water and dichloromethane. The organic portion was washed (water, brine), dried (Na₂SO₄), and concentrated to a crude oil that was chromatographed with 20% acetonitrile in dichloromethane. The resulting solid was triturated with diethyl ether to give the product as an off-white solid (43 mg, 38%). ¹H NMR (300 MHz, CDCl₃): δ(ppm) 8.36 (s, 2H), 7.55-7.29 (m, 5H), 3.78-3.60 (m, 4H), 3.45-3.33 (m, 4H), 2.82-2.68 (m, 4H), 1.43-1.27 (m, 2H), 0.77 (t, 3H). LC/MS (METHOD A): 524 (m+H) at 4.95 min.

Compounds of Examples 497 and 498 were synthesized by a method analogous to the procedure of Example 496, using 4-bromo-5-bromomethyl-2-phenyl-1-propyl-1,2-dihydro-pyrazol-3-one and an amine.

| | | | LS/MS | |
|---|---|---|---|---|
| | | | (METHOD | |
| | | | A) | m/z |
| Example | Structure | Name | (min.) | (M + H) |
| 497 | 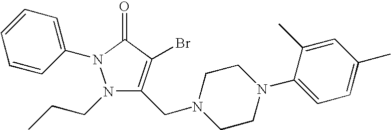 | 4-bromo-5-[4-(2,4-dimethyl-phenyl)-piperzin-1-ylmethyl]-2-phenyl-1-propyl-1,2-dihydro-pyrazol-3-one | 5.08 | 483 |
| 498 | 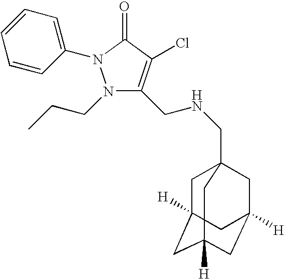 | 5-{[Adamantan-1-ylmethyl)-amino]-methyl}-4-bromo-2-phenyl-1-propyl-1,2-dihydro-pyrazol-3-one | 4.02 | 458 |

### Example 499

### 5-Bromomethyl-4-methoxy-1-methyl-2-phenyl-1,2-dihydro-pyrazol-3-one

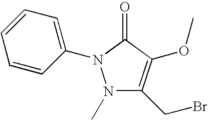

To a solution of 4-hydroxyantipyrine (2.04 g, 10.0 mmol) in acetone (50 mL) was added K₂CO₃ (2.71 g, 19.6 mmol) and iodomethane (915 □L, 14.7 mmol). The reaction was heated to reflux for 1 h, cooled to room temperature, the mixture was filtered through diatomaceous earth, and the filtrates were concentrated. The material was then dissolved in CH₂Cl₂ and Et₂O and filtered through a cotton plug; the filtrates were concentrated to a yellow liquid that was purified by chromatography on silica gel using 20:1 CH₂Cl₂:2M NH₃ in MeOH as eluant to afford 4-methoxy-1,5-dimethyl-2-phenyl-1,2-dihydro-pyrazol-3-one as a yellow solid (2.09 g, 96%). This material was dissolved in CCl₄ (40 mL) and N-bromosuccinimide (1.70 g, 9.58 mmol) was added, followed by additional CCl₄ (10 mL). The reaction was heated to 50° C. for 18 h, cooled to rt, and additional N-bromosuccinimide (900 mg, 5.07 mmol) was added and the heated was resumed for 30 min. The reaction was cooled to rt, filtered through diatomaceous earth and the filtrate was concentrated and purified by silica gel chromatography using 1:1 hexanes:ethyl acetate as eluant to afford 5-bromomethyl-4-methoxy-1-methyl-2-phenyl-1,2-dihydro-pyrazol-3-one as a solid (814 mg, 28%). ¹H NMR (300 MHz, CDCl₃): [symbol] (ppm) 7.49-7.44 (m, 4H), 7.30-7.27 (m, 1H), 4.35 (s, 2H), 4.05 (s, 3H), 3.00 (s, 3H).

### Example 500

### 4-Methoxy-5-[4-(2-methoxy-phenyl)-piperazin-1-ylmethyl]-1-methyl-2-phenyl-1,2-dihydro-pyrazol-3-one

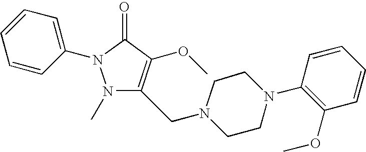

To a solution of 1-(2-methoxy-phenyl)-piperazine (65 mg, 0.34 mmol) in THF (2 mL) was added triethylamine (47 □L, 0.34 mmol) and 5-bromomethyl-4-methoxy-1-methyl-2-phenyl-1,2-dihydro-pyrazol-3-one (100 mg, 0.34 mmol). The reactions were heated to 50° C. for 1 h, cooled to rt and water (3 mL) and CH₂Cl₂ (5 mL) were added, the layers were separated and the organic layer was concentrated. The obtained material was purified by silica gel chromatography using 2% 2M NH₃ in MeOH/CH₂Cl₂-10% 2M NH₃ in MeOH/CH₂Cl₂ as eluant to afford 4-methoxy-5-[4-(2-methoxy-phenyl)-piperazin-1-ylmethyl]-1-methyl-2-phenyl-1,2-dihydro-pyrazol-3-one as a yellow liquid (99 mg, 72%). ¹H NMR (300 MHz, CDCl₃): [symbol] (ppm) 7.45-7.44 (m, 4H), 7.28-7.25 (m, 1H), 7.02-6.96 (m, 1H), 6.96-6.92 (m, 1H), 6.88-6.86 (m, 2H), 3.95 (s, 3H), 3.87 (s, 3H), 3.56 (s, 2H), 3.12 (m, 4H), 3.06 (s, 3H), 2.75 (m, 4H).

Compounds of Examples 501 were synthesized by a method analogous to the procedure of Example 500, using 5-bromomethyl-4-methoxy-1-methyl-2-phenyl-1,2-dihydro-pyrazol-3-one and the appropriate amine.

| | | | LC/MS | |
|---|---|---|---|---|
| | | | (METHOD | M/z |
| Example | Structure | Name | A) (min) | (M + H) |
| 501 | 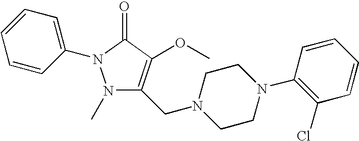 | 5-[4-(2-Chloro-phenyl)-piperazin-1-ylmethyl]-4-methoxy-1-methyl-2-phenyl-1,2-dihydro-pyrazol-3-one | 3.86 | 413 |
| 502 | 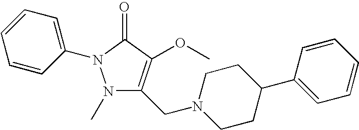 | 4-Methoxy-1-methyl-2-phenyl-5-(4-phenyl-piperidin-1-ylmethyl)-1,2-dihydro-pyrazol-3-one | 3.59 | 378 |
| 503 | 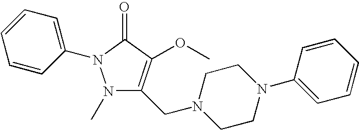 | 4-Methoxy-1-methyl-2-phenyl-5-(4-phenyl-piperazin-1-ylmethyl)-1,2-dihydro-pyrazol-3-one | 3.58 | 379 |
| 504 | 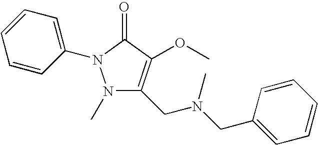 | 5-[(Benzyl-methyl-amino)-methyl]-4-methoxy-1-methyl-2-phenyl-1,2-dihydro-pyrazol-3-one | 3.32 | 338 |

| | | | |
|---|---|---|---|
| 505 | 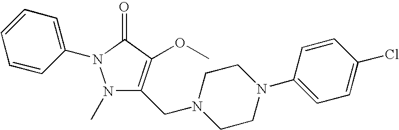 | 5-[4-(4-Chloro-phenyl)-piperazin-1-ylmethyl]-4-methoxy-1-methyl-2-phenyl-1,2-dihydro-pyrazol-3-one | ¹H NMR (300MHz, CDCl₃): [symbol] (ppm) 7.45(m, 4 H), 7.27(m, 1 H), 7.21(d, 2 H), 6.85(d, 2 H), 3.96(s, 3 H), 3.54(s, 2 H), 3.19(m, 4 H), 3.04(s, 3 H), 2.70(s, 4 H). |
| 506 | 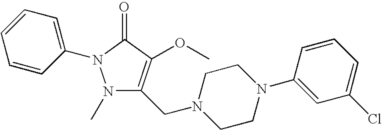 | 5-[4-(3-Chloro-phenyl)-piperazin-1-ylmethyl]-4-methoxy-1-methyl-2-phenyl-1,2-dihydro-pyrazol-3-one | ¹H NMR (300MHz, CDCl₃): [symbol] (ppm) 7.45-7.44(m, 4 H),7.30-7.27(m,1 H), 7.19-7.14(m, 1 H), 6.88(m, 1 H), 6.83-6.77(m, 2 H),3.96(s, 3 H),3.54(s, 2 H),3.24-3.21(m,4 H), 3.04(s,3 H), 2.71-2.68(m, 4 H). |

### Example 507

### 4,5-Dimethyl-2-phenyl-1,2-dihydro-pyrazol-3-one

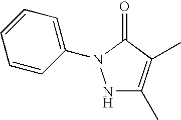

Phenylhydrazine (2.11 g, 19.5 mmol) in toluene (37 mL) was treated with ethyl 2-methylacetoacetate (2.85 g, 19.8 mmol) and heated at 70° C. for 4.5 hours followed by 110° C. for 2 hours. The mixture was concentrated and chromatographed with 20% acetonitrile in dichloromethane to give the product as an off-white solid (2.86 g, 78%). ¹H NMR (300 MHz, d₆-DMSO): δ(ppm) 10.46 (br s, 1H), 7.78-7.66 (m, 2H), 7.47-7.36 (m, 2H), 7.22-7.12 (m, 1H), 2.09 (s, 3H), 1.90-1.62 (br s, 3H).

### Example 508

### 1,4,5-Trimethyl-2-phenyl-1,2-dihydro-pyrazol-3-one

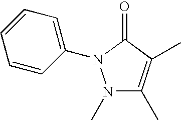

4,5-Dimethyl-2-phenyl-1,2-dihydro-pyrazol-3-one (2.86 g, 15.2 mmol) in acetonitrile (17 mL) was treated with iodomethane (3.0 mL, 48.2 mmol) and heated at 80° C. for 8 hours. The mixture was concentrated and chromatographed with 5% 2.0M ammonia in methanol and dichloromethane, followed by chromatography with diethyl ether to give the product as a solid (1.14 g, 37%). ¹H NMR (300 MHz, d₆-DMSO): δ(ppm) 7.52-7.42 (m, 2H), 7.39-7.22 (m, 3H), 2.95 (s, 3H), 2.18 (s, 3H), 1.72 (s, 3H).

### Example 509

### 5-Bromomethyl-1,4-dimethyl-2-phenyl-1,2-dihydro-pyrazol-3-one

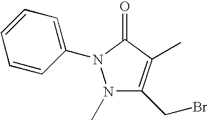

1,4,5-Trimethyl-2-phenyl-1,2-dihydro-pyrazol-3-one (230 mg, 1.1 mmol) in carbon tetrachloride (50 mL) was treated with N-bromosuccinimide (198 mg, 1.1 mmol) and refluxed for 20 minutes. The mixture was concentrated and chromatographed with diethyl ether to give the product as a colorless oil (272 mg, 85%). ¹H NMR (300 MHz, d₆-DMSO): δ(ppm) 7.55-7.46 (m, 2H), 7.40-7.26 (m, 3H), 4.73 (s, 2H), 3.05 (s, 3H), 1.81 (s, 3H).

### Example 510

### 5-[4-(2,4-Dimethoxy-phenyl)-piperazin-1-ylmethyl]-1,4-dimethyl-2-phenyl-1,2-dihydro-pyrazol-3-one

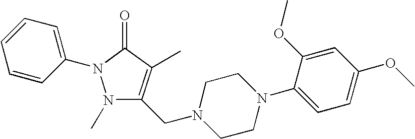

A mixture of 5-bromomethyl-1,4-dimethyl-2-phenyl-1,2-dihydro-pyrazol-3-one (82 mg, 0.29 mmol), 1-(2,4-dimethoxyphenyl)piperazine (80 mg, 0.36 mmol), and triethylamine (90 μL, 0.65 mmol) in tetrahydrofuran (7 mL) was heated at 50° C. for one hour. The mixture was filtered, concentrated, and chromatographed with 5% 2.0M ammonia in methanol and dichloromethane to give the product as an off-white solid (103 mg, 83%). ¹H NMR (300 MHz, d₆-DMSO): δ(ppm) 7.53-7.43 (m, 2H), 7.38-7.25 (m, 3H), 6.83 (d, 1H), 6.55-6.50 (m, 1H), 6.46-6.40 (m, 1H), 3.76 (s, 3H), 3.70 (s, 3H), 3.55 (s, 2H), 3.07 (s, 3H), 2.97-2.83 (br s, 4H), 2.68-2.53 (br s, 4H), 1.80 (s, 3H). LC/MS (METHOD A): 423 (M+H) at 3.48 min.

Compounds of Examples 511 through 514 were synthesized by a method analogous to the procedure of Example 510, using 5-bromomethyl-1,4-dimethyl-2-phenyl-1,2-dihydro-pyrazol-3-one and the appropriate piperazine.

| | | | LC/MS | |
|---|---|---|---|---|
| | | | (METHOD | |
| | | | A) | m/z |
| Example | Structure | Name | (min.) | (M + H) |
| 511 | 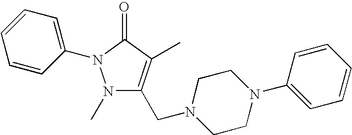 | 1,4-dimethyl-2-phenyl-5-(4-phenyl-piperazin-1-ylmethyl)-1,2-dihydro-pyrazol-3-one | 3.55 | 363 |
| 512 | 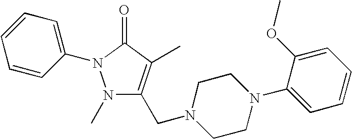 | 5-[4-(2-methoxy-phenyl)-piperazin-1-ylmethyl]-1,4-dimethyl-2-phenyl-1,2-dihydro-pyrazol-3-one | 3.43 | 393 |
| 513 | 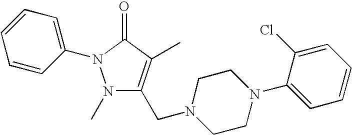 | 5-[4-(2-chloro-phenyl)-piperazin-1-ylmethyl]-1,4-dimethyl-2-phenyl-1,2-dihydro-pyrazol-3-one | 3.81 | 397 |
| 514 | 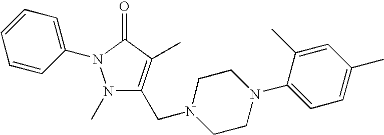 | 5-[4-(2,4-dimethyl-phenyl)-piperazin-1-ylmethyl]-1,4-dimethyl-2-phenyl-1,2-dihydro-pyrazol-3-one | 3.89 | 391 |

### Example 515

### 4-Ethyl-5-methyl-2-phenyl-1,2-dihydro-pyrazol-3-one

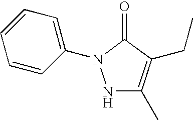

Phenylhydrazine (1.07 g, 9.9 mmol) in toluene (20 mL) was treated with ethyl 2-ethylacetoacetate (1.58 g, 10.0 mmol) and heated at 110° C. for 2 hours followed by 100° C. for 17 hours. The flask was equipped with a Dean-Stark trap and heating continued at 140° C. for 3.5 hours. The mixture was concentrated to an orange oil that was chromatographed with 1:1 diethyl ether/hexane, 2:1 diethyl ether/hexane, and 100% diethyl ether, respectively. The material was triturated with diethyl ether/hexane to give the product as an off-white solid (1.25 g, 62%). ¹H NMR (300 MHz, d₆-DMSO): δ(ppm) 10.42 (br s, 1H), 7.76-7.65 (m, 2H), 7.45-7.35 (m, 2H), 7.20-7.12 (m, 1H), 2.37-2.07 (m, 5H), 1.03 (t, 3H).

### Example 516

### 4-Ethyl-1,5-dimethyl-2-phenyl-1,2-dihydro-pyrazol-3-one

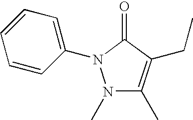

4-Ethyl-5-methyl-2-phenyl-1,2-dihydro-pyrazol-3-one (1.24 g, 6.13 mmol) in acetonitrile (7 mL) was treated with iodomethane (1.2 mL, 19.3 mmol) and heated at 80° C. for 15 hours. Additional iodomethane (1.0 mL, 16.1 mmol) was added and the mixture was refluxed for 3.5 hours. The mixture was concentrated and the residue chromatographed with 20% acetonitrile in dichloromethane and 50% acetonitrile in dichloromethane. Further chromatography with diethyl ether gave the product as a pale yellow oil (620 mg, 46%). ¹H NMR (300 MHz, d₆-DMSO): δ(ppm) 7.52-7.42 (m, 2H), 7.38-7.21 (m, 3H), 2.95 (s, 3H), 2.25-2.13 (m, 5H), 1.02 (t, 3H).

### Example 517

### 5-Bromomethyl-4-ethyl-1-methyl-2-phenyl-1,2-dihydro-pyrazol-3-one

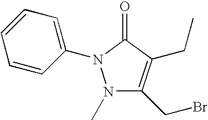

4-Ethyl-1,5-dimethyl-2-phenyl-1,2-dihydro-pyrazol-3-one (618 mg, 2.86 mmol) in carbon tetrachloride (125 mL) was treated with N-bromosuccinimide (509 mg, 2.86 mmol) and refluxed for 20 minutes. The mixture was concentrated. The residue was taken up in diethyl ether and washed (1N NaOH, H₂O, brine), dried (MgSO₄), and evaporated to a crude solid. The crude material was chromatographed with 5% methanol in dichloromethane followed by trituration with 1:1 diethyl ether/hexane to give the product as a white solid (528 mg, 62%). ¹H NMR (300 MHz, d₆-DMSO): δ(ppm) 7.55-7.45 (m, 2H), 7.39-7.28 (m, 3H), 4.74 (s, 2H), 3.05 (s, 3H), 2.31 (q, 2H), 1.08 (t, 3H).

### Example 518

### 5-[4-(2,4-Dimethoxyphenyl)-piperazin-1-ylmethyl]-4-ethyl-1-methyl-2-phenyl-1,2-dihydro-pyrazol-3-one

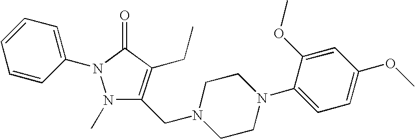

A mixture of 5-bromomethyl-4-ethyl-1-methyl-2-phenyl-1,2-dihydro-pyrazol-3-one (105 mg, 0.36 mmol), 1-(2,4-dimethoxyphenyl)piperazine (101 mg, 0.45 mmol), and triethylamine (100 mL, 0.72 mmol) in tetrahydrofuran (8 mL) was heated at 50° C. for 1.5 hours. The mixture was filtered, concentrated, and chromatographed with 5% 2.0M ammonia in methanol and dichloromethane to give the product as an off-white solid (111 mg, 71%). ¹H NMR (300 MHz, d₆-DMSO): δ(ppm) 7.53-7.43 (m, 2H), 7.38-7.25 (m, 3H), 6.83 (d, 1H), 6.55-6.50 (m, 1H), 6.47-6.40 (m, 1H), 3.76 (s, 3H), 3.70 (s, 3H), 3.55 (s, 2H), 3.08 (s, 3H), 2.97-2.85 (br s, 4H), 2.67-2.56 (br s, 4H), 2.28 (q, 2H), 1.05 (t, 3H). LC/MS (METHOD A): 437 (M+H) at 3.59 min.

Compounds of Examples 519 through 522 were synthesized by a method analogous to the procedure of Example 518, using 5-bromomethyl-4-ethyl-1-methyl-2-phenyl-1,2-dihydro-pyrazol-3-one and the appropriate piperazine.

| | | | LC/MS | |
|---|---|---|---|---|
| | | | (METHOD | |
| | | | A) | m/z |
| Example | Structure | Name | (min) | (M + H) |
| 519 | 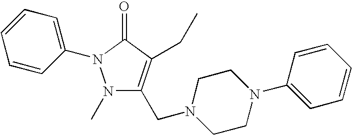 | 4-ethyl-1-methyl-2-phenyl-5-(4-phenyl-piperazin-1-ylmethyl)-1,2-dihydro-pyrazol-3-one | 3.76 | 377 |
| 520 | 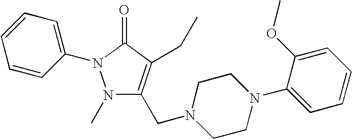 | 4-ethyl-5-[4-(2-methoxy-phenyl)-piperazin-1-ylmethyl]-1-methyl-2-phenyl-1,2-dihydro-pyrazol-3-one | 3.60 | 407 |
| 521 | 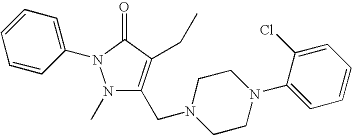 | 5-[4-chloro-phenyl)-piperazin-1-ylmethyl]-4-ethyl-1-methyl-2-phenyl-1,2-dihydro-pyrazol-3-one | 4.09 | 411 |
| 522 | 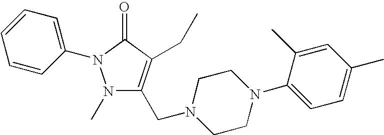 | 5-[4-(2,4-dimethyl-phenyl)-piperazin-1-ylmethyl]-4-ethyl-1-methyl-2-phenyl-1,2-dihydro-pyrazol-3-one | 4.14 | 405 |

### Example 523

### 4-Isopropyl-1-methyl-2-phenyl-5-(4-phenyl-piperidin-1-ylmethyl)-1,2-dihydro-pyrazol-3-one

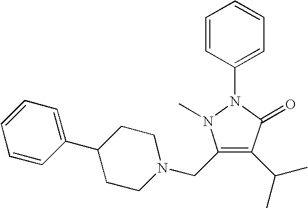

A mixture of 5-bromomethyl-4-isopropyl-1-methyl-2-phenyl-1,2-dihydro-pyrazol-3-one (160 mg, 0.52 mmol), 4-phenyl-piperidine (118 mg, 0.52 mmol), and triethylamine (180□l, 1.3 mmol) in tetrahydrofuran (5 mL) was heated to 50° for several hours. The reaction was then concentrated to an oil. The oil was then taken up in CH₂Cl₂ and washed several times with H₂O. The organics were combined and dried over MgSO₄ then filtered. The filtrates were concentrated on the rotovap then placed on a SiO₂ column and eluted with 5% MeOH in CH₂Cl₂. A light yellow solid was obtained (170 mg, 84%). ¹H NMR (300 MHz, CDCl3): [symbol] (ppm) 7.44-7.42 (d, 2H), 7.37-7.31 (m, 2H), 7.23-7.18 (m, 5H), 3.47 (s, 2H), 3.14 (s, 3H), 3.09-3.05 (d, 2H), 2.93-2.84 (m, 1H), 2.57-2.49 (m, 1H), 2.26-2.13 (m, 2H), 1.90-1.73 (m, 4H), 1.53-1.43 (d, 6H). LC/MS (METHOD A): 390 (M+H) at 3.77 min.

Compounds of Examples 524 and 525 were synthesized by a method analogous to the procedure of Example 523, using 5-bromomethyl-4-isopropyl-1-methyl-2-phenyl-1,2-dihydropyrazol-3-one and the appropriate amine.

| | | | LC/MS | |
|---|---|---|---|---|
| | | | (METHOD | M/z |
| Example | Structure | Name | A) (min) | (M + H) |
| 524 | 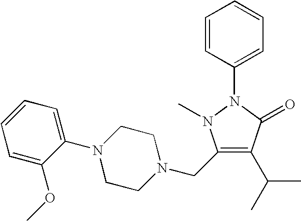 | 4-Isopropyl-5-[4-(2-methoxy-phenyl)-piperazin-1-ylmethyl]-1-methyl-2-phenyl-1,2-dihydro-pyrazol-3-one | 3.81 | 421 |
| 525 | 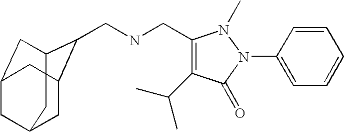 | 5-{[(Adamantan-2-ylmethyl)-amino]-meth-yl}-4-isopropyl-1-methyl-2-phenyl-1,2-dihydro-pyrazol-3-one | 3.94 | 394 |

### Example 526

### 8-(4-Bromo-2-ethyl-5-oxo-1-phenyl-2,5-dihydro-1H-pyrazol-3-ylmethyl)-1-phenyl-1,3,8-triazaspiro[4.5]decan-4-one

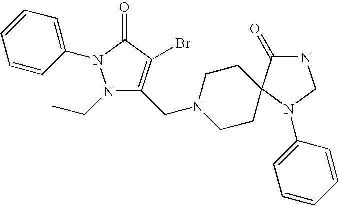

A mixture of 4-bromo-5-bromomethyl-1-ethyl-2-phenyl-1,2-dihydropyrazol-3-one (180 mg, 0.5 mmol) and 1-phenyl-1,3,8-triazaspiro[4,5]decan-4-one (130 mg, 0.55 mmol) containing DIPEA (0.2 mL) in CH₃CN (2 mL) was microwaved at 100° C. for 8 minutes. The crystallized product upon cooling to rt was collected, rinsed with CH₃CN (2×1 mL) and dried under high vacuum to offer 8-[4-Bromo-2-ethyl-5-oxo-1-phenyl-2,5-dihydro-1H-pyrazol-3-ylmethyl]-1-phenyl-1,3,8-triazaspiro[4.5]decan-4-one as an off-white solid, 255 mg, (50%). ¹H NMR (300 MHz, DMSO): □(ppm) 8.64 (s, 1H), 7.54 (m, 2H), 7.39-(m, 3H), 7.2 (m, 2H), 6.83 (d, 2H), 6.73 (t, 1H), 4.58 (s, 2H), 3.85 (q, 2H), 3.67 (s, 2H), 2.87 (m, 4H), 2.56 (m, 2H), 1.62 (d, 2H), 0.92 (t, 3H). LC/MS (Method B): 510 (M+1) at 1.55 min.

### Example 527

### 8-(4-Bromo-2-methyl-5-oxo-1-phenyl-2,5-dihydro-1H-pyrazol-3-ylmethyl)-1-phenyl-1,3,8-triazaspiro[4.5]decan-4-one

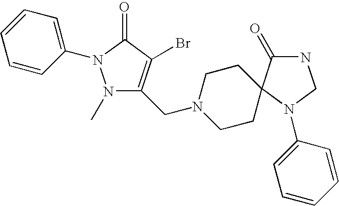

A mixture of 4-bromo-5-bromomethyl-1-methyl-2-phenyl-1,2-dihydropyrazol-3-one (173 mg, 0.5 mmol) and 1-phenyl-1,3,8-triazaspiro[4,5]decan-4-one (231 mg, 1 mmol) containing sym.collidine (0.2 mL) DMF (2.5 mL) was microwaved at 140° C. for 8 minutes. Purified the product by reverse phase HPLC. ¹H NMR (300 MHz, DMSO): □(ppm) 8.66 (s, 1H), 7.54 (m, 2H), 7.39 (m, 3H), 7.26 (m, 2H), 6.87 (d, 2H), 6.64 (t, 1H), 4.59 (s, 2H), 3.69 (s, 2H), 3.28 (s, 3H), 2.87 (m, 4H), 2.59 (m, 2H), 1.65 (d, 2H). LC/MS (Method B): 497 (M+1) at 2.57 min.

By adapting the procedure in Example 527, compounds of Examples 528 through 544 were prepared.

| | | | LC/MS | |
|---|---|---|---|---|
| | | | (Method | |
| | | | B) | m/z |
| Example | Structure | Name | (min.) | (M + 1) |
| 528 | 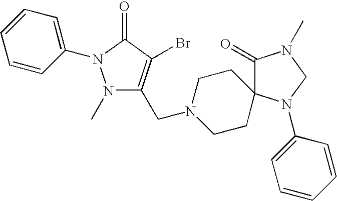 | 8-(4-Bromo-2-ethyl-5-oxo-1-phenyl-2,5-dihydro-1H-pyrazol-3-ylmethyl)-3-methyl-1-phenyl-1,3,8-triazaspiro[4.5]decan-4-one | 2.67 | 524 |
| 529 | 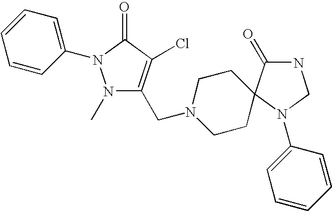 | 8-(4-Chloro-2-methyl-5-oxo-1-phenyl-2,5-dihydro-1H-pyrazol-3-ylmethyl)-1-phenyl-1,3,8-triazaspiro[4.5]decan-4-one | 1.47 | 452 |
| 530 | 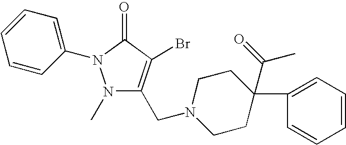 | 5-(Acetyl-4-phenyl-piperidin-1-ylmethyl)-4-bromo-1-methyl-2-phenyl-1,2-dihydro-pyrazol-3-one | 1.65 | 468 |
| 531 | 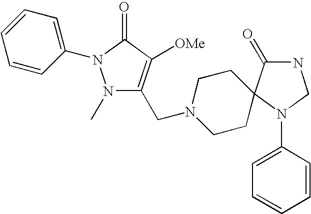 | 8-(4-Methoxy-2-methyl-5-oxo-1-phenyl-2,5-dihydro-1H-pyrazol-3-ylmethyl)-1-phenyl-1,3,8-triazaspiro[4.5]decan-4-one | 1.52 | 448 |
| 532 | 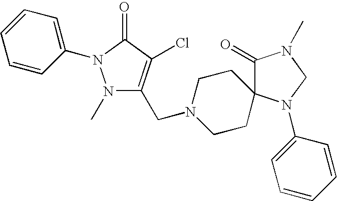 | 8-(4-Chloro-2-methyl-5-oxo-1-phenyl-2,5-dihydro-1H-pyrazol-3-ylmethyl)-methyl-1-phenyl-1,3,8-triazaspiro[4.5]decan-4-one | 2.52 | 466 |
| 533 | 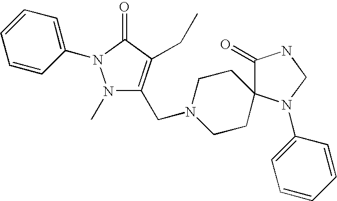 | 8-(4-Ethyl-2-methyl-5-oxo-1-phenyl-2,5-dihydro-1H-pyrazol-3-ylmethyl)-1-phenyl-1,3,8-triazaspiro[4.5]decan-4-one | 1.54 | 446 |
| 534 | 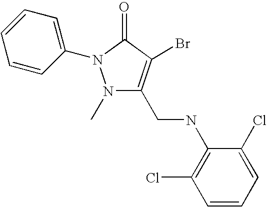 | 4-Bromo-5-[(2,6-dichloro-phenylamino)-methyl]-1-methyl-2-phenyl-1,2-dihydro-pyrazol-3-one | 3.91 | 427 |
| 535 | 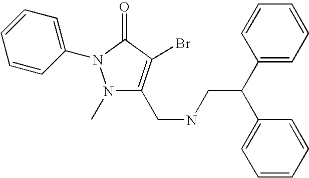 | 4-Bromo-5-[(2,2-diphenyl-ethylamino)-methyl]-1-methyl-2-phenyl-1,2-dihydro-pyrazol-3-one | 2.824 | 462 |
| 536 | 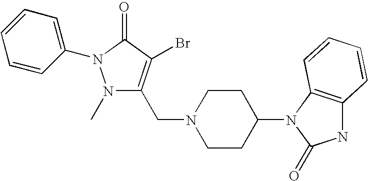 | 1-[1-(4-Bromo-2-methyl-5-oxo-1phenyl-2,5-dihydro-1H-pyrazol-3-ylmethyl)-piperidin-4-yl]-1,3-dihydro-benzoimidazol-2-one | 2.32 | 482 |
| 537 | 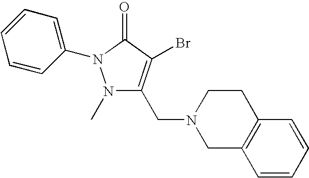 | 4-Bromo-5-(3,4-dihydro-1H-isoquinolin-2-ylmethyl)-1-methyl-2-phenyl-1,2-dihydro-pyrazol-3-one | 2.52 | 398 |
| 538 | 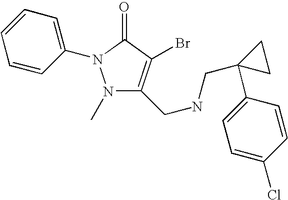 | 4-Bromo-5-({[1-(4-chloro-phenyl)-cyclopropylmethyl]-amino}-methyl)-1-methyl-2-phenyl-1,2-dihydro-pyrazol-3-one | 2.72 | 446 |
| 539 | 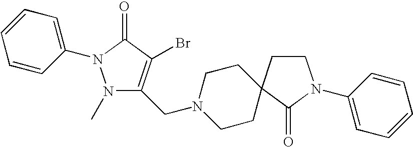 | 8-(4-Bromo-2-methyl-5-oxo-1-phenyl-2,5-dihydro-1H-pyrazol-3-ylmethyl)-2-phenyl-2,8-diaza-spiro[4.5]decan-1-one | 1.41 | 495 |
| 540 | 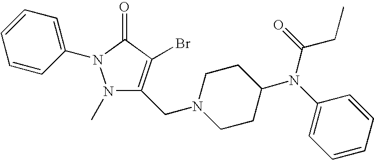 | N-[1-(4-Bromo-2-methyl-5-oxo-1-phenyl-2,5-dihydro-1H-pyrazol-3-ylmethyl)-piperidin-4-yl]-N-phenyl-propionamide | 1.35 | 497 |
| 541 | 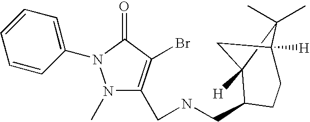 | 4-Bromo-5{[((1S,2R,5S)-6,6-dimethyl-bicyclo[3.1.1]hept-2-ylmethyl)-amino]-methyl}-1-methyl-2-phenyl-1,2-dihydro-pyrazol-3-one | 2.75 | 418 |
| 542 | 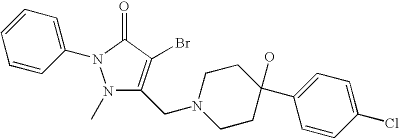 | 4-Bromo-5-[4-(4-chloro-phenyl)-4-hydroxy-piperidin-1-ylmethyl]-1-methyl-2-phenyl-1,2-dihydro-pyrazol-3-one | 2.64 | 476 |
| 543 | 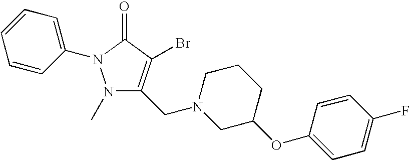 | 4-Bromo-5-[3-(4-fluoro-phenoxy)-piperidin-1-ylmethyl]-1-methyl-2-phenyl-1,2-dihydro-pyrazol-3-one | 2.79 | 460 |
| 544 | 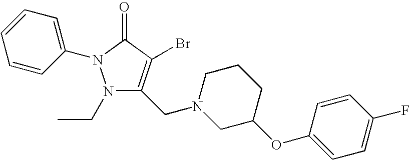 | 4-Bromo-1-ethyl-5-[3-94-fluoro-phenoxy)-piperidin-1-ylmethyl]-2-phenyl-1,2-dihydro-pyrazol-3-one | 2.94 | 474 |

### Example 545

### Spiro(1H-indene-1,4-piperidin)-2-(3H)-one

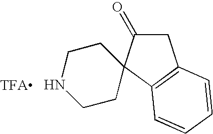

A solution of Spiro(2,3-dihydro-3-oxo-1H-indene-1,4-piperidine)-1-carboxylic acid-1,1-dimethylethyl ester (150 mg) in CH₂Cl₂ (2 mL) was stirred with TFA (2 mL). After 1 h, the volatiles were evaporated and the crude residue of TFA salt of Spiro(1H-indene-1,4-piperidin)-2-(3H)-one was dissolved in DMF and reacted with bromopyrazolones at 100° C. as described in the general procedure.

In a manner similar to the procedure of Example 545 an N-boc group was removed from spiropiperidines used for the compounds of Examples 546 through 549 listed in the following table.

| | | | LC/MS | |
|---|---|---|---|---|
| | | | (Method | |
| | | | B) | m/z |
| Example | Structure | Name | (min.) | (M + 1) |
| 546 | 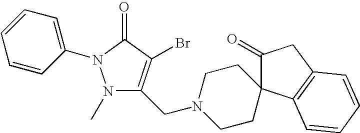 | 4-(4-Bromo-2-methyl-5-oxo-1-phenyl-2,5-dihydro-1-H-pyrazol-3-ylmethyl)-spiro(1H-indene-1,4-piperidin)-2-(3H)-one | 2.51 | 467 |
| 547 | 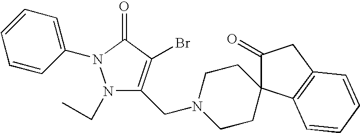 | 4-(4-Bromo-2-ethyl-5-oxo-1-phenyl-2,5-dihydro-1-H-pyrazol-3-ylmethyl)-spiro(1H-indene-1,4-piperidin)-2-(3H)-one | 2.61 | 481 |
| 548 | 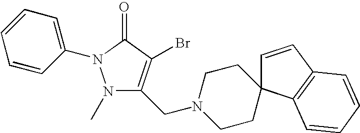 | 4-(4-Bromo-2-ethyl-5-oxo-1-phenyl-2,5-dihydro-1-H-pyrazol-3-ylmethyl)-spiro(1H-indene-1,4-piperidine) | 2.78 | 450 |
| 549 | 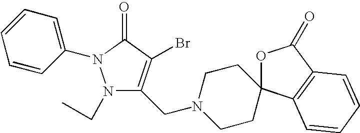 | 4-(4-Bromo-2-ethyl-5-oxo-1-phenyl-2,5-dihydro-1-H-pyrazol-3-ylmethyl)-spiro(pthalan-1,4-piperidine)-3-one | 2.63 | 482 |

### Example 550

### 5-(2-Aza-spiro[4.5]dec-2-ylmethyl)-4-bromo-1-methyl-2-phenyl-1,2-dihydro-pyrazol-3-one

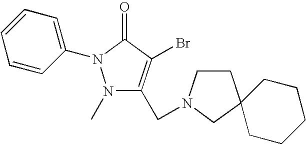

A solution of 2-Aza-spiro[4.5]decan-1-one (459 mg, 3 mmol) in THF (15 mL) was treated with 3.0 mL of LAH solution (1M in THF, 3 mmol). After overnight stirring at rt, heated to reflux for 15 min., cooled to rt, quenched in succession with EtOAc and sat. aq. Na₂SO₄. Extracted with ether, dried over sodium sulfate and evaporated. The crude product was converted into 5-(2-Aza-spiro[4.5]dec-2-ylmethyl)-4-bromo-1-methyl-2-phenyl-1,2-dihydro-pyrazol-3-one as described in the general procedure. LC/MS (Method B): 404 (M+1) at 2.44 min.

### Example 551

### 8-(4-Bromo-2-methyl-5-oxo-1-phenyl-2,5-dihydro-1H-pyrazol-3-ylmethyl)-4-phenyl-2,8-diaza-spiro[4.5]decan-1-one

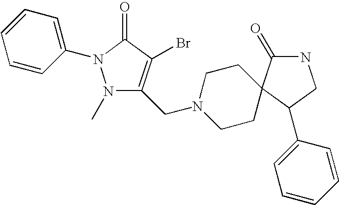

1-Oxo-4-phenyl-2,8-diaza-spiro[4.5]decane-8-carboxylic acid tert-butyl ester (30 mg) was stirred with TFA (2 mL) and CH₂Cl₂ (2 mL). After 1 h, the volatiles were evaporated and the residue was dried under high vacuum (1 h). The crude deprotected material was used as such for making 8-(4-Bromo-2-methyl-5-oxo-1-phenyl-2,5-dihydro-1H-pyrazol-3-ylmethyl)-4-phenyl-2,8-diaza-spiro[4.5]decan-1-one. Thus a mixture of crude 4-Phenyl-2,8-diaza-spiro[4.5]decan-1-one and 4-bromo-5-bromomethyl-1-methyl-2-phenyl-1,2-dihydropyrazol-3-one (35 mg) in CH₃CN (2 mL) containing DIPEA (0.25 mL) was microwaved at 100° C. for 5 min. The cooled reaction mixture was evaporated and the residue was chromatographed on silical gel (3% MeOH/CH₂Cl₂). ¹H NMR (300 MHz, CDCl₃): □(ppm) 7.38-7.28 (m, 10H), 6.02 (s, 1H), 3.8-3.66 (m, 1H), 3.5 (s, 2H), 3.39 (m, 2H), 3.17 (s, 3H), 3 (m, 1H), 2.67 (m, 1H), 2.56 (m, 1H), 2.17 (m, 1H), 1.98 (m, 1H), 1.25 (m, 1H). LC/MS (Method B): 495 (M+1) at 2.29 min.

### Example 552

### 1-Oxo-4-phenyl-2,8-diaza-spiro[4.5]decane-8-carboxylic acid tert-butyl ester was prepared as described below

### 4-[1-(4-Bromo-phenyl)-2-nitro-ethyl]-piperidine-1,4-dicarboxylic acid-1-tert-butyl ester 4-methyl ester

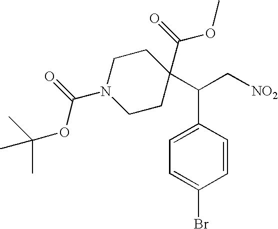

To a cold (−78° C.) solution of N-boc-(Methyl isonipocotate) (486 mg, 2 mmol) [which was readily prepared by esterification of corresponding acid with TMSCHN₂ in MeOH] in THF (5 mL) was added a solution of KHMDS (4.8 mL of 0.5M toluene solution, 2.4 mmol, 1.2 eq.) using a syringe. After 10 min., a solution of 4-bromo-O-nitrostyrene (450 mg, 2 mmol) in THF (5 mL) over 1-2 min. and slowly allowed to attain rt overnight. Carefully quenched with pH 7 aqueous buffer and extracted with CH₂Cl₂. The crude product was chromatographed over silica gel column using 30% EtOAc-hexanes. ¹H NMR (300 MHz, CDCl₃): □(ppm) 7.61 (d, 2H), 6.94 (d, 2H), 4.84 (d, 2H), 3.72 (s, 3H), 3.55 (dd, 1H), 2.6 (m, 2H), 2.5 (m, 1H), 2.45 (m, 1H), 2.2 (m, 1H), 1.85 (m, 2H), 1.6 (m, 1H). LC/MS (Method B): 493 (M+Na) at 4.72 mm.

### Example 553

### 1-Oxo-4-phenyl-2,8-diaza-spiro[4.5]decane-8-carboxylic acid tert-butyl ester

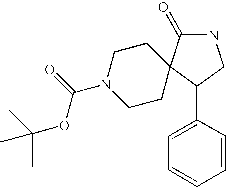

To a mixture of 4-[1-(4-Bromo-phenyl)-2-nitro-ethyl]-piperidine-1,4-dicarboxylic acid-1-tert-butyl ester 4-methyl ester (110 mg, 0.23 mmol) and ammonium formate (130 mg, 2 mmol) in MeOH (2.2 mL) was added 10% Pd—C (20 mg). The resultant suspension was microwaved at 120° C. for 15 min. Filtration, concentration and chromatography on a silicagel column (5% MeOH/CH₂Cl₂) provided 1-Oxo-4-phenyl-2,8-diaza-spiro[4.5]decane-8-carboxylic acid tert-butyl ester. ¹H NMR (300 MHz, CDCl₃): □(ppm) 7.36-7.11 (m, 5H), 6.38 (br s, 1H), 4.03-3.28 (m, 7H), 1.82 (m, 1H), 1.63 (m, 2H), 1.39 (s, 9H), 1.12 (m, 1H). LC/MS (Method B): 353 (M+Na) at 3.71 min.

### Example 554

### 8-(4-Bromo-2-methyl-5-oxo-1-phenyl-2,5-dihydro-1H-pyrazol-3-ylmethyl)-4-(4-dimethylamino-phenyl)-2,8-diaza-spiro[4.5]decan-1-one

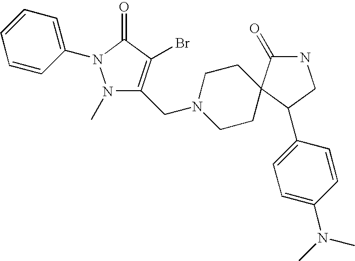

Reaction of 4-bromo-5-bromomethyl-1-methyl-2-phenyl-1,2-dihydropyrazol-3-one and 4-(4-Dimethylamino-phenyl)-2,8-diaza-spiro[4.5]decan-1-one following the procedure described for 8-(4-Bromo-2-methyl-5-oxo-1-phenyl-2,5-dihydro-1H-pyrazol-3-ylmethyl)-4-phenyl-2,8-diaza-spiro[4.5]decan-1-one furnished 8-(4-Bromo-2-methyl-5-oxo-1-phenyl-2,5-dihydro-1H-pyrazol-3-ylmethyl)-4-(4-dimethylamino-phenyl)-2,8-diaza-spiro[4.5]decan-1-one. LC/MS (Method B): 538 (M+1) at 1.85 min.

The intermediate compounds for this synthesis were prepared analogous to the preparation of intermediates for 8-(4-Bromo-2-methyl-5-oxo-1-phenyl-2,5-dihydro-1H-pyrazol-3-ylmethyl)-4-phenyl-2,8-diaza-spiro[4.5]decan-1-one (Example 549).

### Example 555

### 4-(4-Dimethylamino-phenyl)-1-oxo-2,8-diaza-spiro[4.5]decane-8-carboxylic acid tert-butyl ester

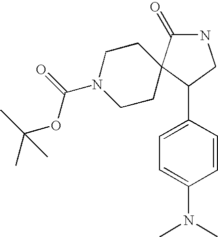

LC/MS (Method B): 396 (M+Na) at 2.46 min.

### Example 556

### 4-[1-(4-Dimethylamino-phenyl)-2-nitro-ethyl]-piperidine-1,4-dicarboxylic acid-1-tert-butyl ester 4-methyl ester

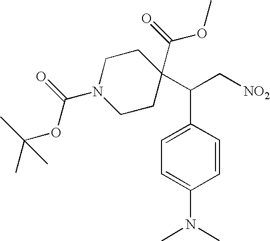

LC/MS (Method B): 458 (M+Na) at 3.42 min.

### Example 557

### 8-(4-Bromo-2-methyl-5-oxo-1-phenyl-2,5-dihydro-1H-pyrazol-3-ylmethyl)-2-hydroxy-4-pyridin-3-yl-2,8-diaza-spiro[4.5]decan-1-one

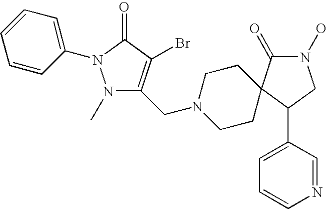

This compound was prepared as described before for the synthesis of 8-(4-Bromo-2-methyl-5-oxo-1-phenyl-2,5-dihydro-1H-pyrazol-3-ylmethyl)-4-phenyl-2,8-diaza-spiro[4.5]decan-1-one (Example 549). LC/MS (Method C): 526 (M+1) at 0.83 min.

### Example 558

### 8-(4-Bromo-2-methyl-5-oxo-1-phenyl-2,5-dihydro-1H-pyrazol-3-ylmethyl)-1-phenyl-1,8-diaza-spiro[4.5]decan-4-one

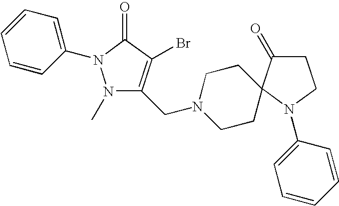

A mixture of 1-phenyl-1,8-diaza-spiro[4.5]decan-4-one (46 mg, 0.2 mmol), 4-bromo-5-bromomethyl-1-methyl-2-phenyl-1,2-dihydropyrazol-3-one (70 mg, 0.2 mmol) and DIPEA (100 □L) in CH₃CN (1.5 mL) was stirred at rt overnight and 50° C. for 15 min. The volatiles were evaporated and the residue was chromatographed on silica gel with 2.5% MeOH/CH₂Cl₂. Further purified by supercritical fluid chromatography. ¹H NMR (300 MHz, CDCl3): □(ppm) 7.48 (m, 2H), 7.44-7.25 (m, 5H), 7.1 (d, 2H), 6.95 (t, 1H), 3.6 (s, 2H), 3.56-3.6 (m, 2H), 3.2 (s, 3H), 2.89-2.8 (m, 4H), 2.67 (t, 2H), 2.25 (m, 2H), 1.64 (m, 2H). LC/MS (Method B): 495 (M+1) at 2.66 min.

### Example 559

### 8-(4-Bromo-2-ethyl-5-oxo-1-phenyl-2,5-dihydro-1H-pyrazol-3-ylmethyl)-1-phenyl-1,8-diaza-spiro[4.5]decan-4-one

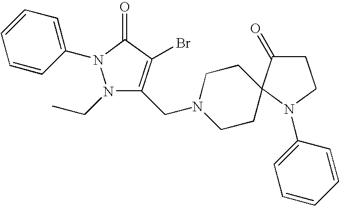

LC/MS (Method C): 510 (M+1) at 1.79 min. 1-phenyl-1,8-diaza-spiro[4.5]decan-4-one was prepared according to the published route of Vandewalle et al (Bull. Soc. Chim. Belges, 1981, 90, 749).

### Example 560

### 8-(4-Iodo-2-methyl-5-oxo-1-phenyl-2,5-dihydro-1H-pyrazol-3-ylmethyl)-1-phenyl-1,3,8-triazaspiro[4.5]decan-4-one

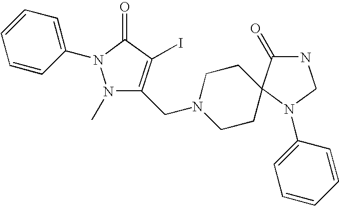

A suspension of 8-(4-Bromo-2-methyl-5-oxo-1-phenyl-2,5-dihydro-1H-pyrazol-3-ylmethyl)-1-phenyl-1,3,8-triazaspiro[4.5]decan-4-one (250 mg, 0.5 mmol), CuI (5 mg), NaI (150 mg) and trans-N,N′-Dimethyl-cyclohexane-1,2-diamine (8 mg) in 1,4-dioxane was purged with N₂ and heated in a sealed tube. After 20 h, the reaction mixture was cooled to rt, added aq. ammonia and extracted with CH₂Cl₂ (25 mL). Dried the extract (Na₂SO₄) and evaporated to a give a solid residue which was triturated with CH₃CN to give a white solid (50 mg). ¹H NMR (300 MHz, DMSO): □(ppm) 8.65 (br s, 1H), 7.53 (m, 2H), 7.4 (m, 3H), 7.26 (m, 2H), 6.85 (d, 2H), 6.73 (t, 1H), 4.58 (s, 2H), 3.67 (br s, 2H), 3.27 (s, 3H), 2.9 (m, 4H), 2.57 (m, 2H), 1.64 (m, 2H). LC/MS (Method B): 543 (M+1) at 2.52 min.

### Example 561

### 8-(4-Bromo-2-methyl-5-oxo-1-phenyl-2,5-dihydro-1H-pyrazol-3-ylmethyl)-1-(4-iodo-phenyl)-1,3,8-triazaspiro[4.5]decan-4-one

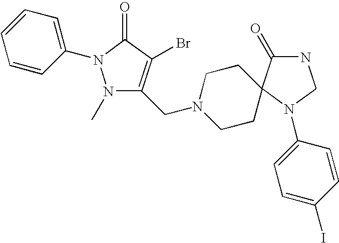

To a suspension of 8-(4-Bromo-2-methyl-5-oxo-1-phenyl-2,5-dihydro-1H-pyrazol-3-ylmethyl)-1-phenyl-1,3,8-triazaspiro[4.5]decan-4-one in MeOH (2 mL) and 10% aq. HCl (2 mL) was added ICl (100 mg). The resultant yellowish suspension was stirred at rt. After 2 h, filtered, rinsed with MeOH and dried under vacuum. ¹H NMR (300 MHz, DMSO): □(ppm) 9.08 (br s, 1H), 7.6-6.8 (m, 9H), 4.6 (s, 2H), 4.5 (br s, 2H), 3.9 (m, 2H), 3.68 (m, 2H), 3.25 (s, 3H), 2.77 (m, 2H), 2 (m, 2H). LC/MS (Method B): 622 (M+1) at 2.91 min.

### Example 562

### p-Tolyl-carbamic acid 1-(4-bromo-2-methyl-5-oxo-1-phenyl-2,5-dihydro-1H-pyrazol-3-ylmethyl)-piperidin-4-yl ester

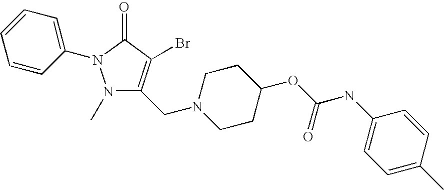

To a solution of 4-bromo-5-(4-hydroxy-piperidin-1-ylmethyl)-1-methyl-2-phenyl-1,2-dihydro-pyrazol-3-one (55 mg, 0.15 mmol) in CH₂Cl₂ (2 mL) was added 25 □L of p-tolylisocyanate (0.2 mmol) using a syringe and stirred at room temperature for 3 h. Stirred with PS-trisamine resin and then purified by reversed phase HPLC. ¹H NMR (300 MHz, DMSO): □(ppm) 9.51 (br s, 1H), 7.58-7.06 (m, 9H), 4.8 (br s, 1H), 3.78 (br s, 2H), 3.22 (s, 3H), 2.23 (s, 3H), 2.08 (m, 4H), 1.83 (m, 4H). LC/MS (Method B): 499 (M+1) at 2.75 min.

The following carbamates of Examples 563 through 566 were prepared in an analogous manner to the procedure of Example 562.

| | | | LC/MS | |
|---|---|---|---|---|
| | | | (Method | |
| | | | B) | m/z |
| Example | Structure | Name | (min.) | (M + 1) |
| 563 | 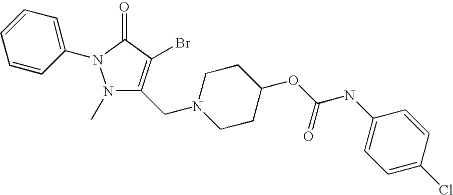 | 4-(Chloro-phenyl)-carbamic acid 1-(4-bromo-2-methyl-5-oxo-1-phenyl-2,5-dihydro-1H-pyrazol-3-ylmethyl)-piperidin-4-yl ester | 2.88 | 519 |
| 564 | 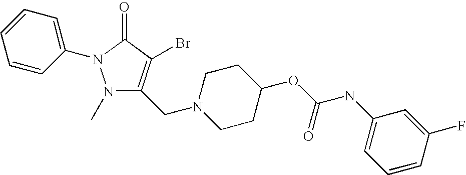 | 3-(Fluoro-phenyl)-carbamic acid 1-(4-bromo-2-methyl-5-oxo-1-phenyl-2,5-dihydro-1H-pyrazol-3-ylmethyl)-piperidin-4-yl ester | 2.76 | 503 |
| 565 | 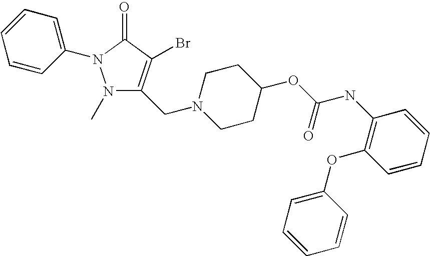 | (2-Phenoxy-phenyl)-carbamic acid 1-(4-bromo-2-methyl-5-oxo-1-phenyl-2,5-dihydro-1H-pyrazol-3-ylmethyl)-piperidin-4-yl ester | 3.08 | 577 |
| 566 | 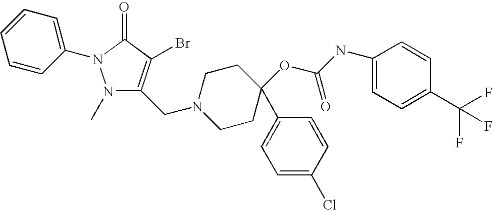 | (4-Trifluoromethyl-phenyl)-carbamic acid1-(4-bromo-2-methyl-5-oxo-1-phenyl-2,5-dihydro-1H-pyrazol-3-ylmethyl)-4-(4-chloro-phenyl)-piperidin-4-yl ester | | |

### Example 567

### 4-Bromo-5-(4-hydroxy-piperidin-1-ylmethyl)-1-methyl-2-phenyl-1,2-dihydro-pyrazol-3-one

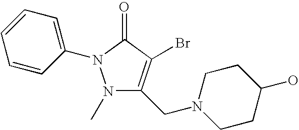

This compound was prepared by following the microwave amination procedure described above. LC/MS (Method B): 366 (M+Na) at 1.7 min.

### Example 568

### Methyl-phenethyl-carbamic acid 1-(4-bromo-2-methyl-5-oxo-1-phenyl-2,5-dihydro-1H-pyrazol-3-ylmethyl)-piperidin-4-yl ester

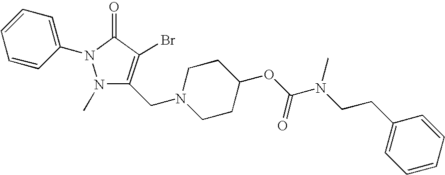

A mixture of 4-Bromo-5-(4-hydroxy-piperidin-1-ylmethyl)-1-methyl-2-phenyl-1,2-dihydro-pyrazol-3-one (146 mg, 0.4 mmol), carbonyldiimidazole (70 mg, 0.44 mmol), DMAP (10 mg) in acetonitrile (2.5 mL) was heated to reflux for 3.5 h. Cooled to rt and phenethylamine (65 □L) was added using the syringe and refluxed for about 15 h. The crude product was purified by flash column chromatography (25% EtOAc-75% hexanes). ¹H NMR (300 MHz, DMSO): □(ppm) 7.75-7.2 (m, 10H), 4.6 (br s, 1H), 3.6 (s, 2H), 3.43 (brs, 2H), 3.22 (s, 3H), 2.85-2.75 (m, 5H), 2.6 (m, 2H), 2.4 (m, 2H), 1.79 (m, 2H), 1.56 (m, 2H). LC/MS (Method C): 499 (M+1) at 1.76 min

Compounds of Examples 569 and 570 were synthesized by a method analogous to the procedure of Example 568.

| | | | LC/MS | |
|---|---|---|---|---|
| | | | (Method | |
| | | | C) | m/z |
| Example | Structure | Name | (min.) | (M + 1) |
| 569 | 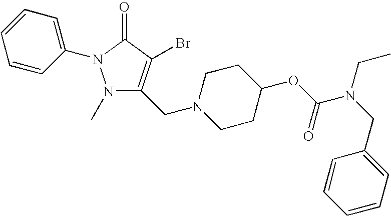 | Benzyl-ethyl-carbamic acid 1-(4-bromo-2-methyl-5-oxo-1-phenyl-2,5-dihydro-1H-pyrazol-3-ylmethyl)-piperidin-4-yl ester | 1.64 | 513 |
| 570 | 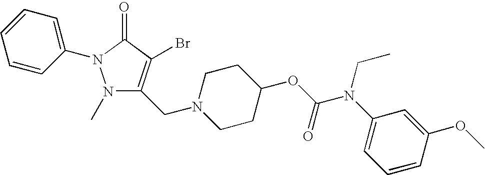 | Ethyl-(2-methoxy-benzyl)-carbamic acid1-(4-bromo-2-methyl-5-oxo-1-phenyl-2,5-dihydro-1H-pyrazol-3-ylmethyl)-piperidin-4-yl ester | 1.87 | 557 |

1. A compound according to Formula I: 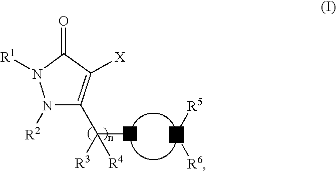
wherein
X is selected from the group consisting of F, Cl, Br, I, cyano, OC₁₋₆-alkyl, C₁₋₆-alkylhalo, OC₁₋₆-alkylhalo;
Q is selected from the group consisting of C, O, S, and N, such that when
Q is C, then at least one of R⁵ and R⁶ is present,
Q is N, then one of R⁵ and R⁶ is present, and
Q is O or S, then R⁵ and R⁶ are both absent; 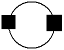
represents a 5- to 7-membered ring, wherein said ring is optionally fused with one or more 5- to 7-membered rings each containing atoms independently selected from the group consisting of C, N, O and S, wherein each of said rings may be substituted by one or more A;
R¹ is selected from the group consisting of C₁₋₆-alkyl, C₂₋₆-alkenyl, C₂₋₆-alkynyl, aryl, heteroaryl, heterocycloalkyl, C₃₋₈-cycloalkyl, C₁₋₆-alkyl-aryl, C₁₋₆-alkyl-heteroaryl, C₁₋₆-alkyl-heterocycloalkyl, C₁₋₆-alkyl-C₃₋₈-cycloalkyl, wherein R¹ may be substituted by one or more A;
R² is selected from the group consisting of H, C₁₋₆-alkyl, C₂₋₆-alkenyl, and C₂₋₆-alkynyl, wherein
R² may be substituted by one or more A;
R³ and R⁴ each are independently selected from the group consisting of H, C₁₋₆-alkyl, C₂₋₆-alkenyl, C₂₋₆-alkynyl, aryl, heteroaryl, heterocycloalkyl, C₃₋₈-cycloalkyl, C₁₋₆-alkyl-aryl, C₁₋₆-alkyl-heteroaryl, C₁₋₆-alkyl-heterocycloalkyl, C₁₋₆-alkyl-C₃₋₈-cycloalkyl, wherein R³ and R⁴ may be substituted by one or more A;
R⁵ and R⁶, when present, are independently selected from the group consisting of H, hydroxy, F, Cl, Br, I, nitro, cyano, C₁₋₆-alkyl, C₁₋₆-alkylhalo, OC₁₋₆alkyl, OC₁₋₆-alkylhalo, C₂₋₆-alkenyl, OC₂₋₆-alkenyl, C₂₋₆-alkynyl, OC₂₋₆-alkynyl, C₃₋₈-cycloalkyl, C₁₋₆-alkyl-C₃₋₈-cycloalkyl, OC₀₋₆-alkyl-C₃₋₈-cycloalkyl, aryl, C₁₋₆-alkylaryl, OC₀₋₆-alkylaryl, heteroaryl, C₁₋₆-alkylheteroaryl, OC₀₋₆-alkylheteroaryl, C(O)H, (CO)R⁷, O(CO)R⁷, O(CO)OR⁷, C(O)OR⁷, OC(NH)OR⁷, C₁₋₆-alkylOR⁷, OC₂₋₆-alkylOR⁷, C₁₋₆-alkyl(CO)R⁷, OC₁₋₆-alkyl(CO)R⁷, C₁₋₆-alkylCO₂R⁷, OC₁₋₆-alkylCO₂R⁷, C₁₋₆-alkylcyano, OC₂₋₆-alkylcyano, C₀₋₆-alkylNR⁷R⁸, OC₂₋₆-alkylNR⁷R⁸, C₀₋₆-alkyl(CO)NR⁷R⁸, OC₀₋₆-alkyl(CO)NR⁷R⁸, C₀₋₆-alkylNR⁷(CO)R⁸, OC₂₋₆-alkylNR⁷(CO)R⁸, C₀₋₆-alkylNR⁷(CO)NR⁷R⁸, C₀₋₆-alkylSR⁷, OC₂₋₆-alkylSR⁷, C₀₋₆-alkyl(SO)R⁷, OC₂₋₆-alkyl(SO)R⁷, C₀₋₆-alkylSO₂R⁷, OC₂₋₆-alkylSO₂R⁷, C₀₋₆-alkyl(SO₂)NR⁷R⁸, OC₂₋₆-alkyl(SO₂)NR⁷R⁸, C₀₋₆-alkylNR⁷(SO₂)R⁸, OC₂₋₆-alkylNR⁷(SO₂)R⁸, C₀₋₆-alkylNR⁷(SO₂)NR⁷R⁸, OC₂₋₆-alkylNR⁷(SO₂)NR⁷R⁸, (CO)NR⁷R⁸, O(CO)NR⁷R⁸, NR⁷OR⁸, C₀₋₆-alkylNR⁷(CO)OR⁸, OC₂₋₆-alkylNR⁷(CO)OR⁸, SO₃R⁷ and a 5- to 7-membered ring containing atoms independently selected from the group consisting of C, N, O and S, wherein R⁵ and R⁶ may be substituted by one or more A, and wherein any cycloalkyl or aryl is optionally fused to a 5- to 7-membered ring containing atoms independently selected from the group consisting of C, N, O and S;
or, optionally, when Q is C, then R⁵ and R⁶, together with Q, may form a 5- to 7-membered ring, which may be unsaturated, containing atoms independently selected from the group consisting of C, N, O and S, wherein
i) said ring is optionally fused with one or more 5- to 7-membered rings each containing atoms independently selected from the group consisting of C, N, O and S, and wherein
ii) said rings each may be substituted by one or more A;
R⁷ and R⁸ are independently selected from the group consisting of hydrogen, C₁₋₆-alkyl, C₃₋₇-cycloalkyl, C(O)C₁₋₆-alkyl, aryl, C₁₋₆-alkylaryl, heterocycloalkyl, and heteroaryl, wherein R⁷ and
R⁸ may be substituted by one or more A;
A is selected from the group consisting of hydroxy, F, Cl, Br, I, nitro, cyano, oxo, C₁₋₆-alkyl, C₁₋₆-alkylhalo, OC₁₋₆alkyl, OC₁₋₆-alkylhalo, C₂₋₆-alkenyl, OC₂₋₆-alkenyl, C₂₋₆-alkynyl, OC₂₋₆-alkynyl, C₃₋₈-cycloalkyl, C₁₋₆-alkyl-C₃₋₈-cycloalkyl, OC₀₋₆-alkyl-C₃₋₈-cycloalkyl, aryl, C₁₋₆-alkylaryl, OC₀₋₆-alkylarylheteroaryl, C₁₋₆-alkylheteroaryl, OC₀₋₆-alkylheteroaryl, (CO)R⁹, O(CO)R⁹, O(CO)OR⁹, OC(NH)OR⁹, C₁₋₆-alkylOR⁹, OC₂₋₆-alkylOR⁹, C₁₋₆-alkyl(CO)R⁹, OC₁₋₆-alkyl(CO)R⁹, C₀₋₆-alkylCO₂R⁹, OC₁₋₆-alkylCO₂R⁹, C₁₋₆-alkylcyano, OC₂₋₆-alkylcyano, C₀₋₆-alkylNR⁹R¹⁰, OC₂₋₆-alkylNR⁹R¹⁰, C₁₋₆-alkyl(CO)NR⁹R¹⁰, OC₁₋₆-alkyl(CO)NR⁹R¹⁰, C₀₋₆-alkylNR⁹(CO)R¹⁰, OC₂₋₆-alkylNR⁹(CO)R¹⁰, C₀₋₆-alkylNR⁹(CO)NR⁹R¹⁰, C₀₋₆-alkylSR⁹, OC₂₋₆-alkylSR⁹, C₀₋₆-alkyl(SO)R⁹, OC₂₋₆-alkyl(SO)R⁹, C₀₋₆-alkylSO₂R⁹, OC₂₋₆-alkylSO₂R⁹, C₀₋₆-alkyl(SO₂)NR⁹R¹⁰, OC₂₋₆-alkyl(SO₂)NR⁹R¹⁰, C₀₋₆-alkylNR⁹(SO₂)R¹⁰, OC₂₋₆-alkylNR⁹(SO₂)R¹⁰, C₀₋₆-alkylNR⁹(SO₂)NR⁹R¹⁰, OC₂₋₆-alkylNR⁹(SO₂)NR⁹R¹⁰, (CO)NR⁹R¹⁰, O(CO)NR⁹R¹⁰, NR⁹OR¹⁰, C₀₋₆-alkylNR⁹(CO)OR¹⁰, OC₂₋₆-alkylNR⁹(CO)OR¹⁰, OC(NH)OR⁹, SO₃R⁹, wherein any ring is optionally substituted with one or more B, and a 5- to 7-membered ring containing atoms independently selected from the group consisting of C, N, O and S, wherein said ring is optionally substituted by one or more of R⁹ and R¹⁰;
R⁹ and R¹⁰ are independently selected from the group consisting of H, hydroxy, F, Cl, Br, I, nitro, cyano, oxo, C₁₋₆-alkyl, C₁₋₆-alkylhalo, OC₁₋₆alkyl, OC₁₋₆-alkylhalo, C₂₋₆-alkenyl, OC₂₋₆-alkenyl, C₂₋₆-alkynyl, OC₂₋₆-alkynyl, C₃₋₈-cycloalkyl, C₁₋₆-alkyl-C₃₋₈-cycloalkyl, OC₀₋₆-alkyl-C₃₋₈-cycloalkyl, aryl, C₁₋₆-alkylaryl, OC₀₋₆-alkylaryl, heterocycloalkyl, and heteroaryl, and any ring is optionally substituted with one or more B;
B is selected from the group consisting of F, Cl, Br, I, C₁₋₆-alkyl and OC₁₋₆alkyl; and
n is selected from the group consisting of 1, 2, 3, 4, 5, and 6;
or a pharmaceutically acceptable salt, hydrate, solvate, optical isomer, or combination thereof.

2. A compound according to claim 1, wherein 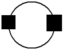
is selected from the group consisting of:

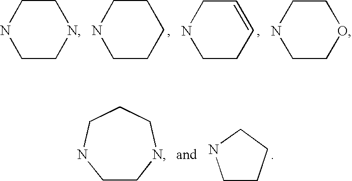

3. A compound according to claim 2, wherein

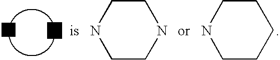

4. A compound according to claim 3, wherein

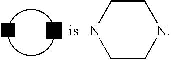

5. A compound according to claim 3, wherein X is selected from the group consisting of Br, Cl, and OC₁₋₆-alkyl.

6. A compound according to claim 5, wherein X is Br or Cl.

7. A compound according to claim 1, wherein R¹ is selected from the group consisting of aryl, C₃₋₈-cycloalkyl, C₁₋₆-alkyl-aryl, and C₁₋₆-alkyl-C₃₋₈-cycloalkyl, wherein R¹ may be substituted by one or more A.

8. A compound according to claim 7, wherein R¹ is selected from aryl and C₃₋₈-cycloalkyl, wherein R¹ may be substituted by one or more A.

9. A compound according to claim 8, wherein R¹ is aryl that may be substituted by one or more A.

10. A compound according to claim 9, wherein R¹ is phenyl that may be substituted by one or more A.

11. A compound according to claim 8, wherein R¹ is C₃₋₈-cycloalkyl that maybe substituted by one or more A.

12. A compound according to claim 11, wherein R¹ is cyclohexyl that may be substituted by one or more A.

13. A compound according to claim 1, wherein R² is selected from the group consisting of H and C₁₋₆-alkyl.

14. A compound according to claim 13, wherein R² is C₁₋₆-alkyl.

15. A compound according to claim 14, wherein R² is selected from methyl and ethyl.

16. A compound according to claim 1, wherein R⁵ and R⁶, when one or both are present, are independently selected from the group consisting of H, aryl, and C₃₋₈-cycloalkyl, wherein R⁵ and R⁶ may be substituted by one or more A.

17. A compound according to claim 1, wherein Q is C.

18. A compound according to claim 17, wherein R⁵ and R⁶ are both present.

19. A compound according to claim 18, wherein R⁵, R⁶, and Q combine to form a 5- to 7-membered ring containing atoms independently selected from the group consisting of C, N, O and S.

20. A compound according to claim 19, wherein the ring is 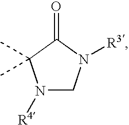 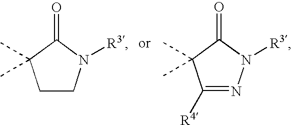
wherein the dashed lines indicate spiro fusion via Q to 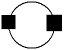
wherein R^{3′} and R^{4′} have the same definitions as R³ and R⁴, respectively, and wherein R^{3′} and R^{4′} may be substituted by one or more A.

21. A compound according to claim 20, wherein the ring is

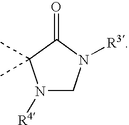

22. A compound according to claim 20, wherein the ring is

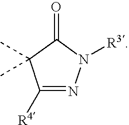

23. A compound according to claim 20, wherein R^{3′} and R^{4′} are independently selected from the group consisting of H, C₁₋₆-alkyl, C₁₋₆-alkyl-aryl, aryl, and heteroaryl, wherein R³ and R⁴ may be substituted by one or more A.

24. A compound according to claim 23, wherein R^{4′} is aryl that may be substituted by one or more A.

25. A compound according to claim 24, wherein R^{4′} is phenyl that may be substituted by one or more A.

26. A compound according to claim 20, wherein the ring is 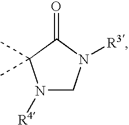
R^{3′} is selected from the group consisting of H, C₁₋₆-alkyl, C₁₋₆-alkyl-aryl, aryl, and heteroaryl; R^{4′} is phenyl; and wherein R^{3′} and R^{4′} may be substituted by one or more A.

27. A compound according to claim 1, wherein
X is selected from the group consisting of Cl, Br, and OC₁₋₆-alkyl; 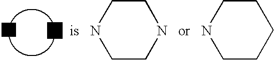
that may be substituted by one or more A;
R¹ is selected from aryl and C₃₋₈-cycloalkyl, wherein R¹ may be substituted by one or more A;
R² is selected from H and C₁₋₆-alkyl;
R⁵ and R⁶, when one or more is present, are independently selected from the group consisting of H, aryl, and C₃₋₈-cycloalkyl, wherein R⁵ and R⁶ maybe substituted by one or more A; and
n is 1.

28. A compound according to Formula II: 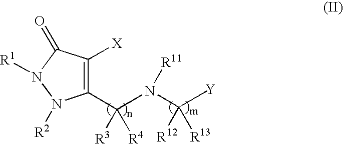
wherein
X is selected from the group consisting of F, Cl, Br, I, cyano, OC₁₋₆-alkyl, C₁₋₆-alkylhalo, OC₁₋₆-alkylhalo;
R¹ is selected from the group consisting of C₁₋₆-alkyl, C₂₋₆-alkenyl, C₂₋₆-alkynyl, aryl, heteroaryl, heterocycloalkyl, C₃₋₈-cycloalkyl, C₁₋₆-alkyl-aryl, C₁₋₆-alkyl-heteroaryl, C₁₋₆-alkyl-heterocycloalkyl, C₁₋₆-alkyl-C₃₋₈-cycloalkyl, wherein R¹ may be substituted by one or more A;
R² is selected from the group consisting of H, C₁₋₆-alkyl, C₂₋₆-alkenyl, and C₂₋₆-alkynyl, wherein
R² may be substituted by one or more A;
R³, R⁴, R¹² and R¹³ are each independently selected from the group consisting of H, C₁₋₆-alkyl, C₂₋₆-alkenyl, C₂₋₆-alkynyl, aryl, heteroaryl, heterocycloalkyl, C₃₋₈-cycloalkyl, C₁₋₆-alkyl-aryl, C₁₋₆-alkyl-heteroaryl, C₁₋₆-alkyl-heterocycloalkyl, C₁₋₆-alkyl-C₃₋₈-cycloalkyl, wherein R³ and R⁴ may be substituted by one or more A;
R¹¹ is selected from the group consisting of H, C₁₋₆-alkyl, C₁₋₆-alkylhalo, C₂₋₆-alkenyl, C₂₋₆-alkynyl, C₃₋₈-cycloalkyl, C₁₋₆-alkyl-C₃₋₈-cycloalkyl, C₃₋₈-heterocycloalkyl, C₁₋₆-alkyl-C₃₋₈-heterocycloalkyl aryl, C₁₋₆-alkylaryl, heteroaryl, C₁₋₆-alkylheteroaryl, C(O)H, (CO)R⁷, C(O)OR⁷, C₁₋₆-alkylOR⁷, C₁₋₆-alkyl(CO)R⁷, C₁₋₆-alkylCO₂R⁷, C₁₋₆-alkylcyano, C₁₋₆-alkylNR⁷R⁸, C₁₋₆ alkyl(CO)NR⁷R⁸, C₁₋₆-alkylNR⁷(CO)R⁸, C₀₋₆-alkylNR⁷(CO)NR⁷R⁸, C₁₋₆-alkylSR⁷, C₀₋₆-alkyl(SO)R⁷, C₀₋₆-alkylSO₂R⁷, C₀₋₆-alkyl(SO₂)NR⁷R⁸, C₀₋₆-alkylNR⁷(SO₂)R⁸, C₀₋₆-alkylNR⁷(SO₂)NR⁷R⁸, (CO)NR⁷R⁸, C₀₋₆-alkylNR⁷(CO)OR⁸, C₀₋₆-alkyl SO₃R⁷ and a 5- to 7-membered ring containing atoms independently selected from the group consisting of C, N, O and S, wherein R¹¹ may be substituted by one or more A, and wherein any cycloalkyl or aryl is optionally fused to a 5- to 7-membered ring containing atoms independently selected from the group consisting of C, N, O and S;
R⁷ and R⁸ are independently selected from the group consisting of hydrogen, C₁₋₆-alkyl, C₃₋₇-cycloalkyl, C(O)C₁₋₆-alkyl, aryl, C₁₋₆-alkylaryl, heterocycloalkyl, and heteroaryl, wherein R⁷ and R⁸ may be substituted by one or more A;
A is selected from the group consisting of hydroxy, F, Cl, Br, I, nitro, cyano, oxo, C₁₋₆-alkyl, C₁₋₆-alkylhalo, OC₁₋₆alkyl, OC₁₋₆-alkylhalo, C₂₋₆-alkenyl, OC₂₋₆-alkenyl, C₂₋₆-alkynyl, OC₂₋₆-alkynyl, C₃₋₈-cycloalkyl, C₁₋₆-alkyl-C₃₋₈-cycloalkyl, OC₀₋₆-alkyl-C₃₋₈-cycloalkyl, aryl, C₁₋₆-alkylaryl, OC₀₋₆-alkylarylheteroaryl, C₁₋₆-alkylheteroaryl, OC₀₋₆-alkylheteroaryl, (CO)R⁹, O(CO)R⁹, O(CO)OR⁹, OC(NH)OR⁹, C₁₋₆-alkylOR⁹, OC₂₋₆-alkylOR⁹, C₁₋₆-alkyl(CO)R⁹, OC₁₋₆-alkyl(CO)R⁹, C₀₋₆-alkylCO₂R⁹, OC₁₋₆-alkylCO₂R⁹, C₁₋₆-alkylcyano, OC₂₋₆-alkylcyano, C₀₋₆-alkylNR⁹R¹⁰, OC₂₋₆-alkylNR⁹R¹⁰, C₁₋₆-alkyl(CO)NR⁹R¹⁰, OC₁₋₆-alkyl(CO)NR⁹R¹⁰, C₀₋₆-alkylNR⁹(CO)R¹⁰, OC₂₋₆-alkylNR⁹(CO)R¹⁰, C₀₋₆-alkylNR⁹(CO)NR⁹R¹⁰, C₀₋₆-alkylSR⁹, OC₂₋₆-alkylSR⁹, C₀₋₆-alkyl(SO)R⁹, OC₂₋₆-alkyl(SO)R⁹, C₀₋₆-alkylSO₂R⁹, OC₂₋₆-alkylSO₂R⁹, C₀₋₆-alkyl(SO₂)NR⁹R¹⁰, OC₂₋₆-alkyl(SO₂)NR⁹R¹⁰, C₀₋₆-alkylNR⁹(SO₂)R¹⁰, OC₂₋₆-alkylNR⁹(SO₂)R¹⁰, C₀₋₆-alkylNR⁹(SO₂)NR⁹R¹⁰, OC₂₋₆-alkylNR⁹(SO₂)NR⁹R¹⁰, (CO)NR⁹R¹⁰, O(CO)NR⁹R¹⁰, NR⁹OR¹⁰, C₀₋₆-alkylNR⁹(CO)OR¹⁰, OC₂₋₆-alkylNR⁹(CO)OR¹⁰, OC(NH)OR⁹, SO₃R⁹, wherein any ring is optionally substituted with one or more B, and a 5- to 7-membered ring containing atoms independently selected from the group consisting of C, N, O and S, wherein said ring is optionally substituted by one or more of R⁹ and R¹⁰;
R⁹ and R¹⁰ are independently selected from the group consisting of H, hydroxy, F, Cl, Br, I, nitro, cyano, oxo, C₁₋₆-alkyl, C₁₋₆-alkylhalo, OC₁₋₆alkyl, OC₁₋₆-alkylhalo, C₂₋₆-alkenyl, OC₂₋₆-alkenyl, C₂₋₆-alkynyl, OC₂₋₆-alkynyl, C₃₋₈-cycloalkyl, C₁₋₆-alkyl-C₃₋₈-cycloalkyl, OC₀₋₆-alkyl-C₃₋₈-cycloalkyl, aryl, C₁₋₆-alkylaryl, OC₀₋₆-alkylaryl, heterocycloalkyl, and heteroaryl, and any ring is optionally substituted with one or more B;
B is selected from the group consisting of F, Cl, Br, I, C₁₋6-alkyl and OC₁₋₆alkyl;
m is selected from the group consisting of 0, 1, 2, 3, 4, 5, and 6;
n is selected from the group consisting of 1, 2, 3, 4, 5, and 6; and
Y is selected from the group consisting of alkyl, alkenyl, alkynyl, aryl, heteroaryl, heterocycloalkyl and C₃₋₁₀-cycloalkyl, wherein Y may be substituted by one or more A;
or a pharmaceutically acceptable salt, hydrate, solvate, optical isomer, or combination thereof with the proviso that said compound is not:
4-Bromo-1-methyl-5-{[methyl-(2-phenyl-propyl)-amino]-methyl}-2-phenyl-1,2-dihydro-pyrazol-3-one
4-Bromo-1-methyl-5-{[methyl-(1-methyl-2-phenyl-ethyl)-amino]-methyl}-2-phenyl-1,2-dihydro-pyrazol-3-one;
4-Bromo-1-methyl-5-{[methyl-(1-methyl-2-phenyl-ethyl)-amino]-methyl}-2-phenyl-1,2-dihydro-pyrazol-3-one;
2-[(4-Bromo-2-methyl-5-oxo-1-phenyl-2,5-dihydro-1H-pyrazol-3-ylmethyl)-amino]-benzoic acid;
2-[(4-Bromo-2-methyl-5-oxo-1-phenyl-2,5-dihydro-1H-pyrazol-3-ylmethyl)-amino]-benzoic acid methyl ester;
4-Bromo-1-methyl-2-phenyl-5-phenylaminomethyl-1,2-dihydro-pyrazol-3-one;
4-Bromo-1-methyl-2-phenyl-5-(p-tolylamino-methyl)-1,2-dihydro-pyrazol-3-one
4-Bromo-1-methyl-5-[(4-nitro-phenylamino)-methyl]-2-phenyl-1,2-dihydro-pyrazol-3-one;
2-[(4-Bromo-2-methyl-5-oxo-1-phenyl-2,5-dihydro-1H-pyrazol-3-ylmethyl)-ethyl-amino]-benzoic acid methyl ester;
4-Bromo-5-[(ethyl-p-tolyl-amino)-methyl]-1-methyl-2-phenyl-1,2-dihydro-pyrazol-3-one:
2-(4-Bromo-2-methyl-5-oxo-1-phenyl-2,5-dihydro-1H-pyrazol-3-ylmethyl)-2-formylamino-succinic acid diethyl ester;
4-Bromo-1-methyl-5-{[(2-methylamino-ethyl)-pyridin-2-yl-amino]-methyl}-2-phenyl-1,2-dihydro-pyrazol-3-one:
4-Bromo-5-{[(4-ethoxy-phenyl)-(2-methylamino-ethyl)-amino]-methyl}-1-methyl-2-phenyl-1,2-dihydro-pyrazol-3-one:
2-(4-Bromo-2-methyl-5-oxo-1-phenyl-2,5-dihydro-1H-pyrazol-3-ylmethyl)-2-carboxyamino-malonic acid diethyl ester:
[2-(4-Bromo-2-methyl-5-oxo-1-phenyl-2,5-dihydro-1H-pyrazol-3-yl)-ethyl]-carbamic acid benzyl ester:
4-Bromo-1-methyl-5-methylaminomethyl-2-phenyl-1,2-dihydro-pyrazol-3-one, or
4-Bromo-5-dimethylaminomethyl-1-methyl-2-phenyl-1,2-dihydro-pyrazol-3-one.

29. A compound selected from those shown in the following table:

| Example No. | Structure |
|---|---|
| 373 | 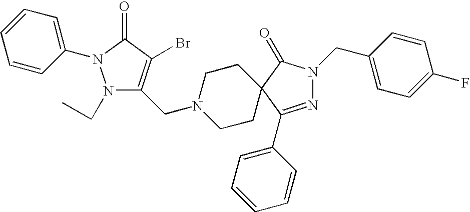 |
| 376 | 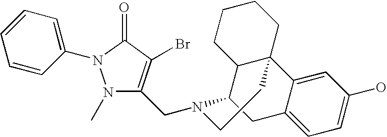 |
| 384 | 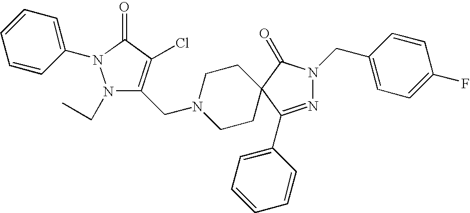 |
| 389 | 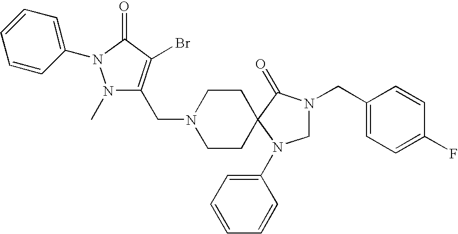 |
| 390 | 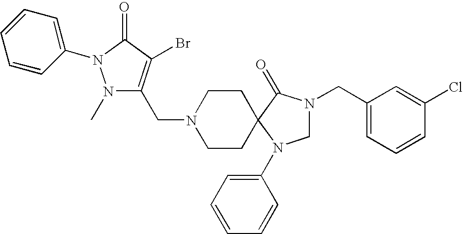 |
| 391 | 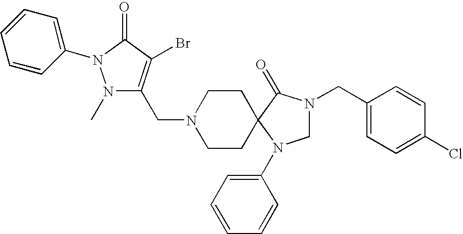 |
| 392 | 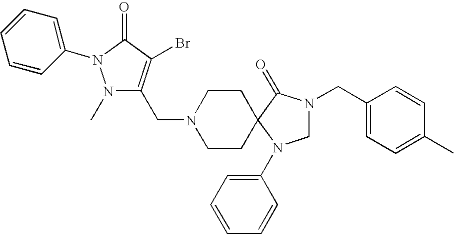 |
| 397 | 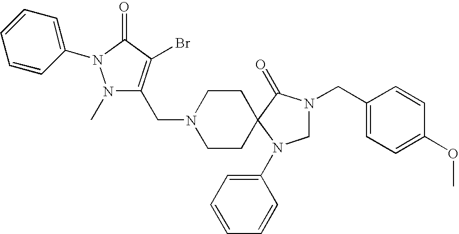 |
| 399 | 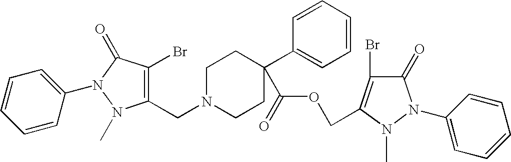 |
| 408 | 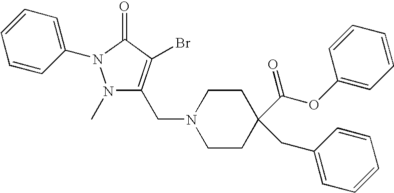 |
| 416a | 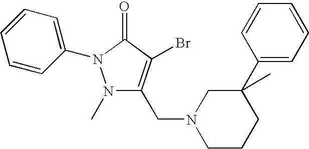 |
| 418 | 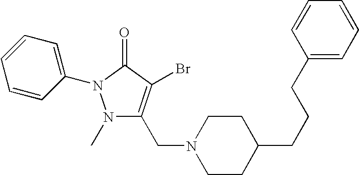 |
| 419 | 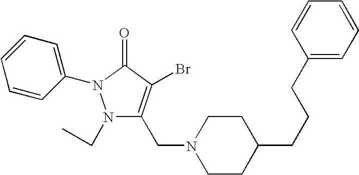 |
| 434 | 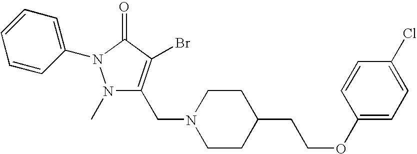 |
| 437 | 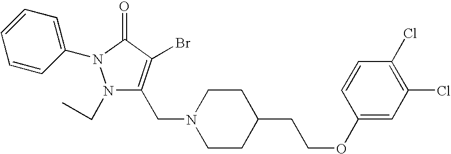 |
| 440 | 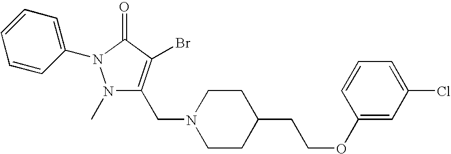 |
| 441 | 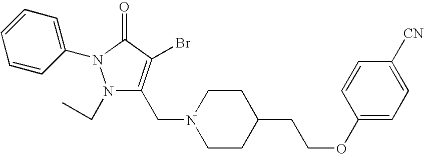 |
| 444 | 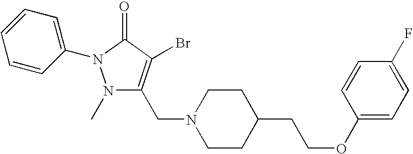 |
| 454 | 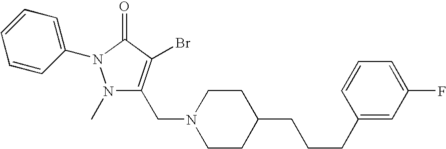 |
| 457 | 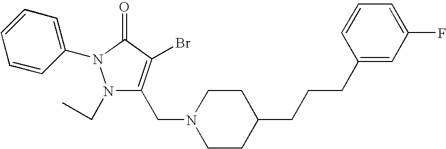 |
| 458 | 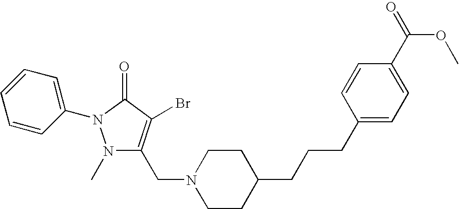 |
| 459 | 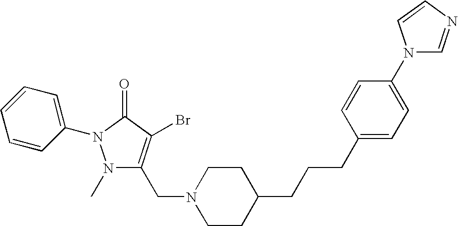 |
| 463 | 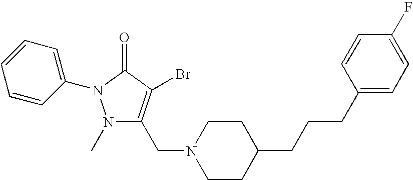 |
| 464 | 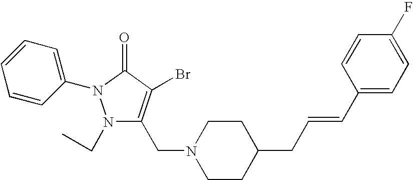 |
| 465 | 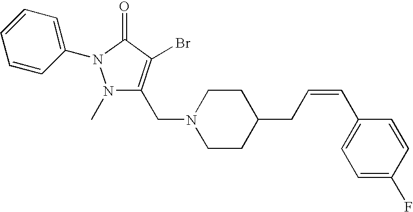 |
| 526 | 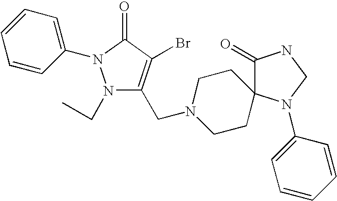 |
| 528 | 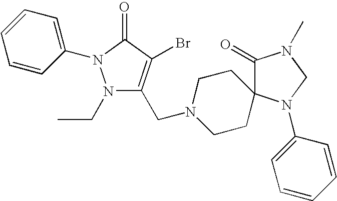 |
| 559 | 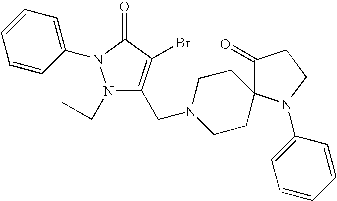 |
| 561 | 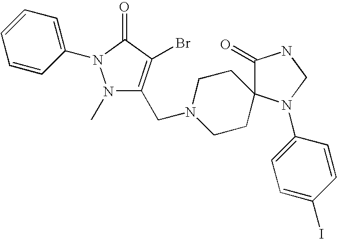 |
| 459 | 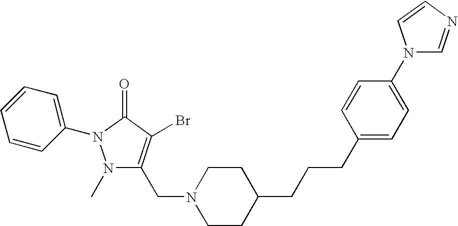 |
| 459a | 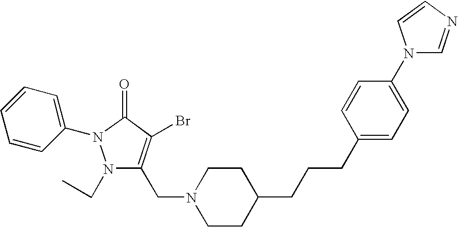 |
| 134 | 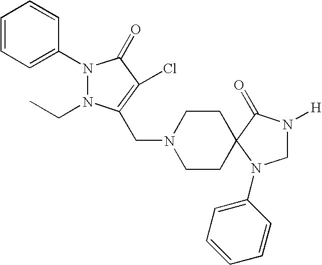 |
| 147 | 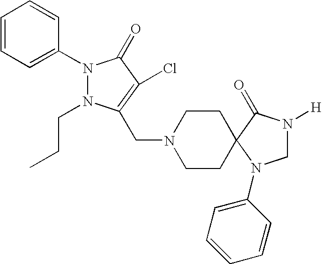 |
| 182 | 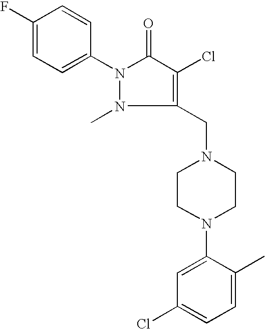 |
| 221 | 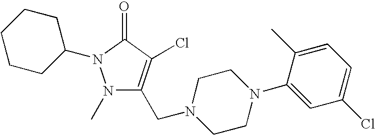 |
| 196 | 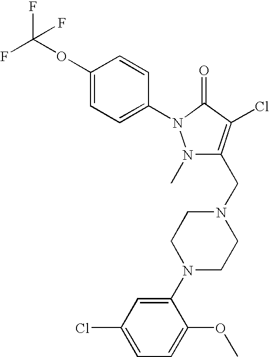 |
| 198 | 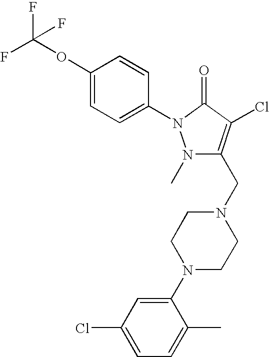 |
| 200 | 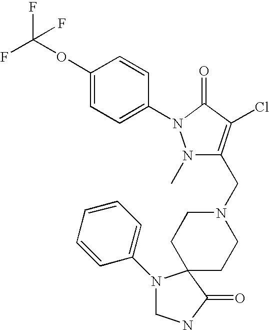 |
| 210 | 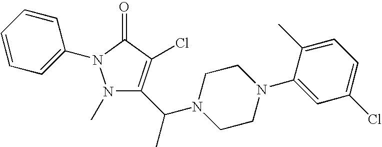 |
| 268 | 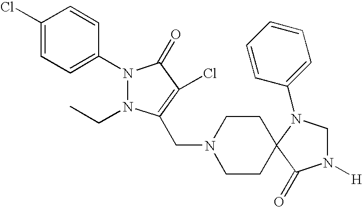 |
| 149 | 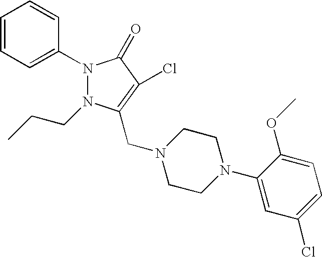 |
| 348 | 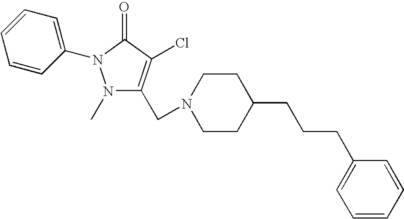 |
| 349 | 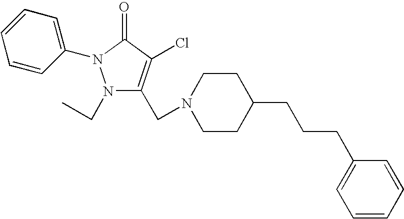 |
| 232 | 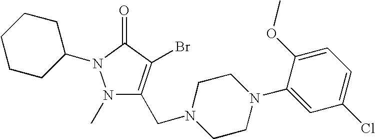 |
| 235 | 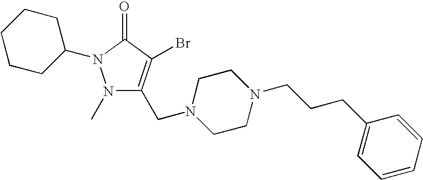 |
| 169 | 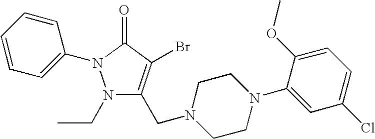 |
| 242 | 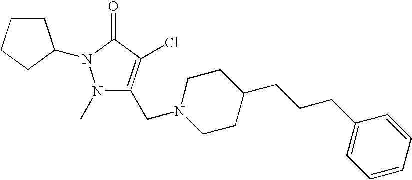 |
| 284 | 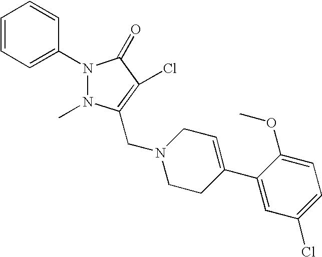 |
| 189 | 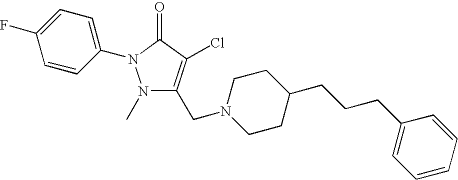 |
| 201 | 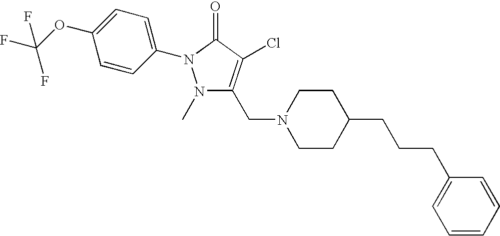 |
| 287 | 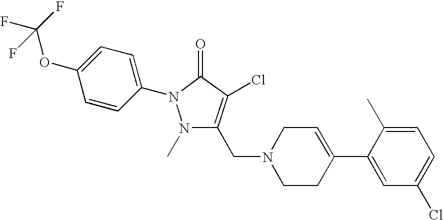 |
| 314 | 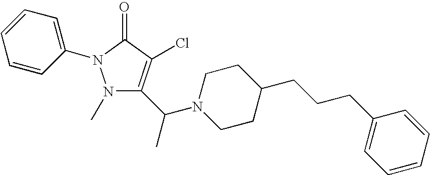 |
| 216 | 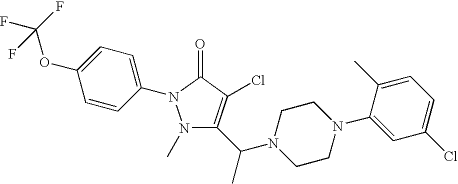 |
| 217 | 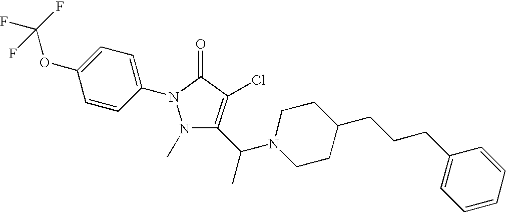 |
| 316 | 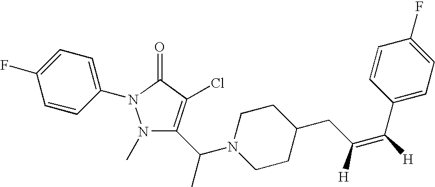 |
| 317 | 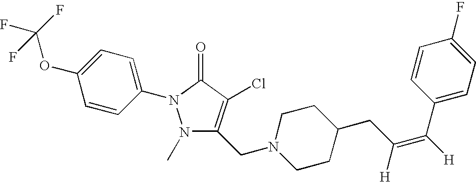 |
| 318 | 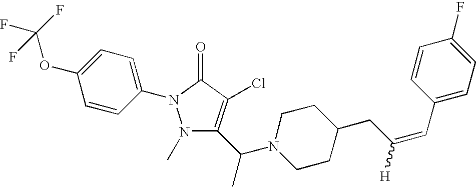 |
| 320 | 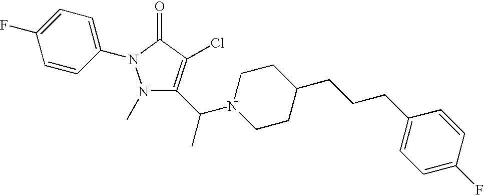 |
| 321 | 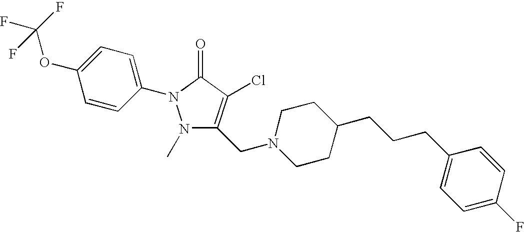 |

30. A compound according to claim 29, wherein the compound is selected from the group consisting of:

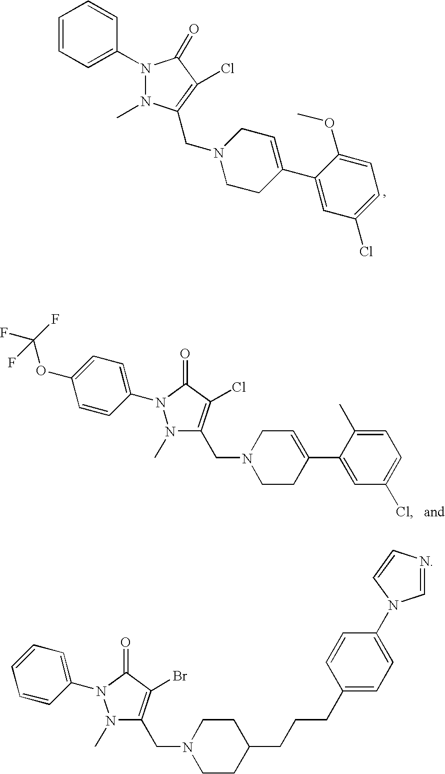

31. A pharmaceutical composition comprising a compound according to claim 1 or 28 and a pharmaceutically acceptable carrier or excipient.

32. A method for the treatment or prevention of neurological and psychiatric disorders associated with glutamate dysfunction in an animal in need of such treatment, comprising the step of administering to said animal a therapeutically effective amount of a compound according to claim 1 or 28.

33. A method for the treatment or prevention of neurological and psychiatric disorders associated with glutamate dysfunction in an animal in need of such treatment, comprising the step of administering to said animal a therapeutically effective amount of a pharmaceutical composition according to claim 31.

34. The method according to claim 32 wherein the neurological and psychiatric disorders are selected from the group consisting of cerebral deficit subsequent to cardiac bypass surgery and grafting, stroke, cerebral ischemia, spinal cord trauma, head trauma, perinatal hypoxia, cardiac arrest, hypoglycemic neuronal damage, dementia, AIDS-induced dementia, Alzheimer's disease, Huntington's Chorea, amyotrophic lateral sclerosis, ocular damage, retinopathy, cognitive disorders, idiopathic and drug-induced Parkinson's disease, muscular spasms and disorders associated with muscular spasticity including tremors, epilepsy, convulsions, migraine, urinary incontinence, substance tolerance, substance withdrawal, psychosis, schizophrenia, anxiety, mood disorders, circadian rhythm disorders, trigeminal neuralgia, hearing loss, tinnitus, macular degeneration of the eye, emesis, brain edema, pain, tardive dyskinesia, sleep disorders, attention deficit/hyperactivity disorder, and conduct disorder.

35. The use of a compound according to Formula I or Formula II, or a pharmaceutically acceptable salt or solvate thereof, for the manufacture of a medicament for the treatment of cerebral deficit subsequent to cardiac bypass surgery and grafting, stroke, cerebral ischemia, spinal cord trauma, head trauma, perinatal hypoxia, cardiac arrest, hypoglycemic neuronal damage, dementia, AIDS-induced dementia, Alzheimer's disease, Huntington's Chorea, amyotrophic lateral sclerosis, ocular damage, retinopathy, cognitive disorders, idiopathic and drug-induced Parkinson's disease, muscular spasms and disorders associated with muscular spasticity including tremors, epilepsy, convulsions, migraine, urinary incontinence, substance tolerance, substance withdrawal, psychosis, schizophrenia, anxiety, mood disorders, circadian rhythm disorders, trigeminal neuralgia, hearing loss, tinnitus, macular degeneration of the eye, emesis, brain edema, pain, tardive dyskinesia, sleep disorders, attention deficit/hyperactivity disorder, and conduct disorder.

36. A compound of Formula I or Formula II, or a pharmaceutically acceptable salt or solvate thereof, for use in therapy of cerebral deficit subsequent to cardiac bypass surgery and grafting, stroke, cerebral ischemia, spinal cord trauma, head trauma, perinatal hypoxia, cardiac arrest, hypoglycemic neuronal damage, dementia, AIDS-induced dementia, Alzheimer's disease, Huntington's Chorea, amyotrophic lateral sclerosis, ocular damage, retinopathy, cognitive disorders, idiopathic and drug-induced Parkinson's disease, muscular spasms and disorders associated with muscular spasticity including tremors, epilepsy, convulsions, migraine, urinary incontinence, substance tolerance, substance withdrawal, psychosis, schizophrenia, anxiety, mood disorders, circadian rhythm disorders, trigeminal neuralgia, hearing loss, tinnitus, macular degeneration of the eye, emesis, brain edema, pain, tardive dyskinesia, sleep disorders, attention deficit/hyperactivity disorder, and conduct disorder.

37. The method according to claim 33 wherein the neurological and psychiatric disorders are selected from the group consisting of cerebral deficit subsequent to cardiac bypass surgery and grafting, stroke, cerebral ischemia, spinal cord trauma, head trauma, perinatal hypoxia, cardiac arrest, hypoglycemia neuronal damage, dementia, AIDS-induced dementia, Alzheimer's disease, Huntington's chorea, amyotrophic lateral sclerosis, ocular damage, retinopathy, cognitive disorders, idiopathic and drug-induced Parkinson's disease, muscular spasms and disorders associated with muscular spasticity including tremors, epilepsy, convulsions, migraine, urinary incontinence, substance tolerance, substance withdrawal, psychosis, schizophrenia, anxiety, mood disorders, circadian rhythm disorders, trigeminal neuralgia, hearing loss, tinnitus, macular degeneration of the eye, emesis, brain edema, pain, tardive dyskinesia, sleep disorders, attention deficit/hyperactivity disorder, and conduct disorder.
